(19) United States
(12) Patent Application Publication
(10) Pub. No.: US 2025/0295798 A1
(43) Pub. Date: Sep. 25, 2025

(54) LIGAND-DRUG CONJUGATE OF EXATECAN ANALOGUE, AND MEDICAL USE THEREOF

(71) Applicant: BeiGene Switzerland GmbH, Basel (CH)

(72) Inventors: Charng-Sheng TSAI, Beijing (CN); Mei-Hsuan TSAI, Beijing (CN); Bing LI, Beijing (CN); Liu XUE, Beijing (CN); Maomao HE, Beijing (CN); Zewei WANG, Beijing (CN); Wei LUO, Beijing (CN); Yi QU, Beijing (CN); Xiaokun YANG, Beijing (CN); Ce WANG, Beijing (CN)

(73) Assignee: Beigene Switzerland GMBH, Basel (CH)

(21) Appl. No.: 19/089,217

(22) Filed: Mar. 25, 2025

Related U.S. Application Data
(63) Continuation of application No. PCT/CN2023/122616, filed on Sep. 28, 2023.

(30) Foreign Application Priority Data
Sep. 30, 2022 (WO) PCT/CN2022/123665

(51) Int. Cl. A61K 47/68; C07D 519/00; C07K 16/32

(52) U.S. Cl. CPC A61K 47/68037; A61K 47/6849; C07D 519/00; C07K 16/32

(57) ABSTRACT

Provided herein are compounds having formulas (I) or (VII):

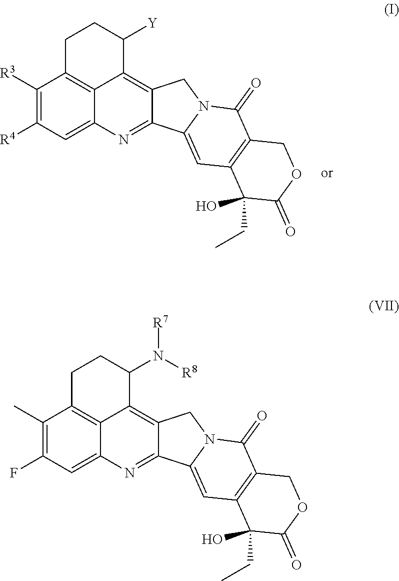

or pharmaceutically acceptable salts, tautomers, isotopologues, stereoisomers, or prodrugs thereof, wherein the substituents are as described herein;
ligand-drug conjugates and pharmaceutically acceptable salts or solvates thereof, wherein the ligand-drug conjugate comprises a residue of the compound provided here; and
methods of treating cancer thereof.

### 1. CROSS-REFERENCE TO RELATED APPLICATIONS

This application is a continuation of International Application No. PCT/CN2023/122616, filed on Sep. 28, 2023, which claims priority to International Application No. PCT/CN2022/123665, filed on Sep. 30, 2022, the disclosures of which are hereby incorporated by reference in their entireties.

### 2. FIELD OF THE INVENTION

The present disclosure relates to ligand-drug conjugates of exatecan analogues with novel structures. Specifically, the present disclosure relates to exatecan analogues, ligand-drug conjugates of exatecan analogues, methods for preparing the same, and medical uses of the conjugates.

### 3. SEQUENCE LISTING

This application contains a Sequence Listing, which has been submitted electronically in XML format. The XML file is entitled “01368-0068-00PCT.xml,” was created on Mar. 10, 2025, and is 9,118 bytes in size. The Sequence Listing is incorporated herein by reference in its entirety.

### 4. BACKGROUND OF THE INVENTION

Chemotherapy remains one of the most important anti-cancer therapies along with surgery, radiotherapy and targeted therapy. Although there are many types of highly efficient cytotoxins, the difference between tumor cells and normal cells is very small, which limits the broad clinical application of these anti-cancer compounds due to the toxic side effect. Antibody drugs have become the frontline drugs for anti-tumor therapy because of the specificity of anti-tumor monoclonal antibody for tumor cell surface antigen. However, when the antibody is used alone as the anti-tumor drug, the efficacy is often unsatisfactory.

Antibody drug conjugates (ADCs) enable the combination a monoclonal antibody or an antibody fragment with a biologically active cytotoxin through a chemically stable linker, taking full advantage of the specificity of antibody binding to the surface antigens of normal cells or tumor cells and the high efficiency of the cytotoxin, while avoiding low efficacy of the antibody and the toxic side effect of the cytotoxin. That means, comparing with conventional chemotherapy drugs, antibody drug conjugates can accurately bind to tumor cells and reduce the affect to normal cells (Mullard A, (2013) Nature Reviews Drug Discovery, 12:329-332; DiJoseph J F, Armellino D C, (2004) Blood, 103:1807-1814).

In 2000, the first antibody drug conjugate Mylotarg (gemtuzumab ozogamicin, Wyeth Pharmaceuticals) was approved by the US Food and Drug Administration (FDA) for the treatment of acute myeloid leukemia (Drugs of the Future (2000) 25(7):686; U.S. Pat. Nos. 4,970,198; 5,079,233; 5,585,089; 5,606,040; 5,693,762; 5,739,116; 5,767,285; 5,773,001).

In August 2011, Adcetris (brentuximab vedotin, Seattle Genetics Inc.) was approved through the US FDA Fast Track for the treatment of Hodgkin lymphoma and relapsed anaplastic large cell lymphoma (Nat. Biotechnol (2003) 21(7):778-784; WO2004010957; WO2005001038; U.S. Pat. Nos. 7,090,843A; 7,659,241; WO2008025020). Adcetris® is a novel target ADC drug, which enables the drug to act directly on the target CD30 of lymphoma cell, trigger endocytosis and consequently induce tumor cell apoptosis.

Both Mylotarg and Adcetris are target therapies for hematologic tumors, the organizational structure of which is relatively simple compared with that of solid tumors. In February 2013, Kadcyla (ado-trastuzumab emtansine, T-DM1) was approved by FDA for the treatment of advanced or metastatic breast cancer patients who are HER2-positive with Trastuzumab (trade name: Herceptin)-resistant and paclitaxel-resistant (WO2005037992; U.S. Pat. No. 8,088,387). Kadcyla is the first ADC drug approved by FDA for the treatment of solid tumors.

There are several types of cytotoxic small molecules used in antibody drug conjugate, one of which is camptothecin derivatives, which show anti-tumor effect by inhibiting topoisomerase I. Documents reporting the use of the camptothecin derivative, exatecan (chemical name: (1S,9S)-1-amino-9-ethyl-5-fluoro-2,3-dihydro-9-hydroxy-4-methyl-1H,12H-benzo[de]pyrano[3′,4′:6,7]imidazo[1,2-b]quinoline-10,13 (9H,15H)-dione) in antibody drug conjugate (ADC) comprise WO2014057687, Clinical Cancer Research (2016) 22 (20): 5097-5108, and Cancer Sci (2016) 107: 1039-1046. However, further development of ADC drugs with better efficacy is still needed.

### 5. SUMMARY

Provided herein is a compound having formula (I):

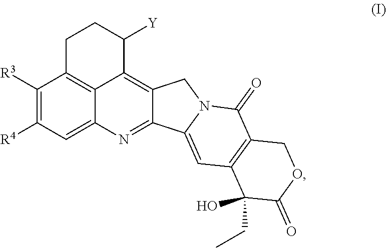

or a pharmaceutically acceptable salt, tautomer, isotopologue, stereoisomer, or prodrug thereof, wherein
Y is -A-B-C-D-H;
A is a bond, CR¹R², or N—R¹;
B is a bond, —C(═O)—; —C(═O)O— or —OC(═O)—;
C is a bond, or a divalent group, wherein the divalent group is unsubstituted or substituted C₁₋₈ alkyl, unsubstituted or substituted cycloalkyl, unsubstituted or substituted heterocyclyl, unsubstituted or substituted aryl, or unsubstituted or substituted heteroaryl;
D is a bond, NH, or O;
each of R¹, and R² is, independently, hydrogen, halogen, substituted or unsubstituted alkyl, or substituted or unsubstituted alkoxyl; or R¹, and R² together with the atom to which they are attached to, form unsubstituted or substituted cycloalkyl, unsubstituted or substituted heterocyclyl, unsubstituted or substituted aryl, or unsubstituted or substituted heteroaryl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl; and
when R³ is methyl, and R⁴ is F, Y is not —NH—C(═O)—C—D—H.

In one embodiment, the compound is a compound having formula (II):

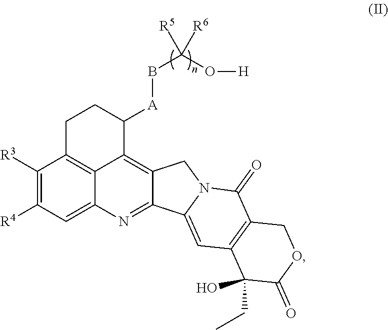

or a pharmaceutically acceptable salt, tautomer, isotopologue, stereoisomer, or prodrug thereof, wherein
A is CR¹R², NH, or N—R¹;
each of R¹, and R² is, independently, H, or C₁₋₄alkyl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl;
each of R⁵, and R⁶ is, independently, hydrogen, halogen, substituted or unsubstituted alkyl, or substituted or unsubstituted alkoxyl; and
n is 1, 2, 3, 4, or 5.

In one embodiment, the ligand-drug conjugate comprises a structure of formula (V):

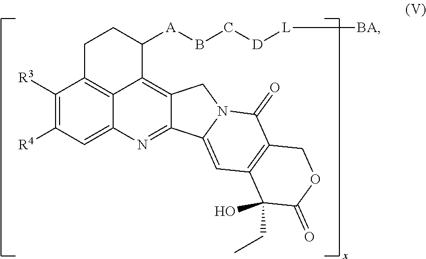

wherein
BA is a binding agent selected from a humanized, chimeric, or human antibody or an antigen binding antibody fragment of an antibody;
L is a covalent linker as described here; and
x is 1 to 10, which can be an integer or a decimal.

In one embodiment, the antibody is patritumab, cofetuzumab, trastuzumab, or mAb10.

Provided herein is a compound having formula (VII):

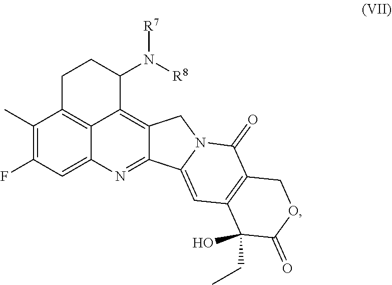

or a pharmaceutically acceptable salt, tautomer, isotopologue, stereoisomer, or prodrug thereof, wherein
each of R⁷, and R⁸ is, independently, hydrogen, or substituted or unsubstituted alkyl; or R, and R⁸ together with the nitrogen atom to which they are attached to, form unsubstituted or substituted heterocyclyl, or unsubstituted or substituted heteroaryl.

In another aspect, provided herein is a ligand-drug conjugate or a pharmaceutically acceptable salt or solvate thereof, wherein the ligand-drug conjugate comprises a residue of the compound provided here.

In another aspect, provided herein are methods of treating cancer comprising administering to a subject in need thereof a ligand-drug conjugate comprising a residue of the compound provided here.

In another aspect, provided herein are kits comprising a ligand-drug conjugate comprising a residue of the compound provided here.

### 6. BRIEF DESCRIPTION OF FIGURES

FIG. 1 illustrates the payload cellular killing potency on MDA-MB-453 cell line.

FIG. 2 illustrates the payload cellular killing potency on A375 cell lines.

FIG. 3 illustrates the payload cellular killing potency on Clau6 cell lines.

FIG. 4 illustrates the payload cellular killing potency on A375 cell lines.

FIG. 5 illustrates the payload cellular killing potency on Clau6 cell lines.

FIG. 6 illustrates the payload cellular killing potency on A375 cell lines.

FIG. 7 illustrates the payload cellular killing potency on Clau6 cell lines.

FIG. 8 illustrates the payload cellular killing potency on A375 cell lines.

FIG. 9 illustrates the payload cellular killing potency on Clau6 cell lines.

FIG. 10 illustrates the payload cellular killing potency on A375 cell lines.

FIG. 11 illustrates the payload cellular killing potency on Clau6 cell lines.

FIG. 12 illustrates the payload cellular killing potency on A375 cell lines.

FIG. 13 illustrates the payload cellular killing potency on Clau6 cell lines.

FIG. 14 illustrates the payload cellular killing potency on A375 cell lines.

FIG. 15 illustrates the payload cellular killing potency on Clau6 cell lines.

FIG. 16 illustrates the payload cellular killing potency on A375 cell lines.

FIG. 17 illustrates the payload cellular killing potency on Clau6 cell lines.

FIG. 18 illustrates the payload cellular killing potency on A375 cell lines.

FIG. 19 illustrates the payload cellular killing potency on Clau6 cell lines.

FIG. 20 illustrates the payload cellular killing potency on A375 cell lines.

FIG. 21 illustrates the payload cellular killing potency on Clau6 cell lines.

FIG. 22 illustrates the payload cellular killing potency on A375 cell lines.

FIG. 23 illustrates the payload cellular killing potency on Clau6 cell lines.

FIG. 24 illustrates the ADC direct cellular killing potency on A375 cell lines.

FIG. 25 illustrates the ADC direct cellular killing potency on Clau6 cell lines.

FIG. 26 illustrates the ADC direct cellular killing potency on A375 cell lines.

FIG. 27 illustrates the ADC direct cellular killing potency on Clau6 cell lines.

FIG. 28 illustrates the ADC direct cellular killing potency on A375 cell lines.

FIG. 29 illustrates the ADC direct cellular killing potency on Clau6 cell lines.

FIG. 30 illustrates the ADC direct cellular killing potency on A375 cell lines.

FIG. 31 illustrates the ADC direct cellular killing potency on Clau6 cell lines.

FIG. 32 illustrates the ADC direct cellular killing potency on A375 cell lines.

FIG. 33 illustrates the ADC direct cellular killing potency on Clau6 cell lines.

FIG. 34 illustrates the ADC direct cellular killing potency on A375 cell lines.

FIG. 35 illustrates the ADC direct cellular killing potency on Clau6 cell lines.

FIG. 36 illustrates the ADC bystander cellular killing activity in the MDA-MB-453 & Calu-6-nanoLuc system.

FIG. 37 illustrates the ADC bystander cellular killing potency in the A375 & Calu-6-nanoLuc system.

FIG. 38 illustrates the ADC bystander cellular killing activity in the Calu-6-nanoLuc only system.

FIG. 39 illustrates the ADC bystander cellular killing potency in the MDA-MB-453 & Calu-6-nanoLuc system.

FIG. 40 illustrates the ADC bystander cellular killing activity in the A375 & Calu-6-nanoLuc system.

FIG. 41 illustrates the ADC bystander cellular killing potency in the Calu-6-nanoLuc only system.

FIG. 42 illustrates the ADC bystander cellular killing activity in the MDA-MB-453 & Calu-6-nanoLuc system.

FIG. 43 illustrates the ADC bystander cellular killing potency in the A375 & Calu-6-nanoLuc system.

FIG. 44 illustrates the ADC bystander cellular killing activity in the Calu-6-nanoLuc only system.

FIG. 45 illustrates the ADC bystander cellular killing potency in the MDA-MB-453 & Calu-6-nanoLuc system.

FIG. 46 illustrates the ADC bystander cellular killing potency in the A375 & Calu-6-nanoLuc system.

FIG. 47 illustrates the ADC bystander cellular killing activity in the Calu-6-nanoLuc only system.

FIG. 48 illustrates the ADC bystander cellular killing potency in the MDA-MB-453 & Calu-6-nanoLuc system.

FIG. 49 illustrates the ADC bystander cellular killing potency in the A375 & Calu-6-nanoLuc system.

FIG. 50 illustrates the ADC bystander cellular killing activity in the Calu-6-nanoLuc only system.

FIG. 51 illustrates the ADC bystander cellular killing potency in the MDA-MB-453 & Calu-6-nanoLuc system.

FIG. 52 illustrates the ADC bystander cellular killing potency in the A375 & Calu-6-nanoLuc system.

FIG. 53 illustrates the ADC bystander cellular killing activity in the Calu-6-nanoLuc only system.

FIGS. 54A-54F depict the in vivo efficacy of different ADCs compared in A-375 melanoma xenografts grown subcutaneously in BALB/c nude mice.

FIGS. 55A-55F depict the in vivo efficacy of different ADCs compared in A-375 melanoma xenografts grown subcutaneously in BALB/c nude mice.

FIGS. 56A-56I depict the in vivo efficacy of different ADCs were compared in A-375 melanoma xenografts grown subcutaneously in BALB/c nude mice.

FIGS. 57A-57D depict the in vivo efficacy of different ADCs were compared in A-375 melanoma xenografts grown subcutaneously in BALB/c nude mice.

FIGS. 58A-58J illustrate that ADC-3, ADC-6, ADC-7, ADC-9, ADC-10, ADC-11, ADC-21, ADC-P1, ADC-P2 or ADC-P3 exhibited comparable ADC or total Ab exposure and clearance with ADC-1.

FIGS. 59A and 59B illustrate the sequences related to mAb10.

### 7. DETAILED DESCRIPTION

Within the present disclosure, it is understood that the disclosure does not limit to particular methods and/or experimental conditions described, as such methods and conditions may vary. It is also to be understood that the terminology used herein is for the purpose of describing particular embodiments only, and is not intended to be limiting,

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 7.1. Definitions

When referring to the compounds provided herein, the following terms have the following meanings unless indicated otherwise. Unless defined otherwise, all technical and scientific terms used herein have the same meaning as is commonly understood by one of ordinary skill in the art to which this invention belongs. When trade names are used herein, the trade name includes the product formulation, the generic drug, and the active pharmaceutical ingredient(s) of the trade name product, unless otherwise indicated by context. In the event that there is a plurality of definitions for a term provided herein, these Definitions prevail unless stated otherwise.

The term “antibody” as used herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. The native form of an antibody is a tetramer and consists of two identical pairs of immunoglobulin chains, each pair having one light chain and one heavy chain. In each pair, the light and heavy chain variable regions (VL and VH) are together primarily responsible for binding to an antigen. The light chain and heavy chain variable domains consist of a framework region interrupted by three hypervariable regions, also called“complementarity determining regions” or “CDRs.” The constant regions may be recognized by and interact with the immune system (see, e.g., Janeway et al, 2001, Immunol. Biology, 5th Ed., Garland Publishing, New York). An antibody can be of any type (e.g., IgG, IgE, IgM, IgD, and IgA), class (e.g., IgG₁, IgG₂, IgG₃, IgG₄, IgA₁ and IgA₂) or subclass thereof. The antibody can be derived from any suitable species. In some embodiments, the antibody is of human or murine origin. An antibody can be, for example, human, humanized or chimeric.

The term “monoclonal antibody” as used herein refers to an antibody obtained from a population of substantially homogeneous antibodies, i.e., the individual antibodies comprising the population are identical except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. Monoclonal antibodies are highly specific, being directed against a single antigenic site. The modifier“monoclonal” indicates the character of the antibody as being obtained from a substantially homogeneous population of antibodies and is not to be construed as requiring production of the antibody by any particular method.

An “intact antibody” is one which comprises an antigen-binding variable region as well as a light chain constant domain (CL) and heavy chain constant domains, OHI, CH2, CH3 and CH4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)2, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is an entity to which an antibody specifically binds.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

The term “inhibits” or “inhibition of” means to reduce by a measurable amount, or to prevent entirely.

The term “therapeutically effective amount” refers to an amount of a conjugate effective to treat a disease or disorder in a mammal. In the case of cancer, the therapeutically effective amount of the conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or“substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The term “cytotoxic activity” refers to a cell-killing effect of a drug or Exatecan derivative Conjugate or an intracellular metabolite of an Exatecan derivative Conjugate. Cytotoxic activity may be expressed as the IC₅₀ value, which is the concentration (molar or mass) per unit volume at which half the cells survive.

The term “cytostatic activity” refers to an anti-proliferative effect of a drug or Exatecan derivative Conjugate or an intracellular metabolite of an Exatecan derivative Conjugate.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant or animal origin, including synthetic analogs and derivatives thereof.

The term “cytostatic agent” as used herein refers to a substance that inhibits a function of cells, including cell growth or multiplication. Cytostatic agents include inhibitors such as protein inhibitors, e.g., enzyme inhibitors. Cytostatic agents have cytostatic activity.

The terms “cancer” and“cancerous” refer to or describe the physiological condition or disorder in mammals that is typically characterized by unregulated cell growth. A “tumor” comprises one or more cancerous cells.

An “autoimmune disease” as used herein refers to a disease or disorder arising from and directed against an individual's own tissues or proteins.

“Patient” as used herein refers to a subject to whom is administered an Exatecan derivative Conjugate of the present invention. Patient includes, but are not limited to, a human, rat, mouse, guinea pig, non-human primate, pig, goat, cow, horse, dog, cat, bird and fowl. Typically, the patient is a rat, mouse, dog, human or non-human primate, more typically a human.

The terms “treat” or “treatment,” unless otherwise indicated by context, refer to therapeutic treatment and prophylactic wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (/._{<?}., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of: killing tumor cells; inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

“Compound” as the term is used herein, refers to and encompasses the chemical compound itself, either named or represented by structure, and salt form(s) thereof, whether explicitly stated or not, unless context makes clear that such salt forms are to be excluded. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; and when a compound can be associated with two or more solvent molecules, the two or more solvent molecules may be the same or different.

In some instances, a compound of the invention will include an explicit reference to one or more of the above forms, e.g., salts and solvates, which does not imply any solid state form of the compound; however, this reference is for emphasis only, and is not to be construed as excluding any other of the forms as identified above. Furthermore, when explicit reference to a salt and/or solvate form of a compound or a Ligand Drug Conjugate composition is not made, that omission is not to be construed as excluding the salt and/or solvate form(s) of the compound or Conjugate unless context make clear that such salt and/or solvate forms are to be excluded.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with amounts, or weight percentage of ingredients of a composition, mean an amount, or weight percent that is recognized by one of ordinary skill in the art to provide a pharmacological effect equivalent to that obtained from the specified amount, or weight percent. In certain embodiments, the terms “about” and “approximately,” when used in this context, contemplate an amount, or weight percent within 30%, within 20%, within 15%, within 10%, or within 5%, of the specified amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degree 2θ) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C═CH, —C═C(CH₃), —C═C(CH₂CH₃), —CH₂C═CH, —CH₂C═C(CH₃) and CH₂C═C(CH₂CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

A “cycloalkyl” group is a saturated, or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3Himidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2 (3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

An “amine” group is a radical of the formula: NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R^{#} is as defined above.

An “alkylamine” group is a radical of the formula: NHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(=O)(R^{#}) or N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R#)C(═O)(R^{#}) or N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})=NH, or —NHC(R^{#})=NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R), wherein each R^{#} is independently as defined above.

A “phosphine” group is a radical of the formula: —P(R^{#})₂, wherein each R^{#} is independently as defined above.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound, particularly a compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

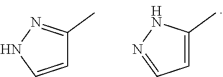

As readily understood by one skilled in the art, a wide variety of functional groups and other structures may exhibit tautomerism and all tautomers of the compounds are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵i), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

The term “effective amount” in connection with a compound means an amount capable of alleviating, in whole or in part, symptoms, or slowing or halting further progression or worsening of those symptoms. As will be apparent to those skilled in the art, it is to be expected that the effective amount of a compound disclosed herein may vary depending on the severity of the indication being treated.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amine (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including Glycine and both the D or L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular peptide may be obtained as an acidic or basic salt, or in neutral form. A peptide may be obtained directly from the source organism, or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab. In one embodiment, trastuzumab can be made and used as described in U.S. Pat. Nos. 6,407,213, and 5,821,337, the entire disclosure of which is incorporated herein by reference. In one embodiment, the sequence of the heavy chain and its matching light chain for trastuzumab and its preparation method are known in the art and can also be found in public databases, e.g., Inxight Drugs developed by The National Center for Advancing Translational Sciences (NCATS).

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference. In one embodiment, the sequence of the heavy chain and its matching light chain for patritumab and its preparation method are known in the art and can also be found in public databases, e.g., Inxight Drugs developed by The National Center for Advancing Translational Sciences (NCATS).

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference. In one embodiment, the sequence of the heavy chain and its matching light chain for cofetuzumab and its preparation method are known in the art and can also be found in public databases, e.g., Inxight Drugs developed by The National Center for Advancing Translational Sciences (NCATS).

As used herein, the term “Ifinatamab” refers to an antibody selectively binding to the B7H3 receptor. In one embodiment, Ifinatamab can be made and used as described in U.S. Ser. No. 10/117,952 or WO2022102695, the entire disclosure of which is incorporated herein by reference. In one embodiment, the sequence of the heavy chain and its matching light chain for Ifinatamab and its preparation method are known in the art and can be found in publica databases, e.g., SAbDab: The Structural Antibody Database—OPIG.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “cell-killing activity” refers to the activity that decreases or reduces the cell viability of the tested cell line.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

### 7.2. Conjugates

Described herein are compounds according to the following formula:

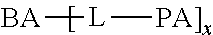

or a pharmaceutically acceptable salt tautomer, solvate, stereoisomer, enantiomer, isotopologue, or prodrug thereof,
wherein BA is a binding agent selected from a humanized, chimeric, or human antibody or an antigen binding antibody fragment of an antibody; L is a covalent linker; PA is a payload residue; and subscript x is from 1 to 30. In some instances, x is from 1 to 4. In some instances, x is about 1. In some instances, x is about 2. In some instances, x is about 3. In some instances, x is about 4.

In one example, BA is an antibody. In one example, the antibody is a humanized, chimeric, or human antibody or an antigen binding fragment of an antibody. In one example, the antibody is a humanized, chimeric, or human anti-HER2, anti-HER3, anti-PTK7, or anti-B7H3 antibody or an antigen binding fragment of an anti-HER2, anti-HER3, anti-PTK7, or anti-B7H3 antibody. In one example, the antibody is a monoclonal antibody.

In one example, BA is an antibody. In one example, the antibody is a humanized, chimeric, or human antibody or an antigen binding fragment of cofetuzumab, patritumab, ifinatamab, or trastuzumab.

In certain embodiments, the antibody as described herein binds to one or more of receptors selected from the group consisting of: CD7, CD19, CD22, CD27, CD30, CD33, CD37, CD70, CD74, CD79b, CD138, CD142, CA6, p-Cadherin, CEACAM5, C4.4a, DLL3, EGFR, EGFRVIII, ENPP3, EphA2, EphrinA, FLOR1, FGFR2, GCC, HER2, HER3, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, and Tn.

In certain embodiments, the antibody as described herein binds to one or more of receptors selected from the group consisting of: B7H3, MUC1, FGFR2b, CLL1, CCR7, GPC1, and GPC3.

In certain embodiments, the antibody as described herein binds to CEA receptors.

In certain embodiments, the antibody as described herein is a bispecific antibody.

In some embodiments, PA is a residue of the exatecan analogues provided herein.

### 7.3. Exatecan Analogue

Provided herein are exatecan analogues.

### Aspect 1

Provided herein is a compound having formula (I):

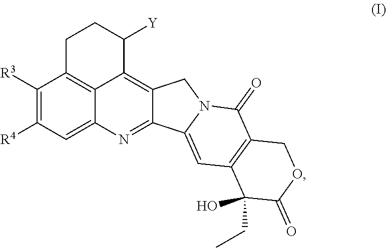

or a pharmaceutically acceptable salt, tautomer, isotopologue, stereoisomer, or prodrug thereof, wherein
Y is -A-B-C-D-H;
A is a bond, CR¹R², or N—R¹;
B is a bond, —C(═O)—; or —C(═O)O—;
C is a bond, or a divalent group, wherein the divalent group is unsubstituted or substituted C₁₋₈alkyl, unsubstituted or substituted cycloalkyl, unsubstituted or substituted heterocyclyl, unsubstituted or substituted aryl, or unsubstituted or substituted heteroaryl;
D is a bond, NH, or O;
each of R¹, and R² is, independently, hydrogen, halogen, substituted or unsubstituted alkyl, or substituted or unsubstituted alkoxyl; or R¹, and R² together with the atom to which they are attached to, form unsubstituted or substituted cycloalkyl, unsubstituted or substituted heterocyclyl, unsubstituted or substituted aryl, or unsubstituted or substituted heteroaryl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl; and
when R³ is methyl, and R⁴ is F, Y is not —NH—C(═O)—C—D-H.

### Aspect 2

In one embodiment, the compound is a compound having formula (II):

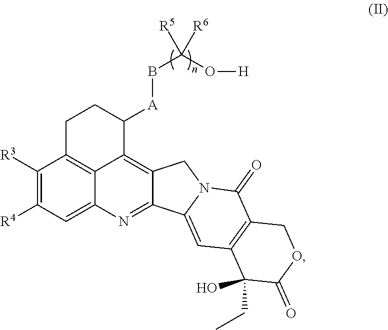

or a pharmaceutically acceptable salt, tautomer, isotopologue, stereoisomer, or prodrug thereof, wherein
A is CR¹R², NH, or N—R¹;
each of R¹, and R² is, independently, H, or C₁₋₄alkyl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl;
each of R⁵, and R⁶ is, independently, hydrogen, halogen, substituted or unsubstituted alkyl, or substituted or unsubstituted alkoxyl; and
n is 1, 2, 3, 4, or 5.

### Aspect 3

In one embodiment, A is —CH₂—, and B is a bond.

In one embodiment, R⁵ and R⁶ are hydrogen, and n is 1, 2, or 3.

In one embodiment, R³ is methyl, and R⁴ is F.

In one embodiment, the compound is

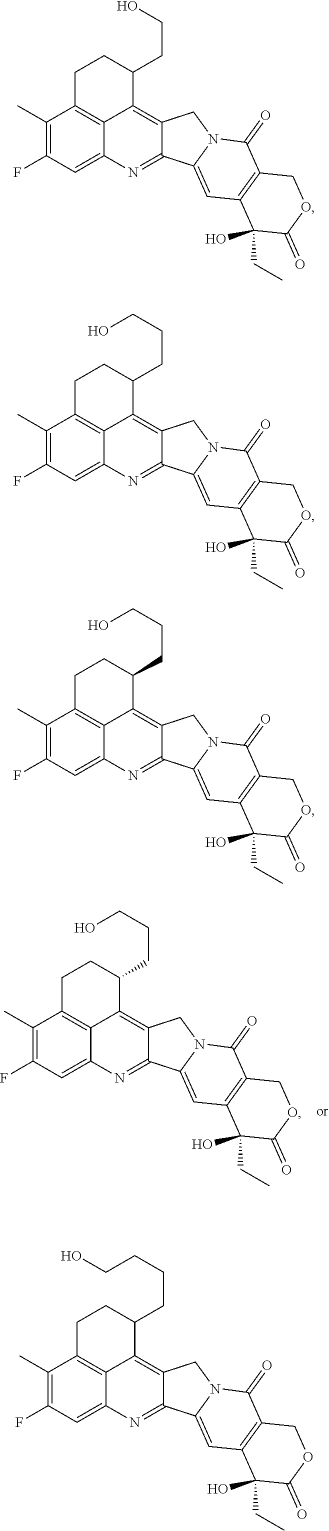 [SMILES: CC[C@@]1(O)C(=O)OCc2c1cc1n(c2=O)Cc2c-1nc1cc(F)c(C)c3c1c2C(CCO)CC3 ; CC[C@@]1(O)C(=O)OCc2c1cc1n(c2=O)Cc2c-1nc1cc(F)c(C)c3c1c2C(CCCO)CC3 ; CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3 ; "or" ; CC[C@@]1(O)C(=O)OCc2c1cc1n(c2=O)Cc2c-1nc1cc(F)c(C)c3c1c2C(CCCCO)CC3]

### Aspect 4

In one embodiment, A is —CH₂—, and B is a bond.

In one embodiment, R⁵ and R⁶ are hydrogen, and n is 1, 2, or 3.

In one embodiment, R³, and R⁴ together with the atoms to which they are attached to, form an unsubstituted or substituted dioxole ring.

In one embodiment, the compound is

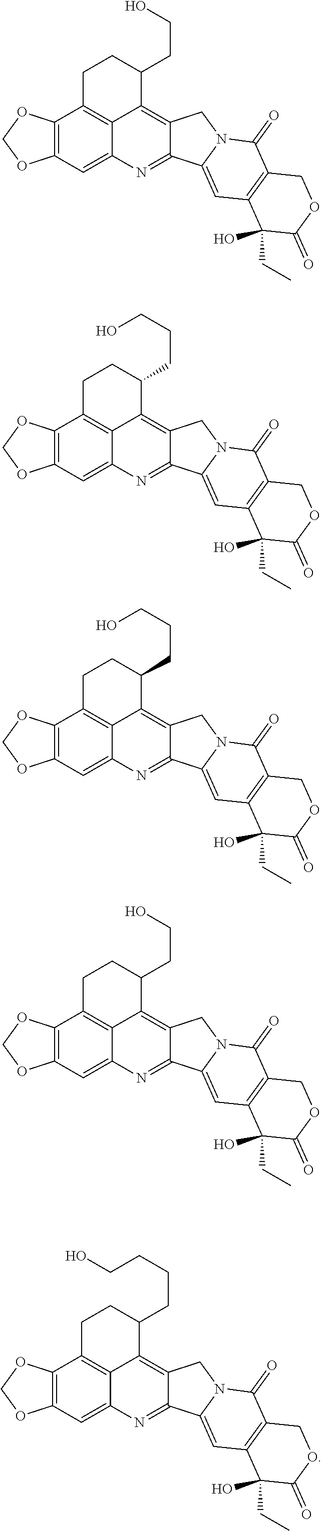 [SMILES: CC[C@@]1(O)C(=O)OCc2c1cc1n(c2=O)Cc2c-1nc1cc3c(c4c1c2C(CCO)CC4)OCO3 ; CC[C@@]1(O)C(=O)OCc2c1cc1n(c2=O)Cc2c-1nc1cc3c(c4c1c2[C@@H](CCCO)CC4)OCO3 ; CC[C@@]1(O)C(=O)OCc2c1cc1n(c2=O)Cc2c-1nc1cc3c(c4c1c2[C@H](CCCO)CC4)OCO3 ; CC[C@@]1(O)C(=O)OCc2c1cc1n(c2=O)Cc2c-1nc1cc3c(c4c1c2C(CCO)CC4)OCO3 ; CC[C@@]1(O)C(=O)OCc2c1cc1n(c2=O)Cc2c-1nc1cc3c(c4c1c2C(CCCCO)CC4)OCO3]

### Aspect 5

In one embodiment, R³ is methyl, and R⁴ is F.

In one embodiment, A is —N(CH₃)—, and B is a bond.

In one embodiment, R and R are hydrogen, and n is 2.

In one embodiment, the compound is

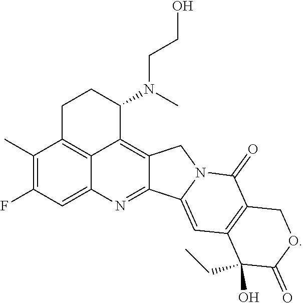 [SMILES: CC[C@@]1(O)C(=O)OCc2c1cc1n(c2=O)Cc2c-1nc1cc(F)c(C)c3c1c2[C@@H](N(C)CCO)CC3]

### Aspect 6

In one embodiment, R³ is methyl, and R⁴ is F.

In one embodiment, A is —NH—, and B is —C(═O)O—.

In one embodiment, R and R are hydrogen, and n is 2.

In one embodiment, the compound is

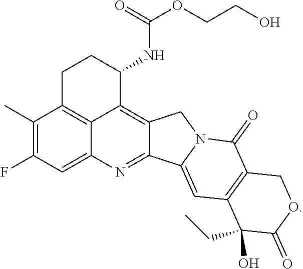 [SMILES: CC[C@@]1(O)C(=O)OCc2c1cc1n(c2=O)Cc2c-1nc1cc(F)c(C)c3c1c2[C@@H](NC(=O)OCCO)CC3]

In one embodiment, A is —NH—, and B is —C(═O)—.

In one embodiment, R⁵ and R⁶ are hydrogen, and n is 2.

In one embodiment, the compound is

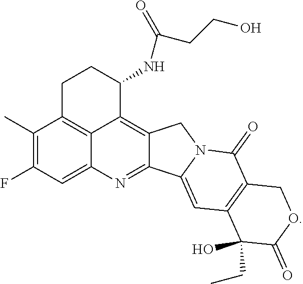 [SMILES: CC[C@@]1(O)C(=O)OCc2c1cc1n(c2=O)Cc2c-1nc1cc(F)c(C)c3c1c2[C@@H](NC(=O)CCO)CC3]

In one embodiment, R³ is Cl, R⁴ is F, and B is —C(═O)—.

In one embodiment, the compound is

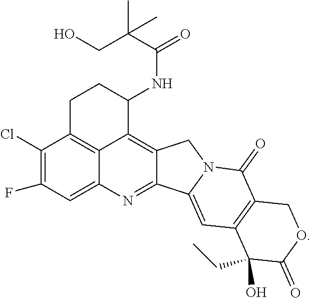 [SMILES: CC[C@@]1(O)C(=O)OCc2c1cc1n(c2=O)Cc2c-1nc1cc(F)c(Cl)c3c1c2C(NC(=O)C(C)(C)CO)CC3]

In one embodiment, R³ is methyl, R⁴ is Cl, and B is —C(═O)—.

In one embodiment, the compound is

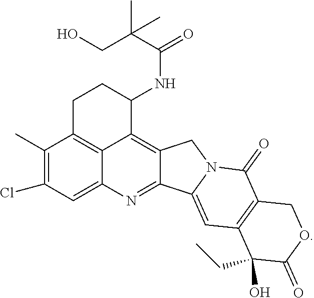 [SMILES: CC[C@@]1(O)C(=O)OCc2c1cc1n(c2=O)Cc2c-1nc1cc(Cl)c(C)c3c1c2C(NC(=O)C(C)(C)CO)CC3]

In one embodiment, R³, and R⁴ together with the atoms to which they are attached to, form unsubstituted or substituted heterocyclyl.

In one embodiment, R³, and R⁴ together with the atoms to which they are attached to, form an unsubstituted or substituted dioxole ring, and B is —C(═O)—.

In one embodiment, the compound is

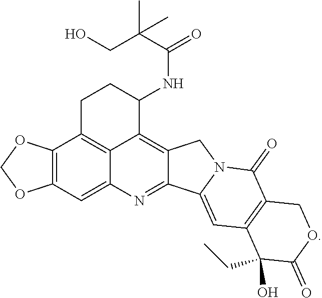 [SMILES: CC[C@@]1(O)C(=O)OCc2c1cc1n(c2=O)Cc2c-1nc1cc3c(c4c1c2C(NC(=O)C(C)(C)CO)CC4)OCO3]

### Aspect 7

In one embodiment, the compound has formula (III):

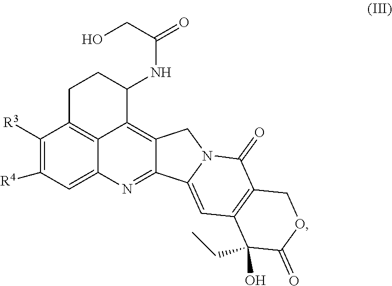

or a pharmaceutically acceptable solvate, stereoisomer, or derivative thereof.

In one embodiment, R³ is methy; and R⁴ is Cl.

In one embodiment, the compound is

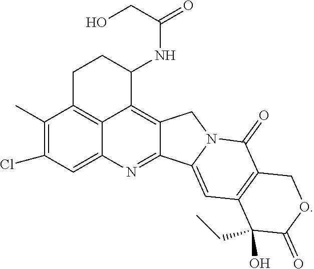 [SMILES: CC[C@@]1(O)C(=O)OCc2c1cc1n(c2=O)Cc2c-1nc1cc(Cl)c(C)c3c1c2C(NC(=O)CO)CC3]

In one embodiment, R³ is Cl; and R⁴ is F.

In one embodiment, the compound is

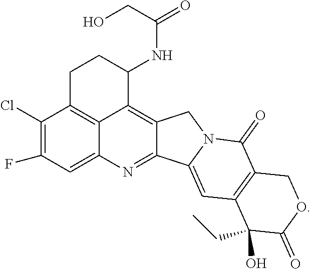 [SMILES: CC[C@@]1(O)C(=O)OCc2c1cc1n(c2=O)Cc2c-1nc1cc(F)c(Cl)c3c1c2C(NC(=O)CO)CC3]

In one embodiment, R³ is F; and R⁴ is F.

In one embodiment, the compound is

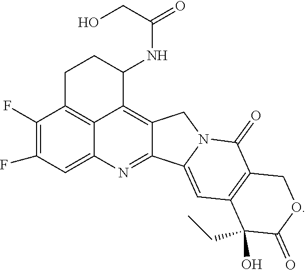 [SMILES: CC[C@@]1(O)C(=O)OCc2c1cc1n(c2=O)Cc2c-1nc1cc(F)c(F)c3c1c2C(NC(=O)CO)CC3]

In one embodiment, R³ is H; and R⁴ is F.

In one embodiment, the compound is

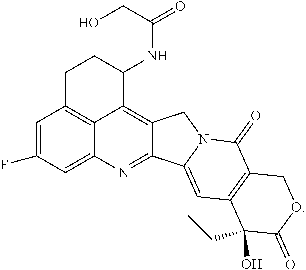 [SMILES: CC[C@@]1(O)C(=O)OCc2c1cc1n(c2=O)Cc2c-1nc1cc(F)cc3c1c2C(NC(=O)CO)CC3]

In one embodiment, R³ is H; and R⁴ is OH.

In one embodiment, the compound is

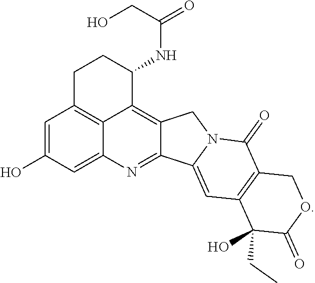 [SMILES: CC[C@@]1(O)C(=O)OCc2c1cc1n(c2=O)Cc2c-1nc1cc(O)cc3c1c2[C@@H](NC(=O)CO)CC3]

In one embodiment, R³ is methyl; and R⁴ is methyl.

In one embodiment, the compound is

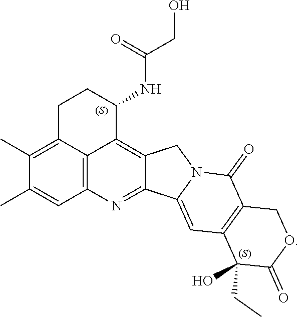

In one embodiment, R³ is methoxyl; and R⁴ is F.

In one embodiment, the compound is

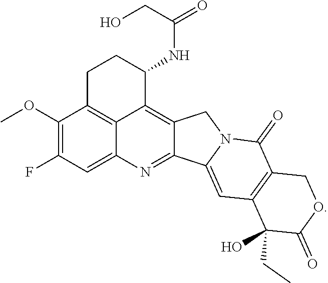 [SMILES: CC[C@@]1(O)C(=O)OCc2c1cc1n(c2=O)Cc2c-1nc1cc(F)c(OC)c3c1c2[C@@H](NC(=O)CO)CC3]

In one embodiment, R³ is H; and R⁴ is methoxyl.

In one embodiment, the compound is

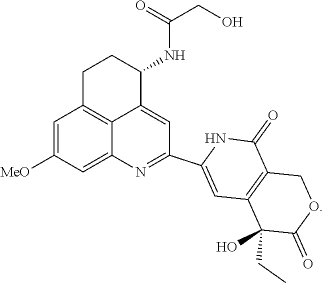 [SMILES: CC[C@@]1(O)C(=O)OCc2c1cc(-c1cc3c4c(cc(OC)cc4n1)CC[C@@H]3NC(=O)CO)[nH]c2=O]

In one embodiment, R³ is H; and R⁴ is Cl.

In one embodiment, the compound is

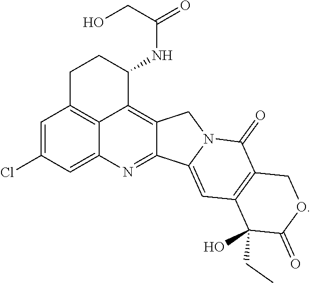 [SMILES: CC[C@@]1(O)C(=O)OCc2c1cc1n(c2=O)Cc2c-1nc1cc(Cl)cc3c1c2[C@@H](NC(=O)CO)CC3]

In one embodiment, R³, and R⁴ together with the atoms to which they are attached to, form unsubstituted or substituted heterocyclyl.

In one embodiment, R³, and R⁴ together with the atoms to which they are attached to, form an unsubstituted or substituted dioxole ring.

In one embodiment, the compound is

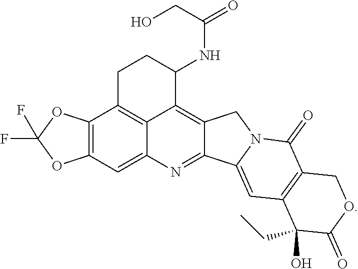 [SMILES: CC[C@@]1(O)C(=O)OCc2c1cc1n(c2=O)Cc2c-1nc1cc3c(c4c1c2C(NC(=O)CO)CC4)OC(F)(F)O3]

In one embodiment, the compound is

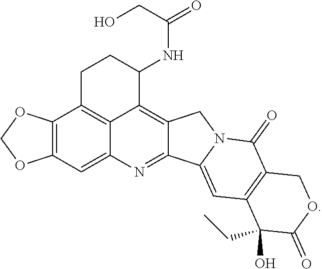 [SMILES: CC[C@@]1(O)C(=O)OCc2c1cc1n(c2=O)Cc2c-1nc1cc3c(c4c1c2C(NC(=O)CO)CC4)OCO3]

### Aspect 8

In one embodiment, the compound is

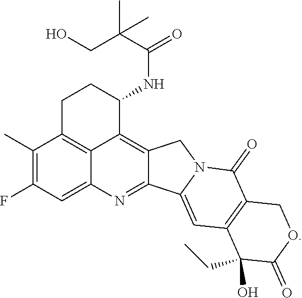 [SMILES: CC[C@@]1(O)C(=O)OCc2c1cc1n(c2=O)Cc2c-1nc1cc(F)c(C)c3c1c2[C@@H](NC(=O)C(C)(C)CO)CC3]

In one embodiment, the compound is

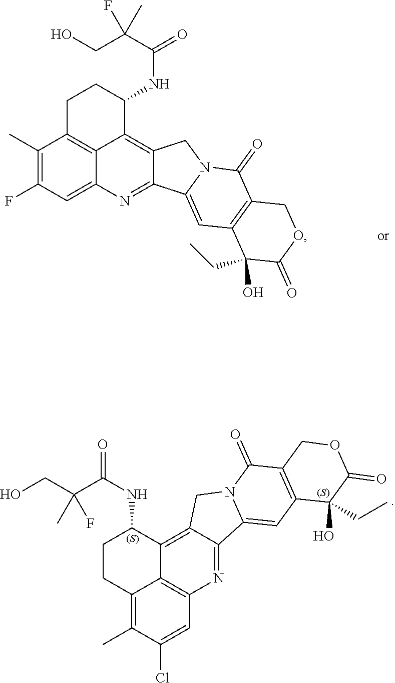

### Aspect 9

In one embodiment, the compound is a compound selected from Table 1 and Table 2.

### Aspect 10

Provided herein is a ligand-drug conjugate or a pharmaceutically acceptable salt or solvate thereof, wherein the ligand-drug conjugate comprises a residue of the compound provided herein.

In one embodiment, the ligand-drug conjugate comprises a structure of formula (V):

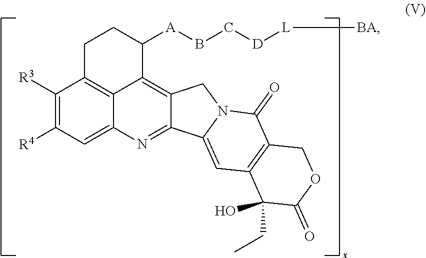

wherein
BA is a binding agent selected from a humanized, chimeric, or human antibody or an antigen binding antibody fragment of an antibody;
L is a covalent linker as described here; and
x is 1 to 10, which can be an integer or a decimal.

In one embodiment, the antibody is patritumab, cofetuzumab, trastuzumab, ifinatamab, or mAb10.

### Aspect 11

Provided herein is a compound having formula (VII):

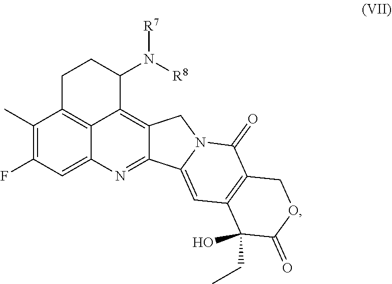

or a pharmaceutically acceptable salt, tautomer, isotopologue, stereoisomer, or prodrug thereof, wherein
each of R⁷, and R⁸ is, independently, hydrogen, or substituted or unsubstituted alkyl; or R⁷, and R⁸ together with the nitrogen atom to which they are attached to, form unsubstituted or substituted heterocyclyl, or unsubstituted or substituted heteroaryl.

In one embodiment, the compound is

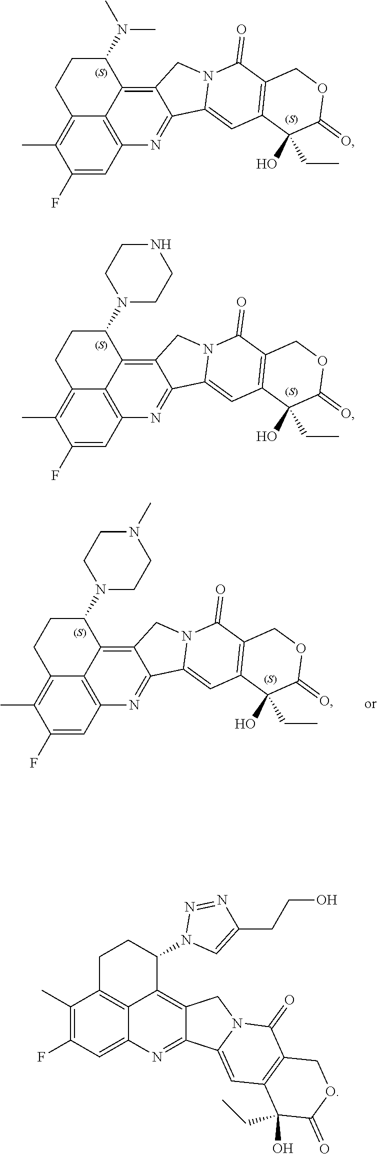

In one embodiment, the ligand-drug conjugate comprises a structure of formulas (VIIIa), (VIIIb), or (VIIIc):

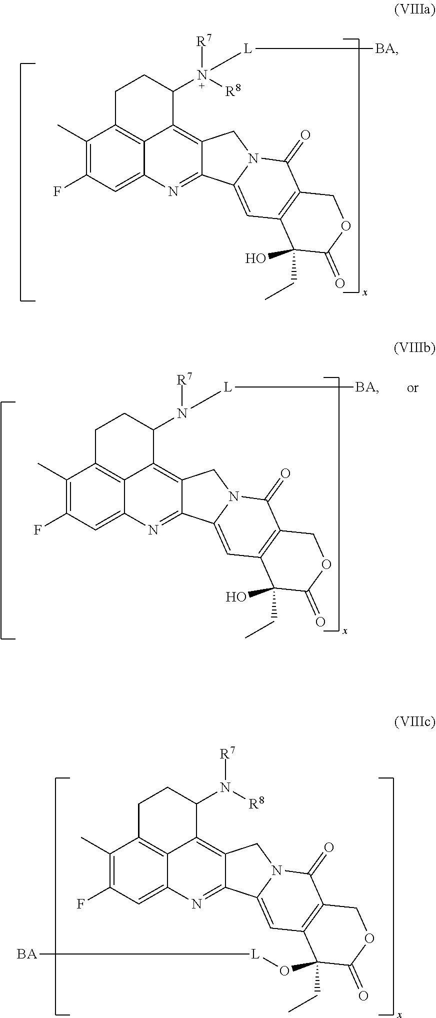

wherein
BA is a binding agent selected from a humanized, chimeric, or human antibody or an antigen binding antibody fragment of an antibody;
L is a covalent linker as described here; and
x is 1 to 10, which can be an integer or a decimal.

In one embodiment, the ligand-drug conjugate comprises a structure of any one of the following formulas:

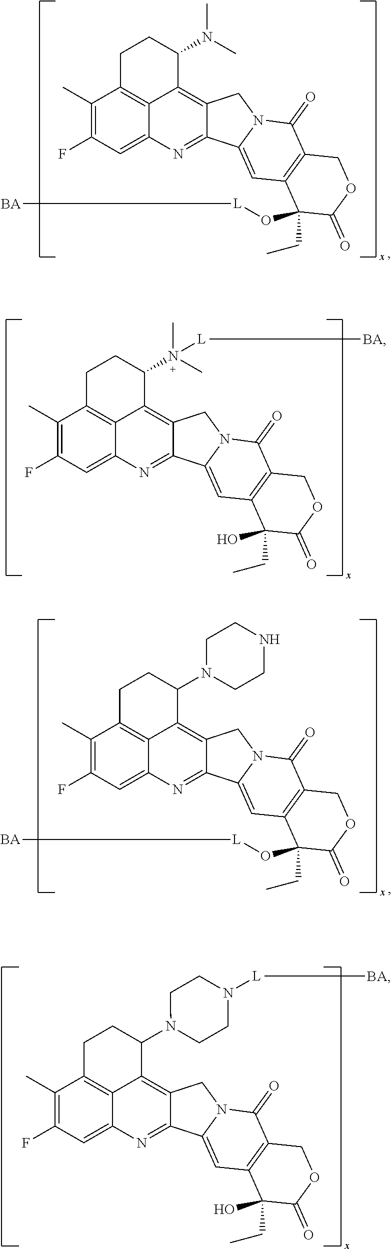

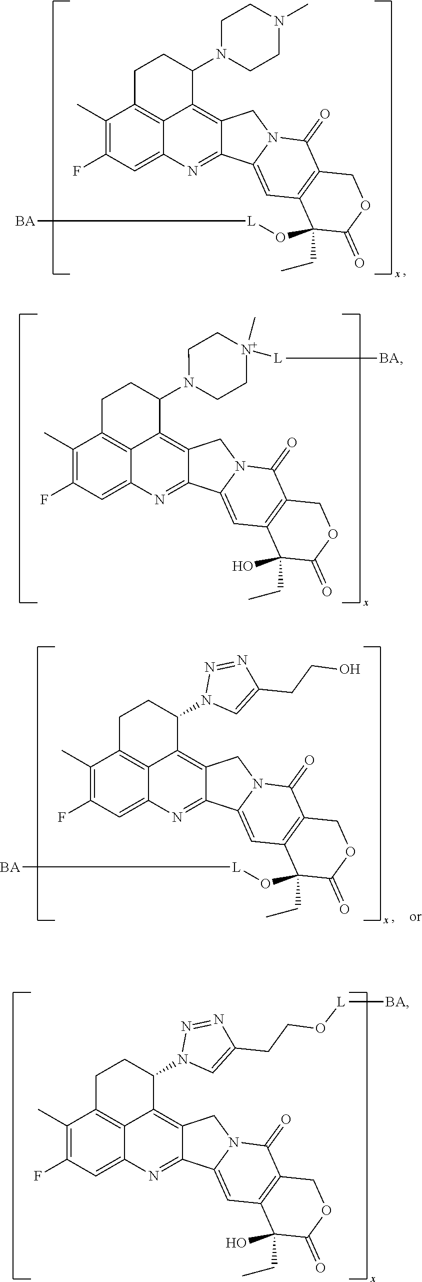

wherein
BA is a binding agent selected from a humanized, chimeric, or human antibody or an antigen binding antibody fragment of an antibody;
L is a covalent linker as described here; and
x is 1 to 10, which can be an integer or a decimal.

In one embodiment, the antibody is patritumab, cofetuzumab, trastuzumab, ifinatamab, or mAb10.

In one embodiment, the L is

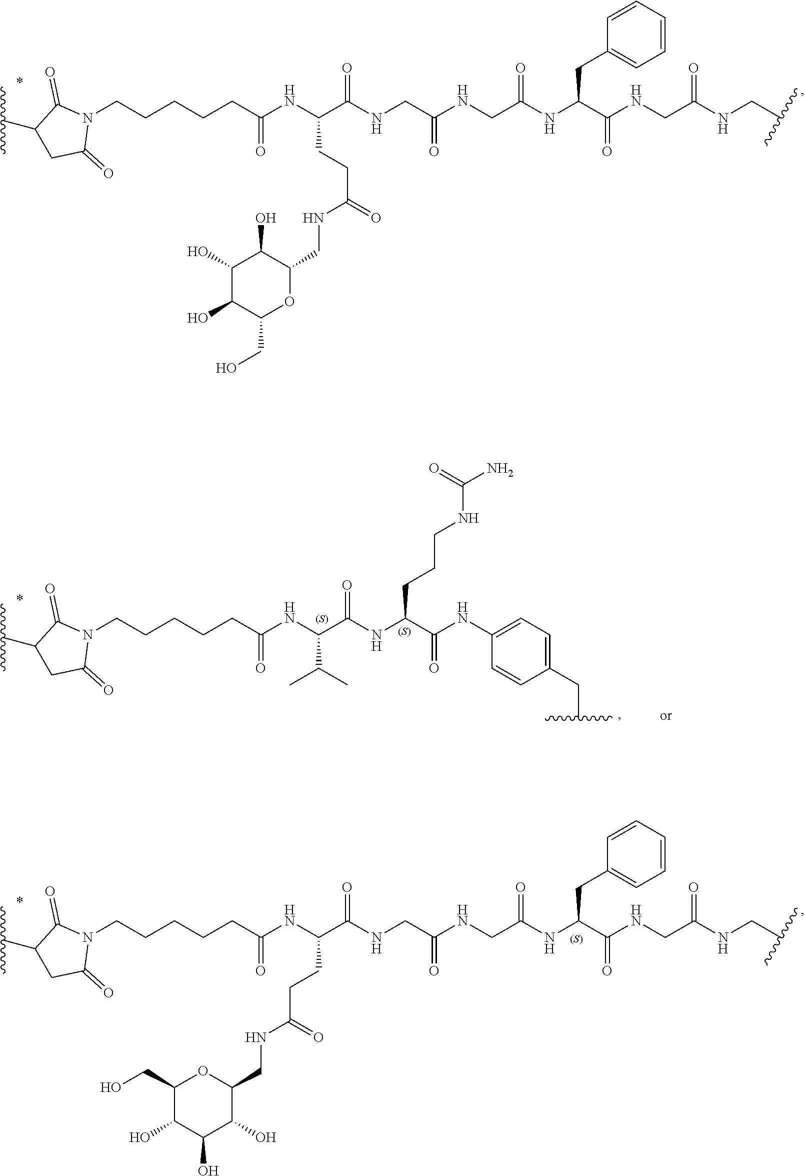

wherein the bond marked with asterisk is connected to BA.

In one embodiment, the L is

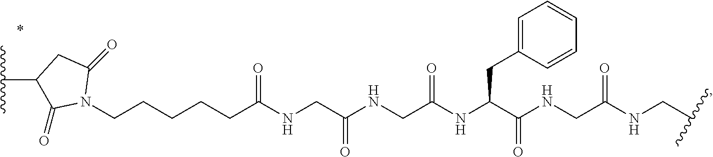 [SMILES: *CNC(=O)CNC(=O)[C@H](Cc1ccccc1)NC(=O)CNC(=O)CNC(=O)CCCCCN1C(=O)CC(*)C1=O]

wherein the bond marked with asterisk is connected to BA.

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

### 7.4. Covalent Linker

### Aspect 12

Described herein are covalent linkers according to Formula (IXa):

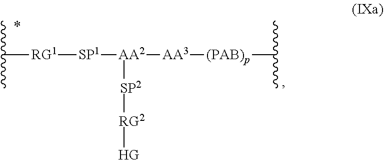

wherein RG¹ is a reactive group residue; RG² is an optional reactive group residue; SP¹ and SP² are independently, in each instance, an optional spacer group residue; HG is a hydrophilic residue; PAB is an optional self-immolative unit; and subscript p is 0 or 1.

In some embodiments, a compound of Formula (IXa) is a compound having P3 modification, wherein AA² comprises formula (W):

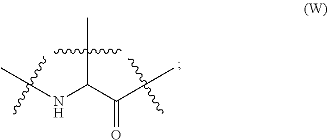 [SMILES: *NC(*)C(*)=O]

and
AA³ is a dipeptide residue of -valine-alanine-, -valine-citrulline-, or

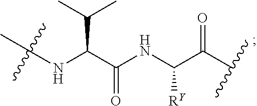

wherein R^{Y} is —CH₃, or —(CH₂)₃—NHC(═O)NH₂.

In one embodiment,

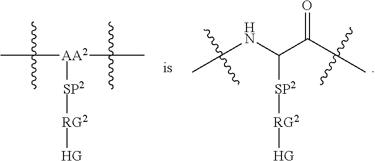

In some embodiments, PAB represents —NH—CH2-O—, formula (Y1):

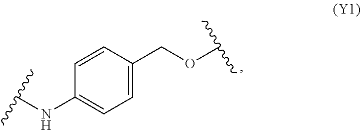 [SMILES: *Nc1ccc(CO*)cc1]

or formula (Y2):

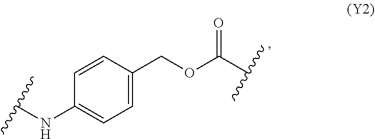 [SMILES: *Nc1ccc(COC(*)=O)cc1]

wherein the

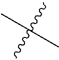

indicates the bond through which the PAB is bonded to the adjacent groups in the formula.

In some embodiments, RG¹ is

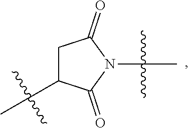 [SMILES: *C1CC(=O)N(*)C1=O]

### -(Succinimid-3-yl-N)—,

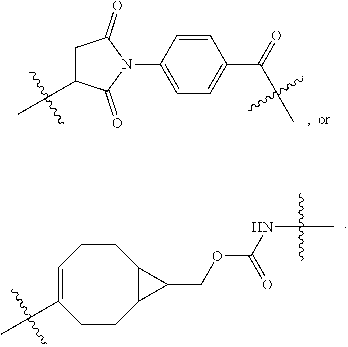 [SMILES: *C(=O)c1ccc(N2C(=O)CC(*)C2=O)cc1 ; *NC(=O)OCC1C2CC/C=C(/*)CCC21]

In some embodiments, RG¹ is

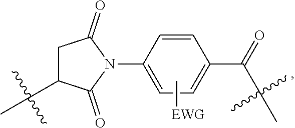

wherein EWG is an electrowithdrawn group, e.g., —CN, —NO₂, halogen, —CF₃, —C(═O)OR^{x}, and —C(═O)R^{x}, and R^{x} is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, RG¹ is

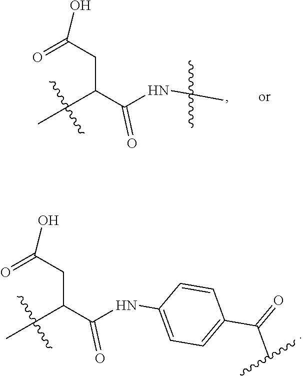 [SMILES: *NC(=O)C(*)CC(=O)O ; *C(=O)c1ccc(NC(=O)C(*)CC(=O)O)cc1]

In some embodiments, RG¹ is

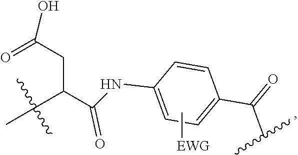

wherein EWG is an electrowithdrawn group, e.g., —CN, —NO₂, halogen, —CF₃, —C(═O)OR^{x}, and —C(═O)R^{x}, and R^{x} is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (IXa) having its ring opened is a compound of Formula (IXa), wherein RG¹ is an opened heterocyclic ring. In some embodiments, RG¹ is

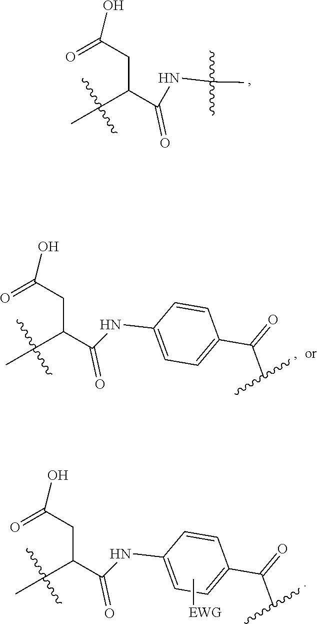

In some embodiments, RG² is a bond, —C(═O)—NH—, or —NHC(═O)—.

In some embodiments, SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)ₙ₃—C(═O)—NH—(CH₂)ₙ₄—C(=O)—, or —C(═O)—(CH₂)ₙ₅—C(═O)—, wherein each of n1, n2, n3, n4, and n5 independently represents an integer of 1 to 8.

In some embodiments, SP² is —(CH₂)ₙ₆—; and n6 represents an integer of 1 to 8.

In some embodiments, HG is

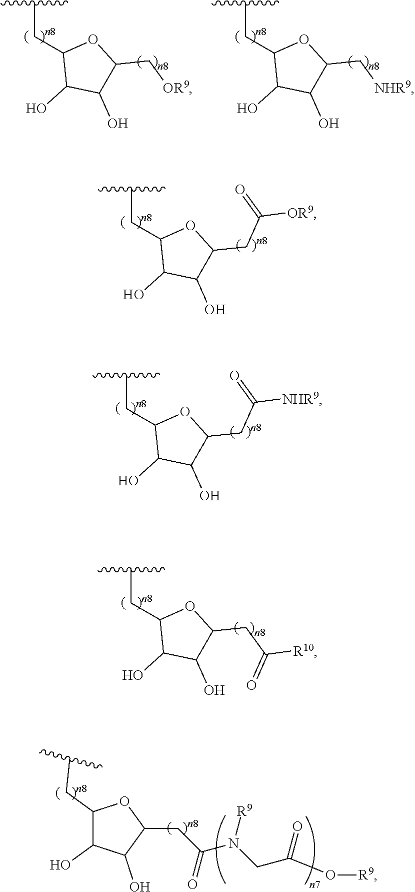

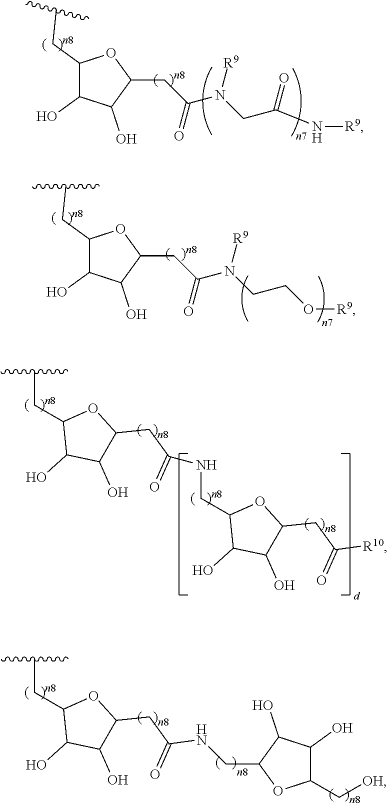

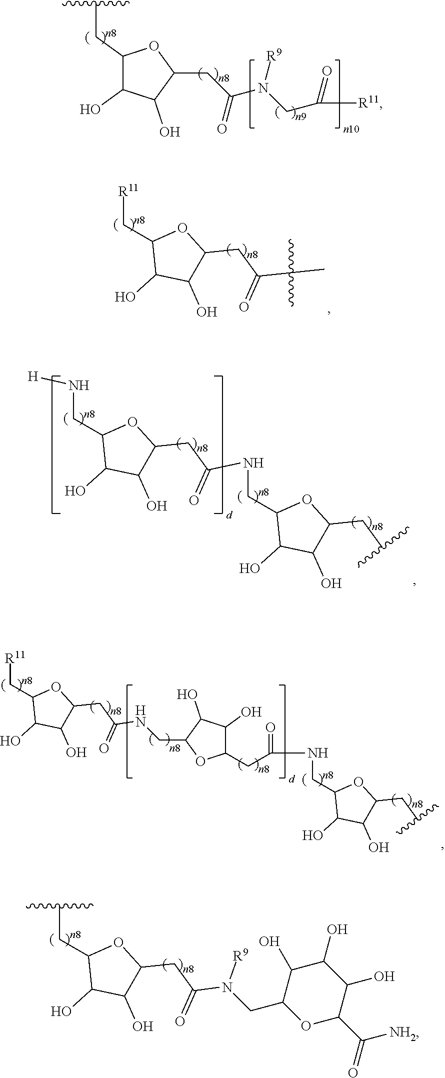

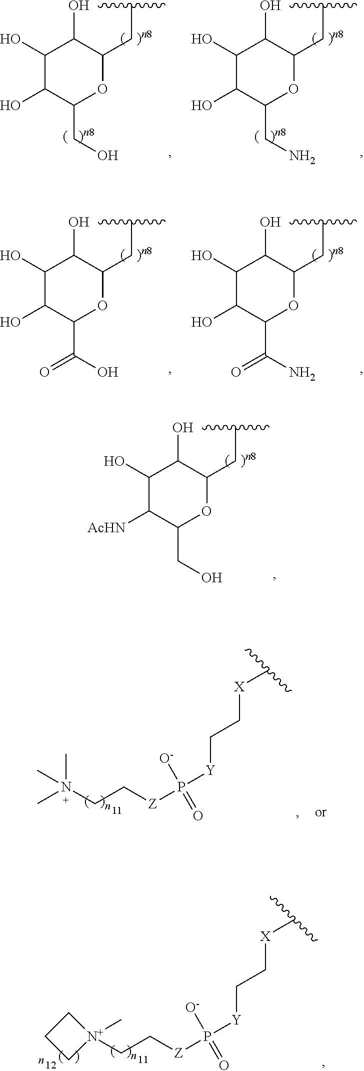

wherein each n7 is independently 1-15; each n8 is independently 0 or 1; each n9 is independently 1 or 2; each n10 is independently an integer of 4 to 16, such as 4, 8, or 12; each n11 is independently an integer of 0 to 5; n12 is an integer of 0 to 3; d is 0-3; R⁹ is H or Me; R¹⁰ is —OH, —NH₂, —NHCH₂—CH₂—(PEG), —OH, or —NHCH₂—CH₂-(PEG)ₓ-OMe; R¹¹ is OH or NH₂; and each of X, Y, and Z is independently —CH₂—, —NH—, —S— or —O—.

In some embodiments, HG is

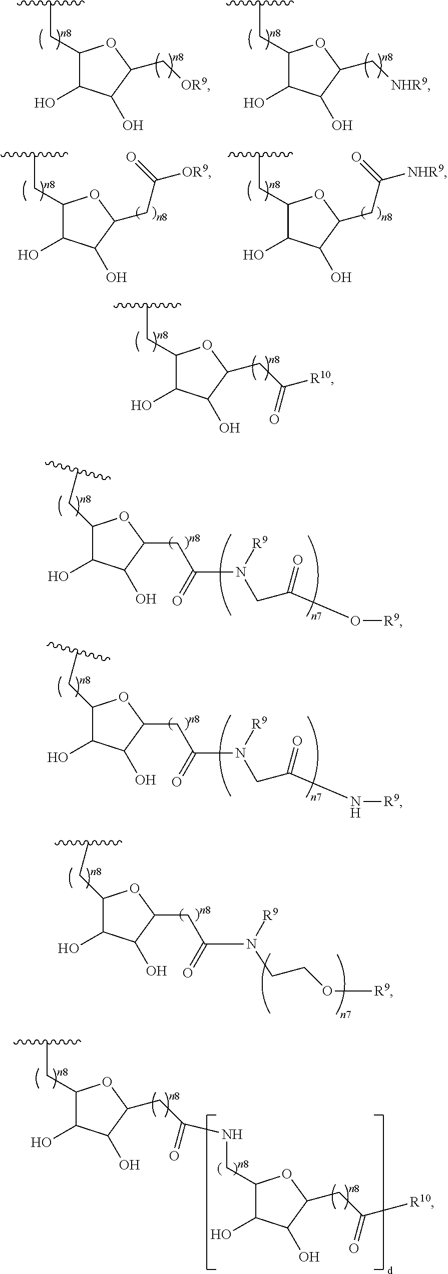

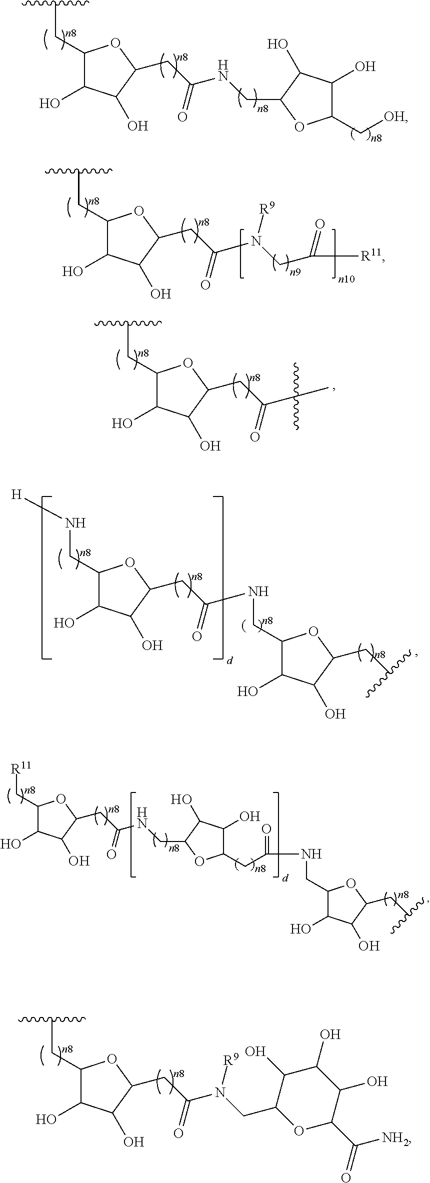

wherein each n7 is independently 1-15; each n8 is independently 0 or 1; each n9 is independently 1 or 2; each n10 is independently an integer of 4 to 16, such as 4, 8, or 12; d is 0-3; R⁹ is H or Me; R¹⁰ is —OH, —NH₂, —NHCH₂—CH₂—(PEG)ₓ-OH, or —NHCH₂—CH₂—(PEG)ₓ-OMe; R¹¹ is OH or NH₂.

In some embodiments, HG is

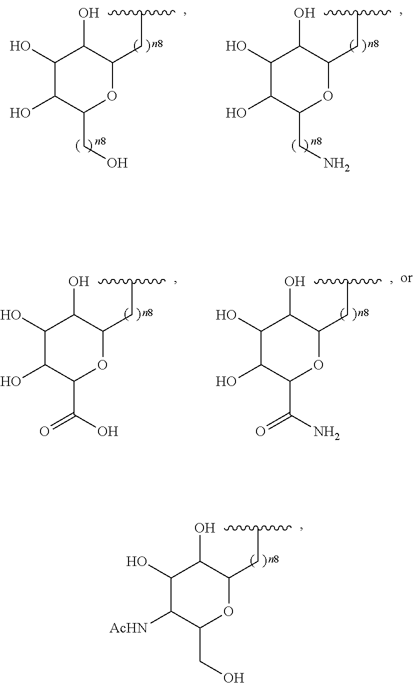

wherein each n8 is independently 0 or 1; and R¹ is H or Me.

In some embodiments, HG is

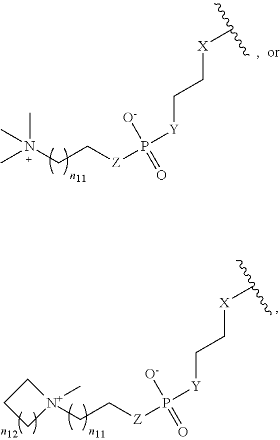

wherein each n11 is independently an integer of 0 to 5; n12 is an integer of 0 to 3; and each of X, Y, and Z is independently —CH₂—, —NH—, —S— or —O—.

In some embodiments, HG is —NHSO₂NH₂, —SO₃H, —SO₂NH₂, —PO₃H₂, and RG² is a bond.

In some embodiments, each PA is independently selected from the compounds provided herein.

In some embodiments, R⁴ is hydrogen,

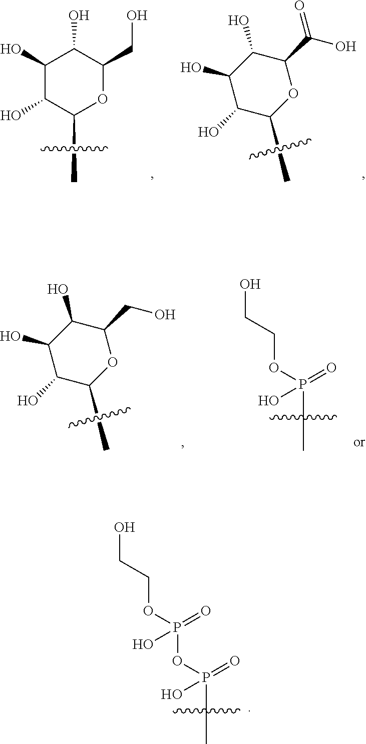

In some embodiments, R⁴ is hydrogen,

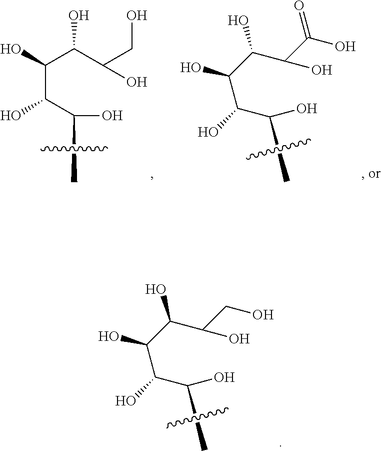

In some embodiments,

AA² is an amino acid residue of glycine or

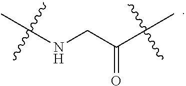 [SMILES: *NCC(*)=O]

### Aspect 13

Described herein are compounds of Formula (IXa) or a pharmaceutically acceptable salt tautomer, solvate, stereoisomer, enantiomer, isotopologue, or prodrug thereof, wherein AA² comprises formula (W):

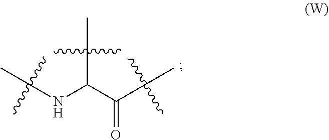 [SMILES: *NC(*)C(*)=O]

and
AA³ is a tetrapeptide residue of -glycine-glycine-phenylalanine-glycine- or

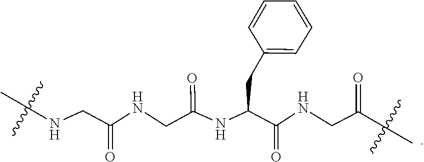 [SMILES: *NCC(=O)NCC(=O)N[C@@H](Cc1ccccc1)C(=O)NCC(*)=O]

### Aspect 14

Described herein are covalent linkers according to Formula (IXb):

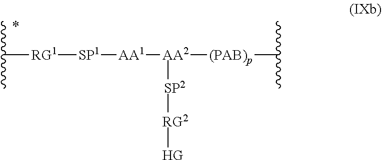

or a pharmaceutically acceptable salt tautomer, solvate, stereoisomer, enantiomer, isotopologue, or prodrug thereof,
wherein AA² comprises formula (W):

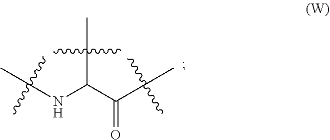 [SMILES: *NC(*)C(*)=O]

and
AA¹ is a dipeptide residue of -valine-alanine-, -valine-citrulline-, or

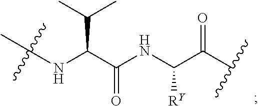

wherein R^{Y} is —CH₃, or —(CH₂)₃—NHC(═O)NH₂.

In one embodiment,

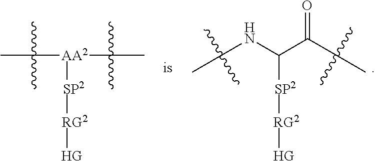

In some embodiments, a compound of Formula (IXb) having its ring opened is a compound of Formula (IXb), wherein RG¹ is an opened heterocyclic ring. In some embodiments, a compound of Formula (Ib) with ring opening is a compound of Formula (IXb), wherein RG¹ is an opened heterocyclic ring. In some embodiments, RG¹ is

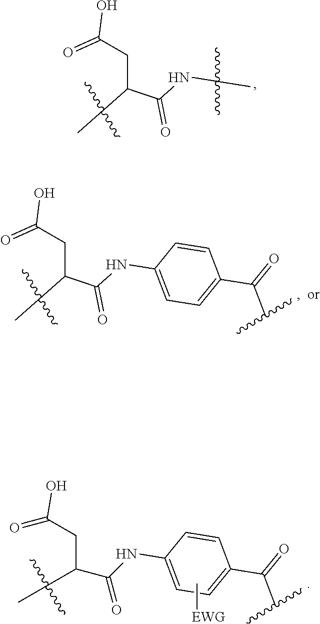

In one embodiment, the BA, RG¹, SP¹, SP², RG², HG, PAB, p, and PA are as provided herein.

### Aspect 15

Described herein are covalent linkers of Formula (IXb), wherein AA² comprises formula (W):

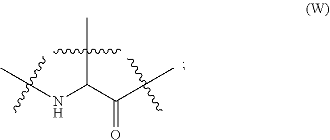 [SMILES: *NC(*)C(*)=O]

and
AA¹ is a tetrapeptide residue of -glycine-glycine-phenylalanine-glycine- or

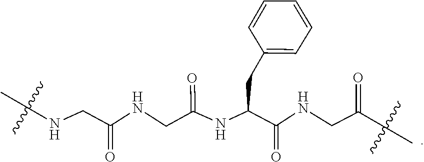 [SMILES: *NCC(=O)NCC(=O)N[C@@H](Cc1ccccc1)C(=O)NCC(*)=O]

In one embodiment, the BA, RG¹, SP¹, SP², RG², HG, PAB, p, x, and PA are as provided herein.

### Aspect 16

Described herein are covalent linkers according to Formula (IXc):

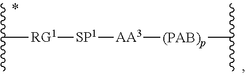

wherein AA³ is a dipeptide residue of -valine-alanine-, -valine-citrulline-, or

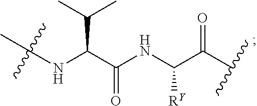

wherein R^{Y} is —CH₃, or —(CH₂)₃—NHC(═O)NH₂.

In some embodiments, a covalent linker of Formula (IXc) having its ring opened is a covalent linker of Formula (IXc), wherein RG¹ is an opened heterocyclic ring. In some embodiments, a covalent linker of Formula (IXc) with ring opening is a covalent linker of Formula (IXc), wherein RG¹ is an opened heterocyclic ring. In some embodiments, RG¹ is

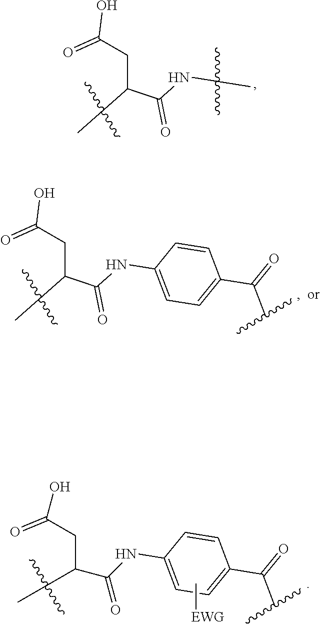

### Aspect 17

Described herein are covalent linkers according to Formula (IXc), wherein AA³ is a tetrapeptide residue of -glycine-glycine-phenylalanine-glycine- or

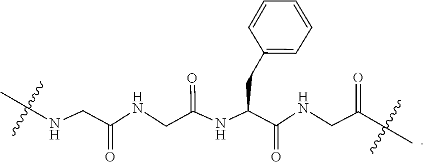 [SMILES: *NCC(=O)NCC(=O)N[C@@H](Cc1ccccc1)C(=O)NCC(*)=O]

### Aspect 18

In some embodiments, the compound is selected from the group consisting of the compounds in Table 4.

### 7.5. Linkers with Payloads (Platform)

In some embodiments, set forth herein is a compound comprises a reactive linker bonded to at least one payload moiety.

In some embodiments, set forth herein is a compound, or a pharmaceutically acceptable solvate, stereoisomer, or derivative thereof, comprising a payload unit linked to at least one hydrophilic residue via a covalent linker unit, wherein the covalent linker is bonded directly or indirectly to each of the payload unit, and the hydrophilic residue.

In one embodiment, the hydrophilic residue comprises a terminal hydrophilic group.

In one embodiment, the hydrophilic residue comprises a sugar residue.

### Aspect 19

In some embodiments, set forth herein is a compound having formula (X):

L-PA (X)

or a pharmaceutically acceptable salt tautomer, solvate, stereoisomer, enantiomer, isotopologue, or prodrug thereof, wherein L is a covalent linker; and PA is a payload residue.

### Aspect 20

Described herein are compounds according to Formula (Xa):

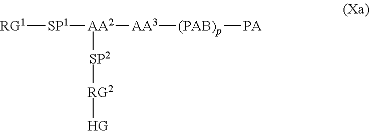

or a pharmaceutically acceptable salt tautomer, solvate, stereoisomer, enantiomer, isotopologue, or prodrug thereof,
wherein AA² comprises formula (W):

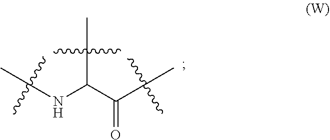 [SMILES: *NC(*)C(*)=O]

and
AA³ is a dipeptide residue of -valine-alanine-, -valine-citrulline-, or

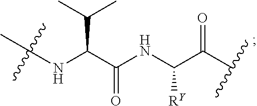

wherein R^{Y} is —CH₃, or —(CH₂)₃—NHC(═O)NH₂.

In one embodiment,

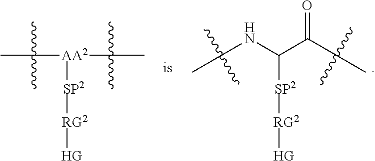

In one embodiment, the RG¹ is

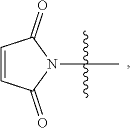 [SMILES: *N1C(=O)C=CC1=O]

### Succinimid-N—,

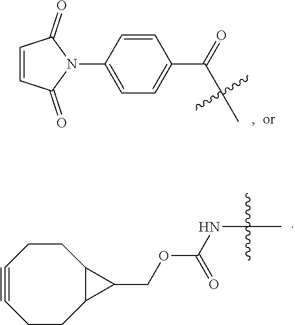 [SMILES: *C(=O)c1ccc(N2C(=O)C=CC2=O)cc1 ; *NC(=O)OCC1C2CCC#CCCC21]

In one embodiment, the RG¹ is

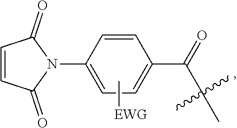

wherein EWG is an electrowithdrawn group, e.g., —CN, —NO₂, halogen, —CF₃, —C(═O)OR^{x}, and —C(═O)R^{x}, and R^{x} is substituted or unsubstituted alky, substituted or unsubstituted cycloalkyl, substituted or unsubstituted aryl, substituted or unsubstituted heterocycloalkyl, or substituted or unsubstituted heteroaryl.

In one embodiment, the RG¹ is

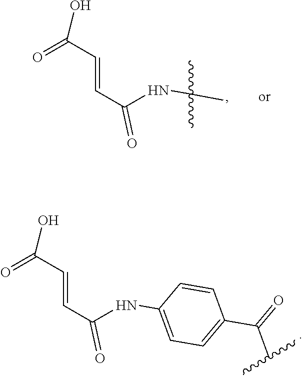 [SMILES: *NC(=O)/C=C/C(=O)O ; *C(=O)c1ccc(NC(=O)/C=C/C(=O)O)cc1]

In one embodiment, the RG¹ is

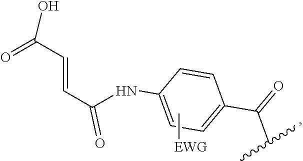

wherein EWG is an electrowithdrawn group, e.g., —CN, —NO₂, halogen, —CF₃, —C(═O)OR^{x}, and —C(═O)R^{x}, and R^{x} is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (X) having its ring opened is a compound of Formula (X), wherein RG¹ is an opened heterocyclic ring. In some embodiments, a compound of Formula (X) with ring opening is a compound of Formula (X), wherein RG¹ is an opened heterocyclic ring. In some embodiments, RG¹ is

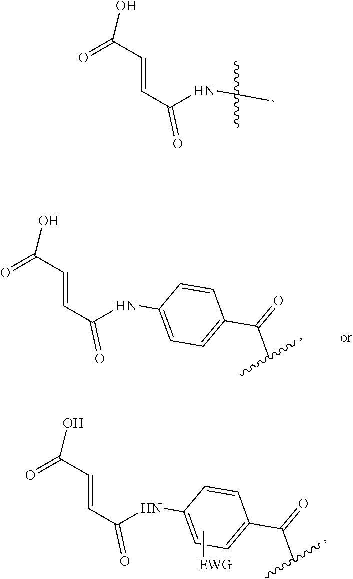

wherein EWG is an electrowithdrawn group, e.g., —CN, —NO₂, halogen, —CF₃, —C(═O)OR^{x}, and —C(═O)R^{x}, and R^{x} is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (Xa) having its ring opened is a compound of Formula (Xa), wherein RG¹ is an opened heterocyclic ring. In some embodiments, a compound of Formula (Xa) with ring opening is a compound of Formula (Xa), wherein RG¹ is an opened heterocyclic ring. In some embodiments, RG¹ is

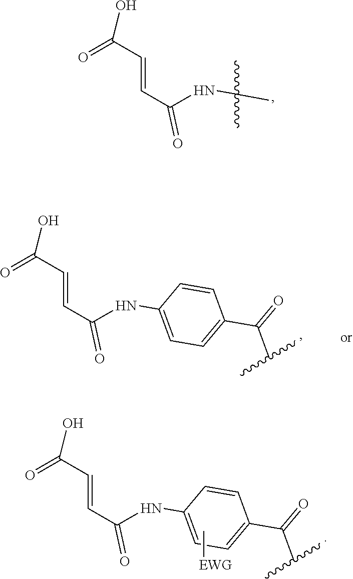

In one embodiment, the RG¹, SP¹, SP², RG², HG, PAB, p, and PA are as provided herein.

### Aspect 21

Described herein are compounds according to Formula (Xa) or a pharmaceutically acceptable salt tautomer, solvate, stereoisomer, enantiomer, isotopologue, or prodrug thereof, wherein AA² comprises formula (W):

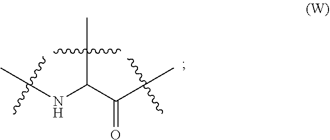 [SMILES: *NC(*)C(*)=O]

and
AA³ is a tetrapeptide residue of -glycine-glycine-phenylalanine-glycine- or

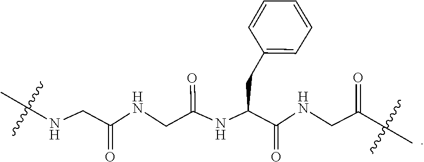 [SMILES: *NCC(=O)NCC(=O)N[C@@H](Cc1ccccc1)C(=O)NCC(*)=O]

In one embodiment, the RG¹, SP¹, SP², RG², HG, PAB, p, and PA are as provided herein.

### Aspect 22

Described herein are compounds according to Formula (Xb):

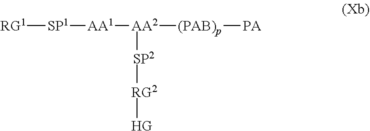

or a pharmaceutically acceptable salt tautomer, solvate, stereoisomer, enantiomer, isotopologue, or prodrug thereof,
wherein AA² comprises formula (W):

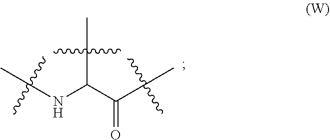 [SMILES: *NC(*)C(*)=O]

AA¹ is a dipeptide residue of -valine-alanine-, -valine-citrulline-, or

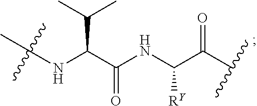

wherein R^{Y} is —CH₃, or —(CH₂)₃—NHC(═O)NH₂.

In one embodiment,

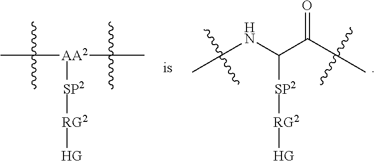

In some embodiments, a compound of Formula (Xb) having its ring opened is a compound of Formula (Xb), wherein RG¹ is an opened heterocyclic ring. In some embodiments, RG¹ is

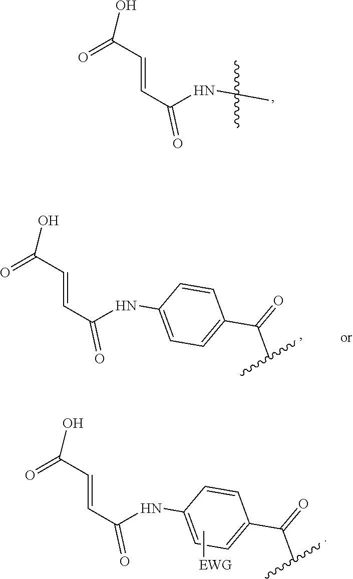

In one embodiment, the RG¹, SP¹, SP², RG², HG, PAB, p, and PA are as provided herein.

### Aspect 23

Described herein are compounds according to Formula (Xb) or a pharmaceutically acceptable salt tautomer, solvate, stereoisomer, enantiomer, isotopologue, or prodrug thereof, wherein AA² comprises formula (W):

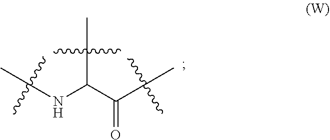 [SMILES: *NC(*)C(*)=O]

and
AA¹ is a tetrapeptide residue of -glycine-glycine-phenylalanine-glycine- or

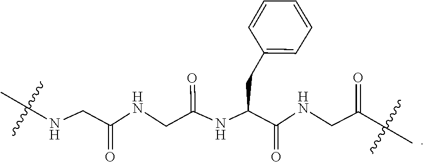 [SMILES: *NCC(=O)NCC(=O)N[C@@H](Cc1ccccc1)C(=O)NCC(*)=O]

In one embodiment, the RG¹, SP¹, SP², RG², HG, PAB, p, and PA are as provided herein.

### Aspect 24

Described herein are compounds according to Formula (Xc):

RG¹-SP¹-AA³-(PAB)ₚ-PA (Xc)

or a pharmaceutically acceptable salt tautomer, solvate, stereoisomer, enantiomer, isotopologue, or prodrug thereof,
wherein AA³ is a dipeptide residue of -valine-alanine-, -valine-citrulline-, or

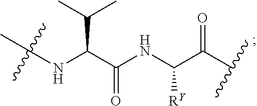

wherein R^{Y} is —CH₃, or —(CH₂)₃—NHC(═O)NH₂.

In some embodiments, a compound of Formula (Xc) having its ring opened is a compound of Formula (Xc), wherein RG¹ is an opened heterocyclic ring. In some embodiments, RG¹ is

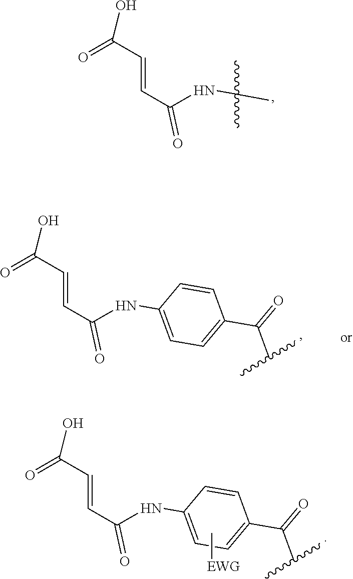

In one embodiment, the RG¹, SP¹, PAB, p, and PA are as provided herein.

### Aspect 25

Described herein are compounds according to Formula (Xc) or pharmaceutically acceptable salts, solvates, stereoisomers, or derivatives thereof,

wherein: RG¹ is a reactive group residue; SP¹ is an optional spacer group residue; PAB is an optional self-immolative unit; subscript p is 0 or 1; and PA is a payload residue;
wherein AA³ is a tetrapeptide residue of -glycine-glycine-phenylalanine-glycine- or

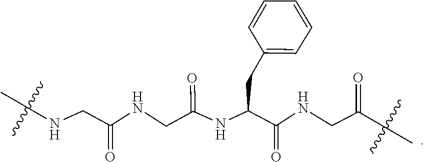 [SMILES: *NCC(=O)NCC(=O)N[C@@H](Cc1ccccc1)C(=O)NCC(*)=O]

In one embodiment, the RG¹, SP¹, PAB, p, and PA are as provided herein.

### Aspect 26

In some embodiments, the compound is selected from the group consisting of the compounds in Table 3.

The covalent linkers can be prepared as described in PCT/CN2021/142037, PCT/CN2022/086931, PCT/CN2022/088762, and PCT/CN2022/097834, the entire disclosure of which are incorporated herein by reference.

### 7.6. Methods of Using

In some embodiments, set forth herein is a method of treating a disease or disorder in a patient in need thereof comprising administering to the patient a compound or pharmaceutical composition set forth herein. In some embodiments, the administered compound is an antibody-drug conjugate set forth herein.

In some embodiments, set forth herein is a method of treating or preventing a disease, disorder, or condition selected from the group consisting of a proliferative disorder, a neurodegenerative disorder, an immunological disorder, an autoimmune disease, an inflammatory disorder, a dermatological disease, a metabolic disease, cardiovascular disease, and a gastrointestinal disease comprising administering to the subject of an effective treatment amount of a compound or pharmaceutical composition set forth herein. In some embodiments, the administered compound is an antibody-drug conjugate set forth herein.

In some embodiments, set forth herein is a method of treating a proliferative disease, a metabolic disease, inflammation, or a neurodegenerative disease in a subject comprising administering to the subject of an effective treatment amount of a compound or pharmaceutical composition set forth herein. In some embodiments, set forth herein is a method of treating a proliferative disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating a metabolic disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating inflammation in a subject comprising administering to the subject of an effective treatment amount of a compound or pharmaceutical composition of set forth herein. In some embodiments, the administered compound is an antibody-drug conjugate set forth herein.

In some embodiments, set forth herein is a method of treating a neurodegenerative disease in a subject comprising administering to the subject of an effective treatment amount of a compound or pharmaceutical composition set forth herein. In some embodiments, the administered compound is an antibody-drug conjugate set forth herein.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 5.7 Antibody

In some embodiments, set forth herein is an antibody or antigen binding fragment thereof which specifically binds human HER3, comprising (i) a heavy chain variable region that comprises an amino acid sequence having at least 90%, 91%, 92%, 93%, 94%, 95%, 96%, 97%, 98%, 99%, 100% identity to the amino acid sequence of SEQ ID NO: 1; and a light chain variable region that comprises an amino acid sequence having at least 90%, 91%, 92%, 93%, 94%, 95%, 96%, 97%, 98%, 99%, 100% identity to the amino acid sequence of SEQ ID NO: 2; wherein the CDRs are the same to that as those of patritumab.

In some embodiments, set forth herein is an antibody or antigen binding fragment thereof which specifically binds human HER3, comprising: (i) a heavy chain variable region that comprises an amino acid sequence of SEQ ID NO: 1; and (ii) a light chain variable region that comprises an amino acid sequence of SEQ ID NO: 2.

In one embodiment, wherein the amino acid sequence of the heavy chain variable region is SEQ ID NO: 1; and the amino acid sequence of the light chain variable region is SEQ ID NO: 2.

In some embodiment, one, two, three, four, five, six, seven, eight, nine, or ten amino acids are further incorporated (inserted, deleted or substituted) into the framework region of the above-mentioned antibodies, such as SEQ ID NO: 1 or SEQ ID NO: 2.

In some embodiments, wherein the antibody or antigen binding fragment thereof comprises a heavy chain constant region of the subclass of IgG1, IgG2, IgG3, or IgG4, and/or a light chain constant region of the type of kappa or lambda.

In some embodiments, wherein the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

In some embodiments, wherein the antibody or antigen-binding fragment thereof has antibody dependent cellular cytotoxicity (ADCC) or complement dependent cytotoxicity (CDC).

In some embodiments, wherein the Fc domain is an IgG1 with reduced effector function.

In some embodiments, set forth herein is an antibody or antigen binding fragment thereof which specifically binds human HER3, comprising (i) a heavy chain that comprises an amino acid sequence having at least 90%, 91%, 92%, 93%, 94%, 95%, 96%, 97%, 98%, 99%, 100% identity to the amino acid sequence of SEQ ID NO: 3; and a light chain that comprises an amino acid sequence having at least 90%, 91%, 92%, 93%, 94%, 95%, 96%, 97%, 98%, 99%, 100% identity to the amino acid sequence of SEQ ID NO: 4; wherein the CDRs are the same to that as those of patritumab.

In some embodiments, the antibody or antigen binding fragment thereof comprises (i) a heavy chain that comprises an amino acid sequence of SEQ ID NO: 3; and (ii) a light chain that comprises an amino acid sequence of SEQ ID NO: 4.

In some embodiments, the amino acid of a heavy chain is SEQ ID NO: 3; and the amino acid of a light chain is SEQ ID NO: 4.

In some embodiments, set forth herein is an isolated nucleic acid that encodes the antibody or antigen-binding fragment thereof of the present disclosure.

In some embodiments, set forth herein is a vector comprising the nucleic acid of the present disclosure.

In some embodiments, set forth herein is a host cell comprising the nucleic acid or the vector of the present disclosure.

In some embodiments, set forth herein is a process for producing the antibody or antigen-binding fragment thereof comprising cultivating the host cell and recovering the antibody or antigen-binding fragment thereof from the culture.

The antibody or antigen-binding fragment thereof of the present disclosure (such as the antibody or antigen binding fragment thereof specifically binding human HER3 comprises: (i) a heavy chain variable region that comprises an amino acid sequence of SEQ ID NO: 1; and (ii) a light chain variable region that comprises an amino acid sequence of SEQ ID NO: 2) unexpectedly shows better biophysical properties over Patritumab, such as, higher aggregation temperature and reduced self-association, indicating superior isothermal stability and further extended half-lives. Meanwhile, the antibody or antigen-binding fragment thereof of the present disclosure maintains comparable binding affinity to Patritumab.

### 8. EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilent 1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent amino acid residues. The amino acids and their corresponding three letter and single letter abbreviations are as follows:

### List of Abbreviations for Amino Acids

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

### List of Abbreviations

| | |
|---|---|
| TEA | Triethyl amine |
| THF | tetrahydrofuran |
| MeOH | Methanol |
| TEMPO | 2,2,6,6-Tetramethylpiperidine 1-oxyl |
| PhI(OAc)₂ | (Diacetoxyiodo)benzene |
| PyBOP | Benzotriazol-1-yl-oxytripyrrolidino-phosphonium |
| | Hexafluorophosphate |
| HOBt | 1-Hydroxybenzotriazole |
| DIPEA | N,N-Diisopropylethylamine |
| DMF | N,N-Dimethylformamide |
| NH4Cl | Ammonium chloride |
| LiOH | Lithium hydroxide |
| HCl | Hydrochloric acid |
| PNP | Bis(4-nitrophenyl)carbonate |
| TFA | Trifluoroacetic acid |
| K₂CO₃ | Potassium carbonate |
| MeI | Methyl iodide |
| Na₂SO₄ | Sodium sulfate |
| EtOAc | Ethyl acetate |
| MeONa | Sodium methoxide |
| prep-HPLC | Preparative high performance liquid chromatography |
| TBSCl | t-Butyldimethylchlorosilane |
| Cu(OAc)₂ | Anhydrous Cupric Acetate |
| Pb(OAc)₄ | lead tetraacetate |
| TSTU | 2-Succinimido-1,1,3,3-tetra-methyluronium tetrafluor |
| Et₂N | Diethylamine |
| r.t. | room temperature |
| MS | mass spectrometry |
| ESI | electron spray ionization |
| FA | formic acid |
| NHS | N-hydroxysuccinimide |
| MTBE | methyl tert-butyl ether |
| NHS | N-hydroxysuccinimide |
| Fmoc | 9-fluorenylmethoxycarbonyl protecting group |
| DBU | 1,8-diazabicyclo[5.4.0]undec-7-ene |
| DCC | dicyclohexylcarbodiimide |
| HPLC | high pressure liquid chromatography |
| Mc-OSu | maleimidocaproyl N-hydroxysuccimidyl ester |
| DMSO | dimethylsulfoxide;. |
| MMAE | mono-methyl auristatin E or N-methylvaline-valine- |
| | dolaisoleuine-dolaproine-phenylalanine |
| AEVB | auristatin E valeryl benzylhydrazone, acid labile linker |
| | through the C-terminus of AE |

### UPLC Analysis Method:

Method A: Mobile phase A: 0.100 FA in water, B: MeCN; Gradient: 10% B maintain 0.2 min, 1000-95% B, 5.8 min, 95% B maintain 0.5 min; Flow rate: 0.6 mL/min; Column: ACQUITY UPLC® BEH C18 1.7 m;

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm;

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm;

### Example 1-1 & 1-2

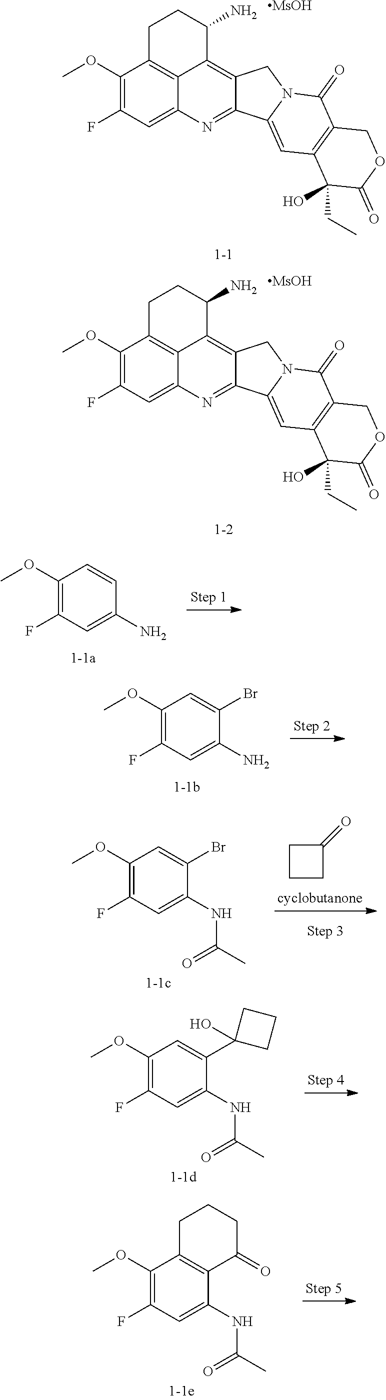

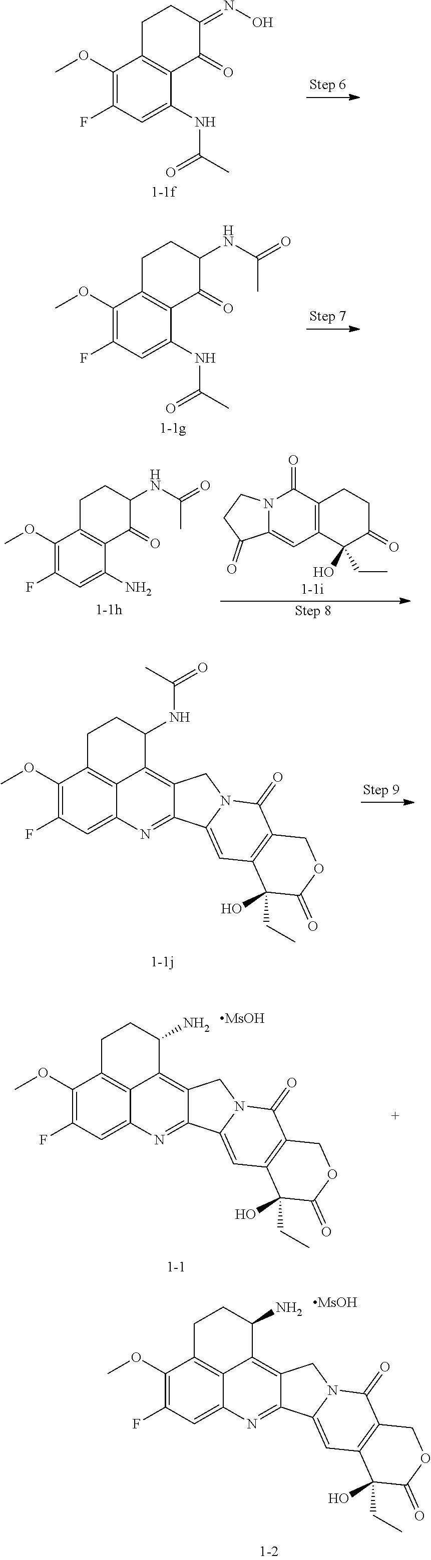

### Step 1

### 2-bromo-5-fluoro-4-methoxyaniline (1-1b)

To a solution of compound 1-1a (15 g, 106 mmol) in DCM (200 mL) was added K₂CO₃ (19.1 g, 138.16 mmol). The mixture was cooled to −15° C. and Br₂ (16.98 g, 106.27 mmol) was added dropwise into the mixture. The mixture was stirred at −15° C. for 30 min. The mixture was filtered and washed by sat. Na₂S₂O₃ (100 mL*3), brine (100 mL*3). The organic layer was concentrated to give the crude product 1-1b (21 g, crude) as brown solid.

MS (ESI) m/z: 221.9 [M+H]⁺

¹H NMR (400 MHz, CDCl₃) δ 7.03 (d, J=8.4 Hz, 1H), 6.56 (d, J=12.4 Hz, 1H), 3.89-3.85 (br s, 2H), 3.80 (s, 3H).

### Step 2

### N-(2-bromo-5-fluoro-4-methoxyphenyl)acetamide (1-1c)

To a solution of compound 1-1b (21 g, 95.4 mmol) in THF (200 mL) were added Ac₂O (19.5 g, 190.9 mmol), and Et₃N (19.5 g, 381.8 mmol). The mixture was stirred at RT for 16 h. The mixture was concentrated, the residue was triturated with MTBE (50 mL) to give compound 1-1c (20 g, crude).

MS (ESI) m/z: 263.9 [M+H]⁺

¹H NMR (400 MHz, CDCl₃) δ 8.15 (d, J=13.2 Hz, 1H), 7.49 (br s, 1H), 7.12 (d, J=8.4 Hz, 1H), 3.88 (s, 3H), 2.24 (s, 3H).

### Step 3

### N-(5-fluoro-2-(1-hydroxycyclobutyl)-4-methoxyphenyl)acetamide (1-1d)

A solution of compound 1-1c (14 g, 53.42 mmol) in THF (300 mL) was cooled to −78° C., nBuLi (53 mL, 133.55 mmol) was added into the mixture dropwise. The internal temperature was kept under −78° C. The mixture was stirred at −78° C. for 30 min. A solution of cyclobutanone (7.49 g, 106.84 mmol) in THF (50 mL) was added into the mixture dropwise. The mixture was stirred at −78° C. for 30 min and warmed to room temperature for 1 h. The mixture was quenched by sat. NH₄Cl solution (5 mL), diluted with EtOAc (150 mL), washed by brine (50 mL*3). The organic layer was dried and concentrated. The residue was purified by silica column gel chromatography (eluent: PE/EA=100/0 to 40/60) to afford 1-1d (2.7 g, 20.0% yield).

MS (ESI) m/z: 236.1 [M+H]⁺

¹H NMR (400 MHz, CDCl₃) δ 8.54 (s, 1H), 7.83 (d, J=12.8 Hz, 1H), 6.91 (d, J=8.8 Hz, 1H), 3.87 (s, 3H), 2.59-2.52 (m, 2H), 2.40-2.33 (m, 2H), 2.13 (s, 3H), 2.11-2.04 (m, 1H), 1.72-1.65 (m, 1H).

### Step 4

### N-(3-fluoro-4-methoxy-8-oxo-5,6,7,8-tetrahydronaphthalen-1-yl)acetamide (1-1e)

To a solution of compound 1-1d (2.7 g, 10.66 mmol) in DCM (80 mL) and H₂O (80 mL) were added AgNO₃ (3.2 mL, 3.198 mmol), K₂S₂O₈ (8.64 g, 31.98 mmol). The mixture was stirred at r.t. for 16 h. The mixture was diluted with EtOAc (300 mL), washed by brine (100 mL*3). The organic layer was dried, concentrated. The organic layer was dried and concentrated. The residue was purified by silica column gel chromatography (eluent: PE/EtOAc=100/0 to 40/60) to afford 1-1e (1.55 g, 57.8% yield) was obtained as light-yellow solid.

MS (ESI) m/z: 252.1 [M+H]⁺

¹H NMR (400 MHz, CDCl₃) δ 12.19 (s, 1H), 8.50 (d, J=14.8 Hz, 1H), 3.87 (s, 3H), 2.98 (t, J=6.0 Hz, 2H), 2.66 (t, J=6.4 Hz, 2H), 2.22 (s, 3H), 2.09-2.03 (m, 2H).

### Step 5

### (Z)—N-(3-fluoro-7-(hydroxyimino)-4-methoxy-8-oxo-5,6,7,8-tetrahydronaphthalen-1-yl)acetamide (1-1f)

To a solution of compound 1-1e (1.5 g, 5.97 mmol) in THF (20 mL) were added t-BuOK (7.76 mL, 7.76 mmol, 1 N) dropwise at 0° C., followed by isopentyl nitrite (699 mg, 5.97 mmol). The mixture was stirred at r.t. for 2 h. The mixture was adjusted to pH=7, diluted with EtOAc (100 mL), washed by brine (30 mL*3). The organic layer was dried over anhydrous Na₂SO₄, filtered and concentrated to give the residue. The residue was purified by a silica gel column chromatography (eluent: PE/EtOAc=100/0 to 40/60) to give the title compound 1-1f (1.1 g, 65.9% yield) as off-white solid.

MS (ESI) m/z: 281.1 [M+H]⁺

¹H NMR (400 MHz, CDCl₃) δ 11.93 (s, 1H), 8.53 (d, J=14.4 Hz, 1H), 3.90 (s, 3H), 3.11-3.06 (m, 4H), 2.25 (s, 3H).

### Step 6

### N,N′-(3-fluoro-4-methoxy-8-oxo-5,6,7,8-tetrahydronaphthalene-1,7-diyl)diacetamide (1-1g)

To a solution of compound 1-1f (1.1 g, 3.92 mmol) in Ac₂O (4 mL) and THF (20 mL) was added PtO₂ (80 mg). The mixture was stirred at r.t. under H₂ (15 psi) for 7 h. The mixture was filtered through a pad of celite, concentrated and diluted with EtOAc (150 mL), washed by sat. NaHCO₃ (50 mL*3). The organic layer was dried and concentrated. The residue was purified by a silica gel column chromatography (eluent: PE/EtOAc=100/0 to 40/60) to give the title compound 1-1g (675 mg, 55.8% yield) as off-white solid.

MS (ESI) m/z: 309.2 [M+H]⁺

### Step 7

### N-(8-amino-6-fluoro-5-methoxy-1-oxo-1,2,3,4-tetrahydronaphthalen-2-yl)acetamide (1-1h)

To a solution of compound 1-1g (675 mg, 2.189 mmol) in MeOH (10 mL) was added HCl (2 N, 10 mL). The mixture was stirred at 60° C. for 5 h. The mixture was cooled to r.t. and adjusted to pH=7 by sat. NaHCO₃, extracted by EtOAc (50 mL*3). The organic layer was dried and concentrated. The residue was purified by a silica gel column chromatography (eluent: PE/EtOAc=100/0 to 60/40) to give the title compound 1-1h (370 mg, 63.5% yield) as off-white solid.

MS (ESI) m/z: 267.1 [M+H]⁺

¹H NMR (400 MHz, CDCl₃) δ 6.57 (s, 1H), 6.31 (br s, 2H), 6.25 (d, J=12.8 Hz, 1H), 4.53-4.47 (m, 1H), 3.78 (s, 3H), 3.26-3.20 (m, 1H), 2.91-2.82 (m, 1H), 2.75-2.69 (s, 1H), 2.09 (s, 3H), 1.77-1.67 (m, 1H).

### Step 8

### N-((9S)-9-ethyl-5-fluoro-9-hydroxy-4-methoxy-10,13-dioxo-2,3,9,10,13,15-hexahydro-1H,12H-benzo[de]pyrano[3′,4′:6,7]indolizino[1,2-b]quinolin-1-yl)acetamide (1-1j)

To a solution of compound 1-1h (200 mg, 0.75 mmol) in toluene (40 mL) and o-cresol (0.5 mL) were added 1-1i (208 mg, 0.79 mmol) and PPTS (38 mg, 0.16 mmol). The mixture was refluxed at 140° C. for 48 h. The mixture was concentrated and purified by a silica gel column chromatography (eluent: CH₂Cl₂/MeOH=100/0 to 20/80) to give the title compound 1-1j (350 mg, 94.3% yield, two isomers) was obtained as off-white solid.

MS (ESI) m/z: 494.3 [M+H]⁺

### Step 9

### (1S,9S)-1-amino-9-ethyl-5-fluoro-9-hydroxy-4-methoxy-1,2,3,9,12,15-hexahydro-10H,13H-benzo[de]pyrano[3′,4′:6,7]indolizino[1,2-b]quinoline-10,13-dione methanesulfonate (1-1)

### (1R,9S)-1-amino-9-ethyl-5-fluoro-9-hydroxy-4-methoxy-1,2,3,9,12,15-hexahydro-10H,13H-benzo[de]pyrano[3′,4′:6,7]indolizino[1,2-b]quinoline-10,13-dione methanesulfonate (1-2)

A solution of compound 1-1j (350 mg, 0.709 mmol) in MsOH (10 mL) and H₂O (10 mL) was refluxed at 110° C. for 8 h. The mixture was filtered and purified by prep-HPLC (Method: column: XBridge Prep C18 OBD 5 um 19*150 mm; Mobile phase: A-water (0.1% TFA): B-acetonitrile; Flow rate: 20 mL/min, the fraction was lyophilized to give two isomers:

Compound 1-1 (37 mg, 9.5% yield) was obtained as yellow solid.

MS (ESI) m/z: 452.2 [M+H]⁺. Retention time (1.67 min).

Compound 1-2 (36 mg, 9.3% yield) was obtained as yellow solid.

MS (ESI) m/z: 452.2 [M+H]⁺. Retention time (2.55 min).

### Example 1-3&1-4

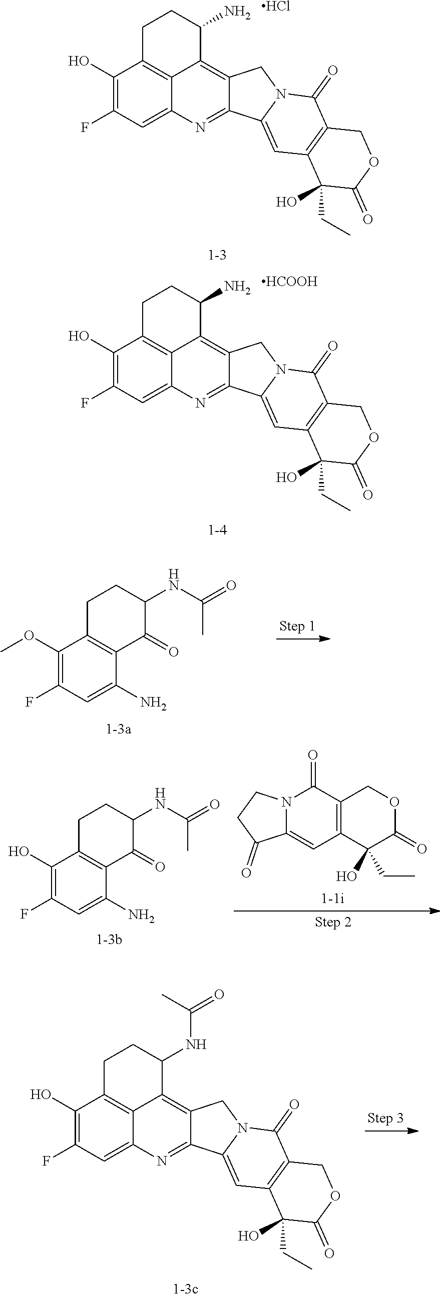

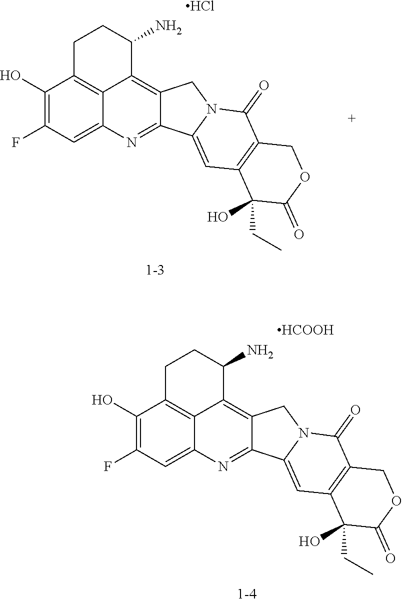 [SMILES: Cl ; CC[C@@]1(O)C(=O)OCc2c1cc1n(c2=O)Cc2c-1nc1cc(F)c(O)c3c1c2[C@@H](N)CC3 ; "·HCOOH" ; CC[C@@]1(O)C(=O)OCc2c1cc1n(c2=O)Cc2c-1nc1cc(F)c(O)c3c1c2[C@H](N)CC3]

### Step 1

### N-(8-amino-6-fluoro-5-hydroxy-1-oxo-1,2,3,4-tetrahydronaphthalen-2-yl)acetamide (1-3b)

To a solution of compound 1-3a (173 mg, 0.64 mmol) in DCM (6 mL) was added BBr₃ (1.92 mL, 1.93 mmol, 1N) dropwise at 0° C. The mixture was stirred at r.t. for 3 h.

The mixture was quenched with MeOH (2 mL) at 0° C. The mixture was concentrated and purified by a silica gel column chromatography (eluent: CH₂Cl₂/MeOH=100/0 to 20/80) to give the title compound 1-3b (151 mg, 92.1% yield) was obtained as off-white solid.

MS (ESI) m/z: 253.1 [M+H]⁺

### Step 2

### N-((9S)-9-ethyl-5-fluoro-4,9-dihydroxy-10,13-dioxo-2,3,9,10,13,15-hexahydro-1H,12H-benzo[de]pyrano[3′,4′:6,7]indolizino[1,2-b]quinolin-1-yl)acetamide (1-3c)

Compound 1-3c (168 mg, 58.1% yield, two isomers) was synthesized according to synthetic procedure of step 8 of compound 1-1.

MS (ESI) m/z: 480.2 [M+H]⁺

### Step 3

### (1S,9S)-1-amino-9-ethyl-5-fluoro-4,9-dihydroxy-1,2,3,9,12,15-hexahydro-10H,13H-benzo[de]pyrano[3′,4′:6,7]indolizino[1,2-b]quinoline-10,13-dione methanesulfonate (1-3)

### (1R,9S)-1-amino-9-ethyl-5-fluoro-4,9-dihydroxy-1,2,3,9,12,15-hexahydro-10H,13H-benzo[de]pyrano[3′,4′:6,7]indolizino[1,2-b]quinoline-10,13-dione methanesulfonate (1-4)

A solution of compound 1-3c (150 mg, 0.31 mmol) in con. HCl (20 mL) was refluxed at 110° C. for 8 h. The mixture was filtered and purified by prep-HPLC (Method: column: XBridge Prep C18 OBD 5 um 19*150 mm; Mobile phase: A-water (0.1% formic acid): B-acetonitrile; Flow rate: 20 mL/min, the fraction was lyophilized to give two isomers:

Compound 1-3 (22.7 mg, 15.3% yield) was obtained as yellow solid.

MS (ESI) m/z: 438.3 [M+H]⁺. Retention time (1.48 min).

Compound 1-4 (22.6 mg, 14.9% yield) was obtained as yellow solid.

MS (ESI) m/z: 438.3 [M+H]⁺. Retention time (1.78 min).

### Example 1-5

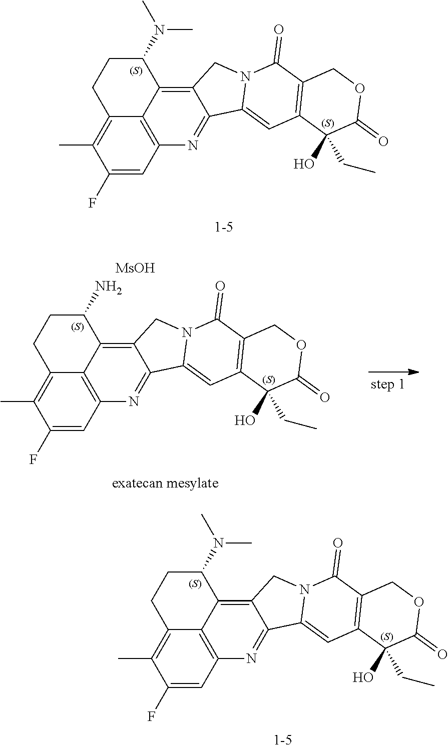

### Step 1

### (1S,9S)-1-(dimethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-5)

To the solution of exatecan mesylate (50 mg, 0.09 mmol) in formic acid (2 mL) was added 38% formaldehyde solution (1 mL), stirred at 50° C. for 18 h. The reaction solution was purified by prep-HPLC (Mobile phase A: 0.1% FA in water, B: MeCN; Gradient: 20%-28% B; Flow rate: 20 mL/min; Column: Xbridge Prep C18 OBD™ 5 μm, 19*150 mm). The desired fraction was lyophilized to give 1-5 (15.4 mg, 35.3% yield) as a pale yellow solid.

MS (ESI) m/z: 464.4 [M+H]⁺

¹H NMR (400 MHz, DMSO-d6) δ 7.70 (d, J=10.9 Hz, 1H), 7.30 (s, 1H), 6.51 (s, 1H), 5.42 (s, 2H), 5.30-5.13 (m, 2H), 4.11-4.22 (m, 1H), 3.33-3.27 (m, 1H), 2.95-2.81 (m, 1H), 2.42-2.22 (m, 9H), 2.00-1.75 (m, 3H), 0.88 (t, J=7.3 Hz, 3H).

### Example 1-6

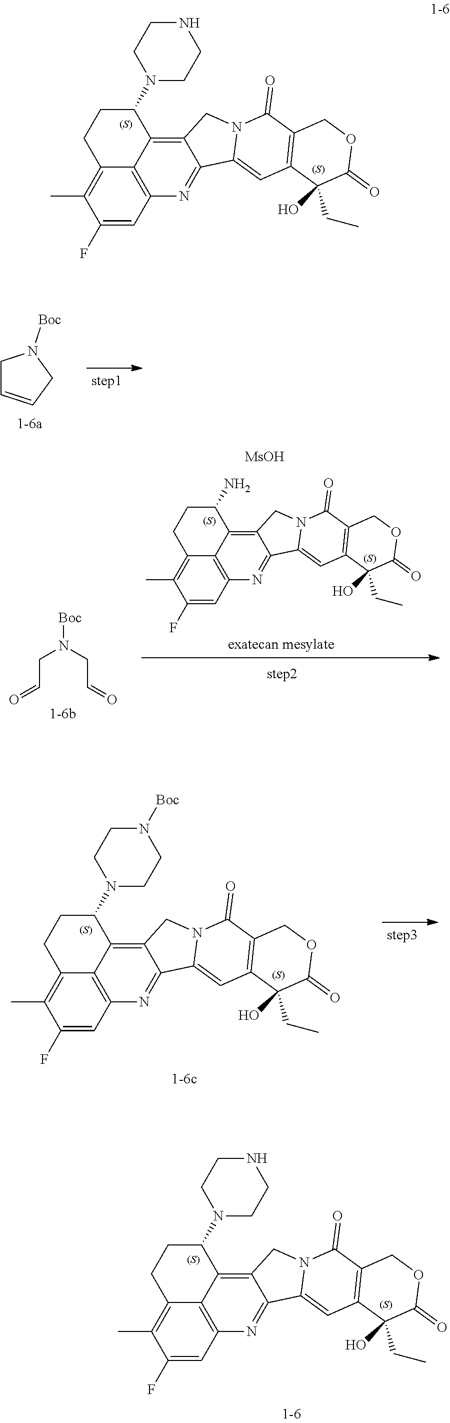

### Step 1

### tert-butyl bis(2-oxoethyl)carbamate (1-6b)

To the solution of 1-6a (500 mg, 2.95 mmol) in t-BuOH/THF/H₂O (5/10/5 mL) were added the solution of potassium osmate(VI) dihydrate (55 mg, 0.15 mmol) and the solution of NaIO₄ (1.6 g, 7.39 mmol) in water (5 mL), stirred at r.t. for 20 h. The reaction solution was extracted with CH₂Cl₂ (15 mL*3). The organic phase was washed with brine (15 mL) and dried over anhydrous Na₂SO₄, concentrated to give compound 1-6b (396 mg, 66.6% yield) as a yellow oil.

¹H NMR (400 MHz, CDCl₃) δ 9.67 (s, 1H), 9.65 (s, 1H), 4.18 (s, 2H), 3.96 (s, 2H), 1.45 (s, 9H).

### Step 2

### tert-butyl 4-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)piperazine-1-carboxylate (1-6c)

To the solution of exatecan mesylate (100 mg, 0.19 mmol), NaBH₃CN (12 mg, 0.19 mmol) and NaHCO₃ (16 mg, 0.19 mmol) in MeOH (1.5 mL) were added the solution of 1-6b (38 mg, 0.19 mmol) and HOAc (11 μL, 0.19 mmol) in MeOH (0.5 mL), stirred at r.t. for 23 h. 1-6b (16 mg, 0.08 mmol) and NaBH₃CN (10 mg, 0.16 mmol) were added, stirred for another 40 min. 1-6b (10 mg, 0.05 mmol) and NaBH₃CN (10 mg, 0.16 mmol) were added, stirred for another 20 min. The reaction solution was added into water (10 mL), extracted with CH₂Cl₂/MeOH (10/1, 5.5 mL*4). The organic phase was concentrated and purified by flash column chromatography (silica gel, CH₂Cl₂/MeOH=100/0 to 91/9) to give compound 1-6c (76 mg, 66.8% yield) as a yellow solid.

MS (ESI) m/z: 627.5 [M+Na]⁺

### Step 3

### (1S,9S)-9-ethyl-5-fluoro-9-hydroxy-4-methyl-1-(piperazin-1-yl)-1,2,3,9,12,15-hexahydro-10H,13H-benzo[de]pyrano[3′,4′:6,7]indolizino[1,2-b]quinoline-10,13-dione (1-6) formate salt

To the solution of 1-6c (51 mg, 0.08 mmol) in CH₂Cl₂ (2 mL) was added TFA (0.4 mL) at 0° C., stirred at r.t. for 2 h. The reaction solution was concentrated and purified by prep-HPLC (Mobile phase A: 0.1% FA in water, B: MeCN; Gradient: 24% B hold; Flow rate: 20 mL/min; Column: Xbridge Prep C18 OBD™ 5 μm, 19*150 mm). The desired fraction was lyophilized to give 1-6 formate salt (21 mg, 50.3% yield) as a yellow solid.

MS (ESI) m/z: 505.4 [M+H]⁺

¹H NMR (400 MHz, DMSO-d6) δ 8.31 (s, 1H), 7.73 (d, J=10.9 Hz, 1H), 7.30 (s, 1H), 5.42 (s, 2H), 5.40 (d, J=19.6 Hz, 1H), 5.30 (d, J=19.4 Hz, 1H), 4.27-4.16 (m, 1H), 3.37-3.22 (m, 2H), 2.99-2.76 (m, 5H), 2.72-2.56 (m, 4H), 2.36 (s, 3H), 2.30-2.18 (m, 1H), 2.16-2.02 (m, 1H), 1.95-1.78 (m, 2H), 0.88 (t, J=7.3 Hz, 3H).

### Example 1-7

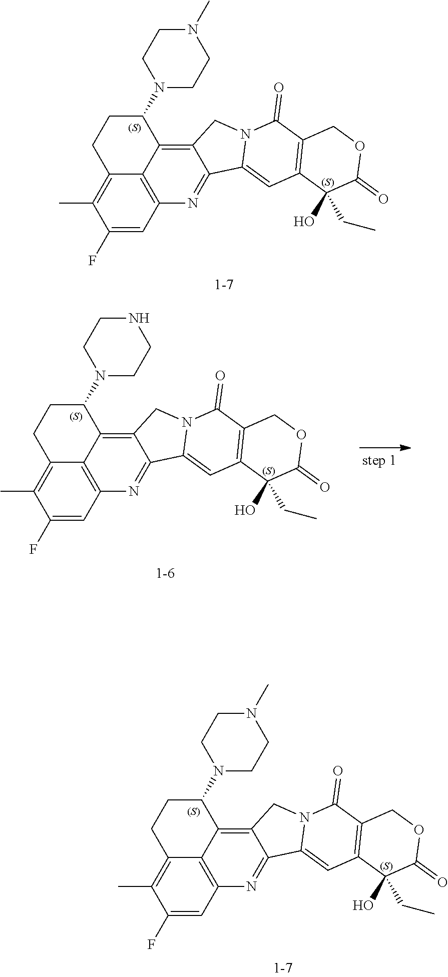

### Step 1

### (1S,9S)-9-ethyl-5-fluoro-9-hydroxy-4-methyl-1-(4-methylpiperazin-1-yl)-1,2,3,9,12,15-hexahydro-10H,13H-benzo[de]pyrano[3′,4′:6,7]indolizino[1,2-b]quinoline-10,13-dione (1-7)

To the solution of 1-6 (TFA salt, 64 mg, 0.13 mmol), NaHCO₃ (11 mg, 0.13 mmol) and NaBH₃CN (12 mg, 0.19 mmol) in CH₂Cl₂/MeOH (2/0.5 mL) was added 37% formaldehyde solution (12 L, 0.14 mmol), stirred at r.t. for 30 min. The solution was added into water (5 mL), extracted with DCM/MeOH (10/1, 5.5 mL*3). The organic phase was concentrated and purified by prep-HPLC (Mobile phase A: 0.1% FA in water, B: MeCN; Gradient: 25% B hold; Flow rate: 20 mL/min; Column: Xbridge Prep C18 OBD™ 5 m, 19*150 mm). The desired fraction was lyophilized to give 1-7 formate salt (16 mg, 24.3% yield) as a beige solid.

MS (ESI) m/z: 519.5 [M+H]⁺

¹H NMR (400 MHz, DMSO-d6) δ 8.31 (s, 1H), 7.72 (d, J=10.7 Hz, 1H), 7.29 (s, 1H), 6.50 (s, 1H), 5.42 (s, 3H), 5.40 (d, J=19.8 Hz, 1H), 5.26 (d, J=19.8 Hz, 1H), 4.32-4.17 (m, 1H), 3.29-3.17 (m, 2H), 2.97-2.82 (m, 1H), 2.74-2.55 (m, 4H), 2.44-2.29 (m, 6H), 2.29-2.22 (m, 1H), 2.21-2.13 (s, 3H), 2.13-1.97 (m, 1H), 1.95-1.77 (m, 2H), 0.88 (t, J=7.1 Hz, 3H).

### Example 1-8 & 1-9

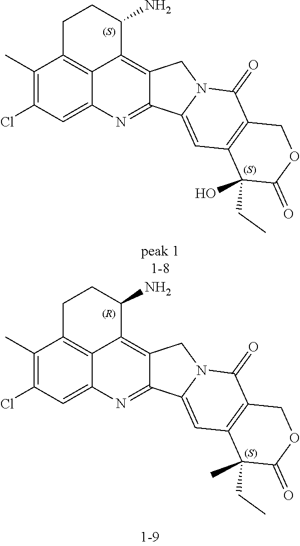

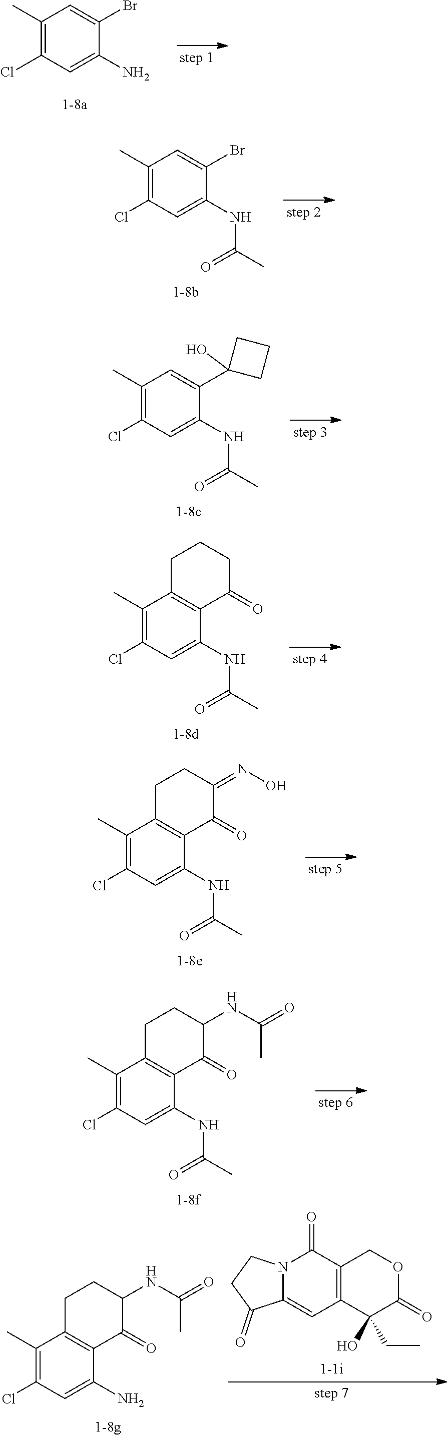

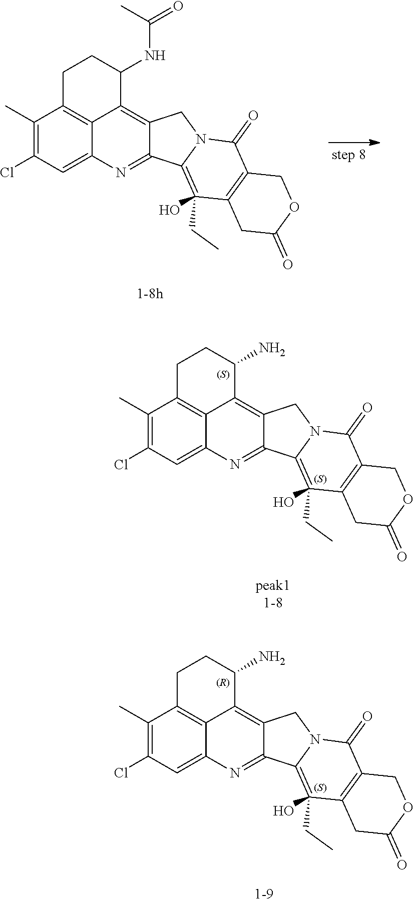

### Step 1

### N-(2-bromo-5-chloro-4-methylphenyl)acetamide (1-8b)

To the solution of 1-8a (12.7 g, 57.5 mmol) and DMAP (355 mg, 2.9 mmol) in EtOAc/DCM (100/50 mL) was added Ac₂O (7.1 mL, 74.7 mmol), stirred at r.t. for 6 h. The turbid solution was concentrated and in slurry with petroleum ether/ethyl acetate (3:1, v/v, 16 mL), filtered to give compound 1-8b (11.6 g, 76.9% yield) as a white solid.

MS (ESI) m/z: 261.9 [M+H]⁺

¹H NMR (400 MHz, CDCl₃) δ 8.40 (s, 1H), 7.49 (s, 1H), 7.39 (s, 1H), 2.31 (s, 3H), 2.23 (s, 3H).

### Step 2

### N-(5-chloro-2-(1-hydroxycyclobutyl)-4-methylphenyl)acetamide (1-8c)

Compound 1-8c (5.6 g, 55% yield) was obtained according to the procedure described in CN111470998B with a fine modification: The crude product was in slurry with MTBE and then filtered to give compound 1-8c.

MS (ESI) m/z: 236.1 [M+H-H₂O]⁺

¹H NMR (400 MHz, CDCl₃) δ 8.59 (s, 1H), 8.12 (s, 1H), 7.14 (s, 1H), 2.64-2.53 (m, 2H), 2.50 (s, 1H), 2.42-2.28 (m, 5H), 2.14 (s, 3H), 2.11-2.00 (m, 1H), 1.73-1.63 (m, 1H).

### Step 3

### N-(3-chloro-4-methyl-8-oxo-5,6,7,8-tetrahydronaphthalen-1-yl)acetamide (1-8d)

Compound 1-8d (4.1 g, 85% purity, 63% yield) was obtained according to the procedure described in CN111470998B.

MS (ESI) m/z: 252.0 [M+H]⁺

¹H NMR (400 MHz, CDCl₃) δ 12.14 (s, 1H), 8.76 (s, 1H), 2.91 (t, J=6.2 Hz, 2H), 2.68-2.60 (m, 2H), 2.32 (s, 3H), 2.22 (s, 3H), 2.16-2.01 (m, 2H).

### Step 4

### (Z)—N-(3-chloro-7-(hydroxyimino)-4-methyl-8-oxo-5,6,7,8-tetrahydronaphthalen-1-yl)acetamide (1-8e)

Compound 1-8e (3.9 g, 84% yield) was obtained according to the procedure described in CN111470998B.

MS (ESI) m/z: 281.1 [M+H]⁺

### Step 5

### N,N′-(3-chloro-4-methyl-8-oxo-5,6,7,8-tetrahydronaphthalene-1,7-diyl)diacetamide (1-8f)

To the solution of 1-8e (3.9 g, 13.9 mmol) in EtOAc/Ac₂O (100/25 mL) was added PtO₂ (322 mg, 1.4 mmol), stirred at H₂ atmosphere for 16 h. The solution was filtered and concentrated to give compound 1-8f (4.15 g, crude) which was used for the next step without further purification.

MS (ESI) m/z: 309.1 [M+H]⁺

### Step 6

### N-(8-amino-6-chloro-5-methyl-1-oxo-1,2,3,4-tetrahydronaphthalen-2-yl)acetamide (1-8g)

To the solution of 1-8f (2.95 g, 9.55 mmol) in MeOH (25 mL) was added 2N HCl (25 mL) under nitrogen atmosphere, stirred at 60° C. for 2 h. The pH of the reaction solution was adjusted to 7 with Na₂CO₃ solid and sat. NaHCO₃ solution. The slurry solution was filtered and the filter cake was purified by flash column chromatography (silica gel, petroleum ether/ethyl acetate=1:3) to give compound 1-8g (750 mg, 29.4% yield) as a light grey solid.

MS (ESI) m/z: 267.1 [M+H]⁺

¹H NMR (400 MHz, DMSO) δ 8.08 (d, J=7.9 Hz, 1H), 7.30 (s, 2H), 6.76 (s, 1H), 4.48 (ddd, J=12.9, 7.9, 4.7 Hz, 1H), 2.99-2.79 (m, 2H), 2.18-2.08 (m, 4H), 1.95-1.80 (m, 4H).

### Step 7

### N-((9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)acetamide (1-8i)

Compound 1-8i (549 mg, 97.2% yield) was obtained according to the procedure described in EP3677568A1.

MS (ESI) m/z: 494.3 [M+H]⁺

¹H NMR (400 MHz, DMSO-d6) δ 8.45 (d, J=8.7 Hz, 1H), 8.16 (s, 1H), 7.31 (s, 1H), 6.53 (s, 1H), 5.61-5.49 (m, 1H), 5.43 (s, 2H), 5.30-5.14 (m, 2H), 3.25-3.13 (m, 2H), 2.52 (s, 3H), 2.18-2.08 (m, 2H), 1.94-1.80 (m, 5H), 0.87 (t, J=7.3 Hz, 3H).

### Step 8

### (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinoline-10,13-dione (1-8)

### (1R,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinoline-10,13-dione (1-9)

To the slurry solution of 1-8i (200 mg, 0.41 mmol) in H₂O (3 mL) was added MsOH (1.5 mL) under nitrogen atmosphere, stirred at 100° C. for 2 h and 90° C. for 14 h. Compound 1-8 (8 mg) was obtained according to the procedure described in CN111470998B. The filtrate was purified by prep-HPLC (Mobile phase A: 0.1% FA in water, B: MeCN; Gradient: 10%-20%-30% B; Flow rate: 20 mL/min; Column: Sunfire Prep C18 OBD™ 5 μm, 19*150 mm). The desired fraction was lyophilized to give 1-8 mesylate salt (38.5 mg, 17.4% yield) as a beige solid and 1-9 formate salt (13 mg, 5.9% yield) as a beige solid.

MS (ESI) m/z: 494.3 [M+H]⁺

UPLC analysis: 1-8, retention time=2.62 min; 1-9, retention time=3.05 min (Mobile phase A: 0.1% FA in water, B: MeCN; Gradient: 15% B maintain 1 min, 15%-95% B, 9 min, 95% B maintain 2 min; Flow rate: 0.6 mL/min; Column: ACQUITY UPLC® BEH C18 1.7 μm)

1-8 (mesylate salt): ¹H NMR (400 MHz, DMSO) δ 8.24 (brs, 3H), 7.35 (s, 1H), 6.57 (s, 1H), 5.70 (d, J=19.4 Hz, 1H), 5.46 (s, 2H), 5.43 (d, J=19.4 Hz, 1H), 5.09-4.98 (m, 1H), 3.19-3.08 (m, 1H), 2.98-2.87 (m, 2H), 2.55 (s, 3H), 2.30 (s, 3H), 2.25-2.12 (m, 1H), 1.96-1.79 (m, 2H), 0.88 (t, J=7.3 Hz, 3H).

1-9 (formate salt): ¹H NMR (400 MHz, DMSO) δ 8.14 (s, 1H), 7.32 (s, 1H), 6.52 (s, 1H), 5.64 (d, J=19.3 Hz, 1H), 5.44 (s, 2H), 5.37 (d, J=19.3 Hz, 1H), 4.59-4.45 (m, 1H), 3.27-3.18 (m, 1H), 3.17-3.06 (m, 1H), 2.21-2.07 (m, 2H), 1.93-1.79 (m, 2H), 0.88 (t, J=7.3 Hz, 3H).

### Example 1-10 and 1-11

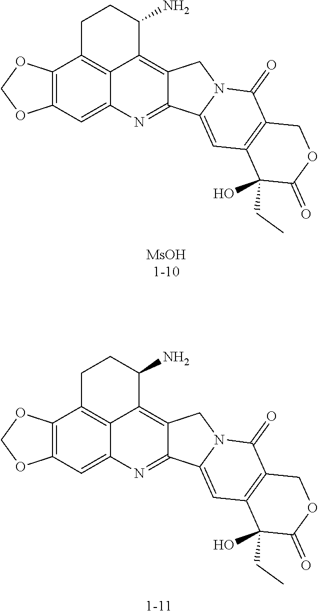 [SMILES: CC[C@@]1(O)C(=O)OCc2c1cc1n(c2=O)Cc2c-1nc1cc3c(c4c1c2[C@@H](N)CC4)OCO3 ; CC[C@@]1(O)C(=O)OCc2c1cc1n(c2=O)Cc2c-1nc1cc3c(c4c1c2[C@H](N)CC4)OCO3]

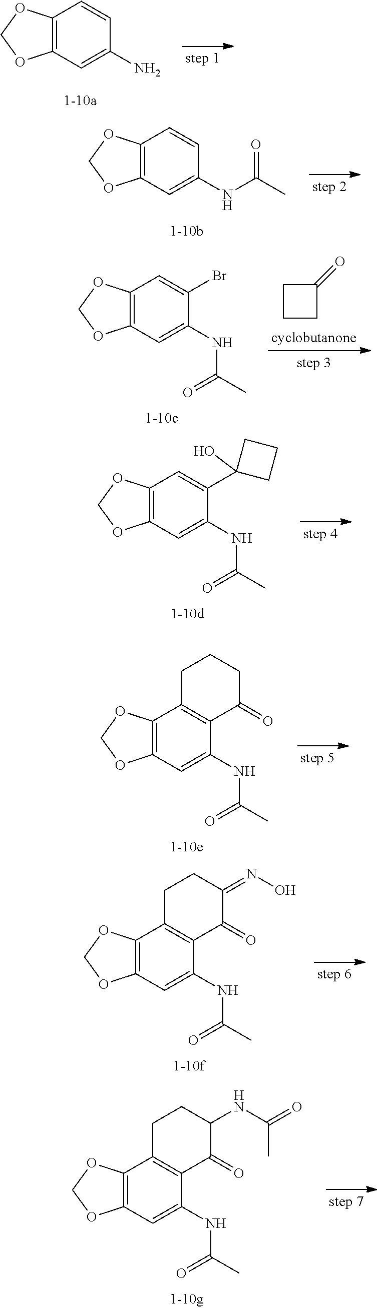

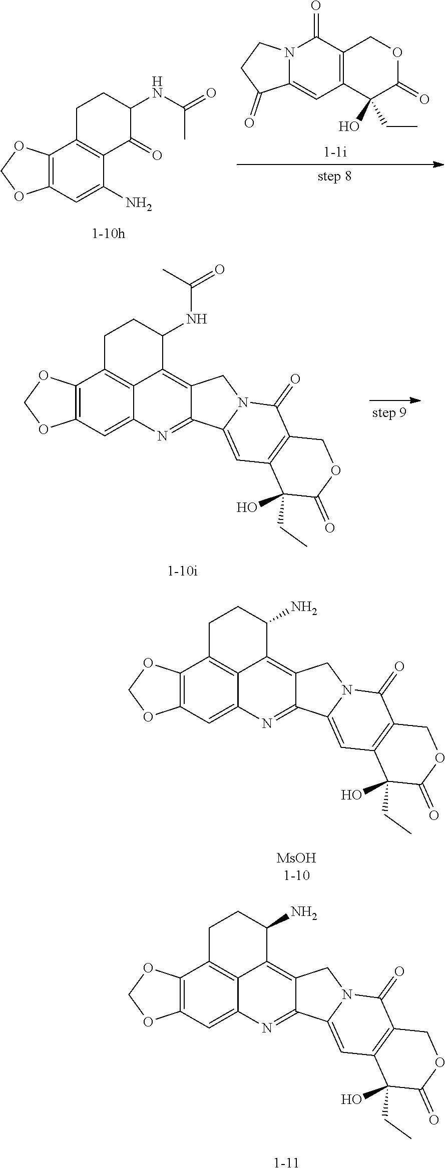

### Step 1

### N-(benzo[d][1,3]dioxol-5-yl)acetamide (1-10b)

To a solution of 1-10a (25.00 g, 182 mmol) in CH₂Cl₂ (200 mL) was added Ac₂O (27.85 g. 273 mmol) and Et₃N (36.76 g, 364 mmol) at 0° C. The reaction mixture was reacted at 20° C. for 3 h. LCMS showed the reaction was completed. The reaction mixture was concentrated to 50 mL. To the residual was added EtOAc (200 mL) and washed with saturated NaHCO₃ (200 mL*3). The organic layer was dried over Na₂SO₄ and filtered. The filtrate was concentrated under vacuum and the residue was used in the next step without further workup and purification (28.25 g, crude).

MS (ESI) m/z: 180.1 [M+H]⁺

### Step 2

### N-(6-bromobenzo[d][1,3]dioxol-5-yl)acetamide (1-10c)

A solution of 1-10b (28.25 g, crude) and sodium acetate (15.4 g, 188.3 mmol) in acetic acid (100 mL) was heated to 60° C., then a mixture of bromine (30.1 g, 188.3 mmol) and acetic acid (60 mL) were added to the reaction solution dropwise. The reaction temperature was raised to 80° C. and stirred at 80° C. for 2 h. LCMS showed the reaction was completed, the reaction solution was poured into ice water, and a yellow solid was generated. The solid was filtered and washed with water three times to obtain a crude product which was recrystallized with EtOH to afford 1-10c (17.1 g, 42% yield).

MS (ESI) m/z: 258.1/260.1 [M+H]⁺

1H NMR (400 MHz, DMSO) δ 9.36 (s, 1H), 7.22 (s, 1H), 7.08 (d, J=3.7 Hz, 1H), 6.07 (s, 2H), 2.02 (s, 3H).

### Step 3

### N-(6-(1-hydroxycyclobutyl)benzo[d][1,3]dioxol-5-yl)acetamide (1-10d)

A solution of compound 1-10c (8.5 g, 33.01 mmol) in THF (200 mL) was cooled to −90° C., n-BuLi (15.84 mL, 39.60 mmol) was added into the mixture over 2 h under N₂ protection. The internal temperature was kept under −85° C. The mixture was stirred at −85° C. for 20 min. A solution of cyclobutanone (2.7 g, 39.60 mmol) in THF (50 mL) was added into the mixture dropwise. The mixture was stirred at −85° C. for 30 min and warmed to room temperature for 1 h. The mixture was quenched by sat. NH₄Cl solution (200 mL), extracted with EtOAc (150 mL*3). The combined organic layer was dried over Na₂SO₄. After filtration and evaporation, the residue was washed with MTBE (5 mL*3) and recrystallized with EtOH to afford 1-10d (3.1 g, 37.5% yield).

MS (ESI) m/z: 250.1 [M+H]⁺

### Step 4

### N-(6-oxo-6,7,8,9-tetrahydronaphtho[1,2-d][1,3]dioxol-5-yl)acetamide (1-10e)

To a solution of compound 1-10d (3.1 g, 12.44 mmol) in CH₂Cl₂ (20 mL) and H₂O (20 mL) were added AgNO₃ (12.4 mL, 6.22 mmol), K₂S₂O₈ (8.4 g, 31.1 mmol). The mixture was stirred at 20° C. for 16 h. The mixture was filtered through celite and the filtration residue was washed with CH₂Cl₂:MeOH=1:1 (50 mL*3). The filtrate was poured into water and extracted with CH₂Cl₂ (300 mL*3). The combined organic layer was dried over Na₂SO₄. After filtration and evaporation, the residue was purified by silica column gel chromatography (eluent: Petroleum ether/CH₂Cl₂=100/0 to 0/100) to afford 1-10e (1.9 g, 61.8% yield) as light-yellow solid.

MS (ESI) m/z: 248.1 [M+H]⁺

### Step 5

### (Z)—N-(7-(hydroxyimino)-6-oxo-6,7,8,9-tetrahydronaphtho[1,2-d][1,3]dioxol-5-yl)acetamide (1-10f)

To a solution of compound 1-10e (1.9 g, 7.69 mmol) in THF (20 mL) and t-BuOH (5 mL) was added t-BuOK (1 M in THF, 9.28 mL, 9.28 mmol) dropwise at 0° C., The atmosphere was purged with N₂ and the mixture was cooled to 0° C. using and ice water bath. After 5 mins, to the stirred mixture was added isopentyl nitrite (1.09 g, 9.28 mmol). LCMS showed the reaction was completed and the reaction mixture was allowed to warm to r.t. 1 N HCl was added to adjusted pH to 1. The aqueous layer was extracted with CH₂Cl₂: THF (2:1, v/v, 50 mL*3). The combined organic layer was washed with brine (100 mL) and dried over Na₂SO₄. After filtration and evaporation, the residue was trituration with MTBE (20 mL*2) to afford 1-10f (1.5 g, 70.6% yield).

MS (ESI) m/z: 277.1 [M+H]⁺

### Step 6

### N,N′-(6-oxo-6,7,8,9-tetrahydronaphtho[1,2-d][1,3]dioxole-5,7-diyl)diacetamide (1-10g)

To a solution of compound 1-10f (1.5 g, 5.43 mmol) in Ac₂O (5 mL) was added PtO₂ (150 mg). The mixture was stirred at 20° C. under H₂ (15 psi) for 16 h. LCMS showed the reaction was completed. The mixture was filtered through a pad of celite. The filtrate was diluted with EtOAc (100 mL) and washed by sat. NaHCO₃ (100 mL*3). The organic layer was dried over Na₂SO₄. After filtration and evaporation, the residue 1-10g (1.3 g, crude) was used in the next step without further workup and purification.

MS (ESI) m/z: 305.2 [M+H]⁺

### Step 7

### N-(5-amino-6-oxo-6,7,8,9-tetrahydronaphtho[1,2-d][1,3]dioxol-7-yl)acetamide (1-10h)

To a solution of compound 1-10g (1.3 g, crude) in MeOH (10 mL) was added HCl (2 N, 10 mL). The mixture was stirred at 60° C. for 3 h. The mixture was cooled to r.t. and adjusted pH to 8 using sat. NaHCO₃. The mixture was extracted with EtOAc (50 mL*3). The organic layer was dried and concentrated. The residue was purified by a silica gel column chromatography (eluent: CH₂Cl₂MeOH=100/0 to 90/10) to give compound 1-10h (670 mg, 59.8% yield) as off-white solid.

MS (ESI) m/z: 263.1 [M+H]⁺

### Step 8

### N-((10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)acetamide (1-10i)

To a solution of compound 1-10h (400 mg, 1.52 mmol) in toluene (40 mL) and o-cresol (0.5 mL) was added 1-1i (479 mg, 1.82 mmol) and PPTS (38 mg, 0.15 mmol). The mixture was refluxed at 140° C. for 24 h. LCMS showed the reaction was completed and black solid precipitated. After filtration, the filter cake was washed with acetone (10 mL*3) to give a dark brown solid 1-10i (580 mg, crude) which was used in the next step without further workup and purification.

MS (ESI) m/z: 490.3 [M+H]⁺

### Step 9

### (1S,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione (1-10)

### (1R,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione (1-11)

A solution of compound 1-10i (580 mg, crude) in MsOH (10 mL) and H₂O (10 mL) was refluxed at 110° C. for 5 h. LCMS showed the reaction was completed. The mixture was filtered and purified by prep-HPLC (Method: column: XBridge Prep C18 OBD 5 um 19*150 mm; Mobile phase: A-water (0.1% TFA): B-acetonitrile; Flow rate: 20 mL/min, the fraction was lyophilized to give two isomers:

Compound 1-10 (110 mg, 20.8% yield) was obtained as yellow solid.

MS (ESI) m/z: 448.2 [M+H]⁺. Retention time (0.82 min).

Compound 1-11 (120 mg, 22.7% yield) was obtained as yellow solid.

MS (ESI) m/z: 448.2 [M+H]⁺. Retention time (1.87 min).

### Example 1-12&1-13

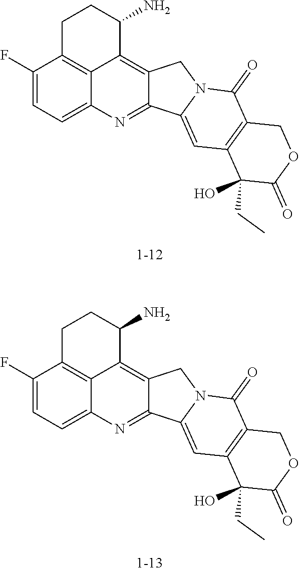 [SMILES: CC[C@@]1(O)C(=O)OCc2c1cc1n(c2=O)Cc2c-1nc1ccc(F)c3c1c2[C@@H](N)CC3 ; CC[C@@]1(O)C(=O)OCc2c1cc1n(c2=O)Cc2c-1nc1ccc(F)c3c1c2[C@H](N)CC3]

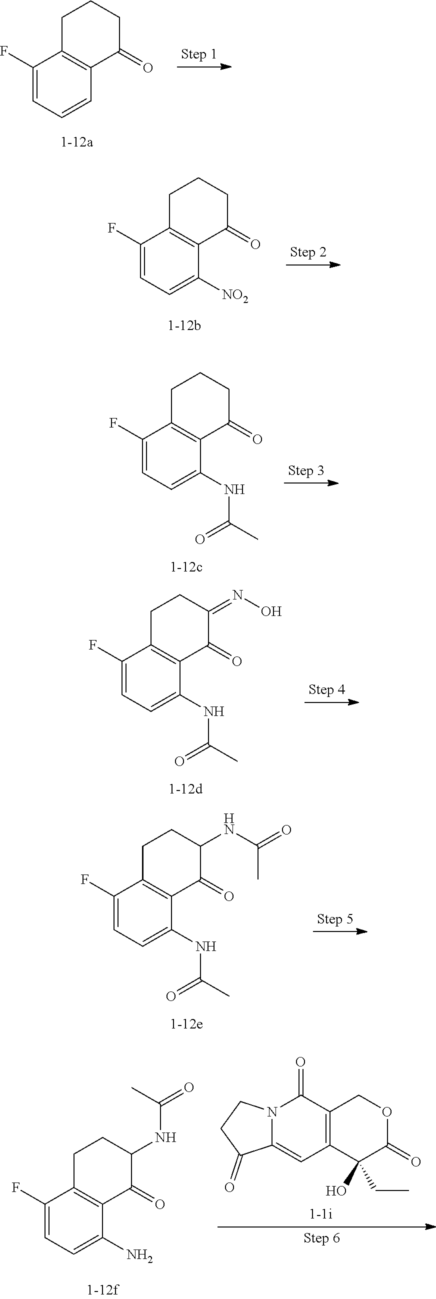

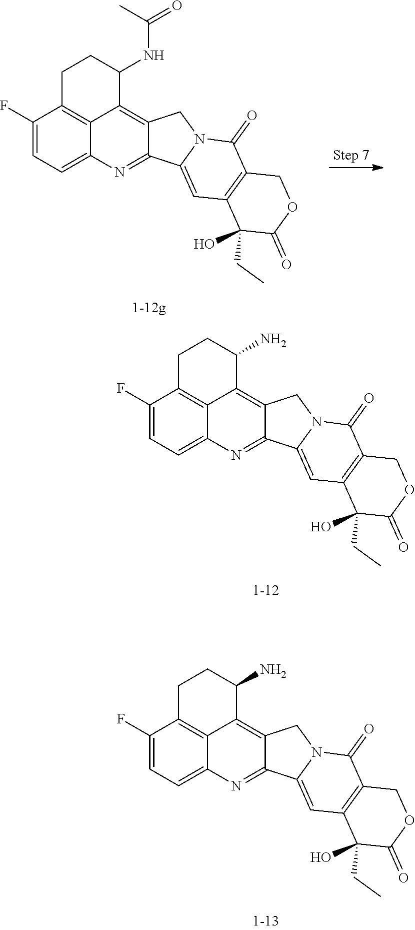 [SMILES: CC[C@@]1(O)C(=O)OCc2c1cc1n(c2=O)Cc2c-1nc1ccc(F)c3c1c2C(NC(C)=O)CC3 ; CC[C@@]1(O)C(=O)OCc2c1cc1n(c2=O)Cc2c-1nc1ccc(F)c3c1c2[C@@H](N)CC3 ; CC[C@@]1(O)C(=O)OCc2c1cc1n(c2=O)Cc2c-1nc1ccc(F)c3c1c2[C@H](N)CC3]

### Step 1

### 5-fluoro-8-nitro-3,4-dihydronaphthalen-1 (2H)-one (1-12b)

1-12a (1.00 g, 6.10 mmol) was added to a stirred suspension of Cu(NO₃)₂ (1.14 g, 6.10 mmol) in H₂SO₄ (20 mL). The reaction mixture was stirred at 0° C. -r.t for 3 h. The mixture was added to H₂O at 0° C. with stirred. Then the filter residue was extracted with EtOAc (30 mL*3). The combined organics were washed with H₂O (30 mL), brine (50 mL), dried over Na₂SO₄ and concentrated. Column chromatography on silica gel (EtOAc/petroleum ether=1/5) to afford 1-12b (320.00 mg, 25.11% yield) as a yellow solid.

MS (ESI) m/z: 210.1 [M+H]⁺

### Step 2

### N-(4-fluoro-8-oxo-5,6,7,8-tetrahydronaphthalen-1-yl)acetamide (1-12c)

Wet PtO₂ (40 mg) was added to a mixture of 1-12b (0.32 g, 1.53 mmol) in Ac₂O (10 mL). The reaction mixture was purged with H₂ balloon for three times and reacted at r.t. under H₂ balloon for 16 h. After the reaction was completed (checked by LCMS), the mixture was filtered off through celite and washed with MeOH. The filtrate was concentrated under vacuum and purified by silica gel column chromatography (A-DCM; B-MeOH) to provide 1-12c (147 mg, 43.44% yield).

MS (ESI) m/z: 222.1 [M+H]⁺

¹H NMR (400 MHz, DMSO-d6) δ 11.76 (s, 1H), 8.46 (dd, J=9.3, 5.1 Hz, 1H), 7.48 (t, J=9.1 Hz, 1H), 2.91 (t, J=6.1 Hz, 2H), 2.75-2.65 (m, 2H), 2.14 (s, 3H), 2.08-1.93 (m, 2H).

### Step 3

### N-(4-fluoro-8-oxo-5,6,7,8-tetrahydronaphthalen-1-yl)acetamide (1-12d)

Compound 1-12d (150.00 mg) was synthesized according to synthetic procedure of step 4 of example 1-14e.

MS (ESI) m/z: 240.0 [M−H]⁻

### Step 4

### N,N′-(4-fluoro-8-oxo-5,6,7,8-tetrahydronaphthalene-1,7-diyl)diacetamide (1-12e)

Compound 1-12e (170.00 mg) was synthesized according to synthetic procedure of step 5 of example 1-14f.

MS (ESI) m/z: 279.1 [M+H]⁺

### Step 5

### N-(8-amino-5-fluoro-1-oxo-1,2,3,4-tetrahydronaphthalen-2-yl)acetamide (1-12f)

Compound 1-12f (127.34 mg, crude) was synthesized according to synthetic procedure of step 6 of example 1-14g.

MS (ESI) m/z: 237.1 [M+H]⁺

### Step 6

### N-((9S)-9-ethyl-4-fluoro-9-hydroxy-10,13-dioxo-2,3,9,10,13,15-hexahydro-1H,12H-benzo[de]pyrano[3′,4′:6,7]indolizino[1,2-b]quinolin-1-yl)acetamide (1-12g)

Compound 1-12g (140.00 mg) was synthesized according to synthetic procedure of step 7 of example 1-14h.

MS (ESI) m/z: 464.3 [M+H]⁺

### Step 7

### (1S,9S)-1-amino-9-ethyl-4-fluoro-9-hydroxy-1,2,3,9,12,15-hexahydro-10H,13H-benzo[de]pyrano[3′,4′:6,7]indolizino[1,2-b]quinoline-10,13-dione (1-12)

### (1R,9S)-1-amino-9-ethyl-4-fluoro-9-hydroxy-1,2,3,9,12,15-hexahydro-10H,13H-benzo[de]pyrano[3′,4′:6,7]indolizino[1,2-b]quinoline-10,13-dione (1-13)

1-12 (20.10 mg, 13.90% yield) and 1-13 (16.00 mg, 11.07% yield) were synthesized according to synthetic procedure of step 8 of example 1-14.

MS (ESI) m/z: 422.3 [M+H]⁺

¹H NMR (400 MHz, DMSO-d6) δ 7.99 (ddd, J=130.6, 14.4, 7.2 Hz, 2H), 7.34 (s, 1H), 6.92 (d, J=216.0 Hz, 1H), 6.52 (s, 1H), 5.68 (d, J=19.4 Hz, 1H), 5.46-5.30 (m, 4H), 3.16 (d, J=6.1 Hz, 2H), 2.15 (s, 2H), 2.02-1.85 (m, 5H), 1.49-1.30 (m, 2H), 0.87 (dd, J=7.0, 3.5 Hz, 3H).

### Example 1-14&1-15

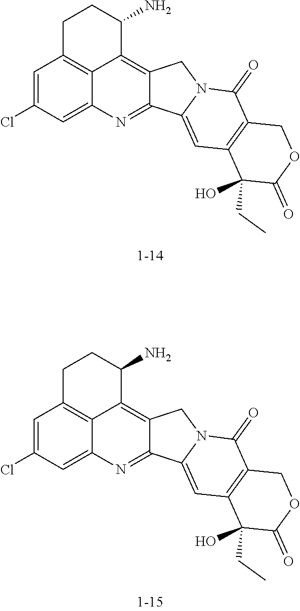 [SMILES: CC[C@@]1(O)C(=O)OCc2c1cc1n(c2=O)Cc2c-1nc1cc(Cl)cc3c1c2[C@@H](N)CC3 ; CC[C@@]1(O)C(=O)OCc2c1cc1n(c2=O)Cc2c-1nc1cc(Cl)cc3c1c2[C@H](N)CC3]

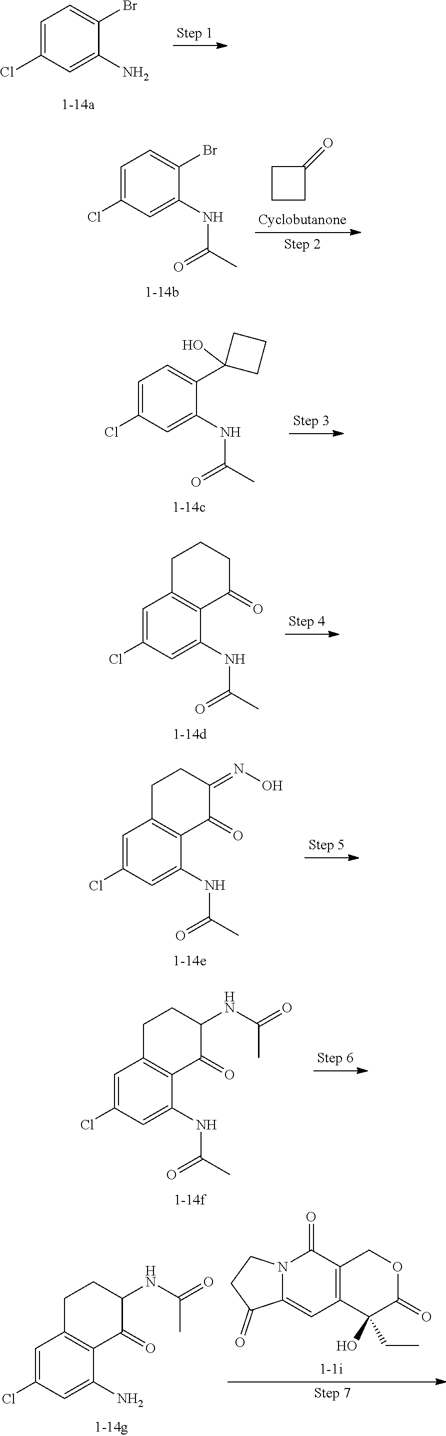

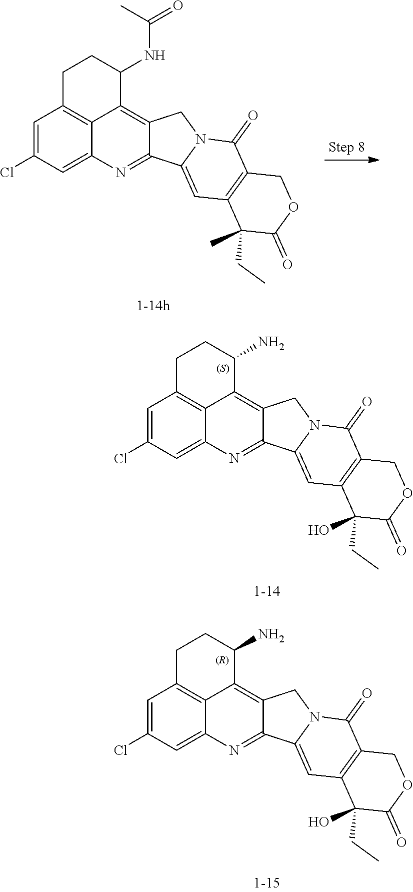

### Step 1

### N-(2-bromo-5-chlorophenyl)acetamide (1-14b)

To a solution of 1-14a (20.00 g, 97.59 mmol) in CH₂Cl₂ (200 mL) was added Ac₂O (14.94 g. 146.39 mmol) and H₂SO₄ (0.96 g. 9.80 mmol). The reaction mixture was reacted at 0° C. for 5 h. The mixture was concentrated to 50 mL. To the residual was added water (200 mL), extracted with EtOAc (200 mL*3), After separation the combined organic layers were washed with sat. Na₂CO₃ (200 mL*3), dried over Na₂SO₄, filtered and the filtrate was concentrated under vacuum. The residue was purified by silica gel column flash chromatography (A—petroleum ether; B—EtOAc) to afford 1-14b (18 g, 74.7% yield).

MS (ESI) m/z: 249.9 [M+H]⁺

### Step 2

### N-(5-chloro-2-(1-hydroxycyclobutyl)phenyl)acetamide (1-14c)

To a solution of 1-14b (10 g, 40.50 mmol) in THF (100 mL) was added Butyllithium (25.60 mL. 85.04 mmol, 2.5 M) at −78° C. and stirred for 1.5 h. Then Cyclobutanone (3.12 g, 44.55 mmol) was added slowly. The reaction mixture was reacted at same temperature for 1 h and warm up to r.t. To the mixture was quenched with sat. NH₄Cl, extracted with EtOAc (100 mL*3), After separation the combined organic layers were washed with brine (50 mL*3), dried over Na₂SO₄, filtered and the filtrate was concentrated under vacuum. The residue was recrystallization with EtOH to afford 1-14c (2.40 g, 24.8% yield).

MS (ESI) m/z: 238.0 [M−H]⁻

### Step 3

### N-(3-chloro-8-oxo-5,6,7,8-tetrahydronaphthalen-1-yl)acetamide (1-14d)

AgNO₃ (284.22 mg, 1.67 mmol) and Na₂S₂O₈ (5.65 g, 20.91 mmol) was added a solution of 1-14c (2.00 g, 8.37 mmol) in 30 mL of CH₂Cl₂/H₂O (v/v=1/1) under a nitrogen atmosphere. After being stirred at 25° C. for 16 h, the reaction mixture was quenched with water, extracted with EtOAc (100 mL*3), washed with brine, dried over anhydrous Na₂SO₄, and concentrated. Column chromatography on silica gel (EtOAc/petroleum ether=1/5) to afford 1-14d (1.70 g, 85.7% yield) as a white solid.

MS (ESI) m/z: 238.0 [M+H]⁺

### Step 4

### (Z)—N-(3-chloro-7-(hydroxyimino)-8-oxo-5,6,7,8-tetrahydronaphthalen-1-yl)acetamide (1-14e)

To a solution of 1-14d (1.00 g, 4.22 mmol) in 25 mL of THF/t-BuOH (v:v=4:1) was added potassium tert-butoxide (1 M in THF, 615.34 mg, 5.48 mmol) at 0° C. The atmosphere was purged with N₂ and the mixture was cooled to 0° C. using and ice water bath. After 5 mins, to the stirred mixture was added Isopentyl Nitrite (592.99 mg, 5.06 mmol), resulting in a color change from yellow to red, and the reaction mixture was allowed to warm to r.t. 1 N HCl was added until the red color faded, and the mixture was transferred to a separatory funnel. The aqueous layer was extracted with EtOAc (50 mL*3). The combined organics were washed with H₂O (50 mL), brine (100 mL), dried over Na₂SO₄ and concentrated in vacuo to give crude. The crude residue was trituration with MTBE (10 mL) to afford 1-14e (820.00 mg, 73% yield).

MS (ESI) m/z: 264.9 [M−H]⁻

### Step 5

### N,N′-(3-chloro-8-oxo-5,6,7,8-tetrahydronaphthalene-1,7-diyl)diacetamide (1-14f)

Wet PtO₂ (100 mg) was added to a mixture of the 1-14e (720.00 mg, 2.71 mmol) in Ac₂O (20 mL). The reaction mixture was purged with H₂ balloon for three times and reacted at r.t. under H₂ balloon for 6 h. The mixture was filtered off through celite and washed with Ac₂0. The filtrate was concentrated under vacuum and purified by silica gel column chromatography (CH₂Cl₂: MeOH) to provide 1-14f (470.00 mg, 59.1% yield) as a gray solid.

MS (ESI) m/z: 295.1 [M+H]⁺

### Step 6

### N-(8-amino-6-chloro-1-oxo-1,2,3,4-tetrahydronaphthalen-2-yl)acetamide (1-14g)

2N HCl (20 mL) was added to a solution of 1-14f (0.80 g, 2.72 mmol) in MeOH (20 mL). The reaction mixture was purged with N₂ for three times and reacted at 60° C. under N₂ for 5 h. The mixture was concentrated and purified by silica gel column chromatography (CH₂Cl₂: MeOH) to provide 1-14g (500 mg, 90.25% yield) as a gray solid.

MS (ESI) m/z: 253.1 [M+H]⁺

### Step 7

### N-((9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)acetamide (1-14h)

The 1-14g (500 mg, 2.45 mmol), 1-11 (787.24 mg, 2.99 mmol) and a catalytic amount of PPTS (200 mg, 0.80 mmol) in toluene (40 mL) was heated to reflux (135˜140° C.) using a Dean-Stark trap for 48 h. The reaction was concentrated under vacuum and purified by silica gel column chromatography (CH₂Cl₂: MeOH) to provide 1-14h (850 mg, 70.98% yield) as a gray solid. UPLC analysis: 1-14h, retention time=2.66 min; retention time=2.77 min (Mobile phase A: 0.1% FA in water, B: MeCN; Gradient: 10% B maintain 1 min, 10%-95% B, 5 min, 95% B maintain 1 min; Flow rate: 0.6 mL/min; Column: ACQUITY UPLC® BEH C18 1.7 μm)

MS (ESI) m/z: 480.3 [M+H]⁺

### Step 8

### (1S,9S)-1-amino-5-chloro-9-ethyl-9-hydroxy-1,2,3,9,12,15-hexahydro-10H,13H-benzo[de]pyrano[3′,4′:6,7]indolizino[1,2-b]quinoline-10,13-dione (1-14)

### (1R,9S)-1-amino-5-chloro-9-ethyl-9-hydroxy-1,2,3,9,12,15-hexahydro-10H,13H-benzo[de]pyrano[3′,4′:6,7]indolizino[1,2-b]quinoline-10,13-dione (1-15)

A solution of 1-14h (0.40 g, 0.83 mmol) in 12N HCl (30 mL). The reaction mixture was purged with N₂ for three times and reacted at 100° C. under N₂ for 9 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min) to afford 1-14 (75.60 mg, 20.72% yield) (MS (ESI) m/z: 438.2 [M+H]⁺) and 1-15 (76.90 mg, 21.7% yield) (MS (ESI) m/z: 438.2 [M+H]⁺) were obtained as white solids. [retention time: 1.98 mins and 2.67 mins]

¹H NMR (400 MHz, DMSO-d6) δ 8.15-8.05 (m, 1H), 7.60 (s, 1H), 7.31 (s, 1H), 6.52 (s, 1H), 5.76 (d, J=19.4 Hz, 1H), 5.47-5.33 (m, 3H), 4.77 (s, 1H), 3.14 (d, J=17.2 Hz, 2H), 2.30-2.09 (m, 2H), 1.91-1.77 (m, 2H), 0.85 (t, J=7.3 Hz, 3H).

¹H NMR (400 MHz, DMSO-d6) δ 8.14 (s, 1H), 8.00 (d, J=2.0 Hz, 1H), 7.51 (d, J=1.8 Hz, 1H), 7.29 (s, 1H), 6.50 (s, 1H), 5.63 (d, J=19.4 Hz, 1H), 5.48-5.24 (m, 3H), 4.48 (t, J=4.7 Hz, 1H), 3.28 (dd, J=10.7, 6.1 Hz, 1H), 3.12-3.04 (m, 1H), 2.19-1.94 (m, 2H), 1.91-1.71 (m, 2H), 0.85 (t, J=7.3 Hz, 3H).

### Example 1-16 and 1-17

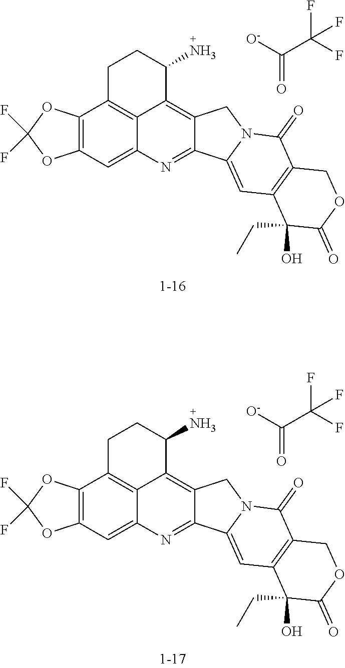 [SMILES: CC[C@@]1(O)C(=O)OCc2c1cc1n(c2=O)Cc2c-1nc1cc3c(c4c1c2[C@@H](N)CC4)OC(F)(F)O3 ; O=C([O-])C(F)(F)F ; CC[C@@]1(O)C(=O)OCc2c1cc1n(c2=O)Cc2c-1nc1cc3c(c4c1c2[C@H](N)CC4)OC(F)(F)O3 ; O=C([O-])C(F)(F)F]

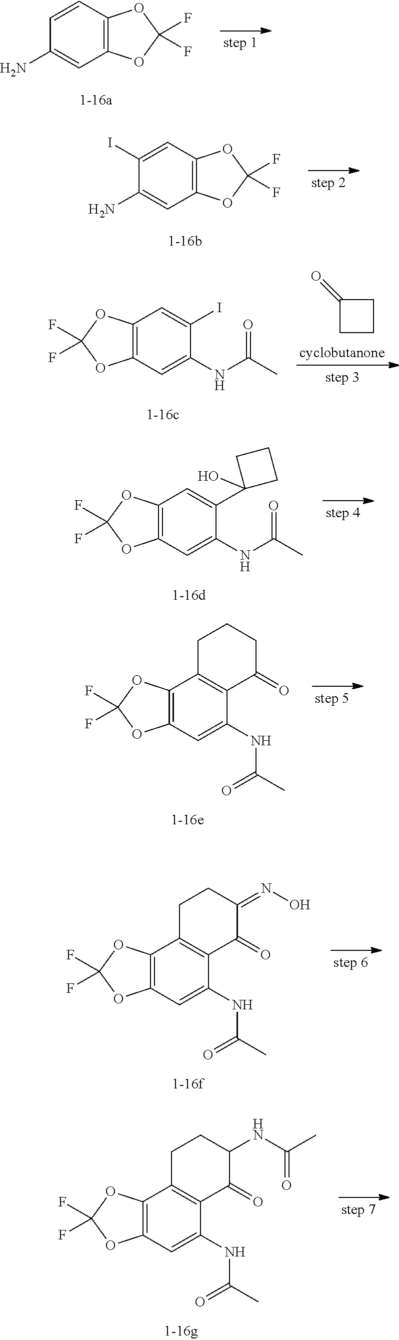

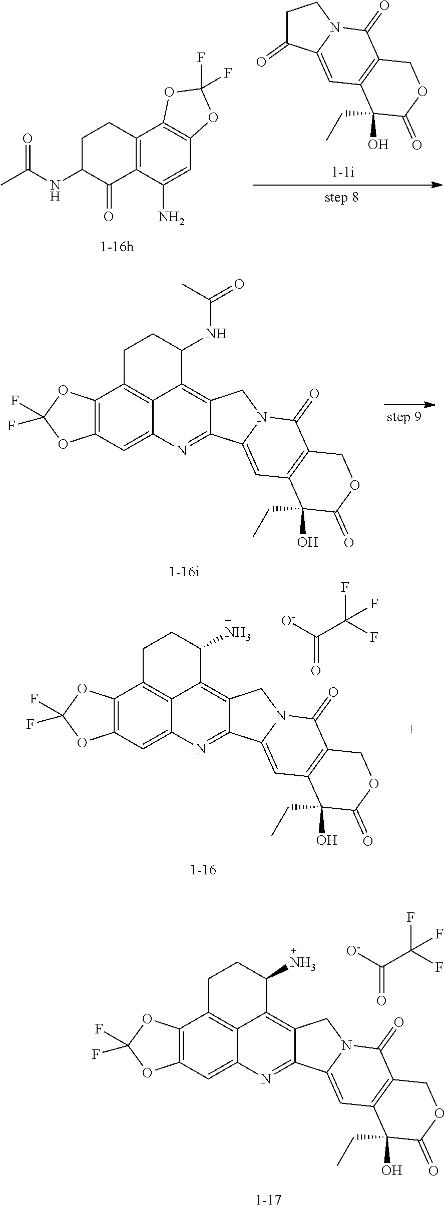

### Step 1

### 2,2-difluoro-6-iodobenzo[d][1,3]dioxol-5-amine (1-16b)

To a solution of 1-16a (10.0 g, 57.76 mmol) in DMF (200 mL) was added NIS (14.30 g, 63.54 mmol) in portions on ice bath and then stirred at r.t. overnight. The mixture was quenched by addition of sat. Na₂SO₃ (50 mL), diluted with EA (500 mL), the organic layer was washed with brine (300 mL*3), dried over Na₂SO₄, filtered and concentrated under vacuum to give a crude product, which was purified by silica gel column chromatography (EA/PE=0%˜20%), fraction was concentrated under vacuum to give 1-16b (14.0 g, 81.1% yield) as brown oil.

MS (ESI) m/z: 300.0 [M+H]⁺

¹HNMR (400 MHz, DMSO-d6) δ 7.60 (s, 1H), 6.78 (s, 1H), 5.34 (s, 3H).

### Step 2

### N-(2,2-difluoro-6-iodobenzo[d][1,3]dioxol-5-yl)acetamide (1-16c)

To a solution of 1-16b (14.0 g, 46.82 mmol) in CH₂Cl₂ (150 mL) were added acetic anhydride (4.8 mL, 51.5 mmol) and DMAP (114.4 mg, 0.94 mmol). The mixture was stirred at r.t. for 1 hr. The suspension was filtered and the cake was washed with CH₂Cl₂ (30 mL), the cake was dried under vacuum to give 1-16c (9.4 g) as a white solid. The mother liquid was purified by silica column chromatography (60 g, EtOAc/PE=10%-40%) to give 1-16c (4.0 g) as a white solid. Overall yield (97.3%)

¹H NMR (400 MHz, CDCl₃) δ 8.07 (s, 1H), 7.45 (s, 1H), 7.37 (s, 1H), 2.24 (s, 3H).

### Step 3

### N-(2,2-difluoro-6-(1-hydroxycyclobutyl)benzo[d][1,3]dioxol-5-yl)acetamide (1-16d)

To a mixture of 1-16c (10.0 g, 29.32 mmol) in THF (200 mL) was added i-PrMgCl (2M, 36.7 mL, 73.3 mmol) dropwise at −40° C. for 1 h, then Cyclobutanone (6.17 g, 87.96 mmol) was added at -40° C. slowly. The resulting mixture was stirred at r.t. for 2 h. The mixture was poured into sat. NH₄Cl (300 mL) and extracted with EtOAc (300 mL). The organic layers were washed with brine (200 mL), dried over Na₂SO₄, filter and concentrated under vacuum to give a residue. It was purified by column chromatography (SiO₂, EtOAc/PE=0%-50%) to give 1-16d (9.5 g, crude) as a white solid.

MS (ESI) m/z: 284.1 [M−H]⁻

### Step 4

### N-(2,2-difluoro-6-oxo-6,7,8,9-tetrahydronaphtho[1,2-d][1,3]dioxol-5-yl)acetamide (1-16e)

To a mixture of 1-16d (10.0g, crude) in CH₂Cl₂/H₂O (1:1, 200 mL) were added K₂S₂O₈ (28.4 g, 105.2 mmol) and 0.5M AgNO₃ aqueous (14 mL, 7.0 mmol) via syringe. The mixture was stirred at r.t. for overnight with darkness. The mixture was filtered through a pad of celite then extracted with CH₂Cl₂ (300 mL*2). The combined organic layers were washed with brine (200 mL), dried over Na₂SO₄, filtered and concentrate under vacuum to give a red oil. It was purified by silica column (SiO₂, EtOAc/PE=O⁰%10%) to give 1-16e (1.45 g, 100% purity) as light-yellow solid.

MS (ESI) m/z: 284.2 [M+H]⁺

¹H NMR (400 MHz, CDCl3) δ 12.42 (s, 1H), 8.58 (s, 1H), 7.28 (s, 1H), 2.96 (t, J=6.2 Hz, 2H), 2.75-2.70 (m, 2H), 2.25 (s, 3H), 2.16-2.09 (m, 3H).

### Step 5

### (Z)—N-(2,2-difluoro-7-(hydroxyimino)-6-oxo-6,7,8,9-tetrahydronaphtho[1,2-d][1,3]dioxol-5-yl)acetamide (1-16f)

To a solution of 1-16e (1.45 g, 5.12 mmol) in THF (20 mL) were added isoamyl nitrite (0.75 mL, 7.17 mmol) and t-BuOK (1M, 6.7 mL, 6.7 mmol via syringe over 10 min at 0° C. The mixture was stirred at 0° C. for 30 min. The mixture was quenched by addition of water (30 mL), Extracted with EtOAc (40 mL*3). Combined the organic layers washed with brine (50 mL), dried over Na₂SO₄, filtered and concentrated under vacuum to give a residue. It was purified by silica column (SiO2, EtOAc/PE=0%˜50%) to give 1-16f (1.04 g, 65.1% yield) as light-yellow solid.

MS (ESI) m/z: 313.2 [M+H]⁺

### Step 6

### N,N′-(2,2-difluoro-6-oxo-6,7,8,9-tetrahydronaphtho[1,2-d][1,3]dioxole-5,7-diyl)diacetamide (1-16g)

To a solution of 1-16f (1.04 g, 3.33 mmol) in THF/Ac₂O (3:1, 12 mL) was added PtO₂ (75.6 mg, 0.33 mmol). The mixture was purged with H₂ balloon for three times. Then stirred at RT for overnight. The mixture was concentrated under vacuum to give a 1-16g (1300 mg, crude) as a black solid.

MS (ESI) m/z: 341.2 [M+H]⁺

### Step 7

### N-(5-amino-2,2-difluoro-6-oxo-6,7,8,9-tetrahydronaphtho[1,2-d][1,3]dioxol-7-yl)acetamide (1-16h)

A mixture of 1-16g (1.3 g crude) in 2N HCl/MeOH (1:3, 12 mL) was stirred at 60° C. for 7 hr. The mixture was basified with sat.Na₂CO₃ to pH=8 then diluted with EtOAc (50 mL*4). Combined organic layers were washed with brine (50 mL), dried over Na₂SO₄, filtered and the filtrate was concentrated under vacuum to give a residue. It was purified by silica gel chromatography (10 g, MeOH/CH₂Cl₂=0%˜20%) to give 1-16h (660 mg) as brown solid.

MS (ESI) m/z: 299.2 [M+H]⁺;

### Step 8

### N-((10S)-10-ethyl-5,5-difluoro-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)acetamide (1-16i)

To a mixture of 1-16h (660 mg, 2.21 mmol) and 1-1i (582.5 mg, 2.21 mmol) in toluene (30 mL) were added O-cresol (2 mL) and PPTS (166.2 mg, 0.664 mmol). The mixture was heated to reflux for 48 h and water was removed by Dean-Stark trap. The mixture was concentrated under vacuum and the crude product was purified by silica gel column chromatography (MeOH/CH₂Cl₂=0%˜10%), fraction was concentrated under vacuum to give 1-16i (610 mg, 55.4% yield) as a gray solid.

MS (ESI) m/z: 526.4 [M+H]⁺;

### Step 9

### (1S,10S)-1-amino-10-ethyl-5,5-difluoro-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione 2,2,2-trifluoroacetate salt (1-16); (1S,10S)-1-amino-10-ethyl-5,5-difluoro-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione 2,2,2-trifluoroacetate salt (1-17)

A mixture of 1-16i (610 mg, 1.225 mmol) in H₂O/MsOH (3:1, 12 mL) was heated to 95° C. and stirred 8 hr. The mixture was centrifuge (4000 r/min, 3 min) the liquid was purified by prep-HPLC (0.05% TFA), The fraction was concentrated under vacuum to give 1-16 (107 mg, RT=1.79 min) as dark yellow solid and 1-17 (123.3 mg, RT=1.89 min) as a yellow solid.

MS (ESI) m/z: 484.3 [M+H]⁺;

### Example 1-18, 1-19

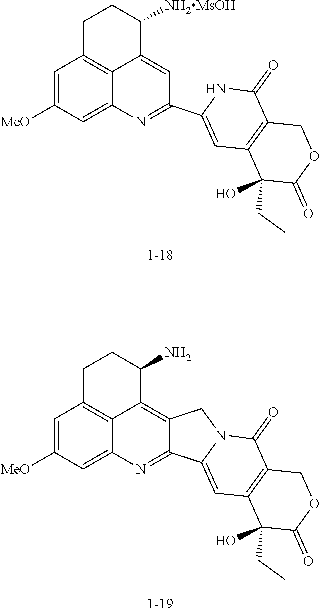

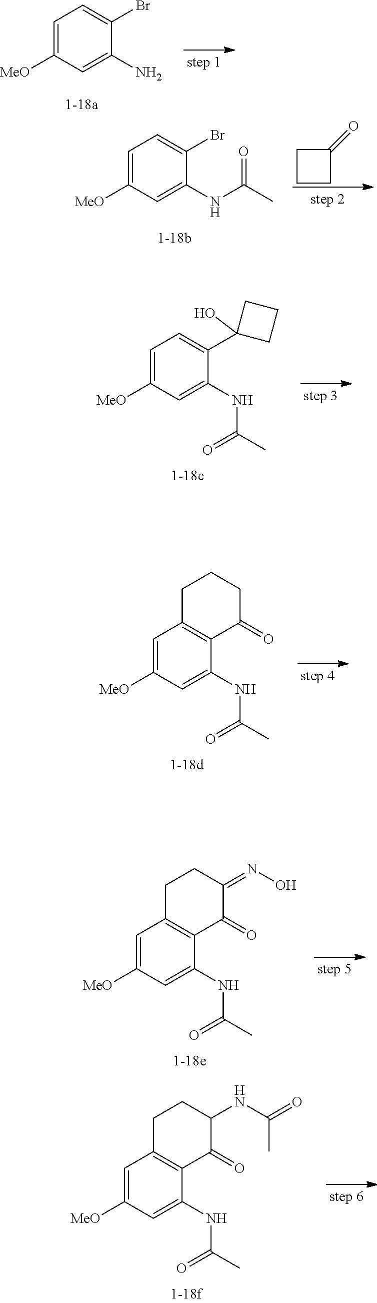

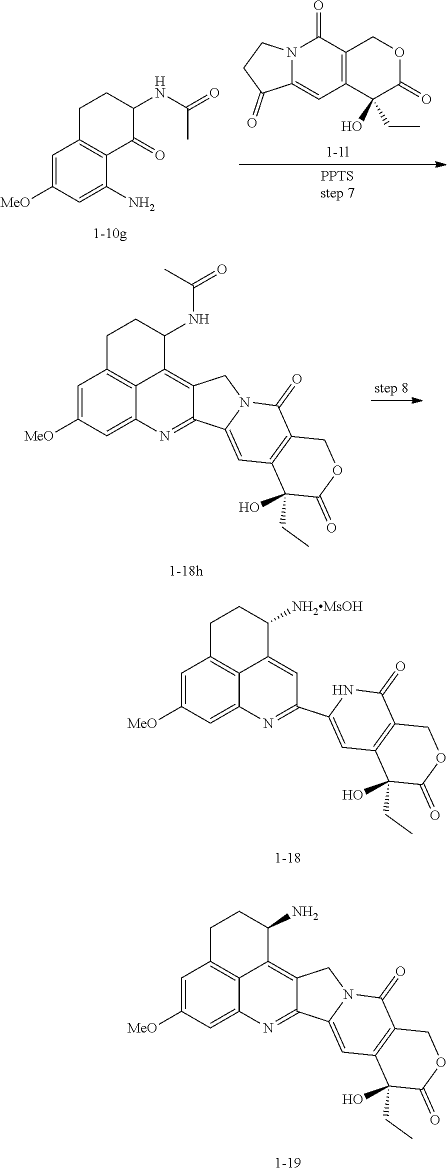

### Step 1

### N-(2-bromo-5-methoxyphenyl)acetamide (1-18b)

To a solution of 9.8 mL Ac₂O and 0.05 mL con. H₂SO₄ was added 1-18a (20 g, 99.0 mmol) in batches at ice water bath. Then the reaction was slowly raised to r.t. and stirred for 1h. The mixture was poured into ice water. The precipitate was filtered and washed with water for three times, the solid was then dried at 70° C. to afford compound 1-18b (20.1 g, 93% yield) as brown solid.

MS (ESI) m/z: 244.1 [M+H]⁺

### Step 2

### N-(2-(1-hydroxycyclobutyl)-5-methoxyphenyl)acetamide (1-18c)

To a solution of 1-18b (12 g, 49.4 mmol, 1.0 equiv) in 120 mL anhydrous THF under N₂ was slowly added n-BuLi (47.4 mL, 118.5 mmol, 2.4 equiv) at −78° C. After the mixture was stirred for 1.5 h at −78° C., cyclobutanone (8.9 mL, 118.5 mmol, 2.4 equiv) was slowly added at −78° C. The mixture was stirred for 0.5 h at −78° C., then slowly warmed to r.t. and stirred for 0.5 h at r.t. The reaction was quenched with saturated NH₄Cl, extracted with EtOAc (150 mL*3). The combined organic layers were washed with brine (50 mL), dried over anhydrous Na₂SO₄, filtered and concentrated under vacuum to give a residue which was triturated with MTBE (40 mL) to afford 1-18c (5.3 g, 46% yield) as a pale solid.

MS (ESI) m/z: 218.1 [M+H-H₂O]⁺

### Step 3

### N-(3-methoxy-8-oxo-5,6,7,8-tetrahydronaphthalen-1-yl)acetamide (1-18d)

1-18c (3.2 g, 13.6 mmol, 1.0 equiv), AgNO₃ (5.44 mL, 2.72 mmol, 0.2 equiv), and K₂S₂O₈ (7.36 g, 27.2 mmol, 2.0 equiv) were loaded in a flask which was subjected to evacuation/flushing with nitrogen three times. CH₂Cl₂/H₂O (1/1 v/v, 190 mL) was added to the mixture and the mixture was then stirred at r.t. overnight until the starting material was consumed as determined by TLC. The mixture was extracted with CH₂Cl₂ (3×80 mL). The combined organic layer was washed by brine, dried over Na₂SO₄, filtered, concentrated, and purified by flash chromatography on silica gel (ethyl acetate/PE) to give product 1-18d (2.03 g, 64% yield).

MS (ESI) m/z: 234.2 [M+H]⁺

### Step 4

### (Z)—N-(7-(hydroxyimino)-3-methoxy-8-oxo-5,6,7,8-tetrahydronaphthalen-1-yl)acetamide (1-18e)

To a solution of 40 mL anhydrous THF and 10 mL t-BuOH was added t-BuOK in THF (11.3 mL, 11.3 mmol, 1.3 equiv) with N₂. 1-18d (2.03 g, 8.71 mmol, 1.0 equiv) was slowly added to the mixture on ice water bath and stirred at 0° C. for 10 min. Isopentyl Nitrite (1.64 mL, 12.2 mmol, 1.4 equiv) was added to the reaction dropwise at 0° C. and then stirred for 1 h at rt. The mixture was adjusted to PH<6 by 2 N HCl, then extracted with EtOAc (50 mL*3). The combined organic layers were washed with brine (20 mL), dried over anhydrous Na₂SO₄, filtered and concentrated under vacuum to give a residue which was triturated with MTBE (20 mL) to afford 1-18e (1.94 g, 85% yield) as a brown solid.

MS (ESI) m/z: 261.0 [M−H]⁻

### Step 5

### N,N′-(3-methoxy-8-oxo-5,6,7,8-tetrahydronaphthalene-1,7-diyl)diacetamide (1-18f)

To a solution of 1-18e (2.0 g, 7.57 mmol) in 90 mL AcOH and 30 mL Ac₂O was added wet Pd/C (300 mg). Then the mixture was purged with H₂ balloon for 3 times and stirred at r.t. overnight. The mixture was filtered through a pad of celite and concentrated to give compound crude 1-18f. The crude product was triturated with MTBE (15 mL) to afford 1-18f (1.87 g, 85% yield) as a pale solid.

MS (ESI) m/z: 291.1 [M+H]⁺

### Step 6

### N-(8-amino-6-methoxy-5-methyl-1-oxo-1,2,3,4-tetrahydronaphthalen-2-yl)acetamide (1-18g)

To a solution of 30 mL MeOH and 30 mL 2 N HCl was added 1-18f (990 mg, 3.41 mmol) under N₂. The mixture was stirred at 60° C. for 2 h. The reaction was then neutralized with sat. NaHCO₃, filtered, washed by H₂O, dried to give 1-18g (734 mg, 87% yield) as a pale solid.

MS (ESI) m/z: 249.1 [M+H]⁺

### Step 7

### N-((9S)-9-ethyl-9-hydroxy-5-methoxy-10,13-dioxo-2,3,9,10,13,15-hexahydro-1H,12H-benzo[de]pyrano[3′,4′:6,7]indolizino[1,2-b]quinolin-1-yl)acetamide (1-18h)

PPTS (534.8 mg, 2.13 mmol, 0.6 equiv) and 1-1i (1.02 g, 3.9 mmol, 1.1 equiv) were added to a suspension of 1-18g (880 mg, 3.55 mmol, 1.0 equiv) in 70 mL toluene, and the mixture was refluxed for 48 h by water separator. The mixture was concentrated and purified by flash chromatography on silica gel (CH₂Cl₂/MeOH) to give product 1-18h (560 mg, 33% yield) as a pale solid.

MS (ESI) m/z: 476.3 [M+H]⁺

### Step 8

### N-((9S)-9-ethyl-5,9-dihydroxy-10,13-dioxo-2,3,9,10,13,15-hexahydro-1H,12H-benzo[de]pyrano[3′,4′:6,7]indolizino[1,2-b]quinolin-1-yl)acetamide (1-18, 1-19)

Compound 1-18h (325 mg, 0.68 mmol) was added to 4 mL MsOH and 9 mL H₂O at ice water bath, and stirred at 90° C. for 10 h under N₂. The solution was then purified by prep-HPLC (FA) (Method: column: XBridge Prep C18 OBD 5 um 19*150 mm; Mobile phase: A—water (0.1% formic acid): B-acetonitrile; Flow rate: 20 mL/min, the fraction was lyophilized to give 1-18 (101 mg, 34% yield) and 1-19 (100 mg, 34% yield) as a yellow solid.

1-18, Retention time (1.12 min)

MS (ESI) m/z: 434.3 [M+H]⁺

¹H NMR (400 MHz, DMSO-d6) δ 8.43 (s, 3H), 7.52 (d, J=2.5 Hz, 1H), 7.35 (s, 1H), 7.31 (s, 1H), 6.55 (s, 1H), 5.70 (d, J=19.3 Hz, 1H), 5.46 (s, 2H), 5.38 (d, J=19.2 Hz, 1H), 5.08 (s, 1H), 3.97 (s, 3H), 3.33-3.17 (m, 1H), 2.45-2.42 (m, 1H), 2.29 (s, 2H), 2.24-2.12 (m, 1H), 1.92-1.85 (m, 2H), 0.89 (t, J=7.3 Hz, 3H).

1-19, Retention time (2.19 min)

MS (ESI) m/z: 434.3 [M+H]⁺

¹H NMR (400 MHz, DMSO-d6) δ 8.14 (s, 1H), 7.43 (d, J=2.5 Hz, 1H), 7.31 (s, 1H), 7.18 (d, J=2.1 Hz, 1H), 6.51 (s, 1H), 5.63 (d, J=19.3 Hz, 1H), 5.44 (s, 2H), 5.35 (d, J=19.2 Hz, 1H), 4.57 (s, 1H), 3.94 (s, 3H), 3.14-3.00 (m, 1H), 2.22-2.02 (m, 2H), 1.86-1.84 (m, 2H), 0.88 (t, J=7.3 Hz, 3H).

### Example 1-20, 1-21

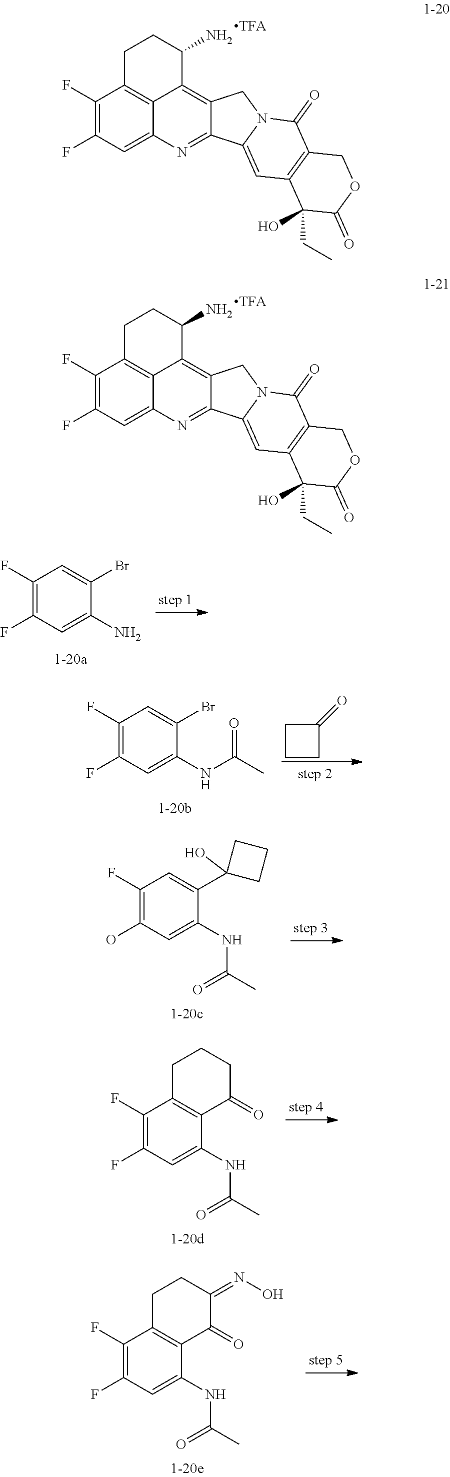

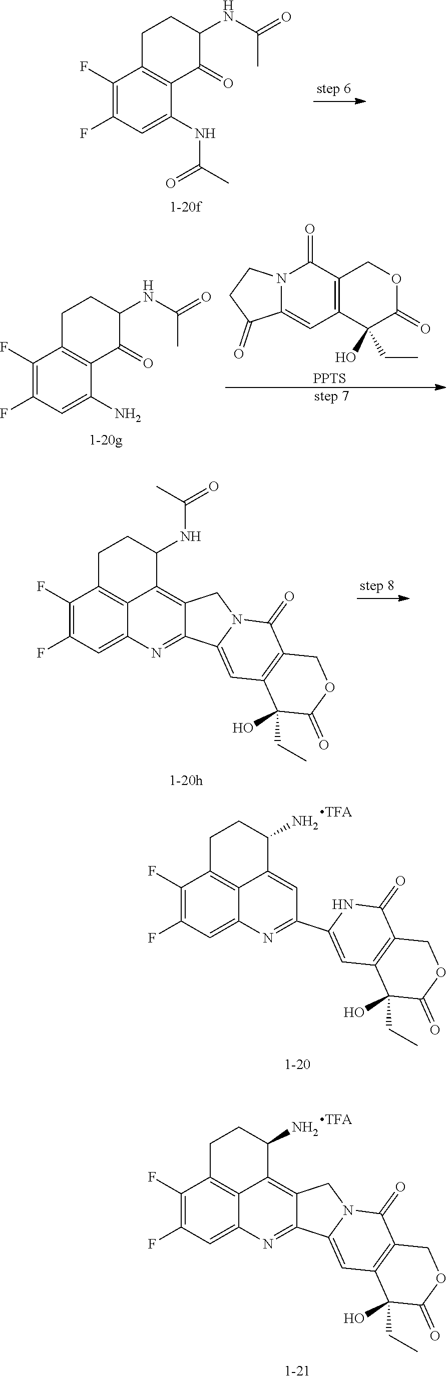

### Step 1˜Step 8

### 2,2,2-trifluoroacetaldehyde-(S)-6-((S)-4-amino-7,8-difluoro-5,6-dihydro-4H-benzo[de]quinolin-2-yl)-4-ethyl-4-hydroxy-1,7-dihydro-3H-pyrano[3,4-c]pyridine-3,8 (4H)-dione (1/1) trifluoroacetate (1-20)

### 2,2,2-trifluoroacetaldehyde-(1R,9S)-1-amino-9-ethyl-4,5-difluoro-9-hydroxy-1,2,3,9,12,15-hexahydro-10H,13H-benzo[de]pyrano[3′,4′:6,7]indolizino[1,2-b]quinoline-10,13-dione (1/1) trifluoroacetate (1-21)

1-20 (17.5 mg, 19% yield) and 1-21 (15.7 mg, 17% yield) was synthesized according to synthetic procedure of example 1-18 and 1-19.

1-20

MS (ESI) m/z: 440.3 [M+H], Retention time (1.30 min).

¹H NMR (400 MHz, DMSO-d6) δ 8.46 (s, 3H), 8.20 (dd, J=11.1, 7.8 Hz, 1H), 7.37 (s, 1H), 6.57 (s, 1H), 5.74 (d, J=19.4 Hz, 1H), 5.46-5.41 (m, 3H), 5.15 (s, 1H), 3.17-3.02 (m, 2H), 2.32-2.08 (m, 2H), 1.92-1.85 (m, 2H), 0.88 (t, J=7.2 Hz, 3H).

¹⁹F NMR (377 MHz, DMSO-d6) δ −73.51 (s), −131.03 (d, J=22.6 Hz), −138.00 (d, J=22.5 Hz).

1-21

MS (ESI) m/z: 440.3 [M+H], Retention time (2.36 min).

¹H NMR (400 MHz, DMSO-d6) δ 8.49 (s, 3H), 8.20 (dd, J=11.2, 7.8 Hz, 1H), 7.37 (s, 1H), 6.56 (s, 1H), 5.74 (d, J=19.4 Hz, 1H), 5.44-5.42 (m, 3H), 5.17 (s, 1H), 3.23-3.01 (m, 2H), 2.19-2.16 (m, 2H), 2.02-1.71 (m, 2H), 0.88 (t, J=7.3 Hz, 3H).

¹⁹F NMR (377 MHz, DMSO-d6) δ −73.63 (s), −131.03 (d, J=22.5 Hz), −138.00 (d, J=22.6 Hz).

### Example 1-22, 1-23

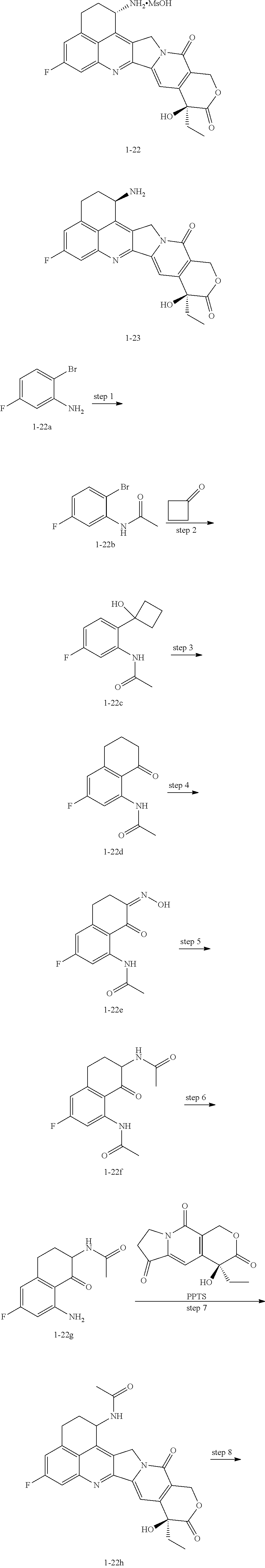

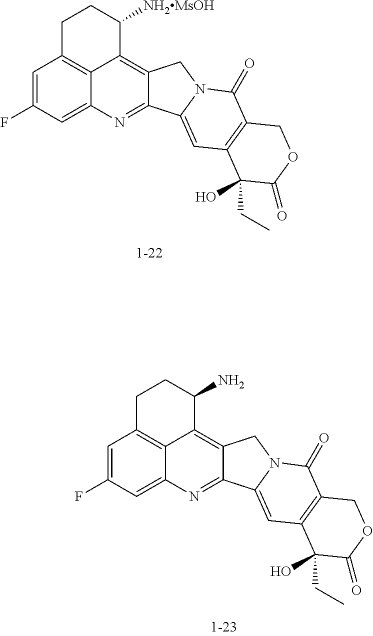

### Step 1˜Step 8

### (1S,9S)-1-amino-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione methanesulfonate (1-22)

### (1R,9S)-1-amino-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione (1-23)

1-22 (19 mg, 17% yield) and 1-23 (14.9 mg, 13% yield) was synthesized according to synthetic procedure of example 1-18 and 1-19.

MS (ESI) m/z: 422.2 [M+H]⁺

### Example 1-24, 1-25

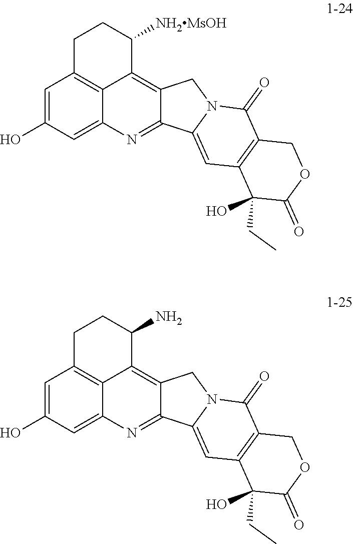

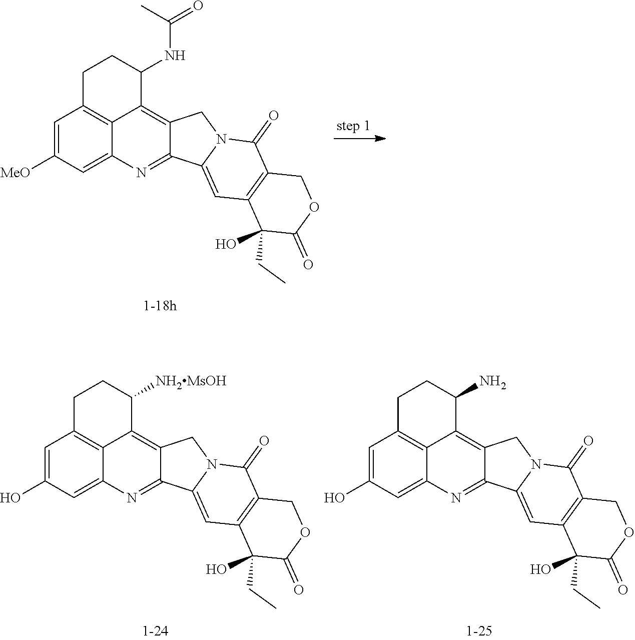

### Step 1

### (1S,9S)-1-amino-9-ethyl-5,9-dihydroxy-1,2,3,9,12,15-hexahydro-10H,13H-benzo[de]pyrano[3′,4′:6,7]indolizino[1,2-b]quinoline-10,13-dione methanesulfonate (1-24)

### (1R,9S)-1-amino-9-ethyl-5,9-dihydroxy-1,2,3,9,12,15-hexahydro-10H,13H-benzo[de]pyrano[3′,4′:6,7]indolizino[1,2-b]quinoline-10,13-dione (1-25)

Compound 1-18h (250 mg) in 48% aqueous HBr (10 ml) was heated to reflux for 24 h. The mixture was purified by prep-HPLC (TFA) (Method: column: XBridge Prep C18 OBD 5 um 19*150 mm; Mobile phase: A—water (0.1% TFA): B-acetonitrile; Flow rate: 20 mL/min, the fraction was lyophilized to give 1-24 (3.6 mg, 3% yield) and 1-25 (6.6 mg, 6% yield) as a yellow solid.

MS (ESI) m/z: 420.3 [M+H]⁺

### Example 1-26 and 1-27

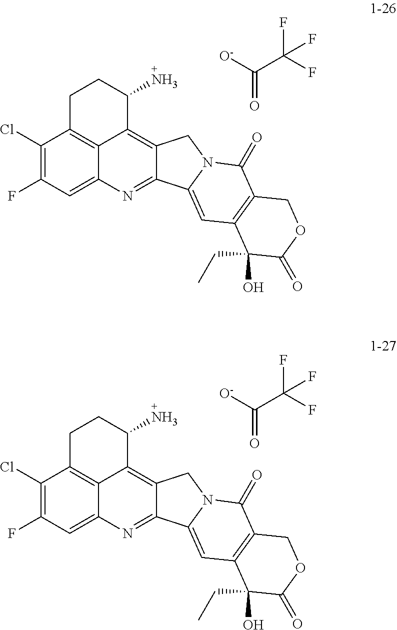 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F ; CC[C@@]1(O)C(=O)OCc2c1cc1n(c2=O)Cc2c-1nc1cc(F)c(Cl)c3c1c2[C@@H](N)CC3 ; O=C([O-])C(F)(F)F]

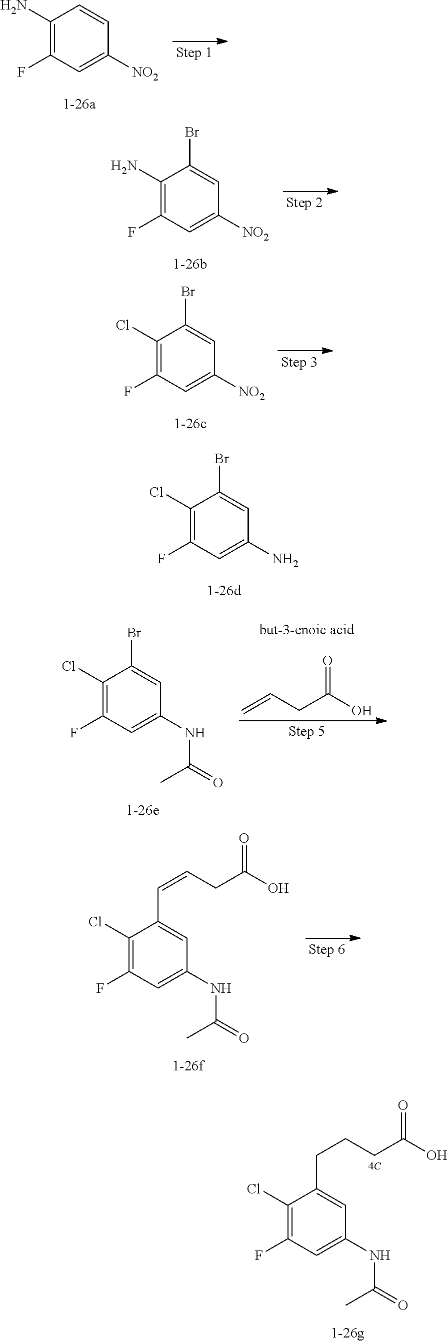

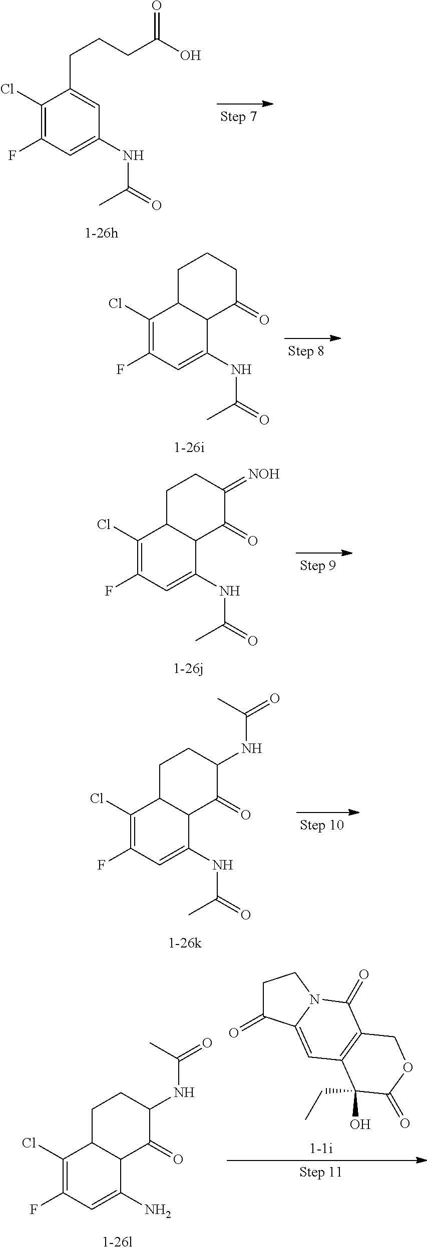

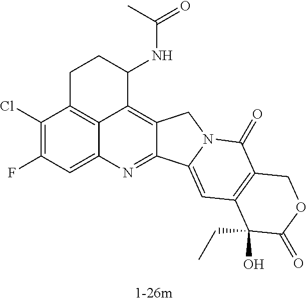 [SMILES: CC[C@@]1(O)C(=O)OCc2c1cc1n(c2=O)Cc2c-1nc1cc(F)c(Cl)c3c1c2C(NC(C)=O)CC3]

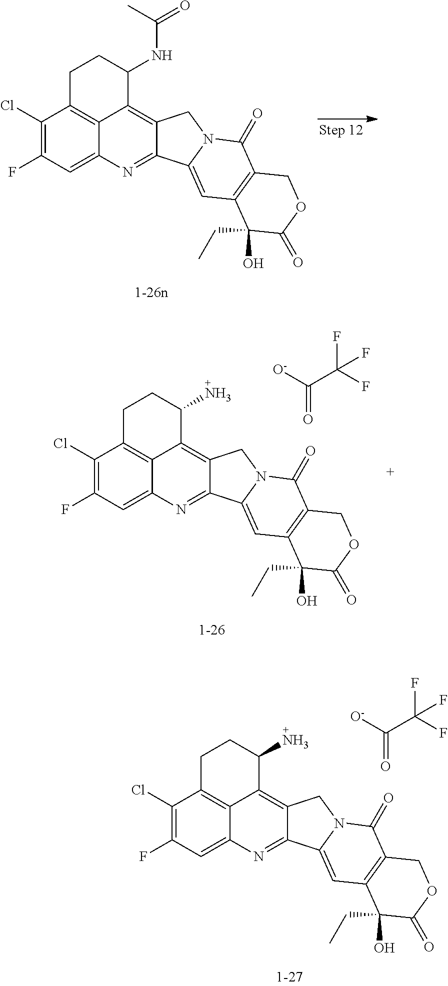 [SMILES: CC[C@@]1(O)C(=O)OCc2c1cc1n(c2=O)Cc2c-1nc1cc(F)c(Cl)c3c1c2C(NC(C)=O)CC3 ; CC[C@@]1(O)C(=O)OCc2c1cc1n(c2=O)Cc2c-1nc1cc(F)c(Cl)c3c1c2[C@@H](N)CC3 ; O=C([O-])C(F)(F)F ; CC[C@@]1(O)C(=O)OCc2c1cc1n(c2=O)Cc2c-1nc1cc(F)c(Cl)c3c1c2[C@H](N)CC3 ; O=C([O-])C(F)(F)F]

### Step 1-Step 12

### ((1S,9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione 2,2,2-trifluoroacetate salt (1-26)

### ((1R,9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione 2,2,2-trifluoroacetate salt (1-27)

1-26 (62 mg, 97% purity) was synthesized according to a reported procedure (US 2020/0384121 A1). Retention time (1.27 min)

MS (ESI) m/z: 456.2 [M+H]⁺

1H NMR (400 MHz, DMSO-d6) δ 8.45 (s, 3H), 8.17 (d, J=10.2 Hz, 1H), 7.38 (s, 1H), 5.74 (d, J=19.4 Hz, OH), 5.46 (dd, J=10.6, 8.7 Hz, 1H), 5.15 (s, 1H), 3.43 (d, J=15.2 Hz, 1H), 3.16 (d, J=13.3 Hz, 1H), 2.07-1.95 (m, 3H), 1.88 (td, J=14.4, 7.1 Hz, 2H), 1.52 (d, J=7.0 Hz, 1H), 1.46 (s, 3H), 0.94-0.83 (m, 3H).

1-27 (75 mg, 95% purity) was synthesized according to a reported procedure (US 2020/0384121 A1). Retention time (1.33 min)

MS (ESI) m/z: 456.2 [M+H]⁺

### Example 2-1

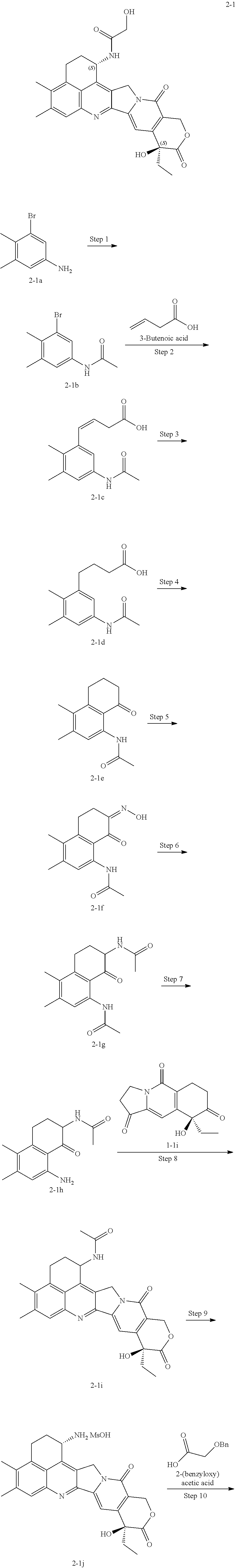

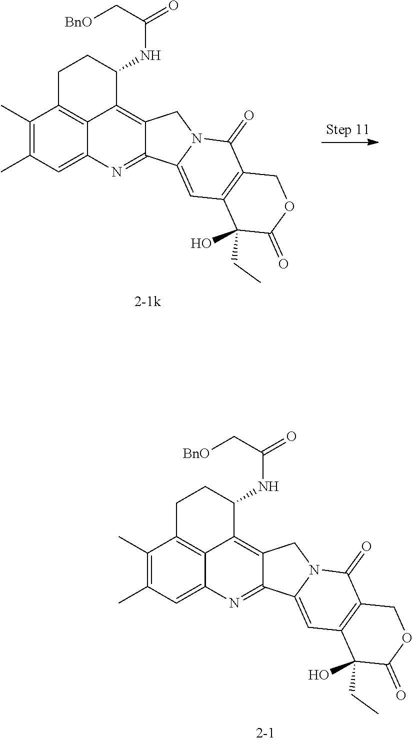 [SMILES: CC[C@@]1(O)C(=O)OCc2c1cc1n(c2=O)Cc2c-1nc1cc(C)c(C)c3c1c2[C@@H](NC(=O)COCc1ccccc1)CC3 ; CC[C@@]1(O)C(=O)OCc2c1cc1n(c2=O)Cc2c-1nc1cc(C)c(C)c3c1c2[C@@H](NC(=O)COCc1ccccc1)CC3]

### Step 1-Step 9

### (1S,9S)-1-amino-9-ethyl-9-hydroxy-4,5-dimethyl-1,2,3,9,12,15-hexahydro-10H,13H-benzo[de]pyrano[3′,4′:6,7]indolizino[1,2-b]quinoline-10,13-dione (2-1j)

2-1j (23 mg, 97% purity) was synthesized according to a reported procedure (US 2020/0384121 A1).

MS (ESI) m/z: 432.4 [M+H]⁺

¹H NMR (400 MHz, d₆-DMSO) δ 8.14 (s, 0.4H), 7.93 (s, 1H), 7.32 (s, 1H), 6.53 (s, 1H), 5.67 (d, J=19.2 Hz, 1H), 5.45 (s, 2H), 5.40 (d, J=19.2 Hz, 1H), 4.95 (s, 1H), 3.26-3.22 (m, 1H), 3.14-3.06 (m, 1H), 2.54 (s, 3H), 2.40 (s, 3H), 2.30-2.84 (m, 1H), 2.19-2.13 (m, 1H), 1.88 (q, J=7.2 Hz, 2H), 0.89 (t, J=7.2 Hz, 3H).

### Step 10 and Step 11

### N-((1S,9S)-9-ethyl-9-hydroxy-4,5-dimethyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-1)

2-1 (7.3 mg, 96% purity, 60% yield) was synthesized according to a reported procedure (CN 112125915 A).

MS (ESI) m/z: 490.4 [M+H]⁺

¹H NMR (400 MHz, d₆-DMSO) δ 8.33 (d, J=8.8 Hz, 1H), 7.86 (s, 1H), 7.28 (s, 1H), 6.50 (s, 1H), 5.58-5.53 (m, 1H), 5.46 (t, J=5.6 Hz, 1H), 5.41 (s, 2H), 5.22 (d, J=19.2 Hz, 1H), 5.14 (d, J=19.2 Hz, 1H), 3.95 (d, J=5.6 Hz, 2H), 3.17-3.12 (m, 2H), 2.51 (s, 3H), 2.37 (s, 3H), 2.20-2.13 (m, 2H), 1.92-1.83 (m, 2H), 0.87 (t, J=7.2 Hz, 3H).

### Example 2-2

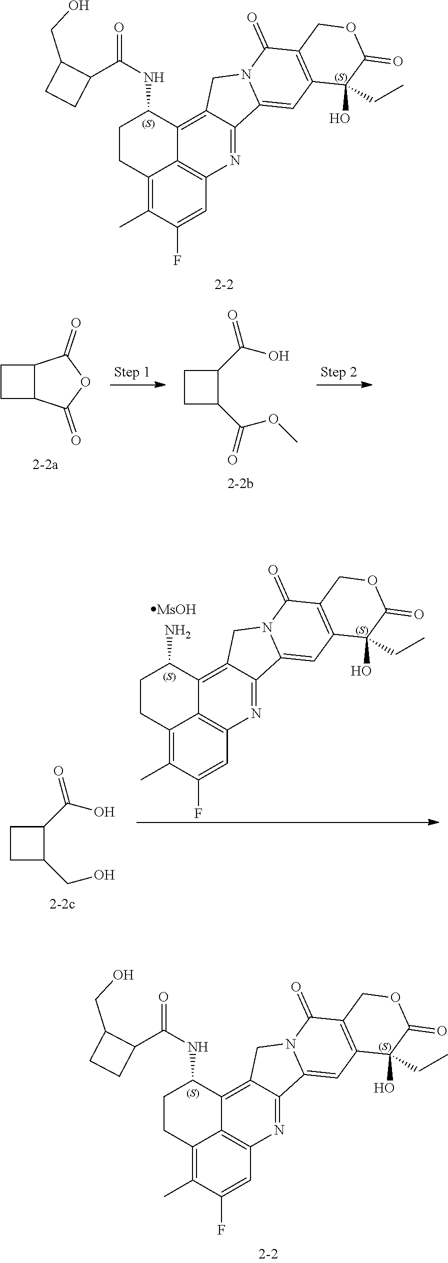

### Step 1

### 2-(methoxycarbonyl)cyclobutane-1-carboxylic acid (2-2b)

A solution of compound 2-2a (2000 mg, 15.86 mmol) in MeOH (20 mL) was refluxed for 4 h. The mixture was concentrated to give the crude product 2-2b (2.5 g, quant. yield), which was used for next step without further purification.

¹H NMR (400 MHz, DMSO-d₆) δ 12.2 (s, 1H), 3.56 (s, 3H), 3.71-3.35 (m, 2H), 2.17-2.09 (m, 4H).

### Step 2

### 2-(hydroxymethyl)cyclobutane-1-carboxylic acid (2-2c)

To a solution of compound 2-2b (200 mg, 1.26 mmol) in THF (4 mL) was added 1 M LiAlH₄ (1.90 mL, 1.90 mmol) at 0° C. The mixture was stirred at r.t. for 2 h. The mixture was quenched by H₂O (72 mg) dropwise at 0° C. The diluted with H₂O (10 mL), extracted with EtOAc (50 mL*3). The organic layer was combined and dried over anhydrous Na₂SO₄, filtered and concentrated to give compound 2-2c (97 mg, crude) as colorless oil.

### Step 3

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-(hydroxymethyl)cyclobutane-1-carboxamide (2-2)

To a solution of compound exatecan mesylate (Purchased from ShangHai HaoYuan MedChemExpress CO. LTD, 50 mg, 0.094 mmol) in DMF (3 mL) were added compound 2-2c (24 mg, 0.18 mmol), HATU (72 mg, 18 mmol) and DIEA (49 mg, 0.38 mmol). The mixture was stirred at r.t. for 1 h. The mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min, the fraction was lyophilized to give compound 2-2 (1.6 mg, 3.0% yield) as white solid.

MS (ESI) m/z: 548.4 [M+H]⁺.

### Example 2-3

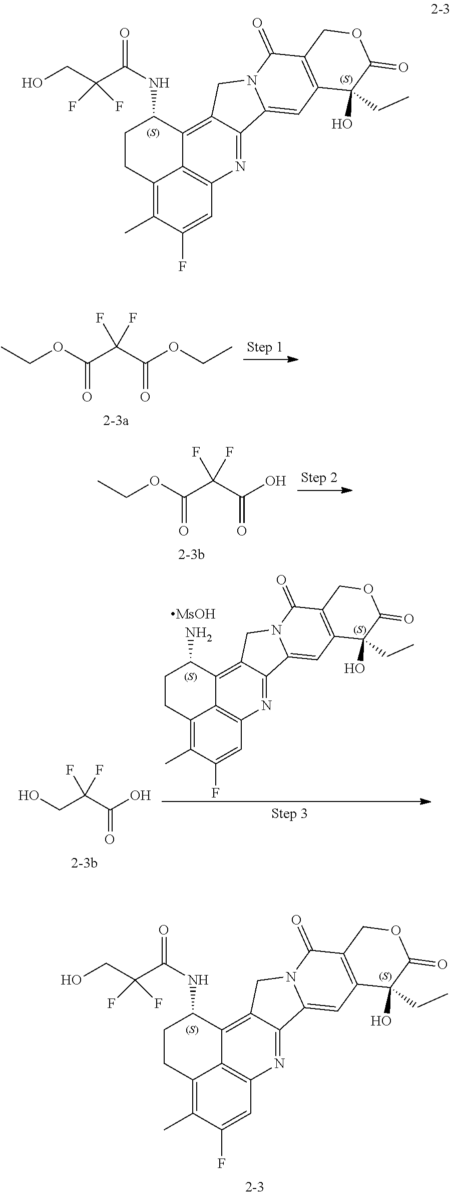

### Step 1

### 3-ethoxy-2,2-difluoro-3-oxopropanoic acid (2-3b)

To a solution of compound 2-3a (2000 mg, 10.2 mmol) in EtOH (10 mL) was added a solution of KOH (572 mg, 10.2 mmol) in H₂O (100 uL) and EtOH (2 mL) dropwise at 0° C. The mixture was warmed up to room temperature and stirred for 2 h. The mixture was diluted with H₂O (20 mL), washed with DCM (20 mL*3). The aqueous solution was adjusted to pH=3 by 1 N HCl. Then extracted by EA (50 mL*3). The organic layer was combined and dried over anhydrous Na₂SO₄, filtered, and concentrated to give compound 2-3b (825 mg, crude) as colorless oil, The crude was used for next step without further purification.

### Step 2

### 2,2-difluoro-3-hydroxypropanoic acid (2-3c)

To a solution of compound 2-2b (800 mg, 4.76 mmol) in isopropanol (10 mL) was added 2 M LiBH₄ (4.76 mL, 9.54 mmol) at 0° C. The mixture was stirred at r.t. for 2 h. The mixture was quenched by 2 N HCl (4.76 mL) dropwise at 0° C. Then diluted with H₂O (20 mL), extracted with EtOAc (50 mL*3). The organic layer was combined and dried over anhydrous Na₂SO₄, filtered and concentrated to give compound 2-3c (417 mg, crude) as colorless oil.

### Step 3

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,2-difluoro-3-hydroxypropanamide (2-3)

Compound 2-3 (8.4 mg, 16.4% yield) was synthesized according to synthetic procedure of step 3 of example 2-2.

MS (ESI) m/z: 544.3 [M+H]⁺

### Example 2-4

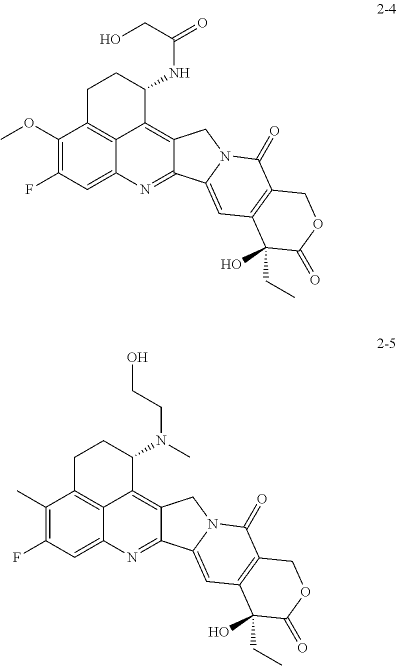 [SMILES: CC[C@@]1(O)C(=O)OCc2c1cc1n(c2=O)Cc2c-1nc1cc(F)c(OC)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](N(C)CCO)CC3]

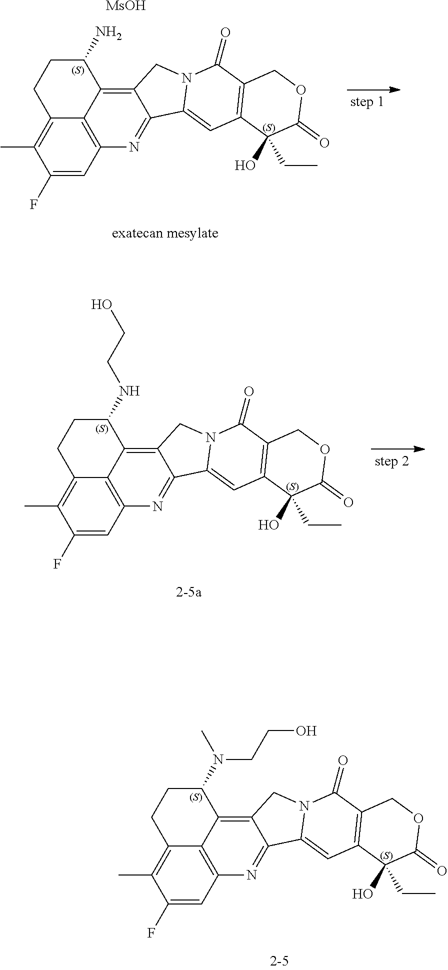

### Step 1

### N-((1S,9S)-9-ethyl-5-fluoro-9-hydroxy-4-methoxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-4)

Compound 2-4 (4.2 mg, 49.5% yield) was synthesized according to synthetic procedure of step 3 of example 2-2.

MS (ESI) m/z: 452.2 [M+H]⁺. Retention time (2.55 min).

### Example 2-5

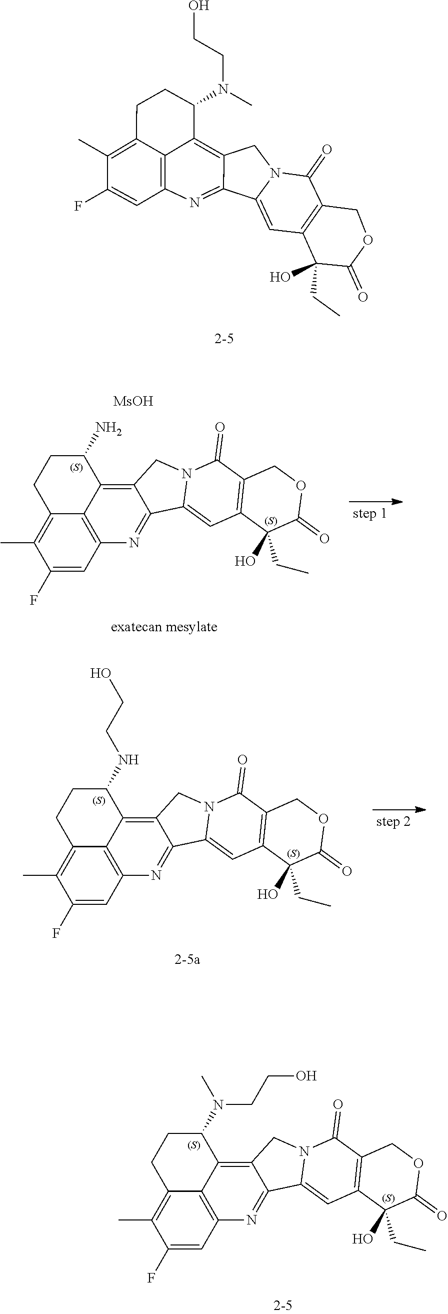

### Step 1

### (1S,9S)-9-ethyl-5-fluoro-9-hydroxy-1-((2-hydroxyethyl)amino)-4-methyl-1,2,3,9,12,15-hexahydro-10H,13H-benzo[de]pyrano[3′,4′:6,7]indolizino[1,2-b]quinoline-10,13-dione (2-5a)

To the turbid solution of exatecan mesylate (200 mg, 0.38 mmol) and glycolaldehyde dimer (50 mg, 0.41 mmol) in CH₂Cl₂/MeOH (2/2 mL) was added NaHCO₃ (32 mg, 0.38 mmol), the mixture was stirred at r.t. overnight. NaBH₃CN (35.8 mg, 0.56 mmol) was added and the solution was stirred for 10 h. The solution was added into half-saturated NH₄Cl solution (6 ml) and filtered. The filtrate was extracted with CH₂Cl₂/MeOH (5/1, 6 mL*3). The organic phase was concentrated and purified by prep-HPLC (Mobile phase A: 0.1% TFA in water, B: CH₃CN; Gradient: 20%-25% B; Flow rate: 20 mL/min; Column: Xbridge Prep C18 OBD™ 5 m, 19*150 mm). The desired fraction was lyophilized to give 2-5a formate salt (52 mg, 28.8% yield) as a light yellow solid.

MS (ESI) m/z: 480.3 [M+H]⁺

¹H NMR (400 MHz, DMSO-d6) δ 9.53 (brs, 1H), 9.32 (brs, 1H), 7.88 (d, J=10.9 Hz, 1H), 7.35 (s, 2H), 6.55 (s, 1H), 5.78 (d, J=18.8 Hz, 1H), 5.45 (s, 3H), 5.31 (s, 1H), 5.11 (s, 1H), 3.76 (s, 2H), 3.31-3.15 (m, 4H), 3.09-2.89 (m, 1H), 2.88-2.74 (m, 1H), 2.40 (s, 3H), 2.23-1.97 (m, 2H), 1.96-1.79 (m, 2H), 0.88 (t, J=7.2 Hz, 3H).

### Step 2

### (1S,9S)-9-ethyl-5-fluoro-9-hydroxy-1-((2-hydroxyethyl)(methyl)amino)-4-methyl-1,2,3,9,12,15-hexahydro-10H,13H-benzo[de]pyrano[3′,4′:6,7]indolizino[1,2-b]quinoline-10,13-dione (2-5)

To the solution of 2-5a (20 mg, 0.04 mmol) in CH₂Cl₂/MeOH (1/0.1 mL) were added HOAc (1 μL, 0.021 mmol), 37% HCHO solution (5 μL, 0.063 mmol) and NaBH₃CN (4 mg, 0.063 mmol), stirred at r.t. for 2 h. The reaction solution was added into half-saturated NH₄Cl (5 mL), extracted with CH₂Cl₂/MeOH (5/1, 3 mL*3). The organic phase was concentrated and purified by prep-HPLC (Mobile phase A: 0.1% FA in water, B: MeCN; Gradient: 20%-28% B; Flow rate: 20 mL/min; Column: Xbridge Prep C18 OBD™ 5 μm, 19*150 mm). The desired fraction was lyophilized to give 2-5 (4 mg, 19.4% yield) as a pale yellow solid.

MS (ESI) m/z: 494.3 [M+H]⁺

¹H NMR (400 MHz, DMSO-d6) δ 7.74 (d, J=11.0 Hz, 1H), 7.31 (s, 1H), 6.49 (s, 1H), 5.53-5.35 (m, 4H), 4.55 (s, 1H), 4.49-4.37 (m, 1H), 3.70-3.58 (m, 2H), 3.01-2.86 (m, 1H), 2.83-2.74 (m, 1H), 2.72-2.62 (m, 2H), 2.37 (s, 3H), 2.35-2.27 (m, 1H), 2.25 (s, 3H), 2.06-1.93 (m, 1H), 1.93-1.77 (m, 2H), 0.88 (t, J=7.3 Hz, 3H).

### Example 2-6

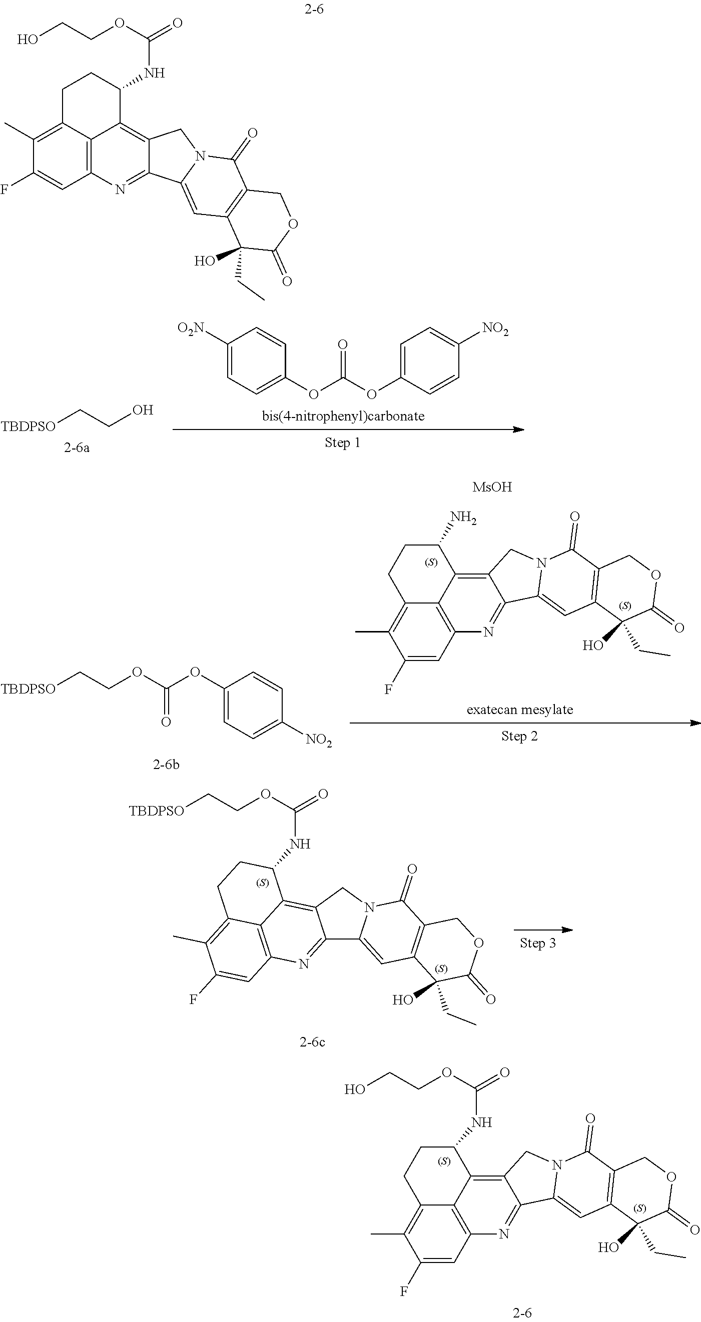

### Step 1

### 2-((tert-butyldiphenylsilyl)oxy)ethyl (4-nitrophenyl) carbonate (2-6b)

To the solution of 2-6a (100 mg, 0.33 mmol) and bis(4-nitrophenyl) carbonate (123 mg, 0.40 mmol) in dry CH₂Cl₂ (2 mL) was added DIEA (176 μL, 1.0 mmol) and DMAP (4.1 mg, 0.033 mmol), stirred at r.t. overnight. The solution was poured into 1N HCl (2 mL), extracted with CH₂Cl₂ (2 mL*3). The organic phase was concentrated and purified by flash column chromatography (silica gel, petroleum ether/ethyl acetate=5:1) to give compound 2-6b (148 mg, 95.5% yield) as a colorless oil.

¹H NMR (400 MHz, CDCl₃) δ 8.27 (d, J=9.1 Hz, 2H), 7.69 (d, J=6.5 Hz, 4H), 7.50-7.31 (m, 8H), 4.47-4.36 (m, 2H), 3.99-3.89 (m, 2H), 1.07 (s, 9H).

### Step 2

### 2-((tert-butyldiphenylsilyl)oxy)ethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (2-6c)

To the solution of exatecan mesylate (50 mg, 0.094 mmol), 2-6b (53 mg, 0.11 mmol) and HOBt (1.3 mg, 0.009 mmol) in dry DMF (1 mL) was added DIEA (50 μL, 0.28 mmol), stirred at r.t. overnight. The solution was poured into sat. NH₄Cl (5 mL), extracted with EtOAc (5 mL*3). The organic phase was concentrated and purified by flash column chromatography (silica gel, petroleum ether/ethyl acetate=1:3) to give compound 2-6c (68 mg, 94.9% yield) as a yellow solid.

MS (ESI) m/z: 762.4 [M+H]⁺

¹H NMR (400 MHz, CDCl₃) δ 7.70-7.60 (m, 5H), 7.59-7.52 (m, 1H), 7.36-7.27 (m, 7H), 5.68 (d, J=16.2 Hz, 1H), 5.33 (d, J=16.3 Hz, 1H), 5.25-5.15 (m, 1H), 5.09-4.96 (m, 2H), 4.56-4.44 (m, 1H), 4.33-4.21 (m, 1H), 4.04-3.89 (m, 2H), 3.70 (s, 1H), 3.17-2.99 (m, 2H), 2.47-2.34 (m, 4H), 2.19-2.07 (m, 1H), 2.05-1.88 (m, 2H), 1.08 (t, J=7.3 Hz, 3H), 1.04 (s, 9H).

### Step 3

### 2-hydroxyethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (2-6)

To the solution of 2-6c (65 mg, 0.085 mmol) in dry THF (2 mL) was added 1M TBAF in THF (102 μL, 0.102 mmol) at 0° C., stirred at r.t. for 40 min. The solution was poured into sat. NH₄Cl (5 mL), extracted with CH₂Cl₂/MeOH (5/1, 6 mL*3). The organic phase was concentrated and purified by flash column chromatography (silica gel, CH₂Cl₂/MeOH=10:1) to give compound 2-6 (39 mg, 87.3% yield) as a pale yellow solid.

MS (ESI) m/z: 524.4 [M+H]⁺

¹H NMR (400 MHz, DMSO-d₆) δ 7.98 (d, J=8.8 Hz, 1H), 7.77 (d, J=10.9 Hz, 1H), 7.31 (s, 1H), 6.52 (s, 1H), 5.43 (s, 2H), 5.33-5.17 (m, 3H), 4.78 (t, J=5.4 Hz, 1H), 4.19-4.01 (m, 2H), 3.68-3.57 (m, 2H), 3.30-3.20 (m, 1H), 3.17-3.06 (m, 1H), 2.38 (s, 3H), 2.26-2.07 (m, 2H), 1.95-1.78 (m, 2H), 0.87 (t, J=7.4 Hz, 3H).

### Example 2-7

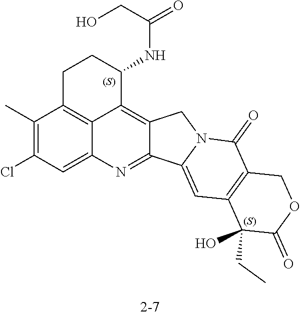

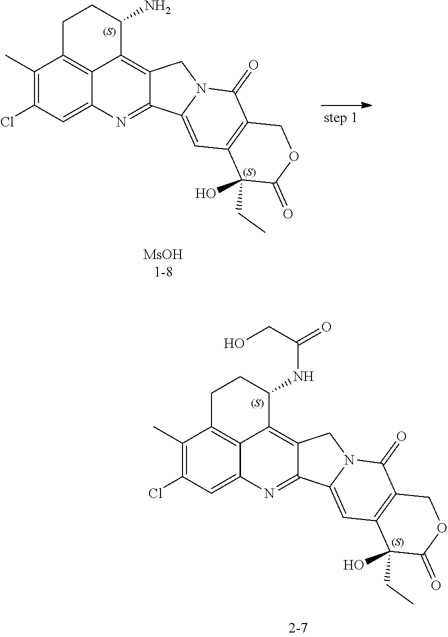

### Step 1

### N-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-7)

To the solution of 2-hydroxyacetic acid (4.7 mg, 0.06 mmol) and HATU (20 mg, 0.05 mmol) in dry DMF (0.4 mL) was added NMM (7 μL, 0.06 mmol), stirred at r.t. for 5 min. The solution was added into the solution of 1-8 masylate (22 mg, 0.04 mmol) and NMM (7 μL, 0.06 mmol) in dry DMF (0.6 mL), stirred for 40 min. The solution was purified by prep-HPLC (Mobile phase A: 0.1% FA in water, B: MeCN; Gradient: 20%-30%-60% B; Flow rate: 20 mL/min; Column: Sunfire Prep C18 OBD™ 5 m, 19*150 mm). The desired fraction was lyophilized to give 2-7 (3.1 mg, 15.1% yield) as a beige solid.

MS (ESI) m/z: 510.3 [M+H]⁺

¹H NMR (400 MHz, DMSO) δ 8.40 (d, J=8.8 Hz, 1H), 8.16 (s, 1H), 7.31 (s, 1H), 6.53 (s, 1H), 5.63-5.54 (m, 1H), 5.48 (t, J=5.8 Hz, 1H), 5.44-5.40 (m, 2H), 5.24 (d, J=19.1 Hz, 1H), 5.18 (d, J=19.0 Hz, 1H), 3.95 (d, J=5.8 Hz, 2H), 3.25-3.12 (m, 2H), 2.53 (s, 3H), 2.27-2.11 (m, 2H), 1.95-1.78 (m, 2H), 0.87 (t, J=7.3 Hz, 3H).

### Example 2-8

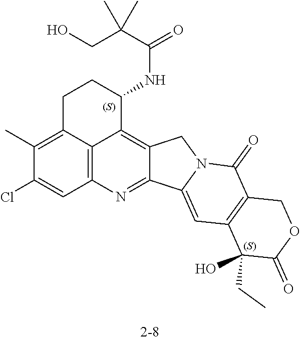

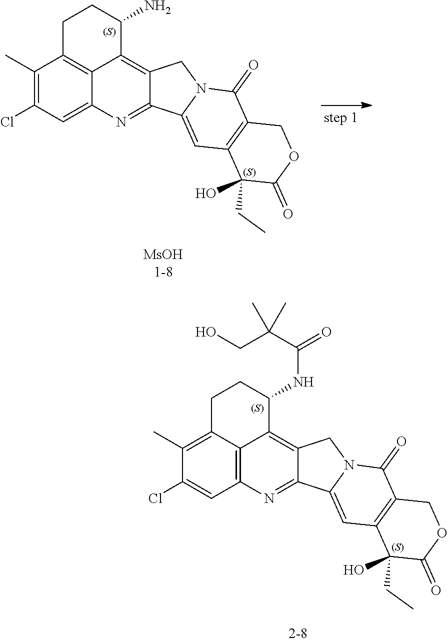

### Step 1

### N-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (2-8)

To the solution of 3-hydroxy-2,2-dimethylpropanoic acid (4.3 mg, 0.036 mmol) and HATU (12.7 mg, 0.033 mmol) in dry DMF (0.5 mL) was added DIEA (7 μL, 0.041 mmol), stirred at r.t. for 5 min. The solution was added into the solution of 1-8 masylate (15 mg, 0.027 mmol) and DIEA (7 μL, 0.041 mmol) in dry DMF (0.5 mL), stirred for 30 min. The solution was purified by prep-HPLC (Mobile phase A: 0.1% FA in water, B: MeCN; Gradient: 35%-55% B; Flow rate: 20 mL/min; Column: Xbridge Prep C18 OBD™ 5 μm, 19*150 mm). The desired fraction was lyophilized to give 2-8 (10.1 mg, 66.8% yield) as an off-white solid.

MS (ESI) m/z: 552.3 [M+H]⁺

¹H NMR (400 MHz, DMSO-d6) δ 7.99 (d, J=8.4 Hz, 1H), 7.31 (s, 1H), 6.53 (s, 1H), 5.60-5.50 (m, 1H), 5.42 (s, 2H), 5.23 (d, J=19.1 Hz, 1H), 5.16 (d, J=19.0 Hz, 1H), 4.88 (t, J=5.1 Hz, 1H), 3.45 (dd, J=10.4, 5.2 Hz, 1H), 3.41-3.35 (m, 1H), 3.22-3.11 (m, 2H), 2.53 (s, 3H), 2.22-2.05 (m, 2H), 1.93-1.78 (m, 2H), 1.12 (s, 3H), 1.10 (s, 3H), 0.87 (t, J=7.3 Hz, 3H).

### Example 2-9 and 2-10

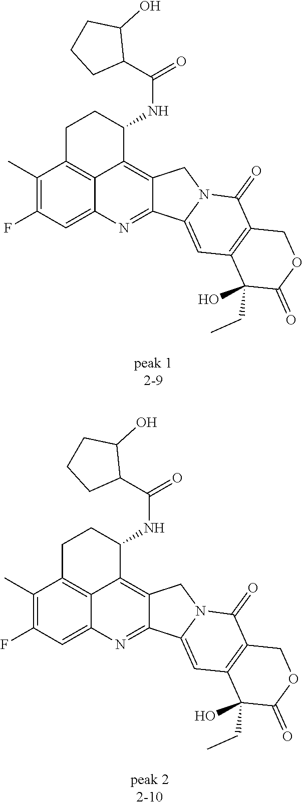 [SMILES: CC[C@@]1(O)C(=O)OCc2c1cc1n(c2=O)Cc2c-1nc1cc(F)c(C)c3c1c2[C@@H](NC(=O)C1CCCC1O)CC3 ; CC[C@@]1(O)C(=O)OCc2c1cc1n(c2=O)Cc2c-1nc1cc(F)c(C)c3c1c2[C@@H](NC(=O)C1CCCC1O)CC3]

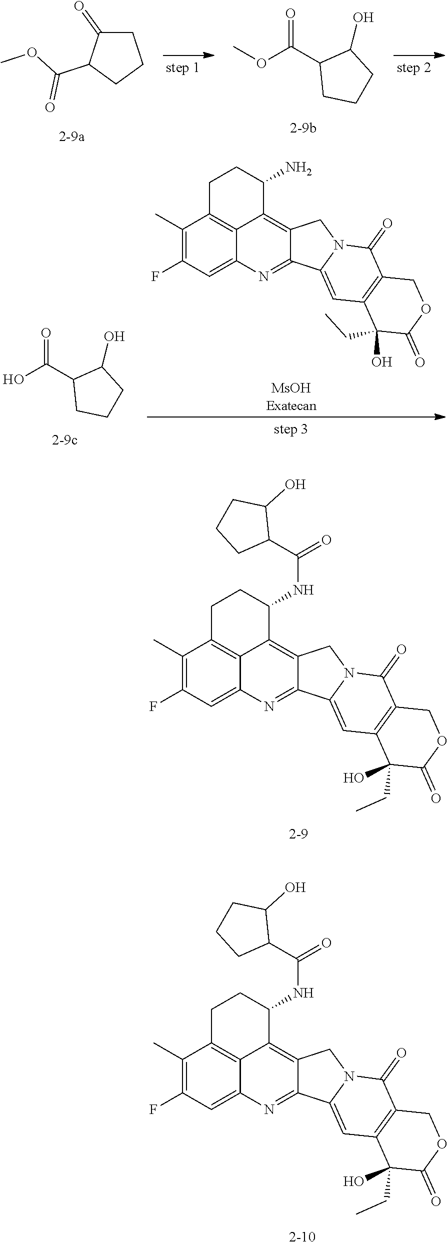

### Step 1

### methyl 2-hydroxycyclopentane-1-carboxylate (2-9b)

A solution of compound 2-9a (3000 mg, 21.12 mmol) in MeOH (20 mL) was cooled to 0° C., then NaBH₄ (1043 mg, 27.46 mmol) was added portionwise. The mixture was stirred at 0° C. for 1 h. TLC (SiO₂, Petroleum ether:EtOAc=3:1, v/v) showed the reaction was completed. The reaction was quenched with 1N HCl. pH was adjusted to 8 using saturated NaHCO₃ aq. and extracted with EtOAc (35 mL*3). The combined organic layer was dried over Na₂SO₄ and concentrated to give the residue which was purified by silica column gel chromatography (eluent: Petroleum ether/EtOAc=100/0 to 40/60) to afford 2-9b (2 g, 65.7% yield) as a colorless oil.

1H NMR (400 MHz, DMSO-d6) δ 4.89 (d, J=4.9 Hz, 1H), 4.21-4.12 (m, 1H), 3.59 (s, 3H), 2.55 (dd, J=14.8, 6.4 Hz, 1H), 1.71-1.57 (m, 6H).

### Step 2

### 2-hydroxycyclopentane-1-carboxylic acid (2-9c)

To a solution of compound 2-9b (500 mg, 3.52 mmol) in THF (10 mL) and H₂O (3 mL) was added LiOH (0.731 g, 30.54 mmol) at 0° C. The mixture was stirred at r.t. for 16 h. TLC (SiO₂, EtOAc) showed the reaction was completed. 1N HCl was added to the mixture dropwise to adjust pH to 2. The aqueous was extracted with MTBE (15 mL*3). The combined organic layer was dried over anhydrous Na₂SO₄, filtered and concentrated to give the crude product compound 2-9c (380 mg, crude) as colorless oil.

### Step 3

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxycyclopentane-1-carboxamide (2-9) and N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxycyclopentane-1-carboxamide (2-10)

To a solution of compound 2-9c (50 mg, 0.38 mmol) in DMF (3 mL) was added Exatecan mesylate (170 mg, 0.39 mmol), HATU (159 mg, 0.42 mmol) and DIEA (98 mg, 0.76 mmol). The mixture was stirred at r.t. for 0.5 h. The mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min, the fraction was lyophilized to give 2-9 (15.6 mg, 7.5% yield) as a yellow solid and 2-10 (30.8 mg, 14.8% yield) as a yellow solid.

MS (ESI) m/z: 548.2 [M+H]⁺.

UPLC analysis: 2-9, peak 1, retention time=3.66 min; 2-10, peak 2, retention time=4.00 min (Mobile phase A: 0.1% FA in water, B: MeCN; Gradient: 15% B maintain 1 min, 15%-95% B, 9 min, 95% B maintain 2 min; Flow rate: 0.6 mL/min; Column: ACQUITY UPLC® BEH C18 1.7 μm).

¹H NMR (400 MHz, DMSO-d6) δ 8.48 (d, J=8.8 Hz, 1H), 7.77 (d, J=10.9 Hz, 1H), 7.30 (s, 1H), 6.52 (s, 1H), 5.56 (dd, J=12.4, 7.5 Hz, 1H), 5.42 (s, 2H), 5.14 (d, J=6.5 Hz, 2H), 4.84 (s, 1H), 4.22 (q, J=6.1 Hz, 1H), 3.25-3.08 (m, 4H), 2.39 (s, 3H), 2.21-2.09 (m, 2H), 2.02-1.92 (m, 2H), 1.90-1.78 (m, 3H), 1.76-1.59 (m, 3H), 1.52-1.42 (m, 2H), 0.86 (d, J=7.4 Hz, 4H).

¹H NMR (400 MHz, DMSO-d6) δ 8.44 (d, J=8.7 Hz, 1H), 7.79 (d, J=11.0 Hz, 1H), 7.32 (s, 1H), 6.52 (s, 1H), 5.62-5.52 (m, 1H), 5.43 (s, 2H), 5.24 (s, 2H), 4.78 (d, J=4.9 Hz, 1H), 4.29-4.16 (m, 1H), 3.17 (d, J=5.2 Hz, 2H), 2.46 (dd, J=8.5, 6.0 Hz, 1H), 2.40 (s, 3H), 2.22-2.05 (m, 2H), 1.85 (td,J=13.8, 6.8 Hz, 4H), 1.67 (ddd, J=20.8, 14.4, 7.1 Hz, 3H), 1.51-1.39 (m, 1H), 0.87 (t, J=7.3 Hz, 3H).

### Example 2-11

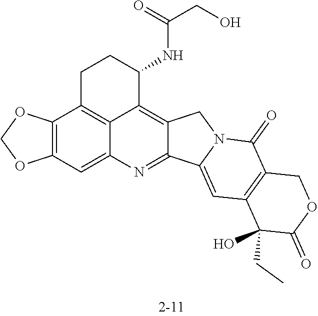 [SMILES: CC[C@@]1(O)C(=O)OCc2c1cc1n(c2=O)Cc2c-1nc1cc3c(c4c1c2[C@@H](NC(=O)CO)CC4)OCO3]

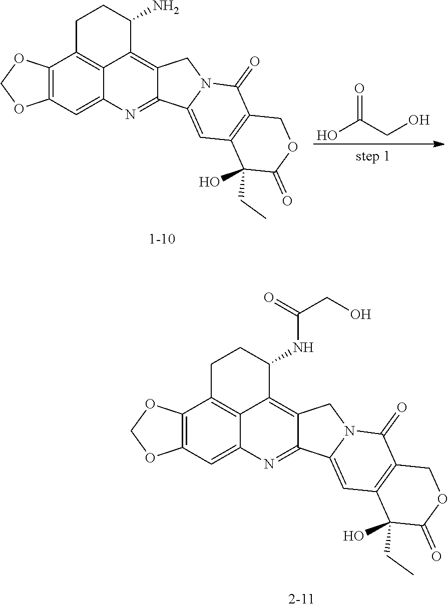

### Step 1

### N-((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-2-hydroxyacetamide (2-11)

To a solution of 2-hydroxyacetic acid (4.47 mg, 0.06 mmol) and 1-10 (15 mg, 0.03 mmol) in DMF (3 mL) was added HATU (26.6 mg, 0.07 mmol) and DIEA (15.48 mg, 0.12 mmol). The resulting mixture was stirred at 20° C. for 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min) to give 2-11 (3.3 mg, 19.8% yield) as a white solid.

MS (ESI) m/z: 506.2 [M+H]⁺.

### Example 2-12&2-13

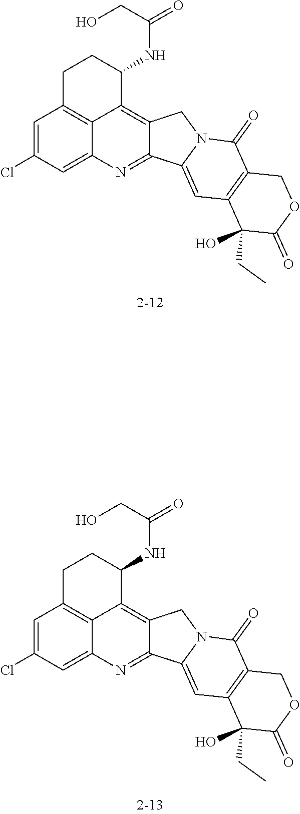 [SMILES: CC[C@@]1(O)C(=O)OCc2c1cc1n(c2=O)Cc2c-1nc1cc(Cl)cc3c1c2[C@@H](NC(=O)CO)CC3 ; CC[C@@]1(O)C(=O)OCc2c1cc1n(c2=O)Cc2c-1nc1cc(Cl)cc3c1c2[C@H](NC(=O)CO)CC3]

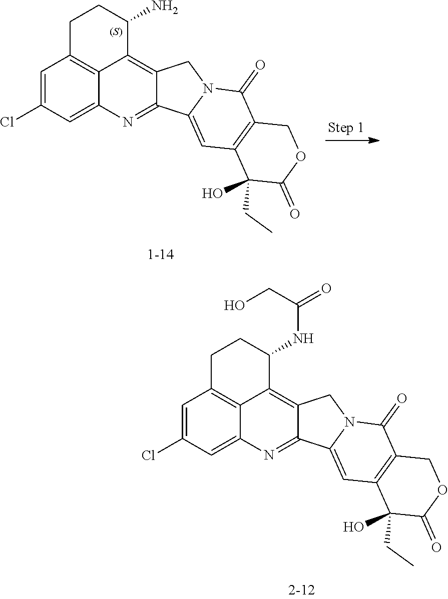

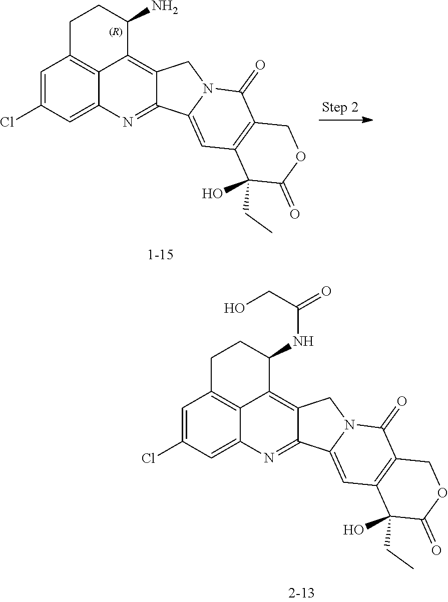

### Step 1

### N-((1S,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-12)

To a solution of 2-hydroxyacetic acid (6.52 mg, 0.09 mmol) in DMF (3 mL) and 1-14 (25 mg, 0.06 mmol) were added HBTU (32.54 mg, 0.09 mmol) and DIEA (22.18 mg, 0.17 mmol), reacted at r.t. for 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 2-12 (5.30 mg, 18.72% yield) was obtained as a white solid.

MS (ESI) m/z: 496.2 [M+H]⁺

¹H NMR (400 MHz, DMSO-d6) δ 8.48 (d, J=8.9 Hz, 1H), 8.05 (d, J=2.1 Hz, 1H), 7.56 (d, J=1.9 Hz, 1H), 7.30 (s, 1H), 6.51 (s, 1H), 5.63-5.47 (m, 2H), 5.39 (s, 2H), 5.15 (q, J=19.2 Hz, 2H), 3.94 (d, J=5.2 Hz, 2H), 3.24-3.07 (m, 2H), 2.20-2.06 (m, 2H), 1.93-1.74 (m, 2H), 0.83 (t, J=7.3 Hz, 3H).

### Step 2

### N-((1R,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-13)

To a solution of 2-hydroxyacetic acid (5.22 mg, 0.07 mmol) in DMF (3 mL) and 1-15 (20 mg, 0.05 mmol) were added HBTU (26.03 mg, 0.07 mmol) and DIEA (17.74 mg, 0.14 mmol), reacted at r.t. for 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 2-13 (5.40 mg, 23.84% yield) was obtained as a white solid.

MS (ESI) m/z: 496.2 [M+H]⁺

¹H NMR (400 MHz, DMSO-d6) δ 8.50 (d, J=9.0 Hz, 1H), 8.04 (d, J=2.1 Hz, 1H), 7.55 (d, J=1.8 Hz, 1H), 7.30 (s, 1H), 6.50 (s, 1H), 5.57 (dd, J=16.5, 9.7 Hz, 2H), 5.39 (d, J=2.2 Hz, 2H), 5.14 (dd, J=37.6, 19.1 Hz, 2H), 3.95 (d, J=4.5 Hz, 2H), 3.17 (s, 2H), 2.25-2.06 (m, 2H), 1.92-1.74 (m, 2H), 0.84 (t, J=7.3 Hz, 3H).

### Example 2-14

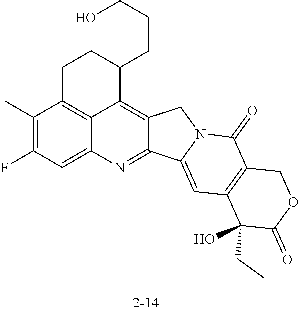 [SMILES: CC[C@@]1(O)C(=O)OCc2c1cc1n(c2=O)Cc2c-1nc1cc(F)c(C)c3c1c2C(CCCO)CC3]

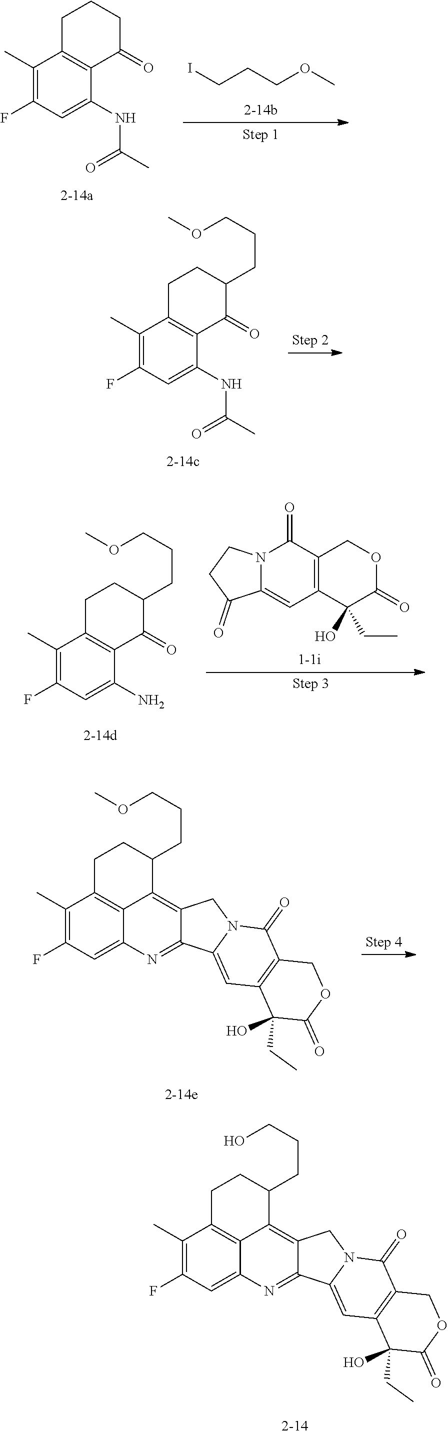

### Step 1

### N-(3-fluoro-7-(3-methoxypropyl)-4-methyl-8-oxo-5,6,7,8-tetrahydronaphthalen-1-yl)acetamide (2-14c)

To a solution of LDA (1.40 mL, 2.81 mmol, 2M in THF) in THF (19 mL) was added 2-14a (300.00 mg, 1.28 mmol) at −78° C., and the mixture was stirred at the same temperature for 2 h before a solution of 2-14b (0.38 g, 1.91 mmol) in THF (1 mL) was added dropwise. The reaction mixture was then slowly warmed up to 0° C. and stirred continuously for 4 h. The reaction was subsequently quenched with saturated aqueous NH₄Cl solution (50 mL), and the organic materials were extracted with EtOAc (30 mL*3) The combined organic layers were washed with brine (50 mL) and dried over MgSO₄ before the combined extracts were concentrated in vacuo. The resulting crude residue was purified by flash column chromatography (silica gel, Hex:EtOAc=97:3) to give 2-14c (80.00 mg, 20.41% yield) as a colorless oil.

MS (ESI) m/z: 308.2 [M+H]⁺

### Step 2

### 8-amino-6-fluoro-2-(3-methoxypropyl)-5-methyl-3,4-dihydronaphthalen-1 (2H)-one (2-14d)

Compound 2-14d (69.06 mg, crude) was synthesized according to synthetic procedure of step 6 of example 1-14.

MS (ESI) m/z: 266.2[M+H]⁺

### Step 3

### (9S)-9-ethyl-5-fluoro-9-hydroxy-1-(3-methoxypropyl)-4-methyl-1,2,3,9,12,15-hexahydro-10H,13H-benzo[de]pyrano[3′,4′:6,7]indolizino[1,2-b]quinoline-10,13-dione (2-14e)

Compound 2-14e (80.00 mg, 62.40% yield) was synthesized according to synthetic procedure of step 7 of example 1-14.

MS (ESI) m/z: 493.1 [M+H]⁺

### Step 4

### (9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (2-14)

To a solution of 2-14e (70.00 mg, 0.14 mmol) in CH₂Cl₂ (20 mL) was added BBr₃ (71.26 mg, 0.28 mmol) was added at −0° C., and the mixture was stirred at the same temperature for 1 h. The reaction mixture was then slowly warmed up to 25° C. and stirred continuously for 3 h. The reaction was subsequently quenched with saturated aqueous NaHCO₃ solution and the organic materials were extracted thrice with EtOAc (30 mL*3). The combined organic layers were washed with brine (50 mL) and dried over MgSO₄ before the combined extracts were concentrated in vacuo. The resulting crude residue was purified by flash column chromatography (silica gel, CH₂Cl₂: MeOH=90:10) to give 2-14 (4.20 mg, 6.18% yield) as a gray solid.

MS (ESI) m/z: 479.4 [M+H]⁺

UPLC analysis: 2-14-1, peak 1, retention time=4.49 min; 2-14-2, peak 2, retention time=4.65 min (Mobile phase A: 0.1% FA in water, B: MeCN; Gradient: 15% B maintain 1 min, 15%-95% B, 9 min, 95% B maintain 2 min; Flow rate: 0.6 mL/min; Column: ACQUITY UPLC® BEH C18 1.7 μm)

2-14-1: ¹H NMR (400 MHz, DMSO-d6) δ 7.74 (d, J=11.1 Hz, 1H), 7.30 (s, 1H), 6.51 (s, 1H), 5.43 (s, 2H), 5.36 (d, J=18.7 Hz, 1H), 5.22 (d, J=18.7 Hz, 1H), 3.52-3.39 (m, 2H), 3.18-2.97 (m, 2H), 2.38 (s, 3H), 2.34-2.25 (m, 2H), 2.01-1.78 (m, 3H), 1.78-1.51 (m, 4H), 0.87 (t, J=7.3 Hz, 3H).

2-14-2: ¹H NMR (400 MHz, DMSO-d6) δ 7.74 (d, J=11.1 Hz, 1H), 7.30 (s, 1H), 6.52 (s, 1H), 5.43 (s, 2H), 5.37 (d, J=18.8 Hz, 1H), 5.23 (d, J=18.7 Hz, 1H), 3.51-3.40 (m, 2H), 3.16-2.98 (m, 2H), 2.38 (s, 3H), 2.35-2.24 (m, 2H), 1.99-1.81 (m, 3H), 1.78-1.53 (m, 4H), 0.87 (t, J=7.3 Hz, 3H).

### Example 2-15

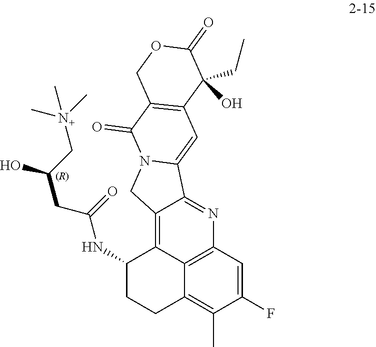

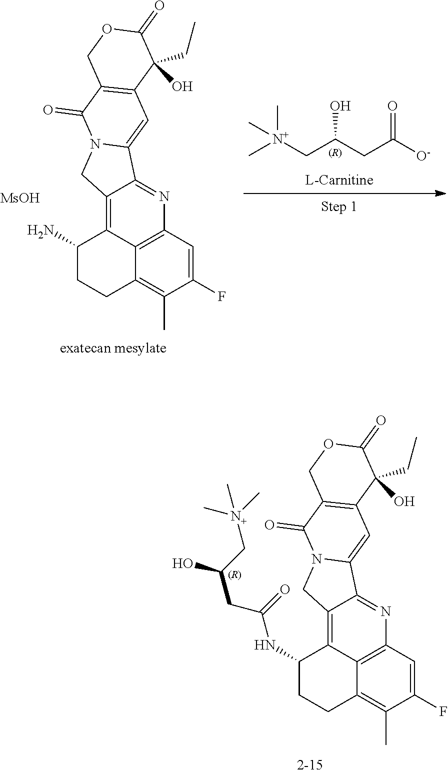

### Step 1

### (R)-4-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-hydroxy-N,N,N-trimethyl-4-oxobutan-1-aminium (2-15)

To a solution of L-Carnitine (30.52 mg, 0.19 mmol) in DMF (3 mL) and exatecan mesylate (50.00 mg, 0.09 mmol) were added HBTU (53.55 mg, 0.14 mmol) and DIEA (36.50 mg, 0.28 mmol) was added into the mixture and reacted at r.t. for another 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 2-15 (15.6 mg, 28.61% yield) was obtained as a white solid.

MS (ESI) m/z: 580.5 [M+H]⁺

### Example 2-16

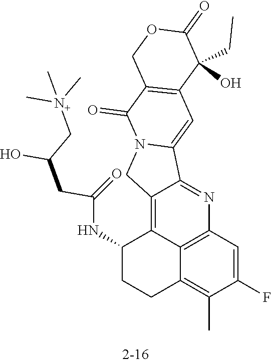 [SMILES: CC[C@@]1(O)C(=O)OCc2c1cc1n(c2=O)Cc2c-1nc1cc(F)c(C)c3c1c2[C@@H](NC(=O)C[C@@H](O)C[N+](C)(C)C)CC3]

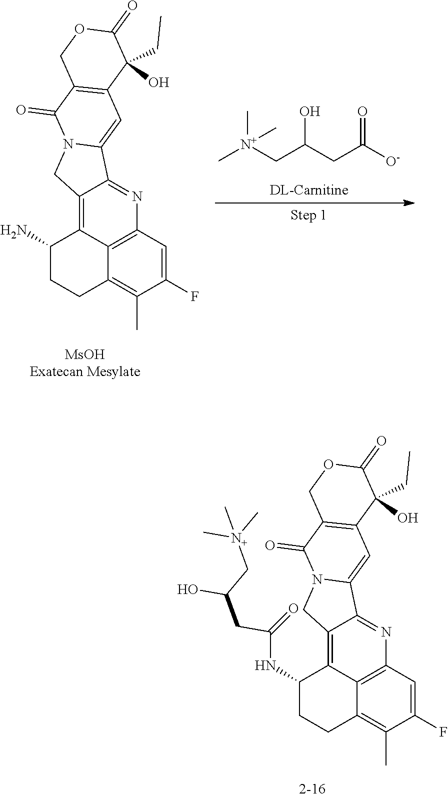 [SMILES: CC[C@@]1(O)C(=O)OCc2c1cc1n(c2=O)Cc2c-1nc1cc(F)c(C)c3c1c2[C@@H](N)CC3 ; C[N+](C)(C)CC(O)CC(=O)[O-] ; CC[C@@]1(O)C(=O)OCc2c1cc1n(c2=O)Cc2c-1nc1cc(F)c(C)c3c1c2[C@@H](NC(=O)C[C@@H](O)C[N+](C)(C)C)CC3]

### Step 2

### 4-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-hydroxy-N,N,N-trimethyl-4-oxobutan-1-aminium (2-16)

Compound 2-16 (19.40 mg, 35.58% yield) was synthesized according to synthetic procedure of step 1 of example 2-15.

MS (ESI) m/z: 579.5 [M+H]⁺

¹H NMR (400 MHz, DMSO-d6) δ 9.02 (dd, J=48.5, 8.5 Hz, 1H), 8.35 (s, 1H), 7.80 (dd, J=10.9, 3.6 Hz, 1H), 7.32 (s, 1H), 6.60 (s, 1H), 5.67-5.55 (m, 1H), 5.42 (s, 2H), 5.37-5.13 (m, 2H), 4.54 (s, 1H), 3.15 (d, J=8.7 Hz, 12H), 2.46-2.36 (m, 5H), 2.16 (d, J=5.5 Hz, 2H), 1.95-1.78 (m, 2H), 0.87 (t, J=7.3 Hz, 3H).

### Example 2-17

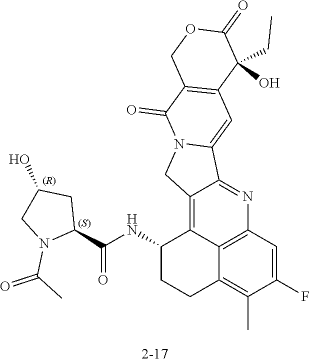

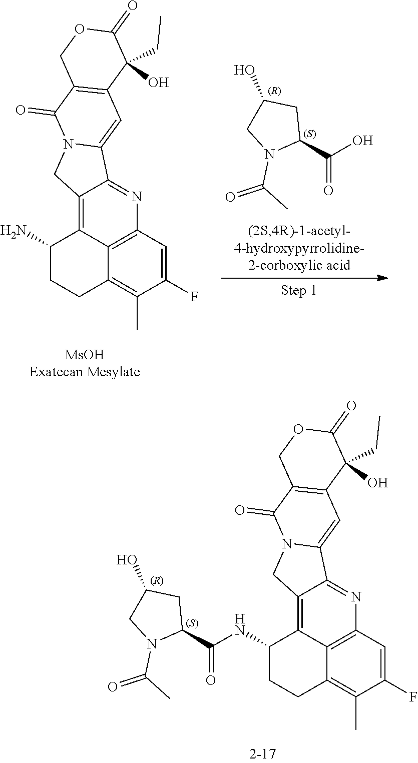

### Step 1

### (2S,4R)-1-acetyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxypyrrolidine-2-carboxamide (2-17)

Compound 2-17 (19.00 mg, 42.75% yield) was synthesized according to synthetic procedure of step 1 of example 2-15.

MS (ESI) m/z: 591.4 [M+H]⁺

### Example 2-18

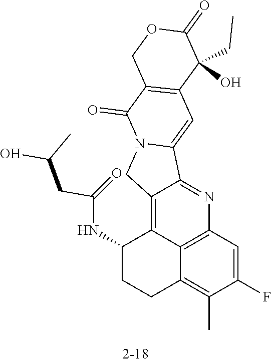 [SMILES: CC[C@@]1(O)C(=O)OCc2c1cc1n(c2=O)Cc2c-1nc1cc(F)c(C)c3c1c2[C@@H](NC(=O)C[C@H](C)O)CC3]

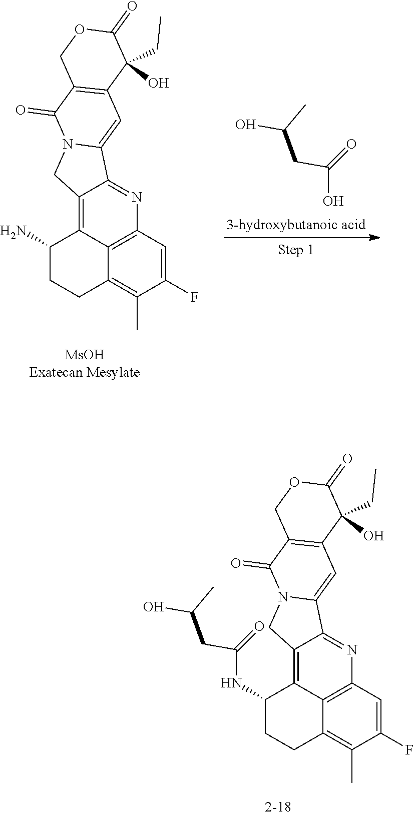

### Step 25

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxybutanamide (2-18)

Compound 2-18 (7.40 mg, 25.14% yield) was synthesized according to synthetic procedure of step 1 of example 2-15.

MS (ESI) m/z: 522.4[M+H]⁺

¹H NMR (400 MHz, DMSO-d6) δ 8.43 (d, J=8.6 Hz, 1H), 7.80 (d, J=11.0 Hz, 1H), 7.31 (s, 1H), 6.53 (s, 1H), 5.56 (d, J=8.2 Hz, 1H), 5.42 (s, 2H), 5.23 (t, J=8.4 Hz, 2H), 4.66 (s, 1H), 4.04 (s, 1H), 3.17 (s, 2H), 2.40 (s, 3H), 2.33-2.08 (m, 5H), 1.92-1.80 (m, 2H), 1.08 (d, J=6.2 Hz, 3H), 0.87 (t, J=7.3 Hz, 3H).

### Example 2-19

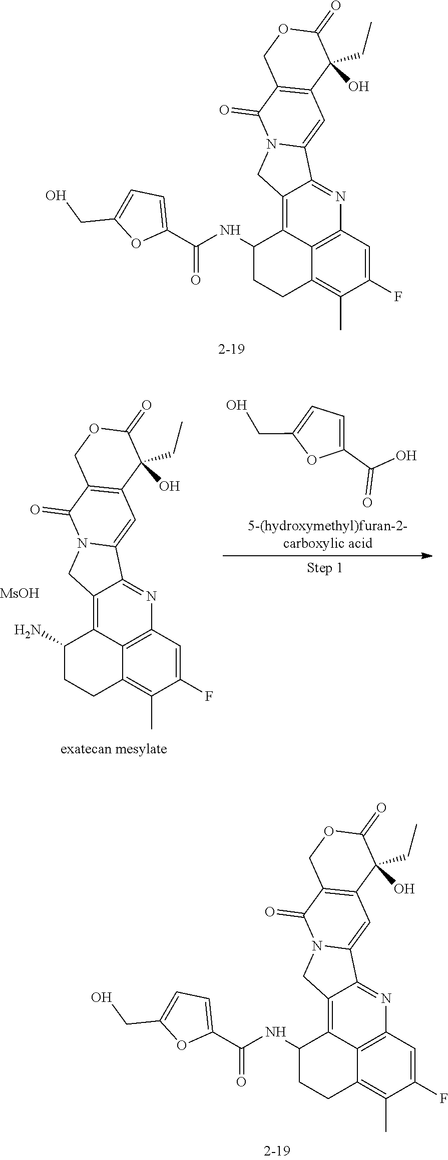 [SMILES: "MsOH" ; CC[C@@]1(O)C(=O)OCc2c1cc1n(c2=O)Cc2c-1nc1cc(F)c(C)c3c1c2C(NC(=O)c1ccc(CO)o1)CC3 ; CC[C@@]1(O)C(=O)OCc2c1cc1n(c2=O)Cc2c-1nc1cc(F)c(C)c3c1c2[C@@H](N)CC3 ; O=C(O)c1ccc(CO)o1 ; CC[C@@]1(O)C(=O)OCc2c1cc1n(c2=O)Cc2c-1nc1cc(F)c(C)c3c1c2C(NC(=O)c1ccc(CO)o1)CC3]

### Step 1

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-5-(hydroxymethyl)furan-2-carboxamide (2-19)

Compound 2-19 (8.80 mg, 41.79% yield) was synthesized according to synthetic procedure of step 1 of example 2-15.

MS (ESI) m/z: 560.3 [M+H]⁺

¹H NMR (400 MHz, DMSO-d6) δ 8.92 (d, J=8.7 Hz, 1H), 7.80 (d, J=11.0 Hz, 1H), 7.31 (s, 1H), 7.17 (d, J=3.4 Hz, 1H), 6.54-6.42 (m, 2H), 5.73 (d, J=8.4 Hz, 1H), 5.43-5.36 (m, 3H), 5.16 (d, J=16.8 Hz, 2H), 4.44 (d, J=5.6 Hz, 2H), 3.18 (s, 2H), 2.24 (d, J=6.0 Hz, 2H), 1.88-1.82 (m, 2H), 0.86 (t, J=7.2 Hz, 3H).

### Example 2-20

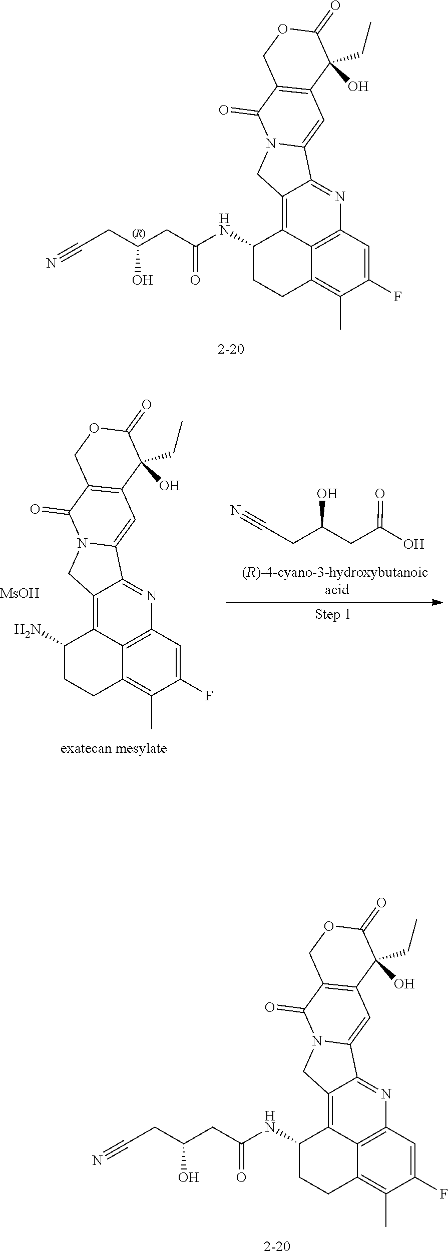

### Step 1

### (R)-4-cyano-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxybutanamide (2-20)

Compound 2-20 (24.2 mg, 47.07% yield) was synthesized according to synthetic procedure of step 1 of example 2-15.

MS (ESI) m/z: 547.4 [M+H]⁺

¹H NMR (400 MHz, DMSO-d6) δ 8.55 (d, J=8.6 Hz, 1H), 7.78 (d, J=11.0 Hz, 1H), 7.30 (s, 1H), 6.54 (s, 1H), 5.58-5.50 (m, 2H), 5.42 (s, 2H), 5.20 (d, J=2.4 Hz, 2H), 4.20 (d, J=5.6 Hz, 1H), 3.17 (s, 2H), 2.74-2.60 (m, 2H), 2.44-2.34 (m, 5H), 2.25-2.06 (m, 2H), 1.92-1.76 (m, 2H), 0.87 (t, J=7.2 Hz, 3H).

### Example 2-21

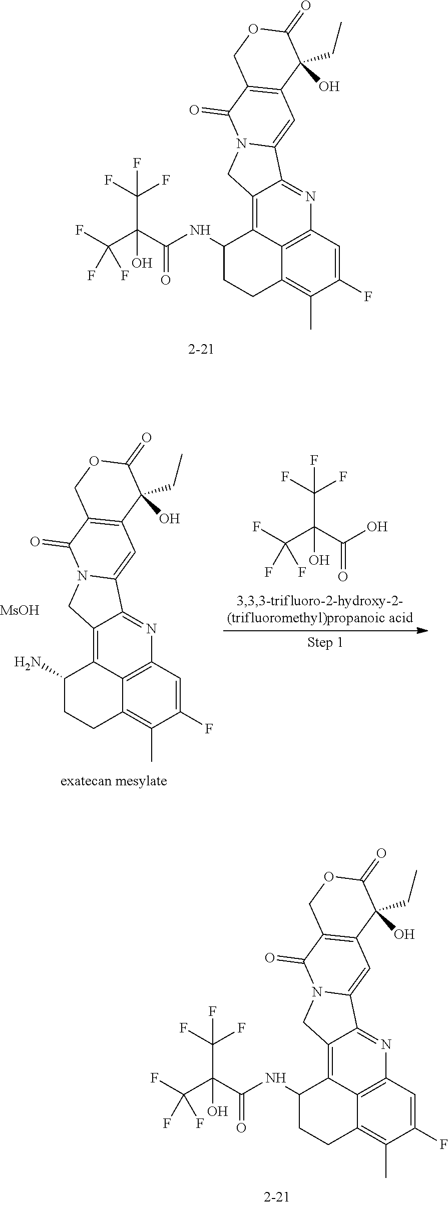 [SMILES: "MsOH" ; CC[C@@]1(O)C(=O)OCc2c1cc1n(c2=O)Cc2c-1nc1cc(F)c(C)c3c1c2C(NC(=O)C(O)(C(F)(F)F)C(F)(F)F)CC3 ; CC[C@@]1(O)C(=O)OCc2c1cc1n(c2=O)Cc2c-1nc1cc(F)c(C)c3c1c2[C@@H](N)CC3 ; O=C(O)C(O)(C(F)(F)F)C(F)(F)F ; CC[C@@]1(O)C(=O)OCc2c1cc1n(c2=O)Cc2c-1nc1cc(F)c(C)c3c1c2C(NC(=O)C(O)(C(F)(F)F)C(F)(F)F)CC3]

### Step 1

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3,3,3-trifluoro-2-hydroxy-2-(trifluoromethyl)propenamide (2-21)

Compound 2-21 (4.00 mg, 16.88% yield) was synthesized according to synthetic procedure of step 1 of example 2-15.

MS (ESI) m/z: 630.4 [M+H]⁺

¹H NMR (400 MHz, DMSO-d6) δ 9.49 (d, J=8.5 Hz, 1H), 9.32 (s, 1H), 7.80 (d, J=10.9 Hz, 1H), 7.31 (s, 1H), 6.94 (d, J=214.5 Hz, 1H), 6.54 (s, 1H), 5.60 (d, J=5.9 Hz, 1H), 5.42 (s, 2H), 5.14 (dd, J=41.5, 18.9 Hz, 2H), 3.16 (s, 2H), 2.40 (s, 3H), 2.23-2.12 (m, 2H), 2.00 (d, J=7.6 Hz, 1H), 1.85 (td, J=14.3, 6.9 Hz, 2H), 1.45 (s, 1H), 0.86 (d, J=7.2 Hz, 4H).

### Example 2-22

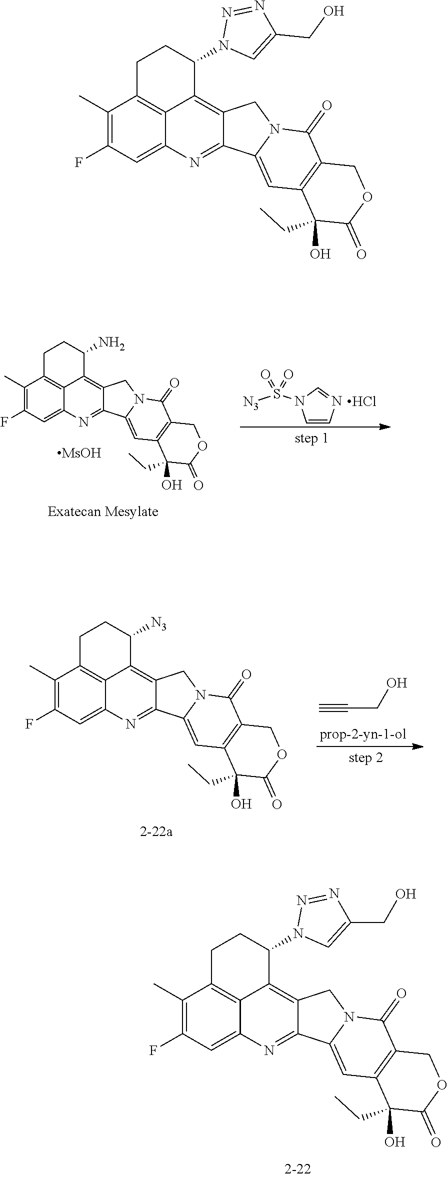

### Step 1

### (1S,9S)-1-azido-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (2-22a)

To a solution of exatecan mesylate (200 mg, 0.38 mmol) in DMF (2 mL) was added Et₃N (80 μL, 57.6 mg, 0.57 mmol) and Imidazole-1-sulfonyl azide hydrochloride (94.3 mg, 0.45 mmol) in 2 mL MeOH. The reaction mixture was stirred at r.t. for 5 min, followed by the addition of K₂CO₃ (105.1 mg, 0.76 mmol) and cat. amount of CuSO₄ (1 mg) in 2 mL H₂O. The mixture was stirred at r.t. for 1 h. On completion of the reaction, the solvent was removed by evaporation. The crude brown solid was purified by silica column gel chromatography (eluent: CH₂Cl₂/MeOH=50/1 to 20/1) to afford 2-22a as a pale white solid (117.2 mg, yield 66.8%).

MS (ESI) m/z: 462.3 [M+H]⁺

¹H NMR (400 MHz, DMSO-d6) δ 7.81 (d, J=10.9 Hz, 1H), 7.44 (s, 1H), 7.32 (s, 1H), 6.52 (s, 1H), 5.56 (t, J=4.9 Hz, 1H), 5.52-5.41 (m, 3H), 5.33 (d, J=19.1 Hz, 1H), 3.71 (s, 2H), 3.17 (s, 2H), 2.39 (s, 3H), 2.32 (m, 2H), 1.87 (m, 2H), 0.88 (t, J=7.3 Hz, 3H).

### Step 2

### (1S,9S)-9-ethyl-5-fluoro-9-hydroxy-1-(4-(hydroxymethyl)-1H-1,2,3-triazol-1-yl)-4-methyl-1,2,3,9,12,15-hexahydro-10H,13H-benzo[de]pyrano[3′,4′:6,7]indolizino[1,2-b]quinoline-10,13-dione (2-22)

To a solution of 2-22a (20 mg, 0.04 mmol) in DMF (1 mL) was added propargyl alcohol (5 mg, 0.08 mmol), CuI (1 mg, cat. amount) and DIPEA (35.1 μL, 25.86 mg, 0.2 mmol). The mixture was purged with N₂ balloon for 15 min, then stirred at r.t. for 2 h. On completion of the reaction, the solvent was removed by evaporation. The crude product was purified by silica column gel chromatography (eluent: CH₂Cl₂/MeOH=10/1 to 5/1) to afford 2-22 as a light yellow solid (10.2 mg, yield 49.3%).

MS (ESI) m/z: 518.4 [M+H]⁺

¹H NMR (400 MHz, DMSO-d6) δ 8.10 (s, 1H), 7.88 (d, J=10.9 Hz, 1H), 7.31 (s, 1H), 6.56 (s, 1H), 5.38 (s, 2H), 5.20 (d, J=19.0 Hz, 1H), 4.51 (s, 2H), 4.14 (d, J=19.0 Hz, 1H), 3.04 (s, 1H), 2.75-2.66 (m, 1H), 2.41 (s, 3H), 1.84 (tt, J=14.2, 7.2 Hz, 2H), 0.85 (t, J=7.2 Hz, 3H).

### Example 2-23

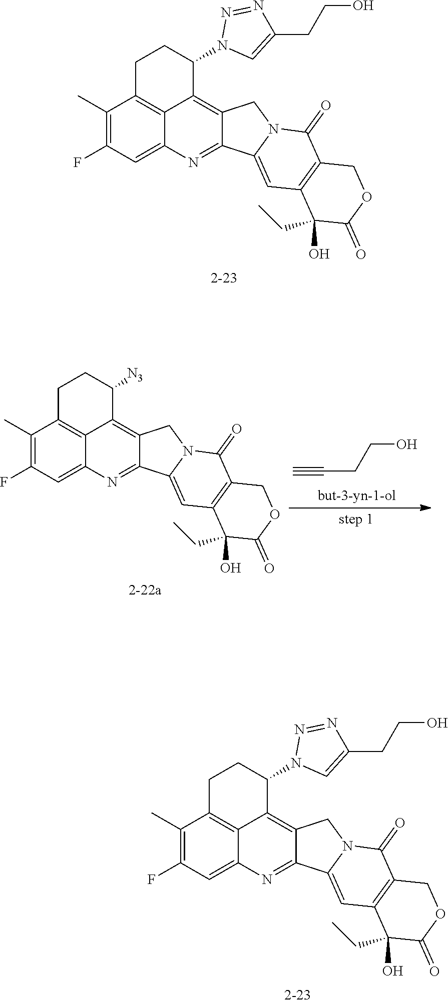

### Step 1

### (1S,9S)-9-ethyl-5-fluoro-9-hydroxy-1-(4-(2-hydroxyethyl)-1H-1,2,3-triazol-1-yl)-4-methyl-1,2,3,9,12,15-hexahydro-10H,13H-benzo[de]pyrano[3′,4′:6,7]indolizino[1,2-b]quinoline-10,13-dione (2-23)

To a solution of 2-22a (20 mg, 0.043 mmol) in DMF (1 mL) was added 3-Butyn-1-ol (3.4 mg, 0.048 mmol), CuI (1 mg, cat. amount) and DIPEA (37.6 μL, 28 mg, 0.21 mmol). The mixture was purged with N₂ balloon for 15 min, then stirred at r.t. for 2 h. On completion of the reaction, the solvent was removed by evaporation. The crude product was purified by silica column gel chromatography (eluent: CH₂Cl₂/MeOH═CH₂Cl₂/MeOH=10/1 to 5/1) to afford 2-23 as a light yellow solid (15.1 mg, yield 65.7%).

¹H NMR (400 MHz, DMSO-d6) δ 8.26 (s, 1H), 8.10 (d, J=10.8 Hz, 1H), 7.54 (s, 1H), 6.76 (s, 2H), 5.62 (s, 2H), 5.32 (d, J=19.0 Hz, 1H), 4.91 (t, J=5.4 Hz, 1H), 4.25 (d, J=19.1 Hz, 1H), 3.87 (dd, J=12.3, 6.3 Hz, 2H), 3.53-3.45 (m, 1H), 3.34 (dd, J=12.7, 8.0 Hz, 1H), 3.02 (t, J=6.9 Hz, 2H), 2.93 (dd, J=12.4, 5.8 Hz, 1H), 2.65 (s, 3H), 2.15-2.00 (m, 2H), 1.09 (t, J=7.2 Hz, 3H).

MS (ESI) m/z: 532.4 [M+H]⁺

### Example 2-24

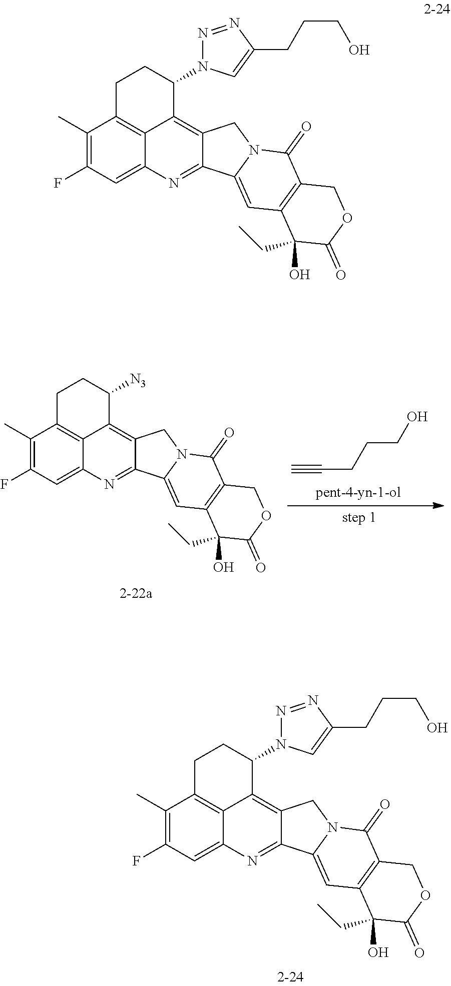

### Step 1

### (1S,9S)-9-ethyl-5-fluoro-9-hydroxy-1-(4-(3-hydroxypropyl)-1H-1,2,3-triazol-1-yl)-4-methyl-1,2,3,9,12,15-hexahydro-10H,13H-benzo[de]pyrano[3′,4′:6,7]indolizino[1,2-b]quinoline-10,13-dione (2-24)

To a solution of 2-22a (30 mg, 0.065 mmol) in DMF (1 mL) was added pent-4-yn-1-ol (10.9 mg, 0.13 mmol), CuI (1 mg, cat. amount) and DIPEA (56.8 μL, 42 mg, 0.33 mmol). The mixture was purged with N₂ balloon for 15 min, then stirred at r.t. under N₂ atmosphere for 2 h. On completion of the reaction, the solvent was removed by evaporation. The crude product was purified by silica column gel chromatography (eluent: CH₂Cl₂M/MeOH═CH₂Cl₂/MeOH=10/1 to 5/1) to afford 2-24 as a white solid (15 mg, yield 42.8%).

¹H NMR (400 MHz, DMSO-d6) δ 8.00 (s, 1H), 7.86 (d, J=10.9 Hz, 1H), 7.30 (s, 1H), 6.51 (s, 2H), 5.37 (s, 2H), 5.09 (d, J=19.0 Hz, 1H), 4.45 (t, J=5.1 Hz, 1H), 4.00 (d, J=19.0 Hz, 1H), 3.43 (dd, J=11.5, 6.1 Hz, 2H), 3.24 (dd, J=11.6, 6.2 Hz, 2H), 3.09 (dd, J=12.8, 7.9 Hz, 1H), 2.72-2.61 (m, 3H), 2.41 (s, 3H), 1.91-1.79 (m, 2H), 1.79-1.68 (m, 2H), 0.85 (t, J=7.3 Hz, 3H).

MS (ESI) m/z: 546.3 [M+H]⁺

### Example 2-25

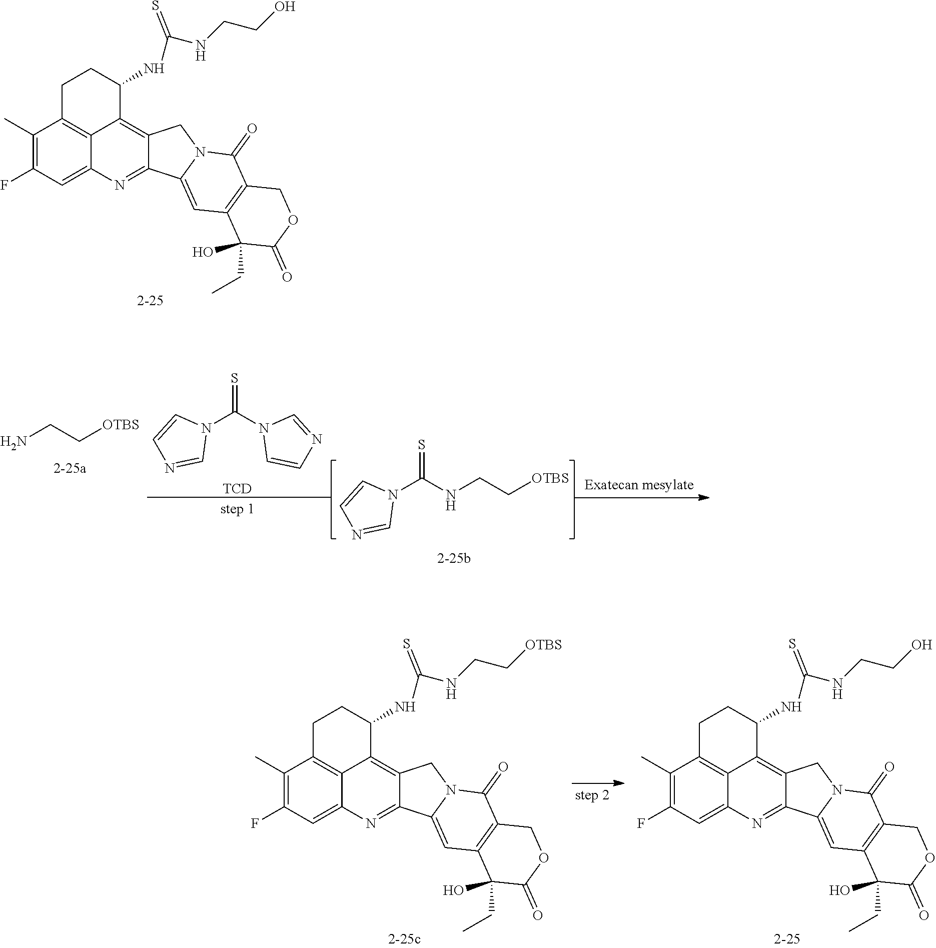

### Step 1 &2

### 1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-(2-hydroxyethyl)thiourea (2-25)

To a solution of 2-25a (65.9 mg, 0.38 mmol) in DMF (0.5 mL) was added TCDI (71.3 mg, 0.4 mmol). The mixture was stirred at r.t. for 30 min. After that exatecan mesylate (100 mg, 0.19 mmol) in 0.5 mL DMF was added dropwise. Upon completion of addition, the mixture was stirred 50° C. for 3 h, LCMS showed the full transformation of 2-25c. Then the reaction solution was cooled to 0° C., TBAF (0.45 mL, 0.45 mmol, 1M in THF) was added and the reaction was warmed to r.t. and stirred at r.t for 1 h. The organic solution was diluted with DCM (10 mL) washed subsequently with sat. NaHCO₃ (2 mL), H₂O (2 mL) and brine (2 mL). The organic phase was collected and dried over Na₂SO₄, filtered and the filtrate was concentrated under vacuum to give crude product 2-25 as a brown oil. The crude product was purified by silica column gel chromatography (eluent: CH₂Cl₂/MeOH=20/1 to 10/1) to afford 2-25 as a brown solid (61 mg, yield 60.4%).

¹H NMR (400 MHz, DMSO-d6) δ 8.14 (d, J=9.0 Hz, 1H), 7.80 (d, J=10.9 Hz, 1H), 7.57 (s, 1H), 7.31 (s, 1H), 6.52 (s, 1H), 6.21 (s, 1H), 5.42 (s, 2H), 5.25 (s, 2H), 4.84 (s, 1H), 3.56 (s, 4H), 3.18 (s, 2H), 2.40 (s, 3H), 2.23 (m, 2H), 1.86 (m, 2H), 0.87 (t, J=7.3 Hz, 3H).

MS (ESI) m/z: 539.3 [M+H]⁺

### Example 2-26

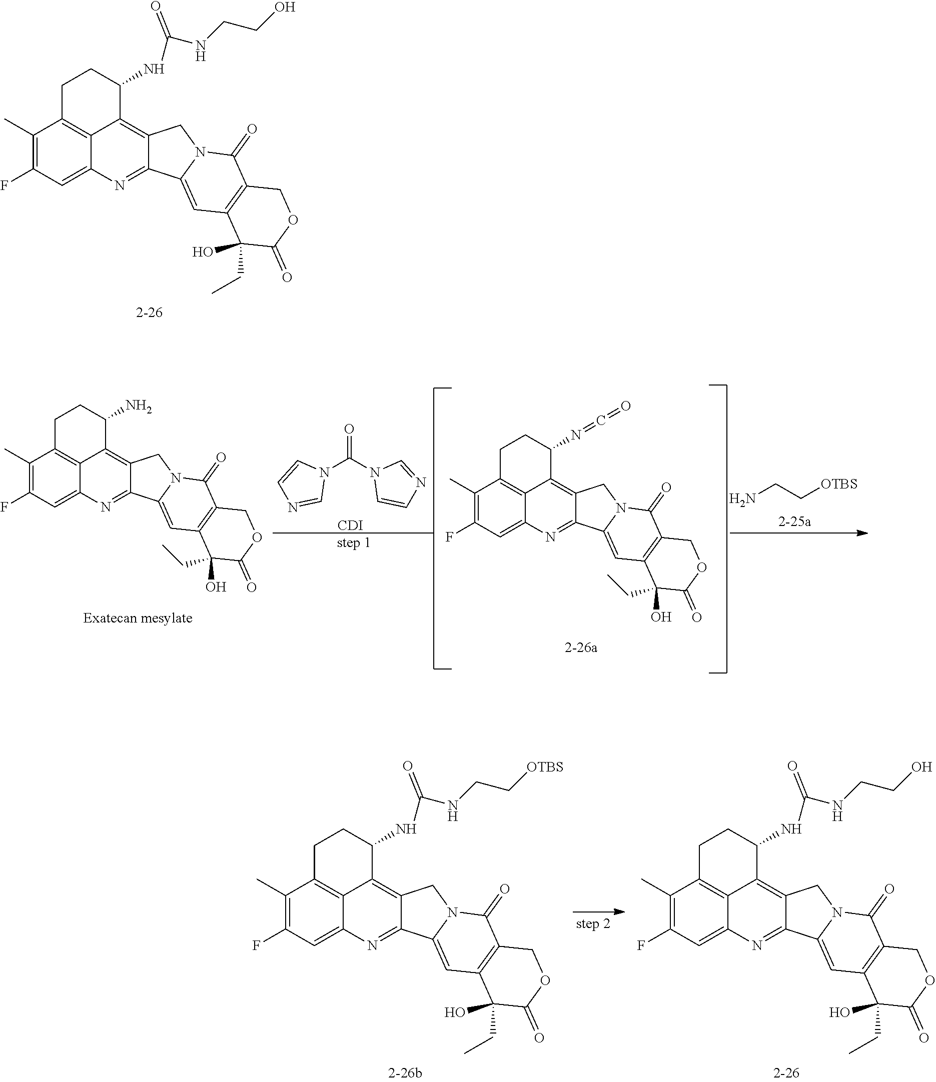

### Step 1 &2

### 1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-(2-hydroxyethyl)urea (2-26)

To a solution of exatecan mesylate (53.2 mg, 0.1 mmol) in DMF (1 mL) was added CDI (17 mg, 0.105 mmol). The mixture was stirred at r.t. for 30 min. After that 2-25a (35 mg, 0.2 mmol) in 0.5 mL DMF was added dropwise. Upon completion of addition, the mixture was stirred r.t. for 1.5 h. Then the reaction solution was cooled to 0° C., TBAF (0.3 mL, 0.3 mmol, 1M in THF) was added and the reaction was warmed to r.t. and stirred at r.t for 1 h. The organic solution was diluted with CH₂Cl₂ (10 mL) washed subsequently with sat. NaHCO₃ (2 mL), H₂O (2 mL) and brine (2 mL). The organic phase was collected and dried over Na₂SO₄, filtered and the filtrate was concentrated under vacuum to give crude product as a yellow oil. The crude product was purified by silica column gel chromatography (eluent: CH₂Cl₂/MeOH=10/1 to 8/1) to afford 2-26 as a brown solid (40 mg, yield 77%).

MS (ESI) m/z: 523.4 [M+H]⁺

### Example 2-27

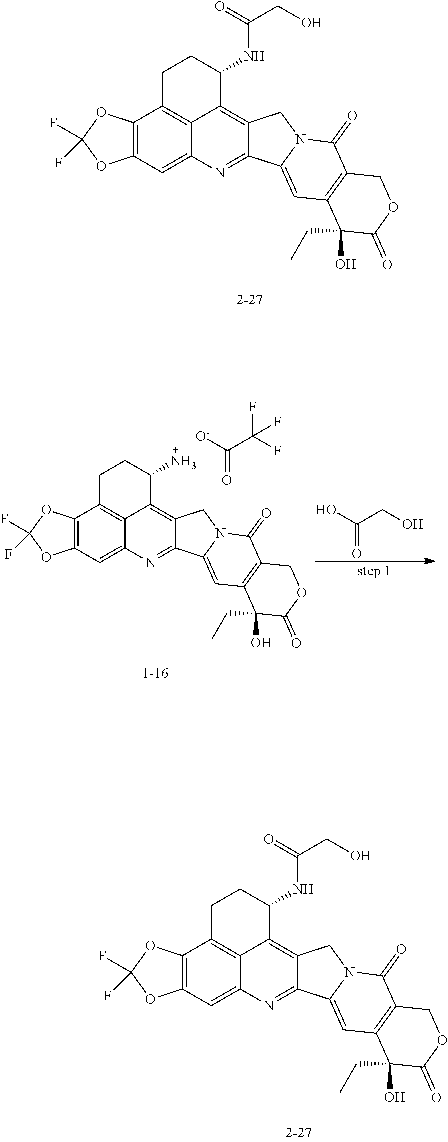 [SMILES: CC[C@@]1(O)C(=O)OCc2c1cc1n(c2=O)Cc2c-1nc1cc3c(c4c1c2[C@@H](NC(=O)CO)CC4)OC(F)(F)O3 ; CC[C@@]1(O)C(=O)OCc2c1cc1n(c2=O)Cc2c-1nc1cc3c(c4c1c2[C@@H]([NH3+])CC4)OC(F)(F)O3 ; O=C([O-])C(F)(F)F ; O=C(O)CO ; CC[C@@]1(O)C(=O)OCc2c1cc1n(c2=O)Cc2c-1nc1cc3c(c4c1c2[C@@H](NC(=O)CO)CC4)OC(F)(F)O3]

### Step 1

### N-((1S,10S)-10-ethyl-5,5-difluoro-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-2-hydroxyacetamide (2-27)

To a mixture of hydroxyacetic acid (3.776 mg, 0.05 mmol) and 1-16 (12.0 mg, 0.025 mmol) in DMF (1 mL) were added HATU (18.88 mg, 0.050 mmol) and DIPEA (8 ul, 6.416 mg, 0.050 mmol). The mixture was stirred at RT for 30 min. The mixture was purified by prep-HPLC (FA, 0.1%), the fraction was lyophilized to give 2-27 (6.8 mg, 100% purity, 50.6% yield) as a pale-yellow solid.

MS (ESI) m/z: 542.3 [M+H]⁺;

¹H NMR (400 MHz, d6-DMSO) δ 8.48 (d, J=9.0 Hz, 1H), 8.00 (s, 1H), 7.31 (s, 1H), 6.53 (s, 1H), 5.65 (dd, J=14.0, 6.0 Hz, 1H), 5.49 (t, J=5.8 Hz, 1H), 5.42 (s, 2H), 5.21 (s, 2H), 3.96 (d, J=5.8 Hz, 2H), 3.24-3.12 (m, 2H), 2.28-2.09 (m, 2H), 2.00 (dd, J=14.4, 6.8 Hz, 1H), 1.86 (qd, J=14.0, 7.2 Hz, 2H), 0.91-0.84 (m, 3H).

### Example 2-28, 2-29

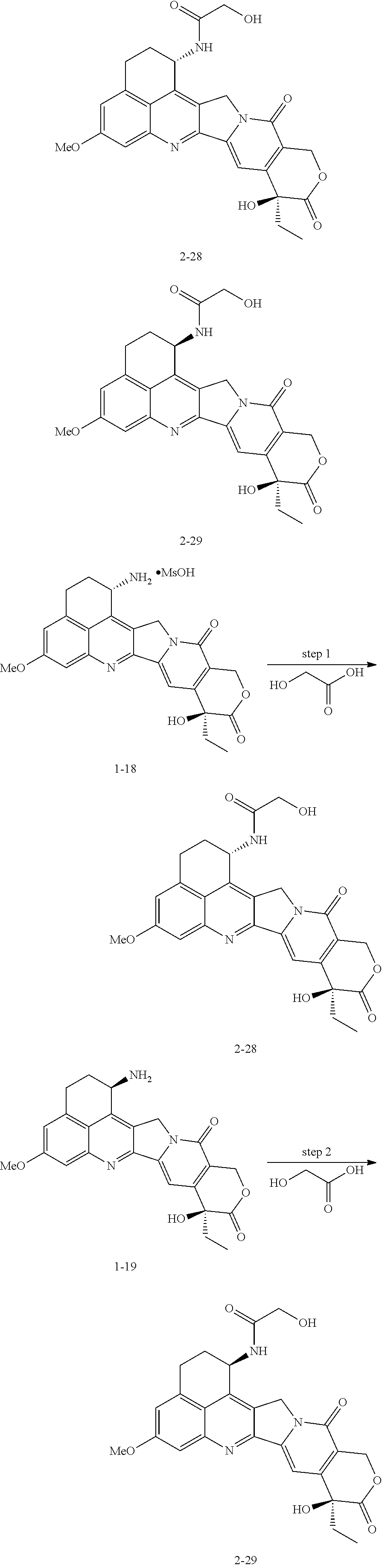

### Step 1

### N-((1S,9S)-9-ethyl-9-hydroxy-5-methoxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-28)

To a solution of reactant 2-hydroxyacetic acid (5.38 mg, 0.07 mmol), HBTU (26.85 mg, 0.07 mmol) and DIEA (18.3 mg, 0.14 mmol) in 2 ml DMF was added 1-18 (25 mg, 0.05 mmol) in 1 ml DMF. The mixture was stirred at rt for 30 min. On the completion of the reaction, the mixture was purified by prep-HPLC (FA) (Method: column: XBridge Prep C18 OBD 5 um 19*150 mm; Mobile phase: A—water (0.1% formic acid): B-acetonitrile; Flow rate: 20 mL/min, the fraction was lyophilized to give product (11 mg, 48% yield) as a yellow solid.

MS (ESI) m/z: 492.3 [M+H]⁺

### Step 2

### N-((1R,9S)-9-ethyl-9-hydroxy-5-methoxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-29)

To a solution of reactant 2-hydroxyacetic acid (6.58 mg, 0.09 mmol), HBTU (32.84 mg, 0.09 mmol) and DIEA (22.38 mg, 0.17 mmol) in 2 ml DMF was added 1-19 (25 mg, 0.06 mmol) in 1 ml DMF. The mixture was stirred at rt for 30 min. Most of SM was remaining as determined by LCMS, and the same eq. Glycolic acid, HBTU and DIEA was added to the mixture. The mixture was continue stirred for 3 h and some SM was remained. The reaction was purified by prep-HPLC (FA) (Method: column: XBridge Prep C18 OBD 5 um 19*150 mm; Mobile phase: A—water (0.1% formic acid): B-acetonitrile; Flow rate: 20 mL/min, the fraction was lyophilized to give product (5.7 mg, 20% yield) as a yellow solid.

MS (ESI) m/z: 492.3 [M+H]⁺

### Example 2-30

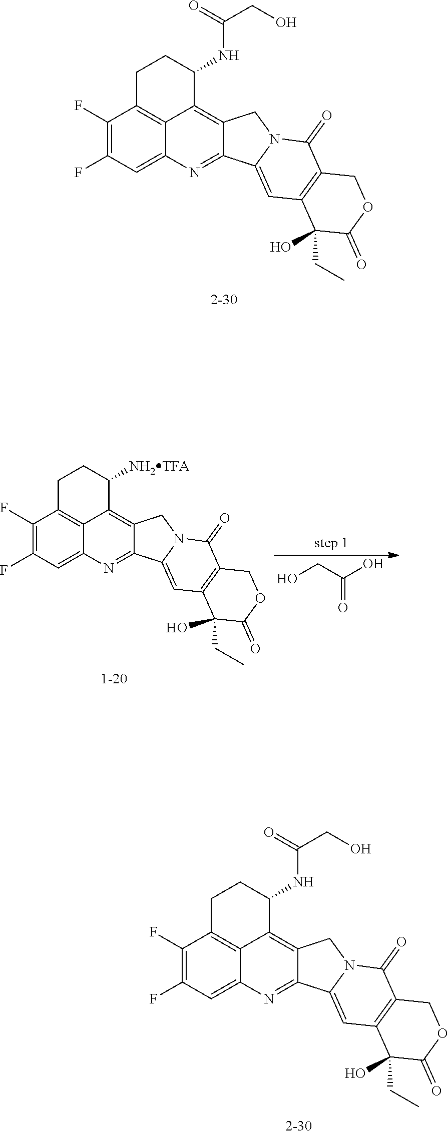

### Step 1

### N-((1S,9S)-9-ethyl-4,5-difluoro-9-hydroxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-30)

2-30 (10.7 mg, 79% yield) was synthesized according to synthetic procedure of example 2-28.

MS (ESI) m/z: 498.3 [M+H]⁺

### Example 2-31

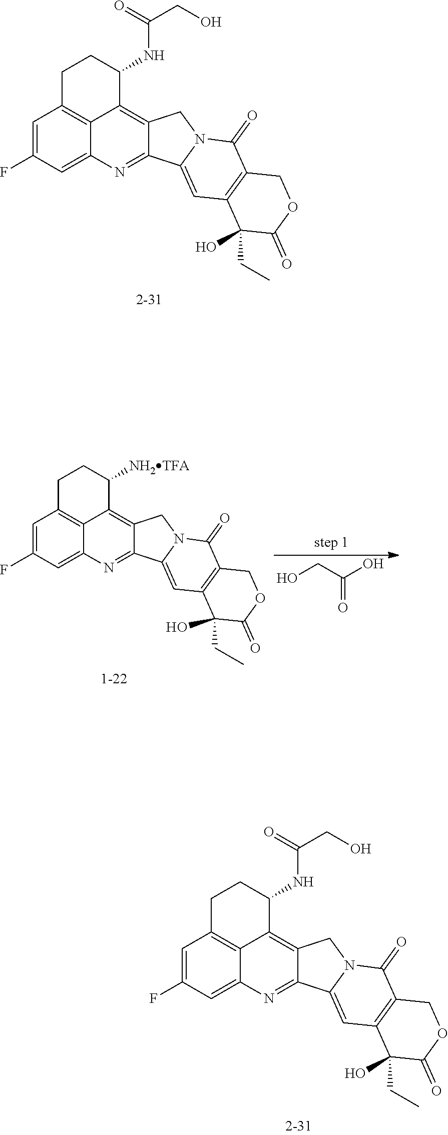

### Step 1

### N-((1S,9S)-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-31)

2-31 (3.2 mg, 25% yield) was synthesized according to synthetic procedure of example 2-28.

MS (ESI) m/z: 480.3 [M+H]⁺

¹H NMR (400 MHz, d6-DMSO) δ 8.52 (d, J=9.2 Hz, 1H), 7.78 (dd, J=10.0 Hz, J=2.4 Hz, 1H), 7.51 (dd, J=8.8 Hz, J=2.4 Hz, 1H), 7.33 (s, 1H), 6.55 (s, 1H), 5.65-5.62 (m, 1H), 5.60 (s, 1H), 5.43 (s, 2H), 5.21 (d, J=18.8 Hz, 1H), 5.15 (d, J=18.8 Hz, 1H), 3.97 (s, 2H), 3.28-3.17 (m, 2H), 2.21-2.11 (m, 2H), 1.92-1.81 (m, 2H), 0.87 (t, J=7.2 Hz, 3H).

### Example 2-32

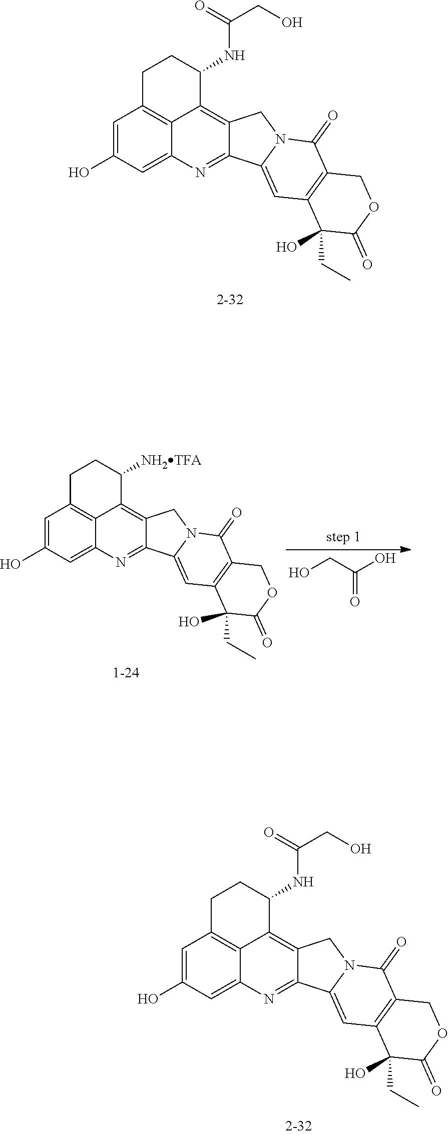

### Step 1

### N-((1S,9S)-9-ethyl-5,9-dihydroxy-10,13-dioxo-2,3,9,10,13,15-hexahydro-1H,12H-benzo[de]pyrano[3′,4′:6,7]indolizino[1,2-b]quinolin-1-yl)-2-hydroxyacetamide (2-32)

2-32 (3.5 mg, 24% yield) was synthesized according to synthetic procedure of example 2-28.

MS (ESI) m/z: 478.4 [M+H]⁺

### Example 2-34

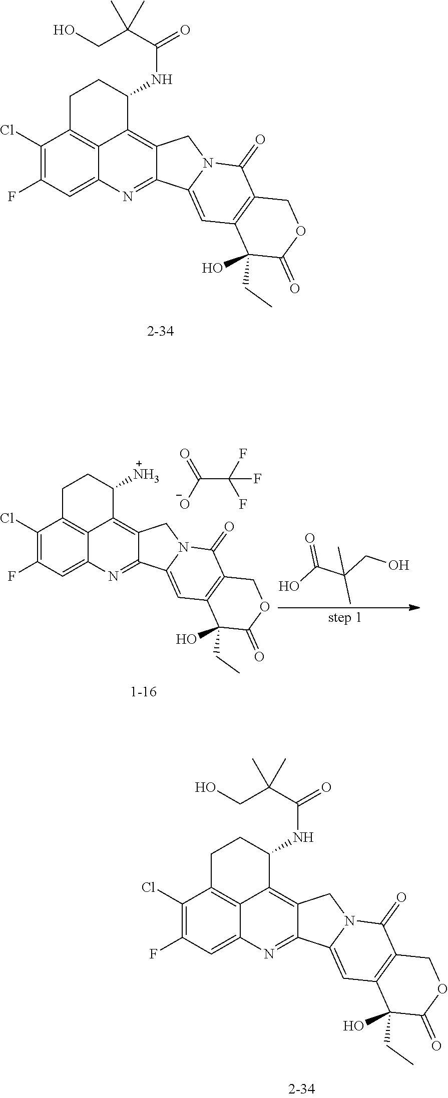

### N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (2-34)

2-34 (5.8 mg, 99.82% purity, 67.9% yield) was synthesized according to a similar procedure as that of example 2-29.

MS (ESI) m/z: 556.3 [M+H]⁺

### Example 2-35

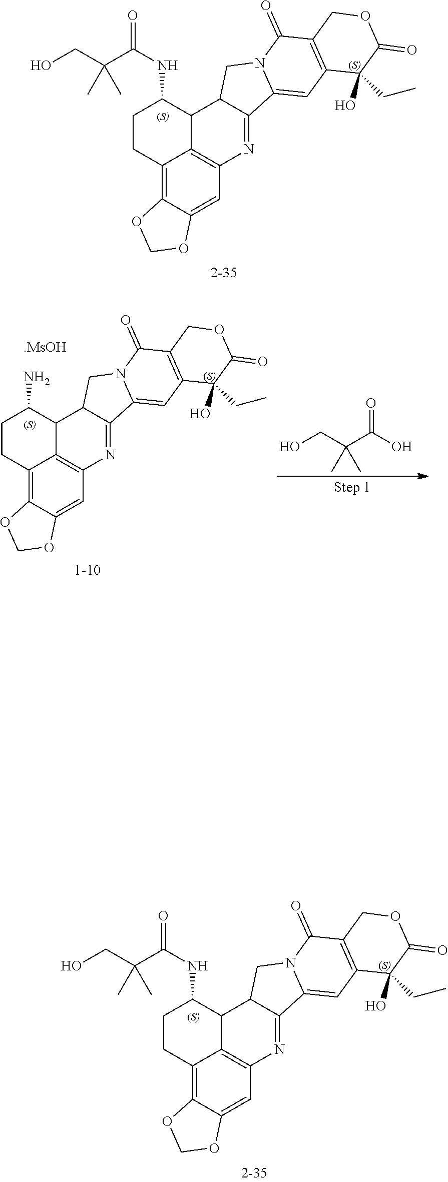

### N-((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-3-hydroxy-2,2-dimethylpropanamide (2-35)

Compound 2-35 (1.8 mg, 17.9% yield) was synthesized according to synthetic procedure of step 3 of example 2-2.

MS (ESI) m/z: 548.4 [M+H]⁺

### Example 2-36

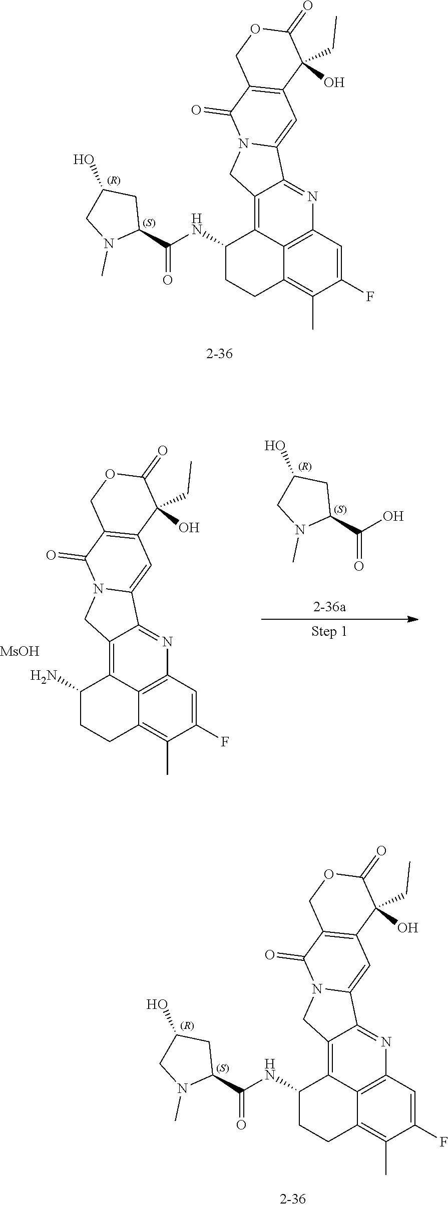

### Step 1

### (2S,4R)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxy-1-methylpyrrolidine-2-carboxamide (2-36)

To a solution of 2-36a (25.0 mg, 0.17 mmol) in DMF (2.0 mL) at room temperature was added exatecan mesylate (45.7 mg, 0.09 mmol), HTBU (48.9 mg, 0.13) and DIEA (27.8 mg, 0.22 mol). The resulting mixture was stirred for 6 h. The resulting white solid was purified by pre-HPLC to give the desired product 2-36 (9.3 mg, 18% yield) yellow solid.

MS (ESI) m/z: 563.4 [M+H]⁺

¹H NMR (400 MHz, DMSO-d6) δ 9.93 (s, 1H), 9.18 (d, J=8.2 Hz, 1H), 7.84 (d, J=11.0 Hz, 1H), 7.32 (s, 1H), 6.54 (s, 1H), 5.73-5.63 (m, 1H), 5.58 (s, 1H), 5.42 (s, 2H), 5.29 (d, J=18.6 Hz, 1H), 5.09 (d, J=18.6 Hz, 1H), 4.38 (s, 1H), 4.22 (s, 1H), 3.82-3.81 (m, 1H), 3.22 (s, 2H), 3.01-3.0 (m, 1H), 2.91 (s, 3H), 2.43 (s, 3H), 2.33-1.98 (m, 5H), 1.94-1.75 (m, 2H), 0.88 (t, J=7.3 Hz, 3H).

### Example 2-37

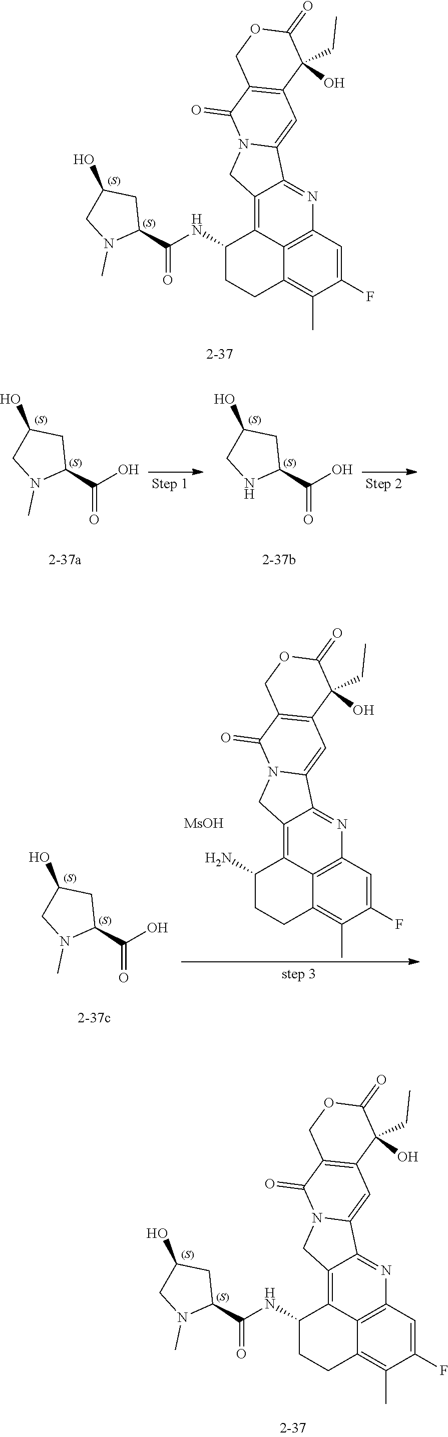

### Step 1

### (2S,4S)-4-hydroxypyrrolidine-2-carboxylic acid (2-37b)

To a solution of 2-37a in 10 ml CH₂Cl₂ was added 10 ml TFA at ice water bath. The mixture was allowed to r.t. and continued stirring. On the completion of the reaction, the mixture was concentrated to give 600 mg crude product which was directly used as the next step.

### Step 2

### (2S,4S)-4-hydroxy-1-methylpyrrolidine-2-carboxylic acid (2-37c)

To a solution of 95 mg 2-37b in 1.7 ml acetic acid and 1 ml H₂O was added 0.13 ml 37% aqueous formaldehyde and 9.5 mg PbO₂. The mixture was stirred at r.t. On completion of the reaction, the mixture was filtered and concentrated to give 70 mg crude product which was used directly as the next step.

### Step 3

### (2S,4S)—N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxy-1-methylpyrrolidine-2-carboxamide (2-37)

To a solution of crude 2-37c (52 mg) in DMF (5.0 mL) at room temperature was added exatecan mesylate (53.2 mg, 0.1 mmol), HTBU (56.9 mg, 0.15 mmol) and DIEA (47 ul, 0.30 mmol). The resulting mixture was stirred for 6 h. The mixture was purified by pre-HPLC to give the desired product 2-37 (23 mg, 23% yield) yellow solid.

MS (ESI) m/z: 563.4 [M+H]⁺

### Example 2-38

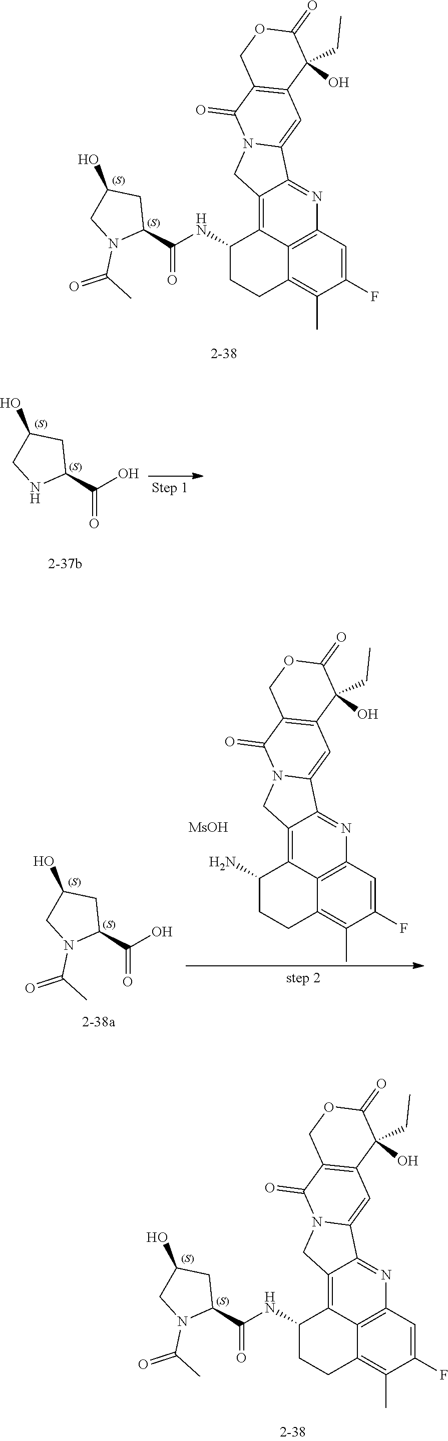

### Step 1

### (2S,4S)-1-acetyl-4-hydroxypyrrolidine-2-carboxylic acid (2-38a)

To a solution of 32 mg 2-37b in 1 ml EA was added 24 ul Ac₂0. The mixture was sonicated for 6 h at r.t. On the completion of the reaction, the mixture was concentrated to give 29 mg crude desired product which was used directly as next step.

### Step 2

### (2S,4S)-1-acetyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-4-hydroxypyrrolidine-2-carboxamide (2-38)

2-38 (7.3 mg, 19% yield) was synthesized according to synthetic procedure of example 2-37.

MS (ESI) m/z: 591.3 [M+H]⁺

### Example 2-39

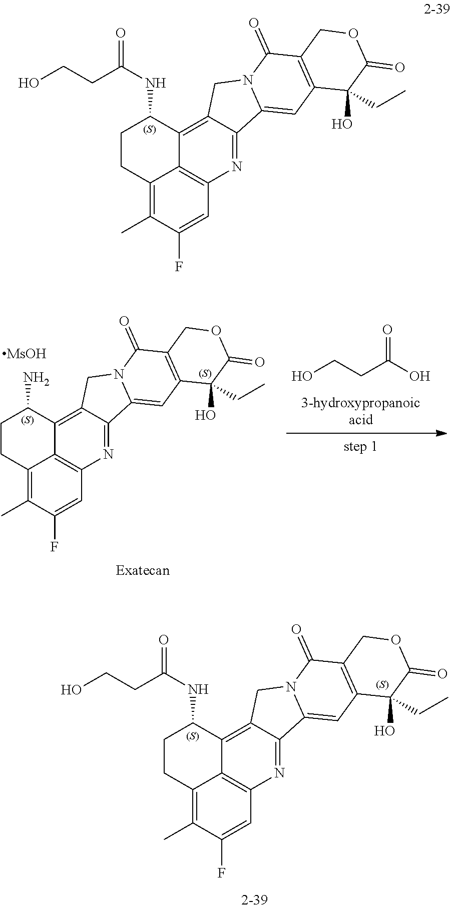

### Step 1

To a solution of compound exatecan mesylate (50 mg, 0.094 mmol) in MeOH (2 mL) were added 3-hydroxypropanoic acid (85 mg, 0.28 mmol, 30% in water), DMT-MM (78 mg, 0.28 mmol) and DIEA (36 mg, 0.28 mmol). The mixture was stirred at r.t. for 2 h. The mixture was filtered and the filtrate was purified using prep-HPLC (Method: column: XBridge Prep C18 OBD 5 um 19*150 mm; Mobile phase: A-water (0.1% trifluoroacetic acid): B-acetonitrile; Flow rate: 20 mL/min to provide compound 2-39 (28.3 mg, 59.3% yield) as white solid.

¹H NMR (400 MHz, DMSO-d₆) δ 8.45 (d, J=8.8 Hz, 1H), 7.80 (d, J=11.2 Hz, 1H), 7.31 (s, 1H), 6.53 (s, 1H), 5.65-5.51 (m, 1H), 5.43 (s, 2H), 5.29-5.09 (m, 2H), 4.60 (t, J=5.2 Hz, 1H), 3.67 (dd, J=11.2, 5.6 Hz, 1H), 3.18 (s, 1H), 2.40 (s, 2H), 2.32 (t, J=6.4 Hz, 1H), 2.22-2.04 (m, 1H), 1.95-1.71 (m, 1H), 0.87 (t, J=7.2 Hz, 2H);

MS (ESI) m/z: 508.3 [M+H]⁺; UPLC-MS Retention time: 3.28 min.

### Example 2-40

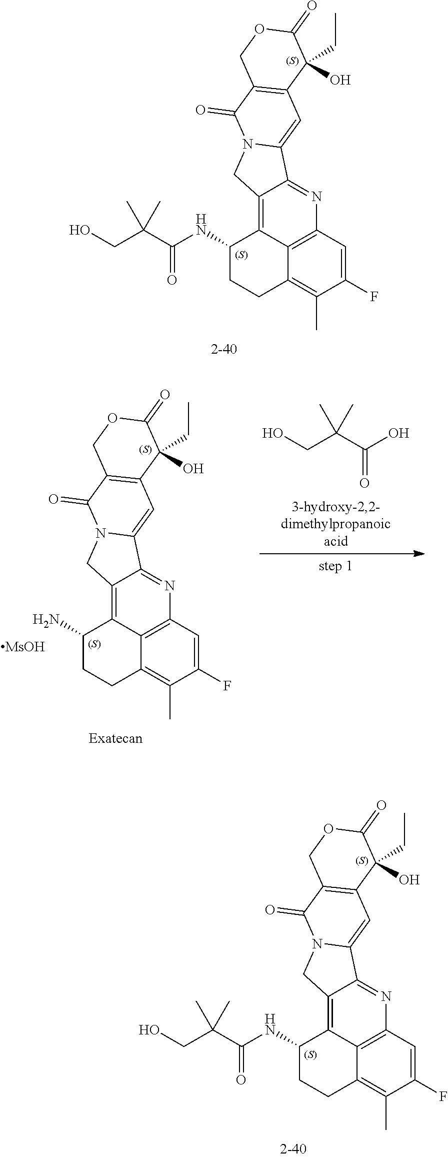

### Step 1

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (2-40)

To a mixture of 3-hydroxy-2,2-dimethylpropanoic acid (5 mg, 0.042 mmol) and HATU (16 mg, 0.042 mmol) in DMF (1 mL) were added DIPEA (21 μL, 16 mg, 0.13 mmol) and exatecan mesylate (23 mg, 0.043 mmol). The resulted brown mixture was stirred at r.t. for 2 h. On completion of the reaction, the mixture was purified by prep-HPLC (TFA) (Method: column: XBridge Prep C18 OBD 5 μm 19*150 mm; Mobile phase: A-water (0.1% TFA): B-acetonitrile; Flow rate: 20 mL/min, the fraction was lyophilized to give 2-40 (15 mg, 65.5% yield) as a white powder.

¹H NMR (400 MHz, DMSO-d6): δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H).

MS (ESI) m/z: 536.4 [M+H]⁺

### Example 2-41

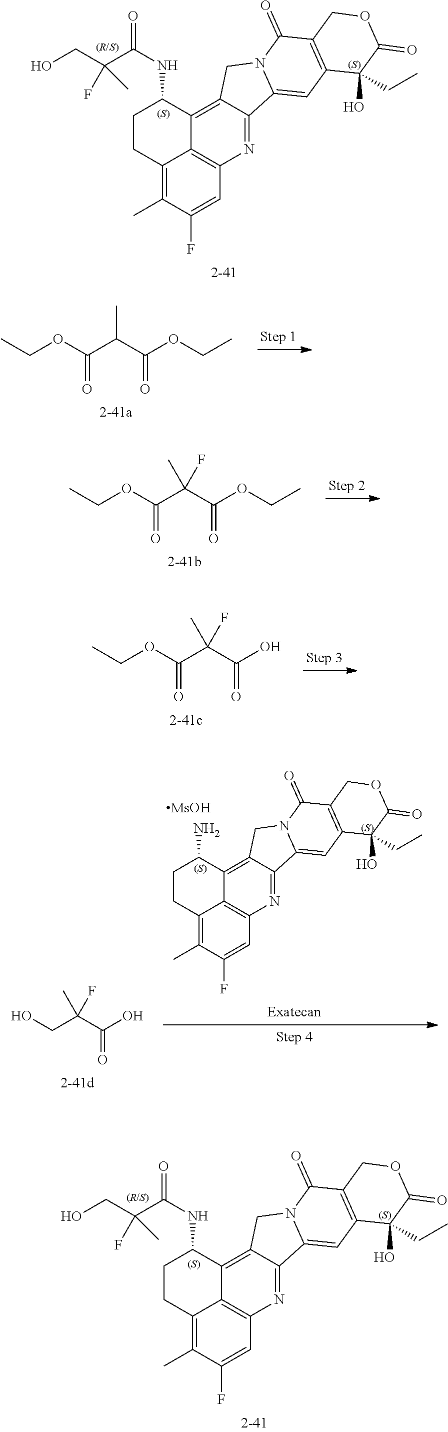

### Step 1

### Diethyl 2-fluoro-2-methylmalonate (2-41b)

A solution of compound 2-41a (10.0 g, 57.4 mmol) in THF (200 mL) was cooled to 0° C. 60% of NaH in oil (3.21 g, 80.37 mmol) was added into the mixture portionwise, stirred at 0° C. for 30 min. Then N-fluoro-N-(phenylsulfonyl)benzenesulfonamide (NSFI, 19.91 g, 63.2 mmol) was added into the mixture portionwise at 0° C. Then warmed up to r.t. and stirred for 16 h. After reaction completed, the suspension was filtered and the filtrated was concentrated. PE (100 mL) was added into the residue, the precipitate was filtered and the filtrated was concentrated to give compound 2-41b (12.5 g, crude) as a light-yellow oil.

¹H NMR (400 MHz, CDCl₃) δ 4.30 (q, J=7.2 Hz, 4H), 1.79 (d, J=22.0 Hz, 3H), 1.31 (t, J=7.2 Hz, 6H);

¹⁹F NMR (376 MHz, CDCl₃) δ −157.50.

### Step 2

### 3-ethoxy-2-fluoro-2-methyl-3-oxopropanoic acid (2-41c)

To a solution of compound 2-41b (1.0 g, 5.2 mmol) in EtOH (5 mL) was added KOH solution (321 mg) in H₂O (50 uL) and EtOH (2 mL) dropwise at 0° C. The mixture was stirred at r.t. for 2 h. The mixture was diluted with 20 (mL), washed with CH₂Cl₂ (20 mL*3). The aqueous solution was adjusted to pH=3 by 1 N HCl. The extracted by EtOAc (50 mL*3). The organic layer was dried combined and dried over anhydrous Na₂SO₄, filtered, and concentrated to give compound 2-41c (470 mg, 55.0% yield) as colorless oil.

¹H NMR (400 MHz, CDCl₃) δ 8.31 (br s, 1H), 4.32 (q, J=7.2 Hz, 2H), 1.83 (d, J=22.0 Hz, 3H), 1.33 (t, J=7.2 Hz, 3H);

¹⁹F NMR (376 MHz, CDCl₃) δ −157.59.

### Step 3

### 2-fluoro-3-hydroxy-2-methylpropanoic acid (2-41d)

To a solution of compound 2-41c (200 mg, 1.22 mmol) in isopropanol (4 mL) was added 2 M LiBH₄ (1.22 mL, 2.44 mmol) at 0° C. The mixture was stirred at r.t. for 2 h. The mixture was quenched by 2 N HCl (1.22 mL) dropwise at 0° C. The diluted with H₂O (10 mL), extracted with EtOAc (50 mL*3). The organic layer was combined and dried over anhydrous Na₂SO₄, filtered and concentrated to give compound 2-41d (92 mg, 61.7% yield) as colorless oi.

¹H NMR (400 MHz, CDCl₃) δ 4.01-3.81 (m, 2H), 1.58 (d, J=21.2 Hz, 3H);

¹⁹F NMR (376 MHz, CDCl₃) δ −163.98.

### Step 4

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-fluoro-3-hydroxy-2-methylpropanamide (2-41)

To a solution of compound 2-41d (23 mg, 0.19 mmol) in DMF (2 mL) were added exatecan mesylate (50 mg, 0.094 mmol), HATU (54 mg, 141 mmol) and DIEA (36 mg, 0.28 mmol). The mixture was stirred at r.t. for 1 h. The mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min, the fraction was lyophilized to give 2-41 as white solid (11 mg, 21.9% yield).

UPLC-MS, RT=3.52 min.

¹H NMR (400 MHz, DMSO-d₆) δ 9.06 (dd, J=9.0, 2.8 Hz, 1H), 8.00 (d, J=10.9 Hz, 1H), 7.54 (s, 1H), 6.75 (s, 1H), 5.82 (d, J=8.0 Hz, 1H), 5.65 (s, 2H), 5.43 (dt, J=77.8, 12.4 Hz, 3H), 4.17-3.91 (m, 1H), 3.83 (ddd, J=18.0, 12.4, 5.6 Hz, 1H), 3.40-3.27 (m, 1H), 2.62 (s, 3H), 2.50-2.34 (m, 2H), 2.22-1.98 (m, 2H), 1.81 (d, J=21.4 Hz, 3H), 1.11 (t, J=7.2 Hz, 3H);

MS (ESI) m/z: 540.3 [M+H]⁺.

### Example 2-42

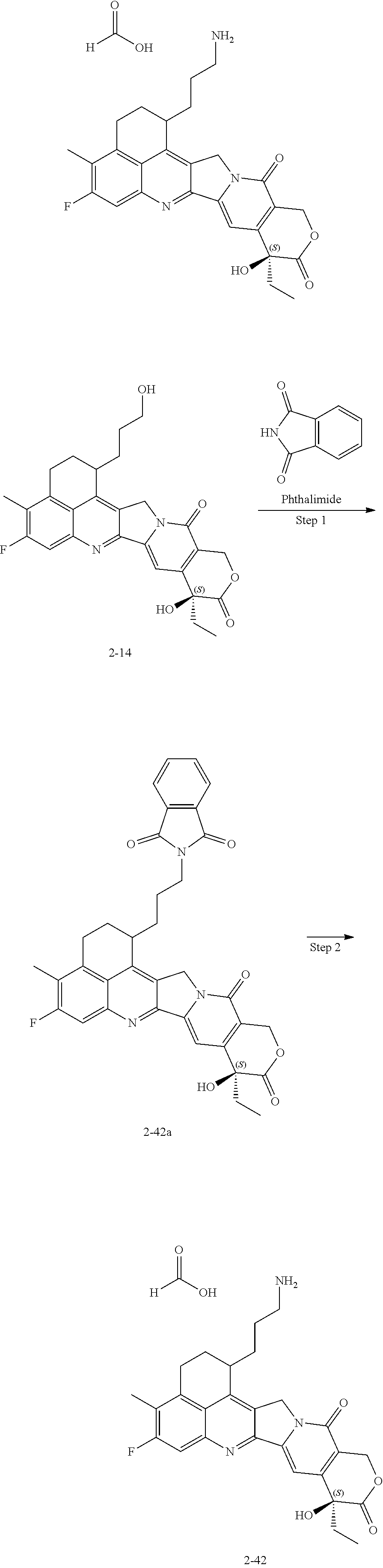

### Step 1

### (9S)-1-(3-(1,3-dioxoisoindolin-2-yl)propyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (2-42a)

A mixture of PPh₃ (49.3 mg, 0.188 mmol) and DIAD (38.0 mg, 0.188 mmol) in THF (1 mL) stirred at r.t. for 5 min then added to a mixture of compound 2-14 (60 mg, 0.125 mmol) and Phthalimide (20.3 mg, 0.138 mmol) in THF (1 mL). The resulted yellow mixture was stirred at r.t. for 1h. The reaction was quenched by addition of water (10 mL), extracted with EtOAc (10 mL*2). Combined organic layer was washed with brine (10 mL), dried over Na₂SO₄, filtered and the filtrate was concentrated under vacuum to give a residue. It was purified by silica gel chromatography (SiO₂ 5 g, EtOAc/PE=20%-70%), fraction was concentrated under vacuum to give a crude product (130 mg, crude) as a yellow solid. Contain most of PPh₃O based on LCMS.

MS (ESI) m/z: 608.5 [M+H]⁺

### Step 2

### (9S)-1-(3-aminopropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione formate (2-42)

To a solution of 2-42a (130 mg, crude) in EtOH (2 mL) was added N₂H₄.H₂O (80%, 20 uL). The mixture was stirred at 60° C. for 60 min. The mixture was acidified with 3N HCl to pH=2. Then purified by prep-HPLC (FA), fraction was lyophilized to give target product 2-42 (12.2 mg, 99.68% purity) as a white solid.

MS (ESI) m/z: 478.4 [M+H]⁺

¹H NMR (400 MHz, DMSO-d6) δ 7.75 (d, J=10.8 Hz, 1H), 7.31 (s, 1H), 5.44 (s, 2H), 5.34 (q, J=18.8 Hz, 3H), 3.10 (m, 1H), 2.76 (s, 2H), 2.38 (s, 3H), 2.27 (d, J=12.8 Hz, 2H), 1.91 (m, 6H), 1.62 (m, 3H), 0.87 (t, J=7.4 Hz, 4H).

### Example 2-43

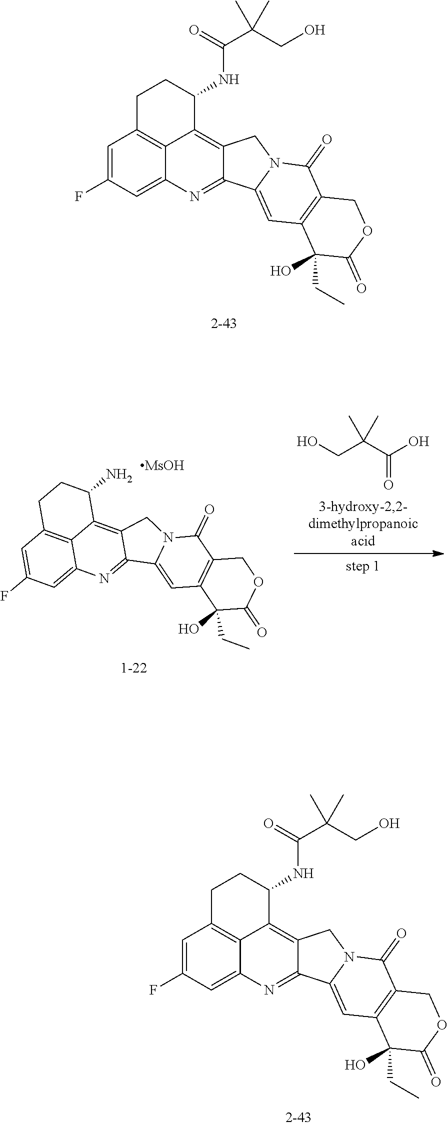

### Step 1

### N-((1S,9S)-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (2-43)

Compound 2-43 (0.3 mg, 95.8% purity) was obtained according to the procedure described in step 1 of example 2-8.

MS (ESI) m/z: 522.4 [M+H]⁺

1H NMR (400 MHz, DMSO-d6) δ 8.04 (d, J=8.4 Hz, 1H), 7.78 (dd, J=10.3, 2.5 Hz, 1H), 7.51 (d, J=9.1 Hz, 1H), 7.33 (s, 1H), 6.52 (s, 1H), 5.60 (d, J=7.9 Hz, 1H), 5.42 (s, 2H), 5.24 (d, J=19.2 Hz, 1H), 5.15 (d, J=19.0 Hz, 1H), 4.87 (t, J=5.1 Hz, 1H), 3.49-3.43 (m, 2H), 3.24-3.12 (m, 2H), 2.13 (d, J=5.8 Hz, 2H), 1.94-1.79 (m, 2H), 1.13 (s, 3H), 1.11 (s, 3H), 0.88 (t, J=7.3 Hz, 3H).

### Example 2-44 & 2-45

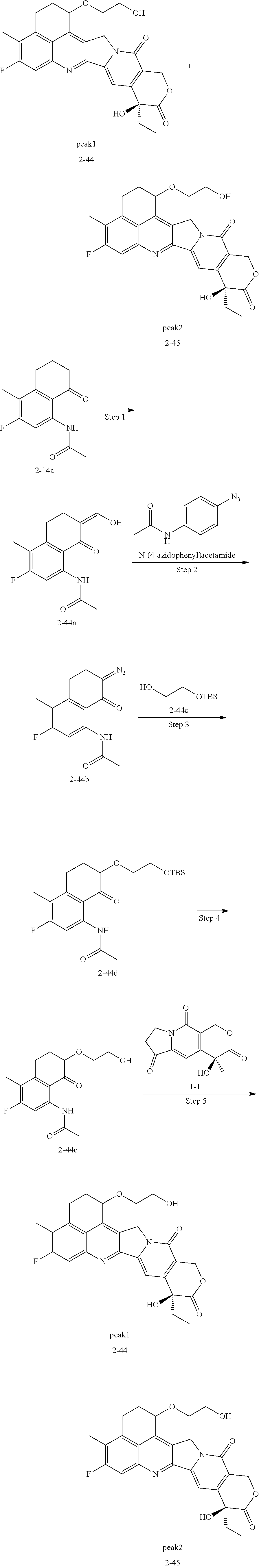

### Step 1

### (Z)—N-(3-fluoro-7-(hydroxymethylene)-4-methyl-8-oxo-5,6,7,8-tetrahydronaphthalen-1-yl)acetamide (2-44a)

To the solution of 2-14a (1 g, 4.25 mmol) in dry THF (30 mL) were added ethyl formate (449 L, 5.53 mmol) and the solution of KOt-Bu in THF (1M, 10.6 mL, 10.6 mmol) at 0° C. under nitrogen atmosphere. The solution was allowed to warm up to r.t. and stirred overnight. The solution was adjusted pH to 3 with 1N HCl, extracted with EtOAc (20 mL*3), dried over anhydrous Na₂SO₄, filtered and concentrated to give the crude product 2-44a (1.1 g, quant.) as a yellow solid.

MS (ESI) m/z: 264.2 [M+H]⁺

### Step 2

### N-(7-diazo-3-fluoro-4-methyl-8-oxo-5,6,7,8-tetrahydronaphthalen-1-yl)acetamide (2-44b)

To the cooled solution of 2-44a (1.1g, 4.2 mmol) in dry CH₂Cl₂ (30 mL) were added TEA (4.8 mL, 20.9 mmol) and N-(4-azidophenyl)acetamide (1.5 g, 6.3 mmol) at −10° C. under nitrogen atmosphere, stirred for 1 h. The solution was gradually reached to 0° C. 2N NaOH solution (10 mL) was added. The solution was extracted with CH₂Cl₂ (15 mL*3). The organic phase was concentrated and purified by flash column chromatography (silica gel, petroleum ether/ethyl acetate=3:1) to give compound 2-44b (700 mg, 98% purity, 64% yield) as a yellow solid.

MS (ESI) m/z: 262.2 [M+H]⁺

¹H NMR (400 MHz, CDCl₃) δ 12.34 (s, 1H), 8.42 (d, J=12.8 Hz, 1H), 3.01-2.95 (m, 2H), 2.94-2.89 (m, 2H), 2.21 (s, 3H), 2.17 (d, J=1.9 Hz, 3H).

### Step 3

### N-(7-(2-((tert-butyldimethylsilyl)oxy)ethoxy)-3-fluoro-4-methyl-8-oxo-5,6,7,8-tetrahydronaphthalen-1-yl)acetamide (2-44d)

To the mixture of 2-44b (824 mg, 3.15 mmol) and 2-44c (3.8 mL, 18.9 mmol) was added BF₃ Et₂O (65 μL, 0.32 mmol) under nitrogen atmosphere with gas evolution. The solution was stirred at r.t. for 1h until no gas evolution. The solution was diluted with EtOAc (10 mL) and sat. NaHCO₃ (10 mL), extracted with EtOAc (10 mL*3). The organic phase was concentrated and purified by flash column chromatography (silica gel, petroleum ether/ethyl acetate=5:1) to give compound 2-44d (328 mg, crude) as a yellow oil.

MS (ESI) m/z: 410.4 [M+H]⁺

### Step 4

### 8-amino-6-fluoro-2-(2-hydroxyethoxy)-5-methyl-3,4-dihydronaphthalen-1 (2H)-one (2-44e)

To the solution of 2-44d (328 mg, 0.8 mmol) in MeOH (3 mL) was added 2N HCl (3 mL) under nitrogen atmosphere, stirred at 60° C. for 4 h. The solution was adjusted pH to 8 with sat. Na₂CO₃, extracted with EtOAc (10 mL*3). The organic phase was concentrated and purified by flash column chromatography (silica gel, petroleum ether/ethyl acetate=1:1) to give compound 2-44e (80 mg, crude) as a brown oil.

MS (ESI) m/z: 254.3 [M+H]⁺

### Step 5

### (9S)-9-ethyl-5-fluoro-9-hydroxy-1-(2-hydroxyethoxy)-4-methyl-1,2,3,9,12,15-hexahydro-10H,13H-benzo[de]pyrano[3′,4′:6,7]indolizino[1,2-b]quinoline-10,13-dione (2-44 & 2-45)

To the solution of 2-44e (85 mg, 0.34 mmol) and 1-1i (99 mg, 0.37 mmol) in toluene (3 mL) was added PPTS (43 mg, 0.17 mmol), refluxed for 36 h. The solution was concentrated and purified by flash column chromatography (silica gel, DCM/MeOH=20:1) to give a mixture (16 mg, 2-44/2−45=1:1) as a yellow solid. The mixture was purified by prep-HPLC (Mobile phase A: 0.1% FA in water, B: MeCN; Gradient: 20%-30%-45% B; Flow rate: 20 mL/min; Column: Xbridge Prep C18 OBD™ 5 m, 19*150 mm). The desired fraction was lyophilized to give 2-44 (0.53 mg, 95% purity, containing 5% 2-45) and 2-45 (0.91 mg, 83% purity, containing 17% 2-44).

MS (ESI) m/z: 481.4 [M+H]⁺

UPLC analysis: 2-44, retention time=2.03 min; 2-45, retention time=2.13 min (Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm).

### Example 2-46

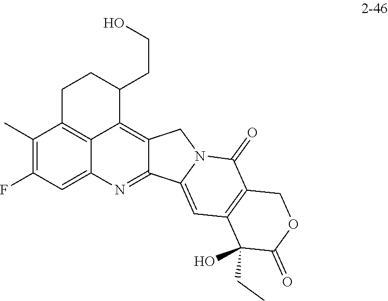 [SMILES: CC[C@@]1(O)C(=O)OCc2c1cc1n(c2=O)Cc2c-1nc1cc(F)c(C)c3c1c2C(CCO)CC3]

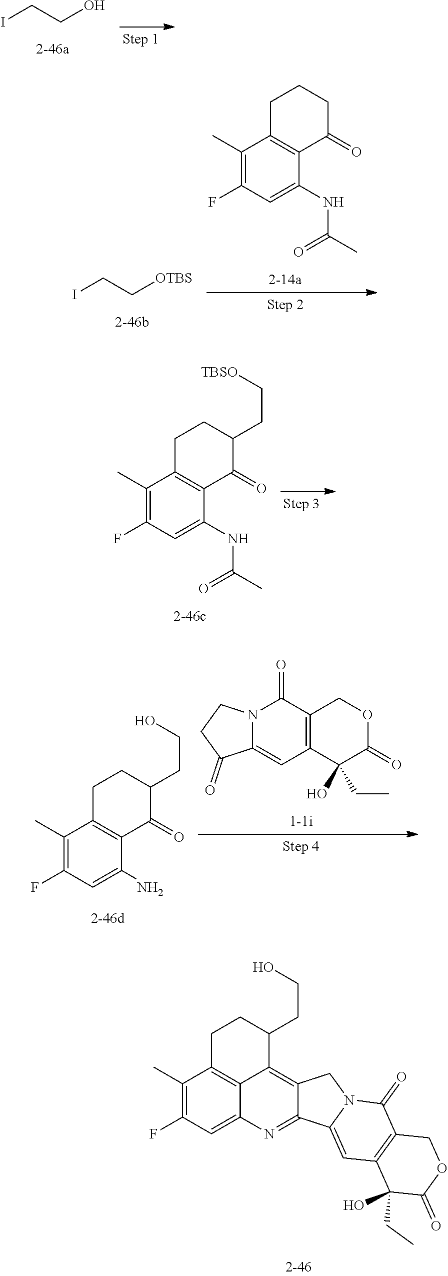

### Step 1

### tert-butyl(2-iodoethoxy)dimethylsilane (2-46b)

A solution of compound 2-46a (3000 mg, 17.44 mmol) in CH₂Cl₂ (40 mL) was cooled to 0° C., then imidazole (1779 mg, 26.16 mmol) and TBSCl (3139 mg, 20.93 mmol) was added portionwise at 0° C. The mixture was stirred at 20° C. for 16 h. TLC (SiO₂, Petroleum ether) showed the reaction was completed. The reaction was poured into water (40 mL) and extracted with CH₂Cl₂ (50 mL*3). The combined organic layer was dried over Na₂SO₄ and concentrated to give the residue which was purified by silica column gel chromatography (eluent: Petroleum ether=100) to afford 2-46b (3.5 g, 70.2% yield) as a colorless oil.

### Step 2

### N-(7-(2-((tert-butyldimethylsilyl)oxy)ethyl)-3-fluoro-4-methyl-8-oxo-5,6,7,8-tetrahydronaphthalen-1-yl)acetamide (20-46c)

To a solution of compound 2-14a (2350 mg, 10 mmol) in THF (40 mL) was added tBuOK (1M in THF) (22 mL, 22 mmol) at 0° C. dropwise and the internal temp. was maintained at 0° C. The mixture was stirred at 0° C. for 30 min, then 2-46b (3432 mg, 12 mmol) was added slowly. LCMS showed the reaction was completed. 1N HCl was added to the mixture dropwise to adjust pH to 2. The mixture was poured into saturated NaHCO₃ aq. (50 mL) and was extracted with EtOAc (60 mL*3). The combined organic layer was dried over anhydrous Na₂SO₄, filtered and concentrated to give the crude product which was purified by silica column gel chromatography (eluent: Petroleum ether/EtOAc=100/0 to 83/17) to afford 2-46c (700 mg, 17.8% yield) as a yellow solid.

MS (ESI) m/z: 394.2 [M+H]⁺.

¹H NMR (400 MHz, DMSO-d6) δ=7.40 (s, 2H), 6.34 (d, J=12.6, 1H), 6.34 (d, J=12.6, 1H), 4.45 (t, J=5.2, 1H), 4.45 (t, J=5.2, 1H), 3.65-3.35 (m, 2H), 3.55-3.45 (m, 2H), 2.83 (s, 1H), 2.83 (s, 1H), 2.72-2.61 (m, 1H), 2.46 (d, J=5.0, 1H), 2.11-1.99 (m, 3H), 1.97 (d, J=1.0, 3H), 1.72-1.60 (m, 1H), 1.43 (d, J=6.2, 1H).

### Step 3

### 8-amino-6-fluoro-2-(2-hydroxyethyl)-5-methyl-3,4-dihydronaphthalen-1 (2H)-one (2-46d)

To a solution of compound 2-46c (700 mg, 1.78 mmol) in MeOH (3 mL) were added 2 N HCl (3 mL). The mixture was stirred at 60° C. for 16 h. LCMS showed the reaction was completed. The mixture was poured into aq. saturated NaHCO₃ (20 mL) and was extracted with EtOAc (30 mL*3). The combined organic layer was dried over anhydrous Na₂SO₄, filtered and concentrated to give the crude product which was purified by silica column gel chromatography (eluent: Petroleum ether/EtOAc=100/0 to 76/24) to afford 2-46d (200 mg, 47% yield) as an off-white solid.

MS (ESI) m/z: 238.2 [M+H]⁺.

### Step 4

### (9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (2-46)

To a solution of compound 2-46d (100 mg, 0.42 mmol) in Toluene (6 mL) and o-cresol (0.35 mL) was added 1-1i (110 mg, 0.42 mmol) and PPTS (16 mg, 0.06 mmol). The mixture was refluxed at 140° C. for 5 h. LCMS showed the reaction was completed. Solvent was evaporated and the residue was purified by silica column gel chromatography (eluent: CH₂Cl₂/MeOH=100/0 to 92/8) to afford 2-46 (80 mg, 41% yield) as an orange solid.

MS (ESI) m/z: 465.2 [M+H]⁺.

¹H NMR (400 MHz, DMSO-d6) δ=7.74 (d, J=11.0, 1H), 7.31 (s, 1H), 6.53 (d, J=1.9, 1H), 5.44 (s, 2H), 5.30 (s, 2H), 4.77 (t, J=4.8, 1H), 3.69-3.56 (m, 4H), 3.10 (t, J=18.6, 2H), 2.38 (s, 3H), 2.29 (d, J=9.7, 1H), 1.99-1.81 (m, 3H), 1.73 (d, J=5.4, 2H), 0.93-0.81 (m, 3H).

### Example 2-47

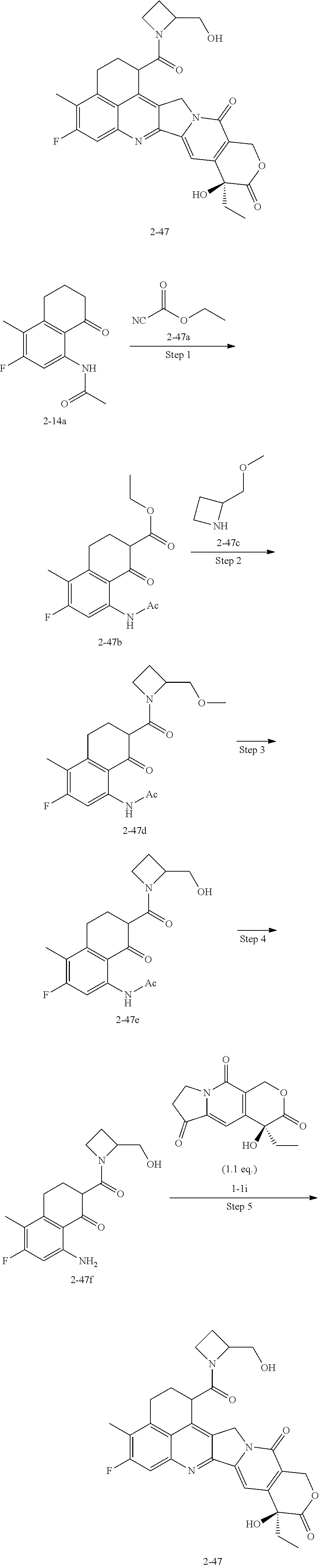

### Step 1

### Ethyl 8-acetamido-6-fluoro-5-methyl-1-oxo-1,2,3,4-tetrahydronaphthalene-2-carboxylate (2-47b)

Compound 2-47b (4 g, 61.24% yield) was synthesized according to synthetic procedure of step 1 of example 2-14c.

MS (ESI) m/z: 308.2 [M+H]⁺

### Step 2

### N-(3-fluoro-7-(2-(methoxymethyl)azetidine-1-carbonyl)-4-methyl-8-oxo-5,6,7,8-tetrahydronaphthalen-1-yl)acetamide (2-47d)

To a suspension of activated 4 Å MS (3 g) in toluene (50 mL) was added 2-47b (3 g, 9.77 mmol) and 2-(methoxymethyl)azetidine (1.97 g, 19.54 mmol) under N₂ atmosphere. The resulting mixture was heated at 70° C. for 18 h. The reaction mixture was then cooled to an ambient temperature, and filtered through a pad of Celite, and the filtrates were concentrated in vacuo. The crude product was purified by chromatography on SiO₂ (EtOAc/hexane, 1/5) to give 2-47d (1.1 g, 31.09% yield) as a yellow solid.

MS (ESI) m/z: 363.3 [M+H]⁺

¹H NMR (400 MHz, CD₃OD) δ 6.29 (dd, J=12.3, 3.5 Hz, 1H), 4.76-4.44 (m, 1H), 4.32-3.48 (m, 6H), 3.09-2.99 (m, 1H), 2.74 (ddd, J=17.3, 11.7, 5.2 Hz, 1H), 2.48-2.14 (m, 4H), 2.07-1.92 (m, 4H).

### Step 3

### N-(3-fluoro-7-(2-(hydroxymethyl)azetidine-1-carbonyl)-4-methyl-8-oxo-5,6,7,8-tetrahydronaphthalen-1-yl)acetamide (2-47e)

Compound 2-47e (150 mg, 14.18% yield) was synthesized according to synthetic procedure of step 4 of example 2-14.

MS (ESI) m/z: 349.3 [M+H]⁺

### Step 4

### 8-amino-6-fluoro-2-(2-(hydroxymethyl)azetidine-1-carbonyl)-5-methyl-3,4-dihydronaphthalen-1 (2H)-one (2-47f)

Compound 2-47f (130 mg, crude) was synthesized according to synthetic procedure of step 2 of example 2-14d.

MS (ESI) m/z: 307.3 [M+H]⁺

### Step 5

### (9S)-9-ethyl-5-fluoro-9-hydroxy-1-(2-(hydroxymethyl)azetidine-1-carbonyl)-4-methyl-1,2,3,9,12,15-hexahydro-10H,13H-benzo[de]pyrano[3′,4′:6,7]indolizino[1,2-b]quinoline-10,13-dione (2-47)

Compound 2-47 (3.2 mg, 6.62% yield) was synthesized according to synthetic procedure of step 3 of example 2-14e.

MS (ESI) m/z: 534.5 [M+H]⁺

### Example 2-48

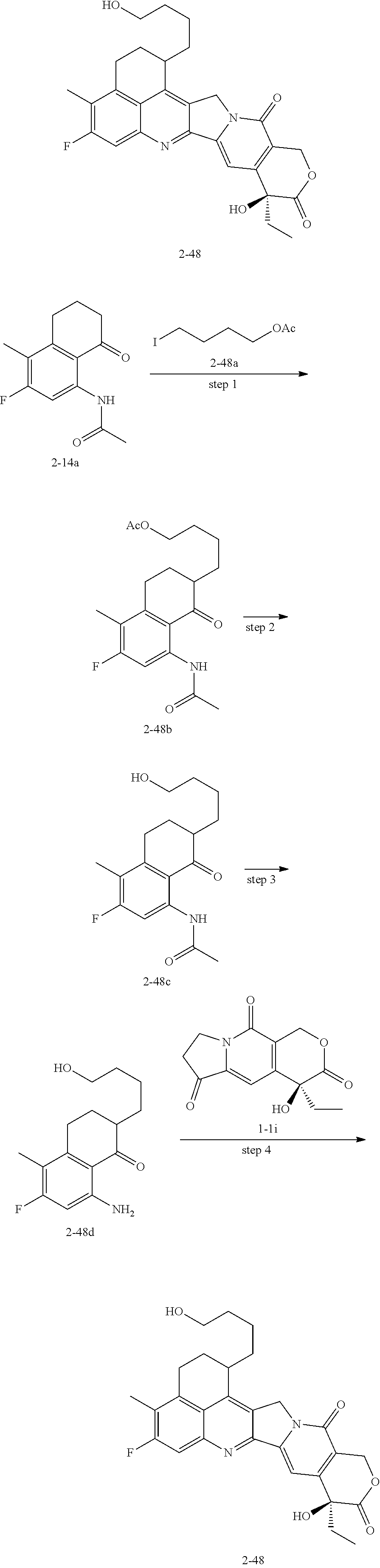

### Step 1

### 4-(8-acetamido-6-fluoro-5-methyl-1-oxo-1,2,3,4-tetrahydronaphthalen-2-yl)butyl acetate (2-48b)

Compound 2-48b (499 mg, 33.6% yield) was synthesized according to synthetic procedure of step 1 of example 2-14c.

MS (ESI) m/z: 350.3 [M+H]⁺

### Step 2

### N-(3-fluoro-7-(4-hydroxybutyl)-4-methyl-8-oxo-5,6,7,8-tetrahydronaphthalen-1-yl)acetamide (2-48c)

To a solution of 2-48b (499 mg, 1.43 mmol) in THF (20 mL) and MeOH (20 mL) was added Na₂CO₃ (302.94 mg, 2.86 mmol) was added at 0° C., and the mixture was stirred at the same temperature for 4 h. The reaction was extracted thrice with EtOAc. The combined organic layers were washed with brine and dried over MgSO₄ before the combined extracts were concentrated in vacuo. The resulting crude residue was purified by flash column chromatography (silica gel, Hexane:EtOAc=50:50) to give 2-48c (410 mg, 93.4% yield) as a colorless oil.

MS (ESI) m/z: 306.1 [M−H]⁺

### Step 3

### 8-amino-6-fluoro-2-(4-hydroxybutyl)-5-methyl-3,4-dihydronaphthalen-1 (2H)-one (2-48d)

Compound 2-48d (240 mg, 67.99% yield) was synthesized according to synthetic procedure of step 2 of example 2-14d.

MS (ESI) m/z: 266.2 [M+H]⁺

### Step 4

### (9S)-9-ethyl-5-fluoro-9-hydroxy-1-(4-hydroxybutyl)-4-methyl-1,2,3,9,12,15-hexahydro-10H,13H-benzo[de]pyrano[3′,4′:6,7]indolizino[1,2-b]quinoline-10,13-dione (2-48)

Compound 2-48 (105 mg, 23.64% yield) was synthesized according to synthetic procedure of step 3 of example 2-14e.

MS (ESI) m/z: 493.5 [M+H]⁺

¹H NMR (400 MHz, DMSO-d6) δ 7.70 (dd, J=11.1, 3.5 Hz, 1H), 7.30 (d, J=2.3 Hz, 1H), 5.44 (s, 2H), 5.25 (dt, J=18.7, 11.4 Hz, 2H), 3.44 (dd, J=9.7, 5.7 Hz, 2H), 3.33 (s, 1H), 3.06 (d, J=20.3 Hz, 2H), 2.36 (s, 3H), 2.30 (d, J=13.3 Hz, 1H), 2.02-1.80 (m, 3H), 1.68-1.36 (m, 6H), 0.90 (td, J=7.2, 2.7 Hz, 3H).

### Example 2-49, 2-50

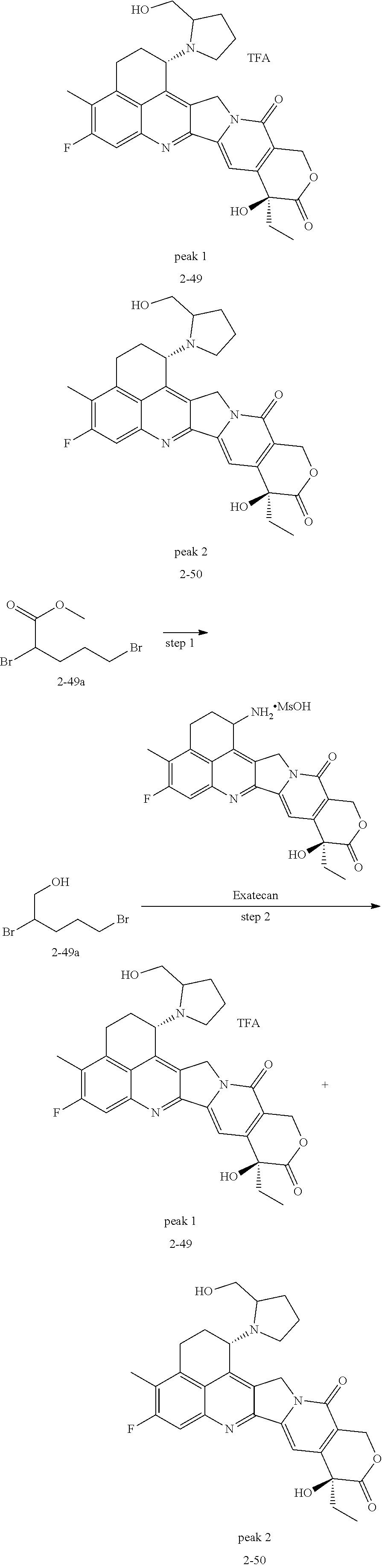

### Step 1

### 2,5-dibromopentan-1-ol (2-49b)

To a solution of ester 2-49a (1 g, 3.65 mmol, 1.00 equiv) in DCM (15 mL) at −78° C. was added a 1.0 M solution of diisobutylaluminum hydride in hexanes (7.5 mL, 7.5 mmol, 2.05 equiv) and the solution was removed from the cooling bath for 45 min at which time the reaction was shown to be complete by TLC analysis. The solution was cooled to −78° C. and quenched with satd Rochelle's salt (30 mL), diluted with DCM 30 mL), removed from the cold bath, and stirred vigorously at rt until the phases became distinct and clear (1.5 h). The phases were then separated and the aq phase was extracted with DCM (2×50 mL) and the combined organic phases were dried over MgSO₄, filtered, and the solvent was removed to yield alcohol 2-49b (880 mg, 98% yield) as a colorless oil.

### Step 2

### (1S,9S)-9-ethyl-5-fluoro-9-hydroxy-1-(2-(hydroxymethyl)pyrrolidin-1-yl)-4-methyl-1,2,3,9,12,15-hexahydro-10H,13H-benzo[de]pyrano[3′,4′:6,7]indolizino[1,2-b]quinoline-10,13-dione (2-49)

### (1S,9S)-9-ethyl-5-fluoro-9-hydroxy-1-(2-(hydroxymethyl)pyrrolidin-1-yl)-4-methyl-1,2,3,9,12,15-hexahydro-10H,13H-benzo[de]pyrano[3′,4′:6,7]indolizino[1,2-b]quinoline-10,13-dione (2-50)

To a solution of 2-49b (250 mg, 0.47 mmol) in 6 ml DMF was added exatecan mesylate (288 mg, 1.176 mmol), NaI (14.1 mg, 0.194 mol) and DIEA (152 mg, 1.176 mmol). The mixture was stirred at 80° C. for 19 h. The mixture was purified by prep-HPLC (TFA) (Method: column: XBridge Prep C18 OBD 5 um 19*150 mm; Mobile phase: A—water (0.1% TFA): B-acetonitrile; Flow rate: 20 mL/min, the fraction was lyophilized to give 2-49 (2.2 mg, 2% yield) as a white solid and 2-50 (7.7 mg, 6% yield) as a white solid.

MS (ESI) m/z: 520.5 [M+H]⁺

### Example 2-51, 2-52

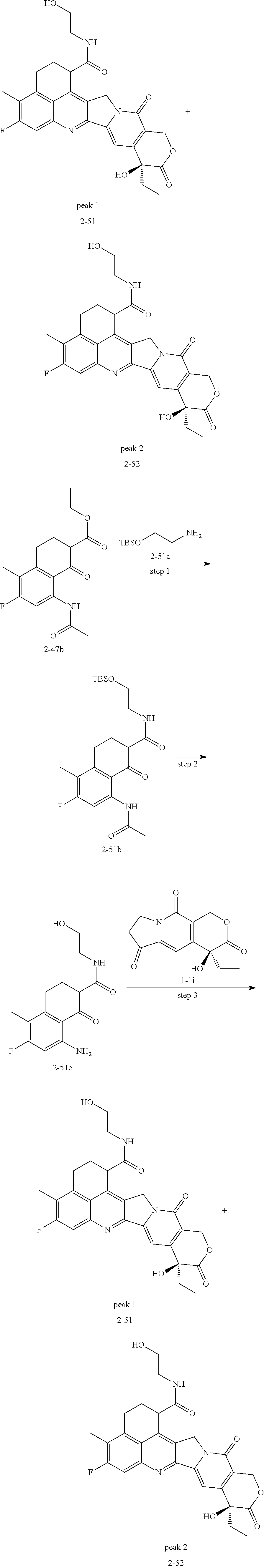

### Step 1

### 8-acetamido-N-(2-((tert-butyldimethylsilyl)oxy)ethyl)-6-fluoro-5-methyl-1-oxo-1,2,3,4-tetrahydronaphthalene-2-carboxamide (2-51b)

2-51b (302 mg, 75% yield) was synthesized according to synthetic procedure of example 2-47d.

MS (ESI) m/z: 436.3 [M+H]⁺

### Step 2

### 8-amino-6-fluoro-N-(2-hydroxyethyl)-5-methyl-1-oxo-1,2,3,4-tetrahydronaphthalene-2-carboxamide (2-51c)

2-51b (190 mg, 58% yield) was synthesized according to synthetic procedure of example 2-47f.

MS (ESI) m/z: 281.3 [M+H]⁺

### Step 3

### (9S)-9-ethyl-5-fluoro-9-hydroxy-N-(2-hydroxyethyl)-4-methyl-10,13-dioxo-2,3,9,10,13,15-hexahydro-1H,12H-benzo[de]pyrano[3′,4′:6,7]indolizino[1,2-b]quinoline-1-carboxamide (2-51, 2-52)

2-51 (4.6 mg, 3% yield) and 2-52 (9.0 mg, 6% yield) was synthesized according to synthetic procedure of example 2-47.

2-51

MS (ESI) m/z: 508.4 [M+H]⁺

¹H NMR (400 MHz, DMSO-d6) δ 8.30 (t, J=5.5 Hz, 1H), 7.77 (d, J=11.0 Hz, 1H), 7.32 (s, 1H), 6.53 (s, 1H), 5.43 (s, 2H), 5.33 (d, J=18.9 Hz, 1H), 5.02 (d, J=18.9 Hz, 1H), 4.72 (t, J=5.2 Hz, 1H), 4.25 (s, 1H), 3.15-3.12 (m, 5H), 2.44-2.43 (m, 2H), 2.38 (s, 3H), 2.13-2.11 (s, 1H), 1.92-1.86 (m, 2H), 0.87 (t, J=7.2 Hz, 3H).

2-52

MS (ESI) m/z: 508.4 [M+H]⁺

¹H NMR (400 MHz, DMSO-d6) δ 8.32 (t, J=5.7 Hz, 1H), 7.77 (d, J=11.0 Hz, 1H), 7.32 (s, 1H), 6.53 (s, 1H), 5.43 (s, 2H), 5.33 (d, J=18.9 Hz, 1H), 5.02 (d, J=18.9 Hz, 1H), 4.71 (t, J=5.3 Hz, 1H), 4.26 (t, J=4.0 Hz, 1H), 3.44-3.41 (m, 2H), 3.18-3.14 (m, 4H), 2.46-2.40 (m, 1H), 2.38 (s, 3H), 2.18-2.01 (m, 1H), 1.95-1.78 (m, 2H), 0.87 (t, J=7.3 Hz, 3H).

### Example 2-53

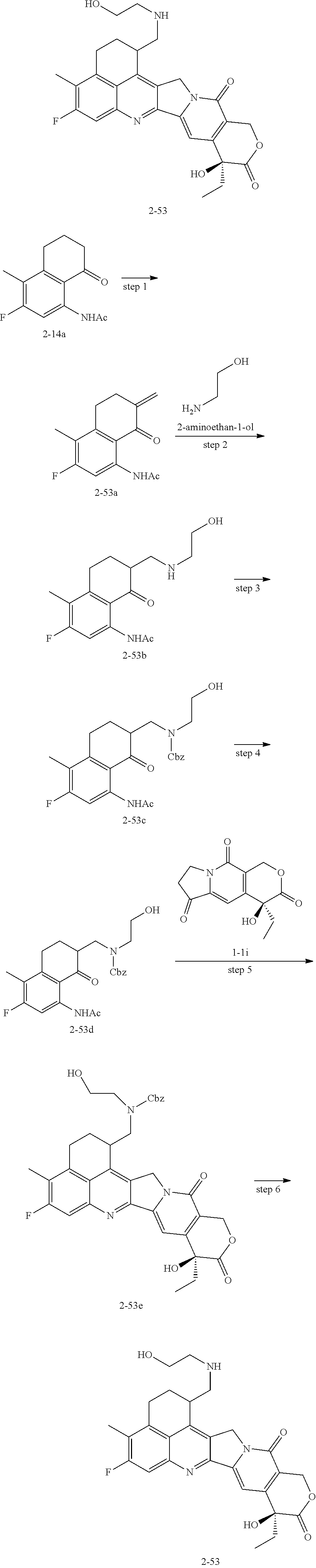

### Step 1

### N-(3-fluoro-4-methyl-7-methylene-8-oxo-5,6,7,8-tetrahydronaphthalen-1-yl)acetamide (2-53a)

To a solution of 2-14a (470.5 mg, 2 mmol) in CH₃CN (5 mL) was added paraformaldehyde (150 mg, 5 mmol) and ZnCl₂ (136.3 mg, 1 mmol). The mixture was stirred at r.t. for 10 min. After that pyrrolidine (287 mg, 4 mmol) was added dropwise. Upon completion of addition, the mixture was stirred at 70° C. for 2 h. Then the reaction solution was cooled to r.t., the solvent was removed by evaporation and the residue was directly purified by silica column gel chromatography (eluent: Hexane/EtOAc=30/1 to 5/1) to afford 2-53a as a white solid (210 mg, yield 42.5%).

¹H NMR (400 MHz, CDCl₃) δ 12.42 (s, 1H), 8.46 (d, J=12.9 Hz, 1H), 6.19 (d, J=1.3 Hz, 1H), 5.51 (d, J=1.6 Hz, 1H), 2.93 (t, J=6.5 Hz, 2H), 2.78 (t, J=6.4 Hz, 2H), 2.25 (s, 3H), 2.17 (d, J=1.9 Hz, 3H).

MS (ESI) m/z: 248.2 [M+H]⁺

### Step 2

### N-(3-fluoro-7-(((2-hydroxyethyl)amino)methyl)-4-methyl-8-oxo-5,6,7,8-tetrahydronaphthalen-1-yl)acetamide (2-53b)

To a solution of 2-53a (200 mg, 0.8 mmol) in THF (10 mL) was added 2-aminoethan-1-ol (108.8 mg, 1.78 mmol). The mixture was stirred at r.t. for 40 min. Then the solvent was removed by evaporation. The crude product, which is not stable under silica gel purification, was used directly in the next step without further purification (crude 290 mg, yield 117%).

MS (ESI) m/z: 309.3 [M+H]⁺

### Step 3

### N-(3-fluoro-7-(((2-hydroxyethyl)amino)methyl)-4-methyl-8-oxo-5,6,7,8-tetrahydronaphthalen-1-yl)acetamide (2-53c)

To a solution of 2-53b (80 mg, 0.26 mmol) in CH₂Cl₂ (2 mL) was added Et₃N (40 mg, 0.4 mmol). The solution was then cooled to 0° C. and Benzyl chloroformate (48.6 mg, 0.28 mmol) was then added dropwise. The mixture was then warmed to r.t. and stirred at r.t. for 2 h. The solvent was removed by evaporation and the residue was directly purified by silica column gel chromatography (eluent: CH₂Cl₂/MeOH=32/1) to afford 2-53c as a colorless oil (110 mg, yield 95.6%).

¹H NMR (400 MHz, CDCl₃) δ 12.09 (s, 1H), 8.39 (d, J=13.0 Hz, 1H), 7.42-7.27 (m, 4H), 7.21 (s, 1H), 5.30 (s, 1H), 5.13 (d, J=13.1 Hz, 2H), 4.94 (d, J=12.0 Hz, 1H), 3.99-3.71 (m, 3H), 3.65-3.53 (m, 1H), 3.52-3.35 (m, 2H), 2.85 (t, J=62.3 Hz, 3H), 2.21 (s, 3H), 2.10 (d, J=27.8 Hz, 3H), 1.79 (s, 2H).

MS (ESI) m/z: 465.4 [M+Na]⁺

Step 4 benzyl ((8-amino-6-fluoro-5-methyl-1-oxo-1,2,3,4-tetrahydronaphthalen-2-yl)methyl)(2-hydroxyethyl)carbamate (2-53d)

To a solution of compound 2-53c (100 mg, 0.23 mmol) in MeOH (3 mL) was added HCl (2 N, 1.5 mL). The mixture was stirred at 60° C. for 3 h. The mixture was cooled to r.t. and adjusted to pH 8 using sat. NaHCO₃. The mixture was extracted with EtOAc (10 mL*3). The organic layer was dried and concentrated. The crude product 2-53d (red oil) was used directly in the next step without further purification (crude: 81 mg, yield 89.6%).

MS (ESI) m/z: 401.4 [M+H]⁺

### Step 5

### Nbenzyl (((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(2-hydroxyethyl)carbamate (2-53e)

To a solution of 2-53d (80 mg, 0.2 mmol) in toluene (10 mL) and o-cresol (0.5 mL) was added 1-1i (60.5 mg, 0.23 mmol) and PPTS (30.2 mg, 0.12 mmol). The mixture was refluxed at 140° C. for 12 h. The solvent was removed by evaporation and the crude product 2-53e was used directly in the next step without further purification (crude: 120 mg, yield 95.6%).

MS (ESI) m/z: 628.5 [M+H]⁺

### Step 6

### Nbenzyl (9S)-9-ethyl-5-fluoro-9-hydroxy-1-(((2-hydroxyethyl)amino)methyl)-4-methyl-1,2,3,9,12,15-hexahydro-10H,13H-benzo[de]pyrano[3′,4′:6,7]indolizino[1,2-b]quinoline-10,13-dione (2-53)

To the solution of 2-53e (crude 120 mg, 0.2 mmol) in MeOH (2 mL) and THF (2 mL) was added wet Pd/C (20%, 24 mg), stirred under H₂ atmosphere at r.t. for 8 h. The solution was filtered through Celite and concentrated under vacuum. The residue was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to give 2-53 as a white solid (33.2 mg, 33.6% yield).

¹H NMR (400 MHz, DMSO-d6) δ 8.21 (s, 1H), 7.74 (d, J=11.0 Hz, 1H), 7.30 (s, 1H), 6.53 (s, 1H), 5.44 (s, 2H), 5.41-5.34 (m, 2H), 5.31 (s, 1H), 3.55 (s, 1H), 3.49 (dd, J=5.6, 3.8 Hz, 2H), 3.05 (dd, J=27.9, 9.3 Hz, 3H), 2.89-2.81 (m, 2H), 2.74-2.67 (m, 2H), 2.37 (s, 3H), 2.03-1.81 (m, 4H), 0.87 (dd, J=11.7, 4.3 Hz, 3H).

MS (ESI) m/z: 494.4 [M+H]⁺

### Example 2-54

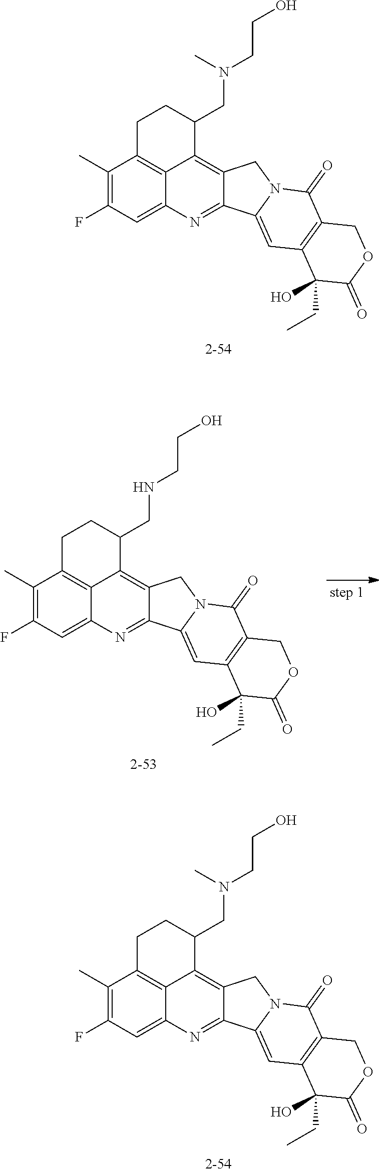 [SMILES: CC[C@@]1(O)C(=O)OCc2c1cc1n(c2=O)Cc2c-1nc1cc(F)c(C)c3c1c2C(CN(C)CCO)CC3 ; CC[C@@]1(O)C(=O)OCc2c1cc1n(c2=O)Cc2c-1nc1cc(F)c(C)c3c1c2C(CNCCO)CC3 ; CC[C@@]1(O)C(=O)OCc2c1cc1n(c2=O)Cc2c-1nc1cc(F)c(C)c3c1c2C(CN(C)CCO)CC3]

### Step 1

### (9S)-9-ethyl-5-fluoro-9-hydroxy-1-(((2-hydroxyethyl)(methyl)amino)methyl)-4-methyl-1,2,3,9,12,15-hexahydro-10H,13H-benzo[de]pyrano[3′,4′:6,7]indolizino[1,2-b]quinoline-10,13-dione (2-54)

To the solution of 2-53 (10 mg, 0.02 mmol) in MeOH (1 mL) was added 37% formaldehyde solution (2.8 μL, 0.04 mmol). The solution was then cooled to 0° C. and NaBH₃CN (2 mg, 0.03 mmol) was then added in one portion. The solution was then warmed to r.t. and stirred for another 2 h. The mixture was then quenched with H₂O and purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to give 2-54 as a white solid (5.5 mg, 54.2% yield).

MS (ESI) m/z: 508.5 [M+H]⁺

### Example 2-55

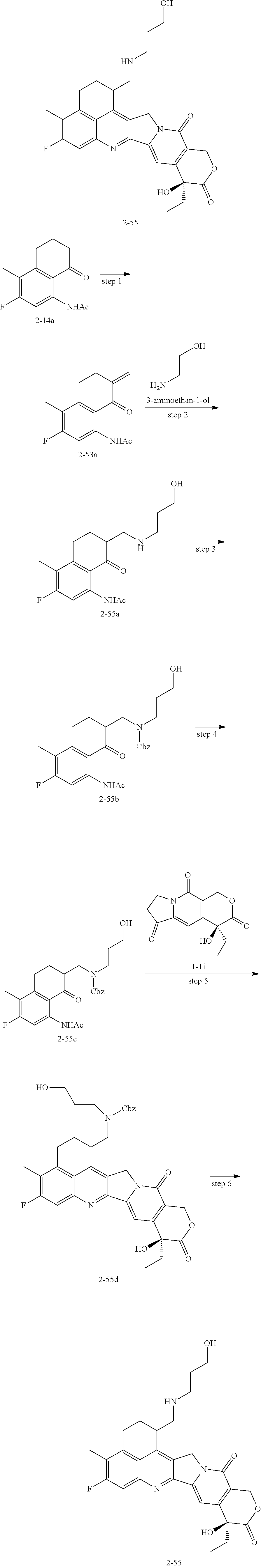

### Step 1˜Step 6

### (9S)-9-ethyl-5-fluoro-9-hydroxy-1-(((3-hydroxypropyl)amino)methyl)-4-methyl-1,2,3,9,12,15-hexahydro-10H,13H-benzo[de]pyrano[3′,4′:6,7]indolizino[1,2-b]quinoline-10,13-dione (2-55)

2-55 (0.6 mg, 6% yield) was synthesized according to synthetic procedure of example 2-53.

MS (ESI) m/z: 508.5 [M+H]⁺

### Example 2-56 and 2-57

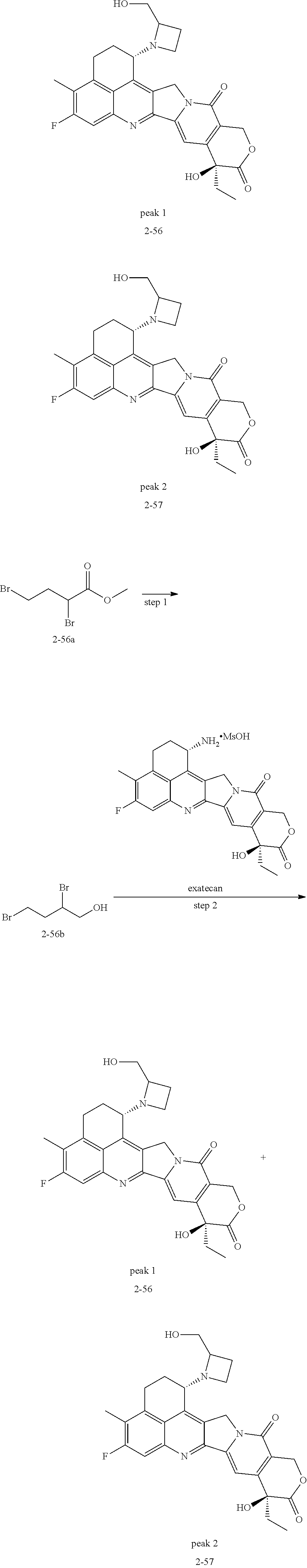

### Step 1

### 2,4-dibromobutan-1-ol (2-56b)

2-56b (brown oil, 1.2 g, yield 67.4%) was synthesized according to the procedure described in Step 1 of Example 2-49b.

### Step 2

### (1S,9S)-9-ethyl-5-fluoro-9-hydroxy-1-(2-(hydroxymethyl)azetidin-1-yl)-4-methyl-1,2,3,9,12,15-hexahydro-10H,13H-benzo[de]pyrano[3′,4′:6,7]indolizino[1,2-b]quinoline-10,13-dione (2-56) and (1R,9S)-9-ethyl-5-fluoro-9-hydroxy-1-(2-(hydroxymethyl)azetidin-1-yl)-4-methyl-1,2,3,9,12,15-hexahydro-10H,13H-benzo[de]pyrano[3′,4′:6,7]indolizino[1,2-b]quinoline-10,13-dione (2-57)

Example 2-56 (white solid, 1.5 mg, yield 2.5%) and 2-57 (white solid, 1.5 mg, yield 2.5%) were synthesized according to the procedure described in example 2-49 and 2-50.

2-56: MS (ESI) m/z: 506.5. [M+H]⁺

2-57: MS (ESI) m/z: 506.5. [M+H]⁺

### Example 2-58

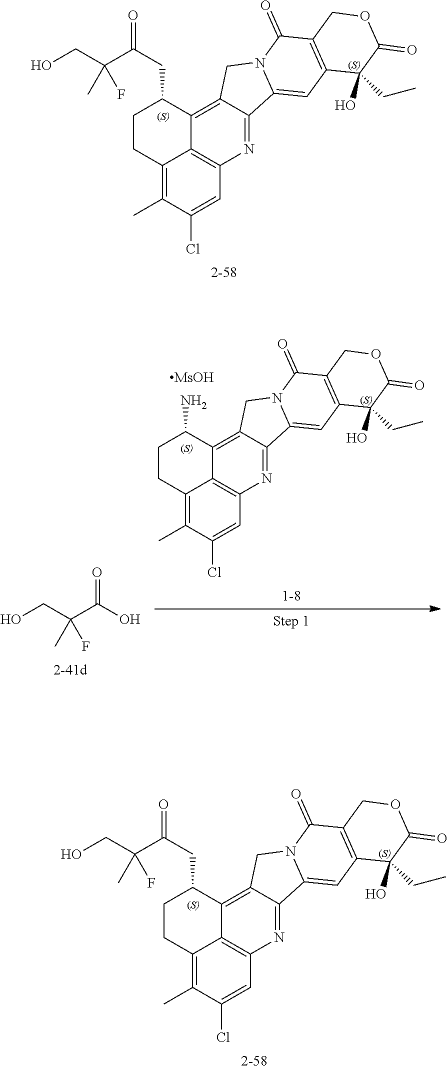

### Step 1

N-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-fluoro-3-hydroxy-2-methylpropanamide (2-58)

Compound 2-58 (9.6 mg, 63.1% yield) was synthesized according to synthetic procedure of step 3 of example 2-2.

MS (ESI) m/z: 556.4 [M+H]⁺

### Example 2-59 & 2-60

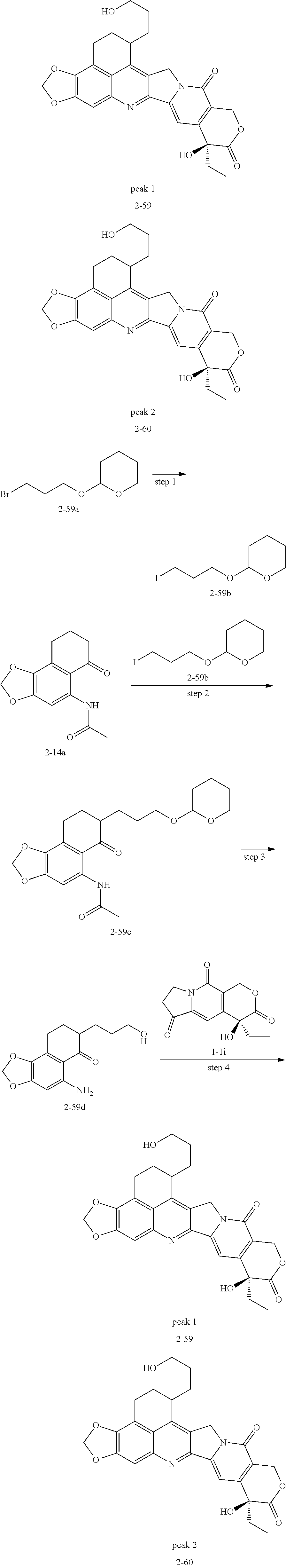

### Step 1

### 2-(3-iodopropoxy)tetrahydro-2H-pyran (2-59b)

A solution of compound 2-59a (2.0 g, 9.01 mmol) in acetone (20 mL) was added NaI (4.0 g, 27 mmol). The mixture was stirred at 60° C. for 3 h. The mixture was diluted with hexane (40 mL) and washed with water (40 mL) and brine (40 mL). The combined organic layer was dried over Na₂SO₄ and concentrated to afford 2-59b (1.3 g, 54% yield) as a colorless oil.

¹H NMR (400 MHz, CDCl₃) δ 4.61 (dd, J=4.4, 2.8 Hz, 1H), 3.91-3.83 (m, 1H), 3.83-3.77 (m, 1H), 3.56-3.49 (m, 1H), 3.45 (dt, J=10.0, 5.9 Hz, 1H), 3.30 (td, J=6.8, 1.0 Hz, 2H), 2.10 (ddd, J=12.7, 6.8, 5.9 Hz, 2H), 1.90-1.63 (m, 3H), 1.61-1.48 (m, 5H).

### Step 2

### N-(6-oxo-7-(3-((tetrahydro-2H-pyran-2-yl)oxy)propyl)-6,7,8,9-tetrahydronaphtho[1,2-d][1,3]dioxol-5-yl)acetamide (2-59c)

To a solution of compound 2-14a (100 mg, 3.52 mmol) in THF (5 mL) was drop-wised added t-BuOK (1.2 mL, 1.21 mmol, 1 M in THF) at −40° C. under N₂. The mixture was stirred at −40° C. for 30 min under N₂. Then compound 2-59b (164 mg, 0.606 mmol, dissolved in 0.2 mL THF) was drop-wised added to the mixture. The mixture was stirred for 16 h with gradually heating up to room-temperature under N₂. The mixture was quenched with sat. NH₄Cl (3 mL) and extracted with EA (3 mL*3). The combined organic layer was dried over anhydrous Na₂SO₄, filtered and concentrated to give the residue. Which was purified by column (Pet.ether/ethyl acetate=1:0 to 4:1) to afford the compound 2-59c (20 mg, 12.7% yield) as yellow solid.

MS (ESI) m/z: 248.1 [M+H]⁺.

¹H NMR (400 MHz, CDCl₃) δ 12.44 (s, 1H), 8.26 (s, 1H), 6.01-5.98 (m, 2H), 4.59-4.53 (m, 1H), 3.90-3.72 (m, 2H), 3.53-3.36 (m, 2H), 3.00-2.88 (m, 1H), 2.79-2.67 (m, 1H), 2.55-2.44 (m, 1H), 2.19 (s, 3H), 2.17-2.09 (m, 1H), 2.06-1.86 (m, 2H), 1.86-1.65 (m, 6H), 1.60-1.54 (m, 2H).

### Step 3

### 5-amino-7-(3-hydroxypropyl)-8,9-dihydronaphtho[1,2-d][1,3]dioxol-6 (7H)-one (2-59d)

To a solution of compound 2-59c (20 mg, 0.05 mmol) in MeOH (5 mL) were added 2 N HCl (5 mL). The mixture was stirred at 60° C. for 3 h. The mixture was concentrated in vacuo to remove MeOH. Then the mixture was adjusted pH=8 using sat. Na₂CO₃ and extracted with CH₂Cl₂ (5 mL*3). The combined organic phase was concentrated in vacuo to give the residue, which was purified by silica column gel chromatography (eluent: Pet.ether/ethyl acetate=100/0 to 0/100) to afford 2-59d (10 mg, 74% yield) was obtained as yellow oil.

MS (ESI) m/z: 264.2 [M+H]⁺.

### Step 4

### (1R,10S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (2-59) and (1S,10S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (2-60)

To a solution of compound 2-59d (80 mg, 0.304 mmol) in toluene (14 mL) and o-cresol (2 mL) was added 1-1i (88 mg, 0.334 mmol) and PPTS (23 mg, 0.091 mmol). The mixture was refluxed at 140° C. for 16 h. The mixture was concentrated in vacuo to remove toluene and purified by silica column gel chromatography (eluent: CH₂Cl₂/MeOH=100/0 to 100/10) to crude product (mixture of 6 and 7). Which was purified by prep-HPLC (FA) (Method: column: XBridge Prep C18 OBD 5 um 19*250 mm; Mobile phase: A-water (0.1% formic acid): B-acetonitrile; Flow rate: 20 mL/min, the fraction was lyophilized to give 2-59 (15 mg, 71% yield) as a white solid and 2-60 (20 mg, 13% yield) as white solid.

Compound 2-59 (retention time: 2.54 min):

MS (ESI) m/z: 491.5 [M+H]⁺

¹H NMR (400 MHz, DMSO-d₆) δ 7.37 (s, 1H), 7.23 (s, 1H), 6.48 (s, 1H), 6.27 (d, J=2.9 Hz, 2H), 5.42 (s, 2H), 5.31 (d, J=18.9 Hz, 1H), 5.19 (d, J=18.8 Hz, 1H), 4.42 (t, J=5.1 Hz, 1H), 3.48-3.42 (m, 2H), 3.01-2.91 (m, 2H), 2.22 (d, J=13.4 Hz, 1H), 1.93-1.75 (m, 4H), 1.73-1.63 (m, 2H), 1.61-1.54 (m, 2H), 0.87 (t, J=7.3 Hz, 3H).

Compound 2-60 (retention time: 2.66 min):

MS (ESI) m/z: 491.5 [M+H]⁺

¹H NMR (400 MHz, DMSO-d₆) δ 7.37 (s, 1H), 7.23 (s, 1H), 6.49 (s, 1H), 6.27 (s, 2H), 5.42 (s, 2H), 5.31 (d, J=18.9 Hz, 1H), 5.20 (d, J=18.9 Hz, 1H), 4.43 (t, J=5.2 Hz, 1H), 3.47-3.43 (m, 2H), 3.02-2.86 (m, 2H), 2.22 (d, J=13.3 Hz, 1H), 1.94-1.78 (m, 3H), 1.73-1.63 (m, 2H), 1.63-1.53 (m, 3H), 0.87 (t, J=7.3 Hz, 3H).

### Example 3-1

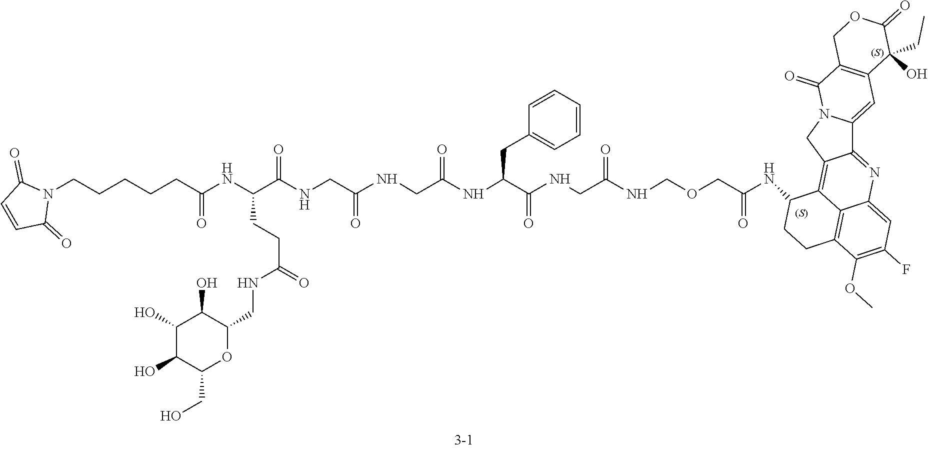

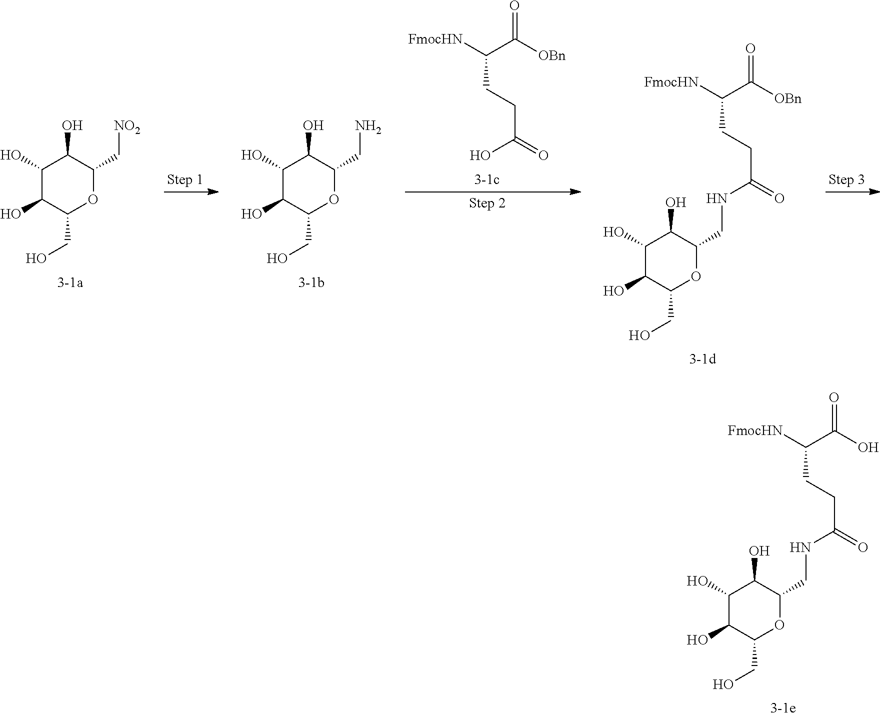

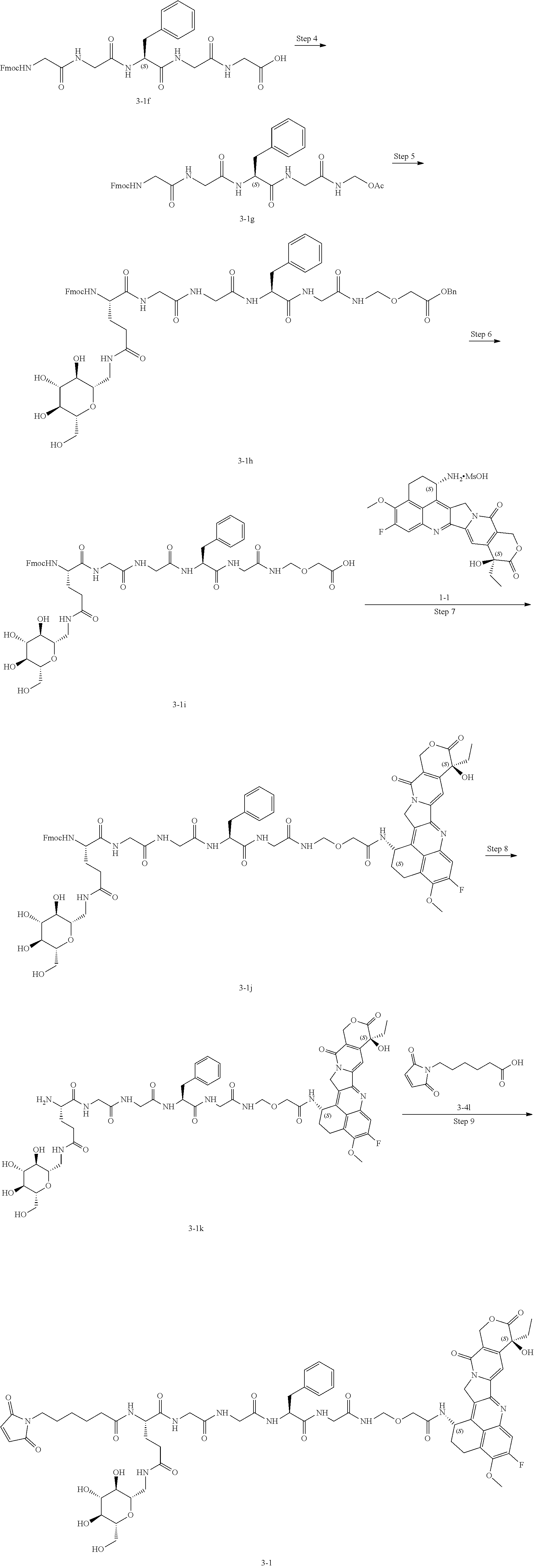

### Step 1

### (2S,3R,4R,5S,6R)-2-(aminomethyl)-6-(hydroxymethyl)tetrahydro-2H-pyran-3,4,5-triol (3-1b)

To a solution of compound 3-1a (200 mg, 0.90 mmol) in MeOH (3 mL) were added HCOONH₄ (226 mg, 3.58 mmol) and Pd/C (20 mg, 10%). The mixture was stirred at 70° C. for 2 h. The mixture was filtered and concentrated to give the product 3-1b (173 mg, crude) as white solid.

MS (ESI) m/z: 194.3 [M+H]⁺

### Step 2

### benzyl N2-(((9H-fluoren-9-yl)methoxy)carbonyl)-N5-(((2S,3R,4R,5S,6R)-3,4,5-trihydroxy-6-(hydroxymethyl)tetrahydro-2H-pyran-2-yl)methyl)-L-glutaminate (3-1d)

To a solution of compound 3-1b (3.0 g, 15.5 mmol) in DMF (100 mL) were added compound 3-1c (7.5 g, 16.3 mmol), HATU (11.8 g, 31.1 mmol) and DIEA (3.0 g, 23.3 mmol). The mixture was stirred at R.T. for 1 h. The mixture was concentrated and was purified by silica column gel chromatography (eluent: CH₂Cl₂/MeOH=100/0 to 85/15) to afford 3-1c (7.6 g, 77.1% yield) as white solid.

MS (ESI) m/z: 635.5 [M+H]⁺

### Step 3

### N2-(((9H-fluoren-9-yl)methoxy)carbonyl)-N5-(((2S,3R,4R,5S,6R)-3,4,5-trihydroxy-6-(hydroxymethyl)tetrahydro-2H-pyran-2-yl)methyl)-L-glutamine (3-1e)

To a solution of 3-1d (1.5 g, 2.36 mmol) in MeOH (20 mL) was added wet Pd/C (150 mg). The black suspension was purged with H₂ balloon for three times then reacted at r.t. for 4 h under H₂ balloon. After the reaction was completed, the black suspension was filtered off through a pad of celite and the cake wash with MeOH, the combined organic layers were concentrated under vacuum to provide 3-1e (1.29 g, Crude).

MS (ESI) m/z: 567.4 [M+Na]⁺

### Step 4

### (S)-11-benzyl-1-(9H-fluoren-9-yl)-3,6,9,12,15-pentaoxo-2-oxa-4,7,10,13,16-pentaazaheptadecan-17-yl acetate (3-1g)

To a solution of compound 3-1f (5.00 g, 8.12 mmol) in DMF (65 mL) were added Cu(OAc)₂ (560 mg, 3.09 mmol), Pb(OAc)₄ (4.11 g, 9.25 mmol) and HOAc (1.11 g, 18.44 mmol). The mixture was stirred at 65° C. under N₂ atmosphere for 2 h. The mixture was concentrated to 1/4 of the original volume, poured into ice-water (150 mL) and stirred for 30 min. The solid was filtered and co-evaporated with toluene (20 mL*4) to give compound 3-1g (5.20 g, crude) as off-white solid.

MS (ESI) m/z: 652.3 [M+Na]⁺

### Step 5

### benzyl (5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatricosan-23-oate (3-1h)

To a solution of compound 3-1g (1.0 g, 2.45 mmol) in DMF were added 3-1e (1.47 mmol, 2.7 mmol), HATU (1.40 g, 3.68 mmol) and DIEA (634 mg, 4.91 mmol). The mixture was stirred at RT for 30 min. The mixture was concentrated and purified by flash column chromatography (eluent: CH₂Cl₂/MeOH=100/0 to 20/80) to give the title compound 3-1h (1.3 g, 51.0% yield) was obtained as off-white solid.

MS (ESI) m/z: 1062.5 [M+Na]⁺

### Step 6

### (5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatricosan-23-oic acid (3-1i)

To a solution of compound 3-1h (35 mg, 0.034 mmol) in MeOH (4 mL) was added Pd/C (10%, 5 mg). The mixture was stirred at H₂ atmosphere (15 psi) for 3 h. The mixture was filtered through a pad of celite, concentrated to give compound 3-1i (32 mg, crude) as white solid.

MS (ESI) m/z: 972.5 [M+Na]⁺

### Step 7

### (9H-fluoren-9-yl)methyl ((6S,15S)-15-benzyl-24-(((1S,9S)-9-ethyl-5-fluoro-9-hydroxy-4-methoxy-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-1j)

To a solution of compound 3-1i (20 mg, 0.021 mmol) in DMF (2 mL) were added compound 1-1 (12 mg, 0.022 mmol), HATU (16 mg, 0.042 mmol) and DIEA (8 mg, 0.063 mmol). The mixture was stirred at r.t. for 30 min. The mixture was filtered, the filtrate was purified using prep-HPLC (Method: column: XBridge Prep C18 OBD 5 um 19*150 mm; Mobile phase: A—water (0.1% trifluoroacetic acid): B-acetonitrile; Flow rate: 20 mL/min to provide compound 3-1j (14.5 mg, 49.5% yield) as white solid.

MS (ESI) m/z: 1405.7 [M+Na]⁺

### Step 8

### (9H-fluoren-9-yl)methyl ((6S,15S)-15-benzyl-24-(((1S,9S)-9-ethyl-5-fluoro-9-hydroxy-4-methoxy-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-1k)

To a solution of compound 3-1j (14.5 mg, 0.011 mmol) in DMF (2 mL) was added Et₂NH (15.3 mg, 0.21 mmol). The mixture was stirred at r.t. for 30 min. The mixture was concentrated under high vacuum and co-evaporated with toluene (3 mL*3) to give 3-1k (12.2 mg, crude) as brown solid.

MS (ESI) m/z: 1183.6 [M+Na]⁺

### Step 9

### (S)—N1-((S)-10-benzyl-1-(((1S,9S)-9-ethyl-5-fluoro-9-hydroxy-4-methoxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-1)

To a solution of compound 3-1k (12 mg, 0.01 mmol) in DMF (2 mL) were added compound 3-11 (4.4 mg, 0.021 mmol), HATU (7.9 mg, 0.021 mmol) and DIEA (2.7 mg, 0.021 mmol). The mixture was stirred at r.t. for 30 min The mixture was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to compound 3-1 (6.3 mg, 45.0% yield).

MS (ESI) m/z: 1376.7 [M+Na]*. Retention time (3.74 min).

### Example 3-2

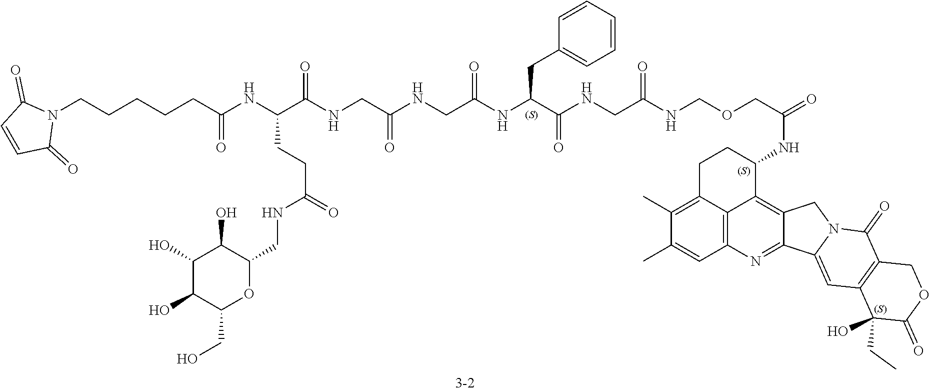

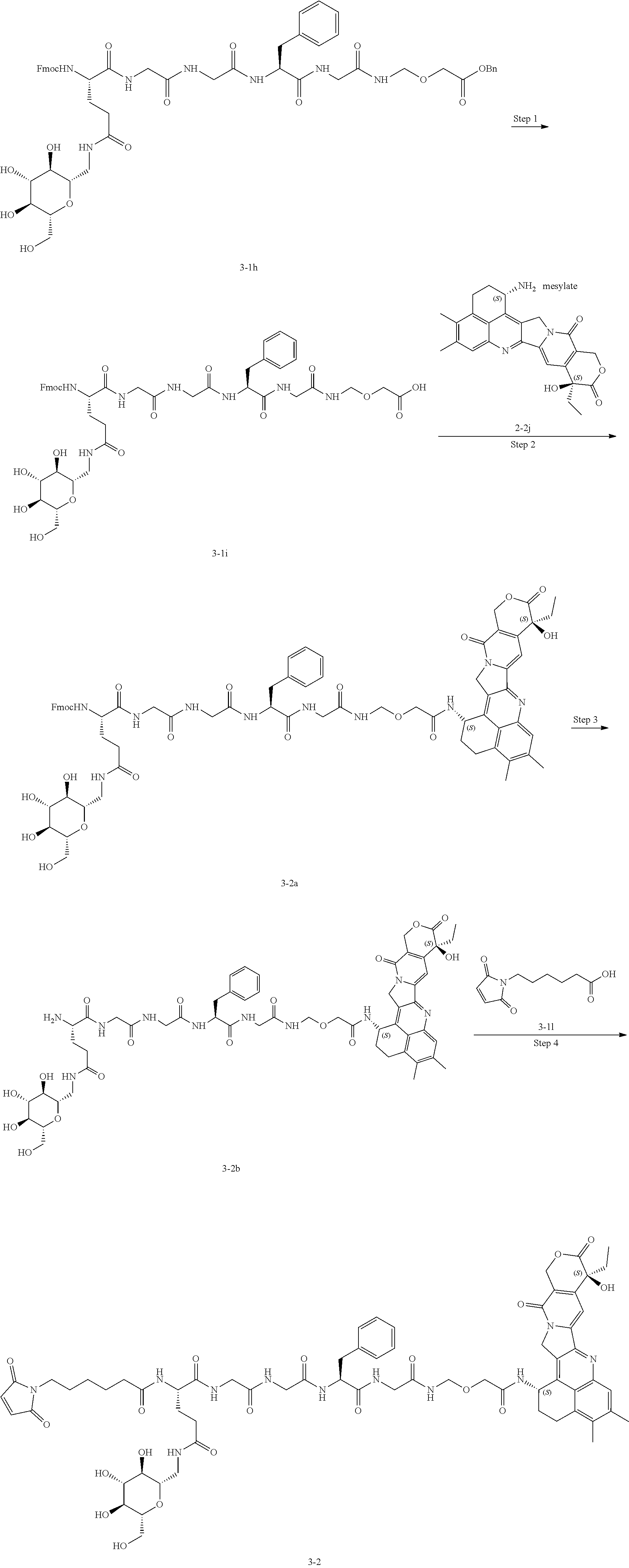

3-2 (15.2 mg, 98% purity, 67.7% yield) was synthesized according to a similar procedure as that of example 3-1.

MS (ESI) m/z: 1356.8 [M+Na]

### Example 3-3

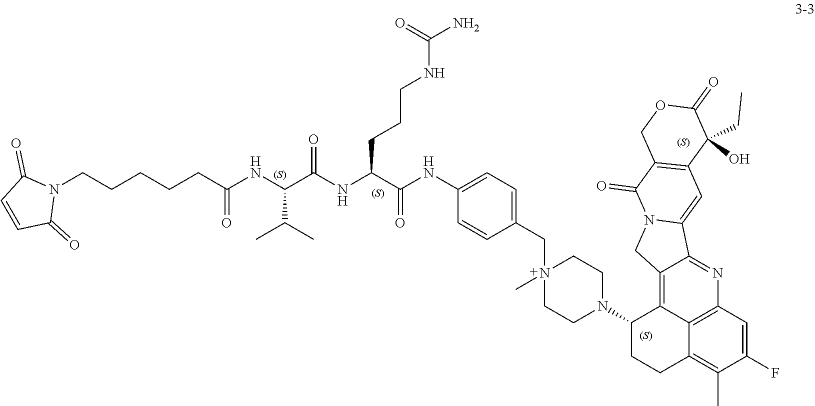

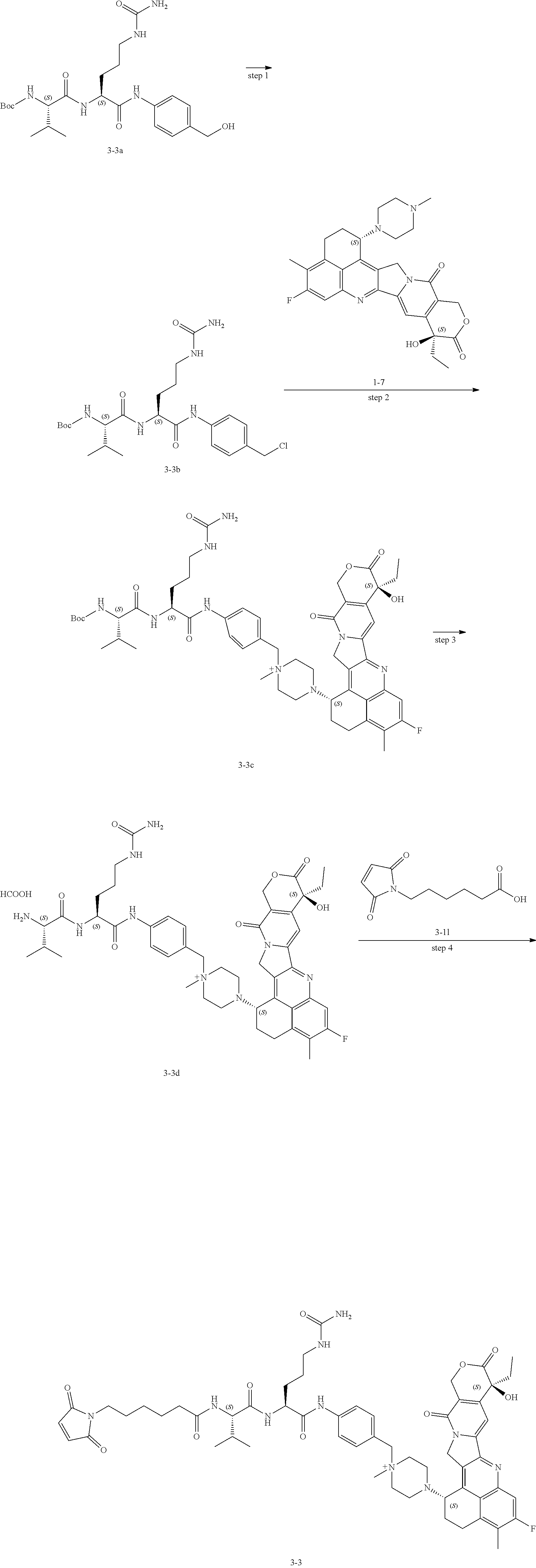

### Step 1

### tert-butyl ((S)-1-(((S)-1-((4-(chloromethyl)phenyl)amino)-1-oxo-5-ureidopentan-2-yl)amino)-3-methyl-1-oxobutan-2-yl)carbamate (3-3b)

To the pre-cooled solution of 3-3a (200 mg, 0.42 mmol) in dry DMF (1 mL) was added SOCl₂ (37 μL, 0.5 mmol) in an ice bath, stirred at 0° C. for 30 min. Water (3 mL) was added slowly to the reaction solution. The turbid solution was filtered and the filter cake was washed with water (3 mL) and MTBE (3 mL), then dried under vacuum to give compound 3-3b (180 mg, 86.7% yield) as an off-white solid.

MS (ESI) m/z: 398.4 [M+H-Boc]⁺

### Step 2

### 1-(4-((S)-2-((S)-2-((tert-butoxycarbonyl)amino)-3-methylbutanamido)-5-ureidopentanamido)benzyl)-4-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-methylpiperazin-1-ium (3-3c)

To the solution of 1-7 (5.5 mg, 11 mol), 3-3b (6.5 mg, 12 mol) and TBAI (2.0 mg, 5 mol) in dry DMF (0.5 mL) was added DIEA (4 μL, 27 mol), stirred at r.t. for 48 h. MTBE (2 mL) was added. The turbid solution was filtered to give compound 3-3c (10 mg, 96.1% yield) which was used directly for the next step.

MS (ESI) m/z: 980.7 [M]⁺

### Step 3

### 1-(4-((S)-2-((S)-2-amino-3-methylbutanamido)-5-ureidopentanamido)benzyl)-4-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-methylpiperazin-1-ium (3-3d) formate salt

To the solution of 3-3c (10 mg, 0.01 mmol) in DCM (0.8 mL) was added TFA (0.2 mL), stirred at r.t. for 20 min. The solution was concentrated and purified by prep-HPLC (Mobile phase A: 0.1% FA in water, B: MeCN; Gradient: 20%-35% B; Flow rate: 20 mL/min; Column: Xbridge Prep C18 OBD™ 5 m, 19*150 mm). The desired fraction was lyophilized to give 3-3d formate salt (8.2 mg, 86.8% yield) as a pale pink solid.

MS (ESI) m/z: 441.1 [M/2+H]⁺

### Step 4

### 1-(4-((S)-2-((S)-2-(6-(2,5-dioxo-2,5-dihydro-1H-pyrrol-1-yl)hexanamido)-3-methylbutanamido)-5-ureidopentanamido)benzyl)-4-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1-methylpiperazin-1-ium (3-3)

To the solution of 3-11 (3.2 mg, 0.015 mmol) and HATU (4.3 mg, 0.011 mmol) in dry DMF (0.2 mL) was added DIEA (4 μL, 0.022 mmol), stirred at r.t. for 15 min. The resulted solution was added into the solution of 3-3d (6.5 mg, 0.007 mmol) in dry DMF (0.5 mL), stirred at r.t. for 20 min. The solution was purified by prep-HPLC (Mobile phase A: 0.1% FA in water, B: MeCN; Gradient: 30%-40% B; Flow rate: 20 mL/min; Column: Xbridge Prep C18 OBD™ 5 μm, 19*150 mm). The desired fraction was lyophilized to give 3-3 (2.6 mg, 32.8% yield) as a white solid.

MS (ESI) m/z: 1073.7 [M]⁺

### Example 3-4

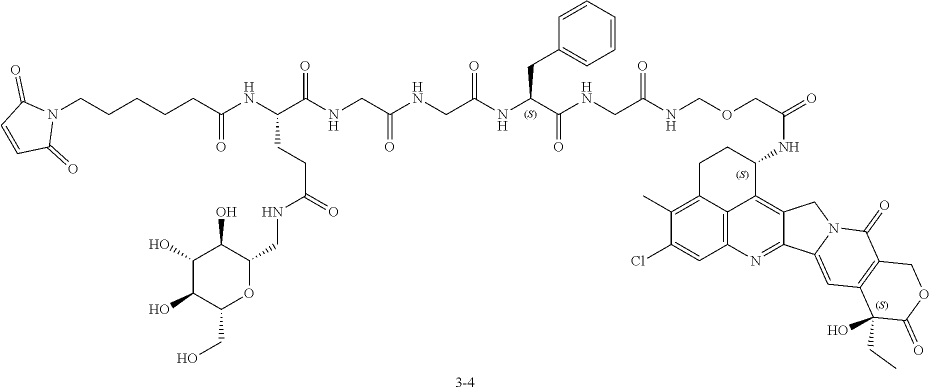

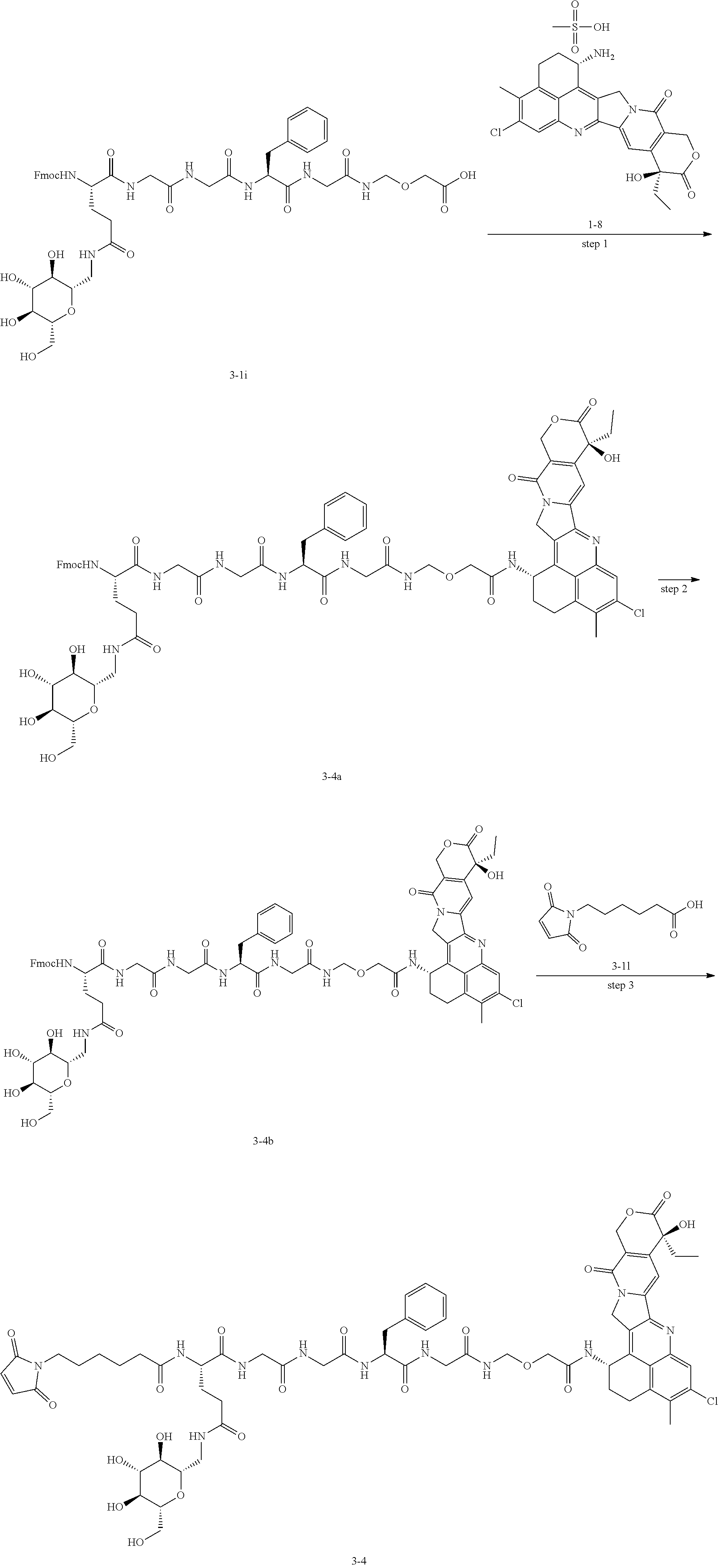

### Step 1

### (9H-fluoren-9-yl)methyl ((6S,15S)-15-benzyl-24-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,20-pentaazatetracosan-6-yl)carbamate (3-4a)

To a solution of compound 3-1i (20.0 mg, 0.02 mmol) in DMF (2 mL) were added 1-8 mesylate (11.6 mg, 0.02 mmol), HATU (8.0 mg, 0.02 mmol) and DIEA (8.2 mg, 10 μL, 0.02 mmol). The mixture was stirred at r.t. for 30 min. The mixture was filtered and the filtrate was purified using prep-HPLC (Mobile phase A: 0.1% TFA in water, B: MeCN; Flow rate: 20 mL/min; Column: Xbridge Prep C18 OBD™ 5 m, 19*150 mm) to provide compound 3-4a (22 mg, 75.5% yield) as white solid.

MS (ESI) m/z: 1405.6 [M+Na]⁺

### Step 2

### (S)-2-amino-N′—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,11,14-triazahexadecan-16-yl)-N⁵-(((2S,3R,4R,5S,6R)-3,4,5-trihydroxy-6-(hydroxymethyl)tetrahydro-2H-pyran-2-yl)methyl)pentanediamide (3-4b)

To a solution of compound 3-4a (22.0 mg, 0.016 mmol) in DMF (2 mL) was added Et₂NH (11.6 mg, 16 μL, 0.16 mmol). The mixture was stirred at r.t. for 30 min. The mixture was concentrated under high vacuum and co-evaporated with toluene (10 mL) to give 3-4b (18 mg, crude) as brown solid.

MS (ESI) m/z: 1161.7 [M+H]⁺

### Step 3

### (S)—N¹—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,11,14-triazahexadecan-16-yl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (3-4)

To a solution of compound 3-4b (18.0 mg, 0.016 mmol) in DMF (2 mL) were added 3-11 (4.9 mg, 0.023 mmol), HATU (8.8 mg, 0.023 mmol) and DIEA (2.0 mg, 3 μL, 0.016 mmol). The mixture was stirred at r.t. for 30 min. The mixture was filtered and the filtrate was purified using prep-HPLC (Mobile phase A: 0.1% TFA in water, B: MeCN; Flow rate: 20 mL/min; Column: Xbridge Prep C18 OBD™ 5 m, 19*150 mm) to provide compound 3-4 (7.5 mg, 23.8% yield) as white solid.

MS (ESI) m/z: 1355.1 [M+H]⁺

### Example 3-5

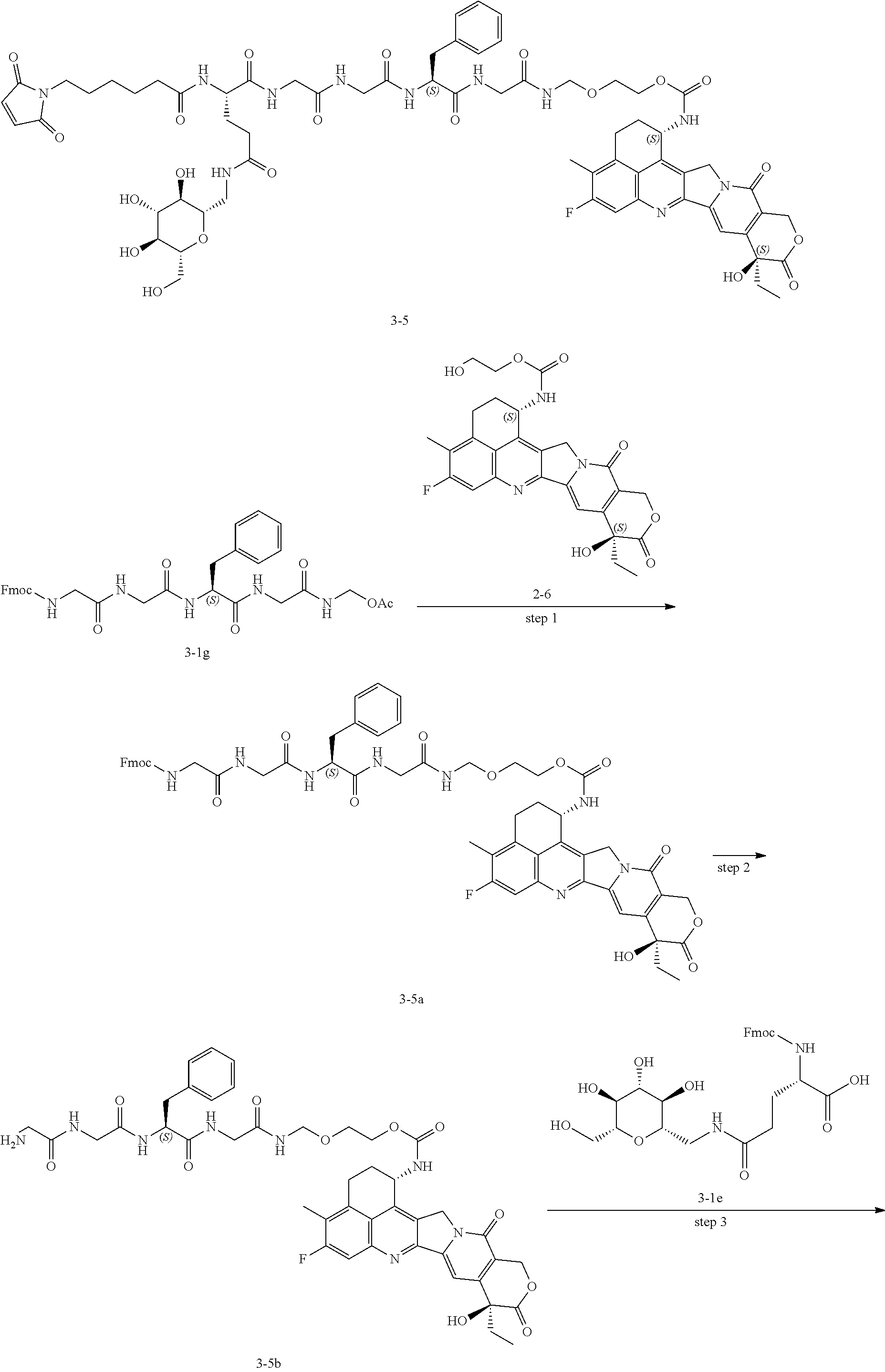

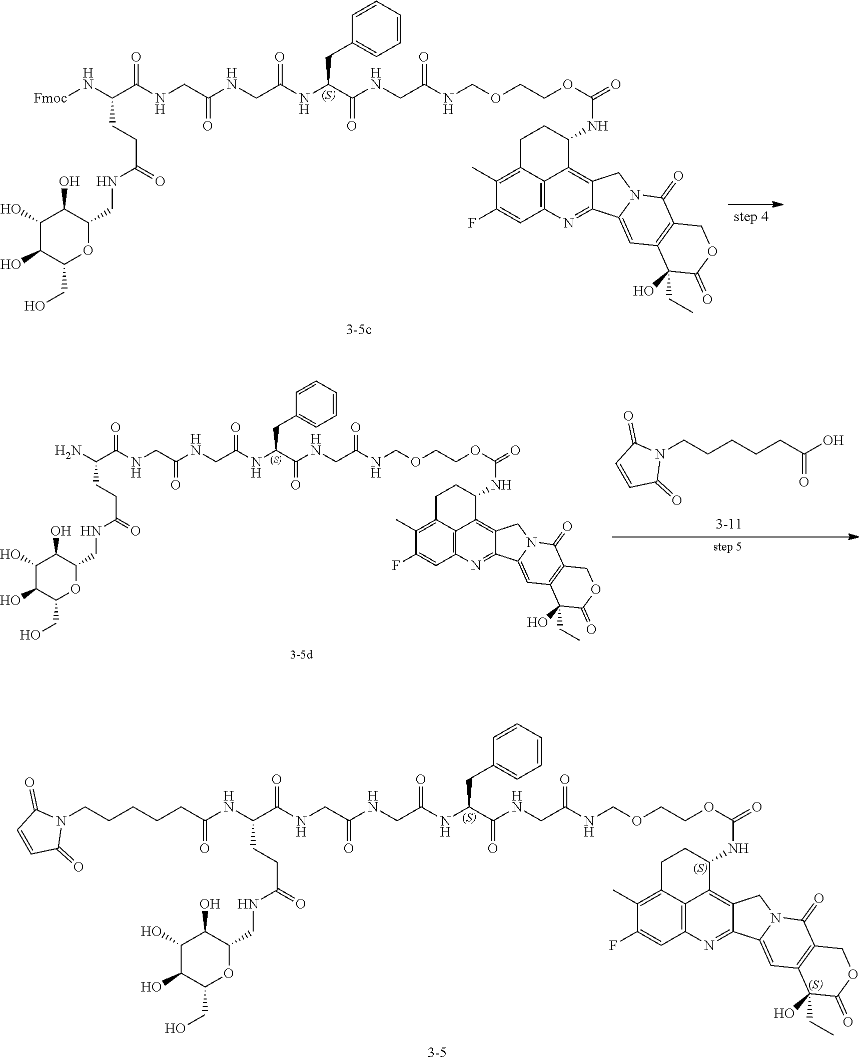

### Step 1

### (9H-fluoren-9-yl)methyl ((S)-12-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11,14,17-pentaoxo-2,5-dioxa-7,10,13,16-tetraazaoctadecan-18-yl)carbamate (3-5a)

To the solution of 3-1g (65 mg, 0.10 mmol) and 2-6 (58 mg, 0.092 mmol) in dry THF (2 mL) was added 4 Å MS (350 mg), stirred at r.t. for 30 min. Sc(OTf)₃ (56 mg, 0.11 mmol) was added under nitrogen atmosphere, stirred for 19 h. The slurry solution was filtered and the filter cake was washed with THF (10 mL). The filtrate was washed with sat. NaHCO₃ (10 mL), extracted with CH₂Cl₂/MeOH (5/1, 12 mL*3). The organic phase was concentrated and purified by flash column chromatography (silica gel, CH₂Cl₂/MeOH=10:1) to give compound 3-5a (81 mg, 87% purity, 70% yield) as a yellow solid.

MS (ESI) m/z: 1093.5 [M+H]⁺

### Step 2

### (S)-16-amino-10-benzyl-6,9,12,15-tetraoxo-3-oxa-5,8,11,14-tetraazahexadecyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (3-5b)

To the solution of 3-5a (81 mg, 0.074 mmol) in DMF (1 mL) was added Et₂NH (154 EL, 1.48 mmol), stirred at r.t. for 10 min. The solution was concentrated to give crude product 3-5b (65 mg, theoretical yield) as a brown solid.

MS (ESI) m/z: 893.5 [M+Na]

### Step 3

### (5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatricosan-23-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (3-5c)

To the solution of 3-5b (65 mg, theoretical yield, 0.075 mmol), 3-1e (46 mg, 0.082 mmol) and HATU (35 mg, 0.09 mmol) in dry DMF (2 mL) was added DIEA (40 μL, 0.22 mmol), stirred at r.t. for 30 min. The solution was purified by prep-HPLC (Mobile phase A: 0.1% FA in water, B: MeCN; Gradient: 35%-80% B; Flow rate: 20 mL/min; Column: Xbridge Prep C18 OBD™ 5 μm, 19*150 mm). The desired fraction was lyophilized to give 3-5c (19 mg, 18.2% yield) as a yellow solid.

MS (ESI) m/z: 1419.9 [M+Na]⁺

### Step 4

### (6S,15S)-6-amino-15-benzyl-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-24-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (3-5d)

To the solution of 3-5c (19 mg, 0.01 mmol) in DMF (1 mL) was added Et₂NH (28 μL, 0.27 mmol), stirred at r.t. for 10 min. The solution was concentrated to give crude product 3-5d (16 mg, theoretical yield) as a brown solid.

MS (ESI) m/z: 1175.8 [M+H]⁺

### Step 5

### (10S,19S)-10-benzyl-26-(2,5-dioxo-2,5-dihydro-1H-pyrrol-1-yl)-6,9,12,15,18,21-hexaoxo-19-(3-oxo-3-((((2S,3R,4R,5S,6R)-3,4,5-trihydroxy-6-(hydroxymethyl)tetrahydro-2H-pyran-2-yl)methyl)amino)propyl)-3-oxa-5,8,11,14,17,20-hexaazahexacosyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (3-5)

To the solution of 3-5d (16 mg, theoretical yield, 0.014 mmol), 3-11 (3.8 mg, 0.018 mmol) and HATU (5.8 mg, 0.015 mmol) in dry DMF (2 mL) was added DIEA (5 μL, 0.027 mmol), stirred at r.t. for 10 min. The solution was purified by prep-HPLC (Mobile phase A: 0.1% FA in water, B: MeCN; Gradient: 30%-45% B; Flow rate: 20 mL/min; Column: Xbridge Prep C18 OBD™ 5 m, 19*150 mm). The desired fraction was lyophilized to give 3-5 (7.9 mg, 42.4% yield) as a white solid.

MS (ESI) m/z: 1390.9 [M+Na]⁺

### Example 3-6

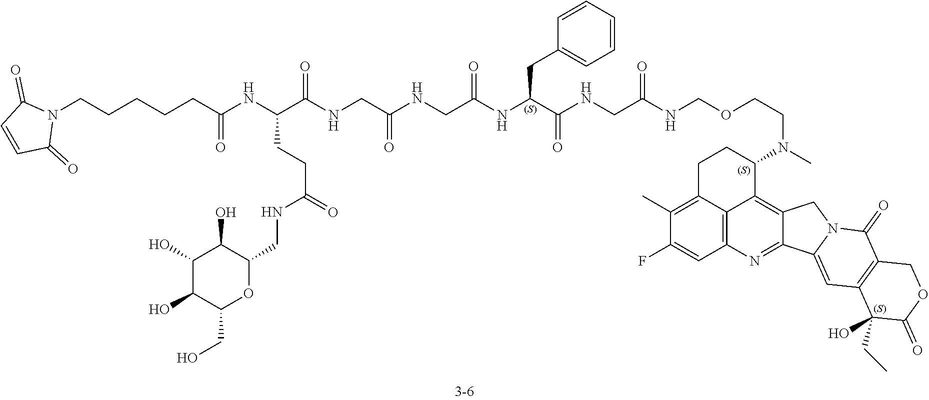

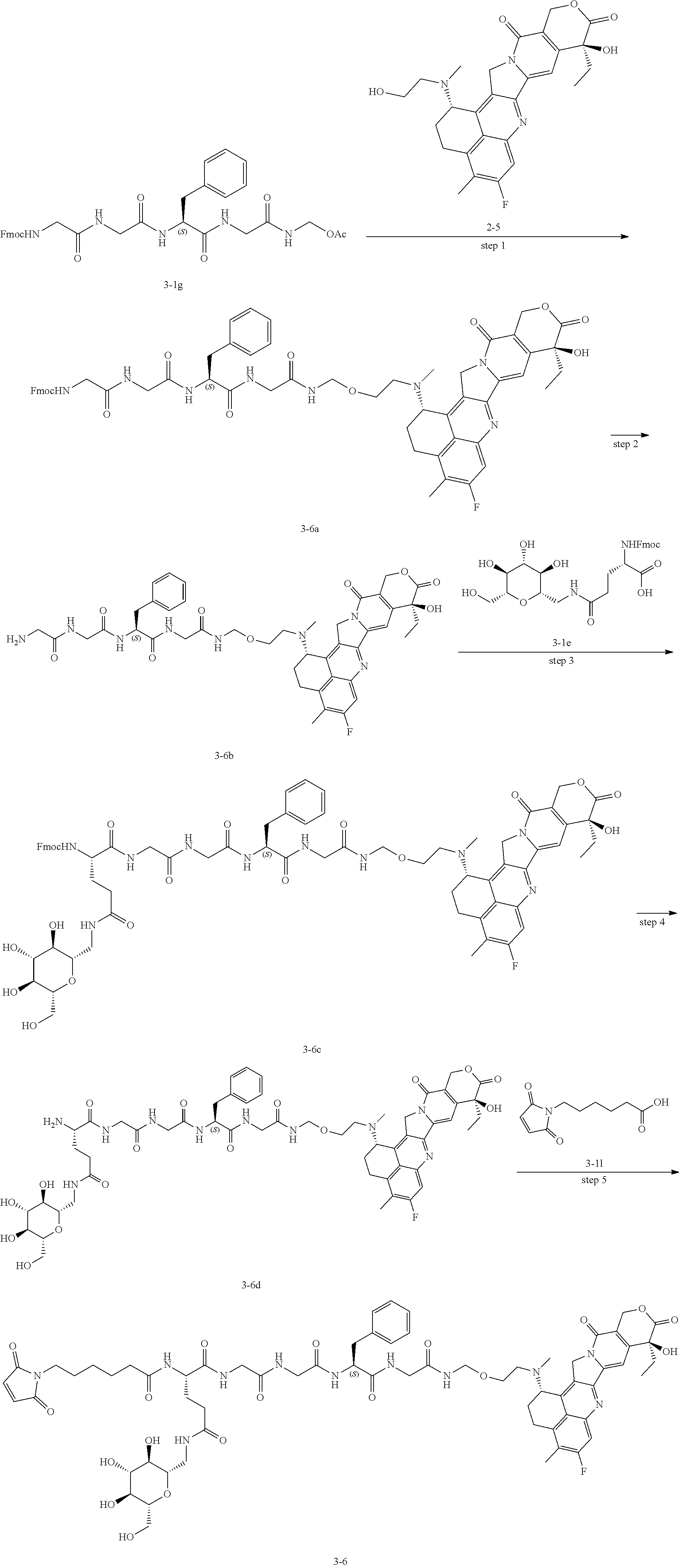

### Step 1

### (9H-fluoren-9-yl)methyl ((S)-12-benzyl-2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-8,11,14,17-tetraoxo-5-oxa-2,7,10,13,16-pentaazaoctadecan-18-yl)carbamate (3-6a)

Compound 3-6a (180 mg, 53% purity) was obtained according to the procedure described in step 1 of example 3-5.

MS (ESI) m/z: 1063.6 [M+H]⁺

### Step 2

### (S)-2-(2-(2-aminoacetamido)acetamido)-N-(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)(methyl)amino)ethoxy)methyl)amino)-2-oxoethyl)-3-phenylpropanamide (3-6b)

Compound 3-6b (185 mg, crude) was obtained according to the procedure described in step 2 of example 3-5.

MS (ESI) m/z: 841.5 [M+H]⁺

### Step 3

### (9H-fluoren-9-yl)methyl ((12S,21S)-12-benzyl-2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-8,11,14,17,20,24-hexaoxo-26-((2S,3R,4R,5S,6R)-3,4,5-trihydroxy-6-(hydroxymethyl)tetrahydro-2H-pyran-2-yl)-5-oxa-2,7,10,13,16,19,25-heptaazahexacosan-21-yl)carbamate (3-6c)

Compound 3-6c (80 mg, 70% purity) was obtained according to the procedure described in step 3 of example 3-5.

MS (ESI) m/z: 1367.7 [M+H]⁺

### Step 4

### (S)-2-amino-N¹—((S)-12-benzyl-2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-8,11,14,17-tetraoxo-5-oxa-2,7,10,13,16-pentaazaoctadecan-18-yl)-N5-(((2S,3R,4R,5S,6R)-3,4,5-trihydroxy-6-(hydroxymethyl)tetrahydro-2H-pyran-2-yl)methyl)pentanediamide (3-6d)

Compound 3-6d (41 mg, crude) was obtained according to the procedure described in step 4 of example 3-5.

MS (ESI) m/z: 1145.7 [M+H]⁺

### Step 5

### (S)—N¹—((S)-12-benzyl-2-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-8,11,14,17-tetraoxo-5-oxa-2,7,10,13,16-pentaazaoctadecan-18-yl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (3-6)

Compound 3-6 (13 mg, 98% purity) was obtained according to the procedure described in step 5 of example 3-5.

MS (ESI) m/z: 1338.8 [M+H]⁺

### Example 3-7

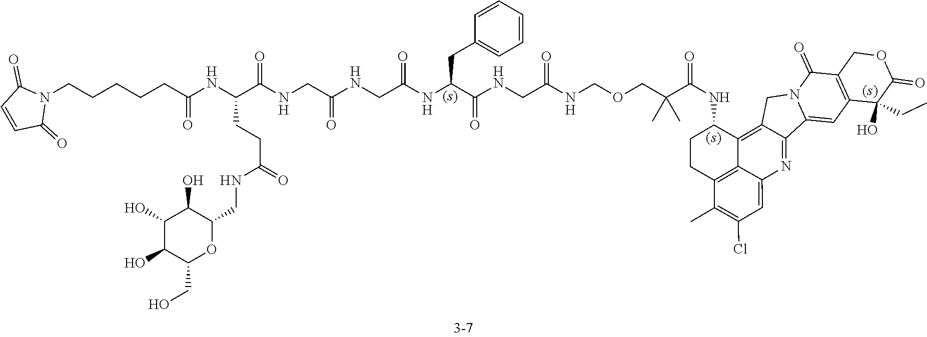 [SMILES: CC[C@@]1(O)C(=O)OCc2c1cc1n(c2=O)Cc2c-1nc1cc(Cl)c(C)c3c1c2[C@@H](NC(=O)C(C)(C)COCNC(=O)CNC(=O)[C@H](Cc1ccccc1)NC(=O)CNC(=O)CNC(=O)[C@H](CCC(=O)NC[C@@H]1O[C@H](CO)[C@@H](O)[C@H](O)[C@H]1O)NC(=O)CCCCCN1C(=O)C=CC1=O)CC3]

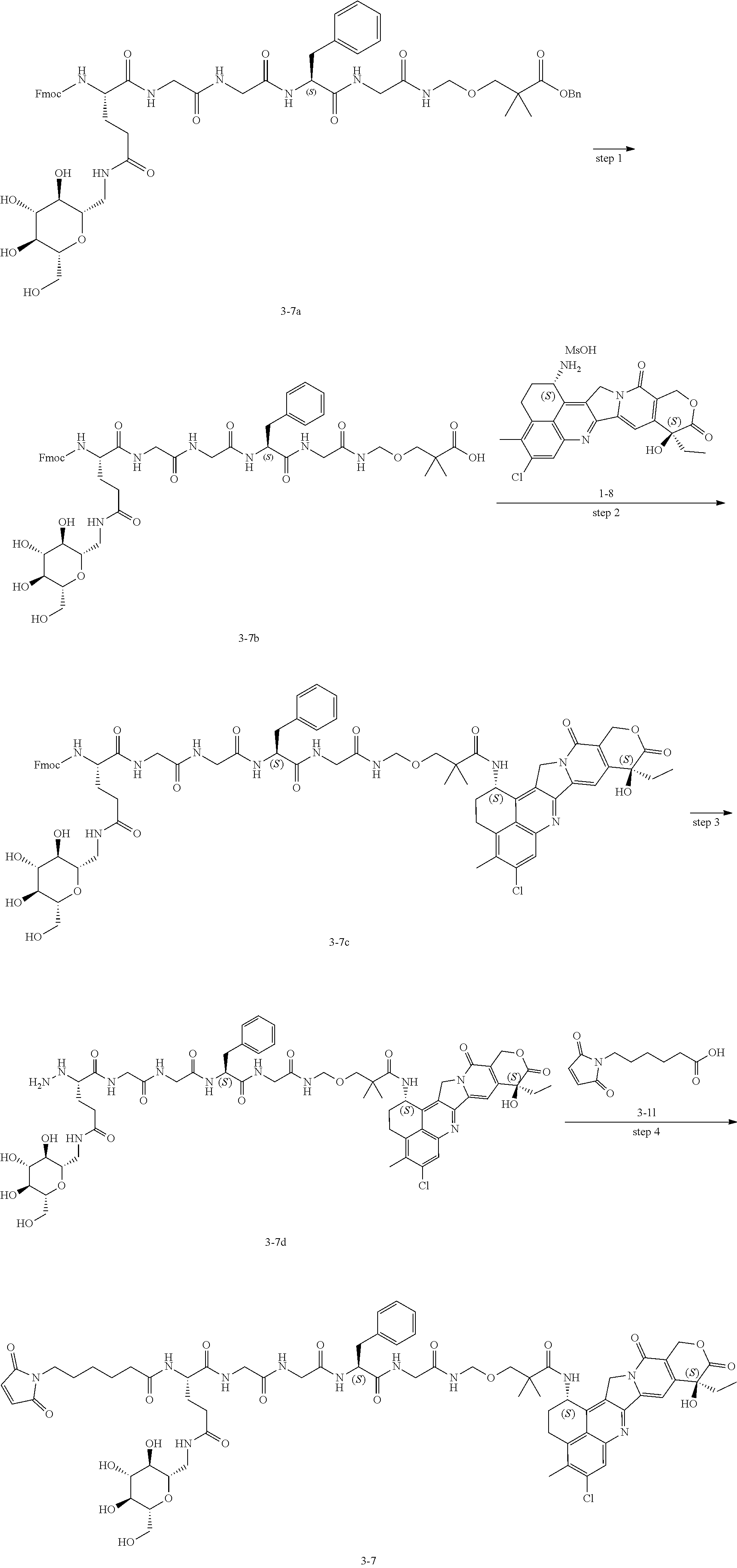

### Step 1

### (5S,14S)-14-benzyl-1-(9H-fluoren-9-yl)-23,23-dimethyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (3-7b)

To the solution of 3-7a (39 mg, 0.036 mmol) in MeOH (2 mL) was added 10% Pd/C (8.5 mg) under nitrogen atmosphere, stirred at H₂ atmosphere with a H₂ balloon for 4 h. The solution was filtered and concentrated to give compound 3-7b (36 mg, 83% purity) which was used for the next step without further purification.

MS (ESI) m/z: 1014.6 [M+H]⁺

### Step 2

### (9H-fluoren-9-yl)methyl ((6S,15S)-15-benzyl-25-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-7c)

Compound 3-7c (24 mg, 98% purity) was obtained according to the procedure described in step 1 of example 3-4.

MS (ESI) m/z: 1447.6 [M+Na]⁺

### Step 3

### (S)-2-amino-N1-((S)-7-benzyl-17-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-7d)

Compound 3-7d (25 mg, crude) was obtained according to the procedure described in step 2 of example 3-4.

MS (ESI) m/z: 1203.6 [M+H]⁺

### Step 4

### (S)—N1-((S)-7-benzyl-17-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-7)

Compound 3-7 (9.7 mg, 96% purity) was obtained according to the procedure described in step 3 of example 3-4.

MS (ESI) m/z: 1418.8 [M+Na]⁺

### Example 3-8

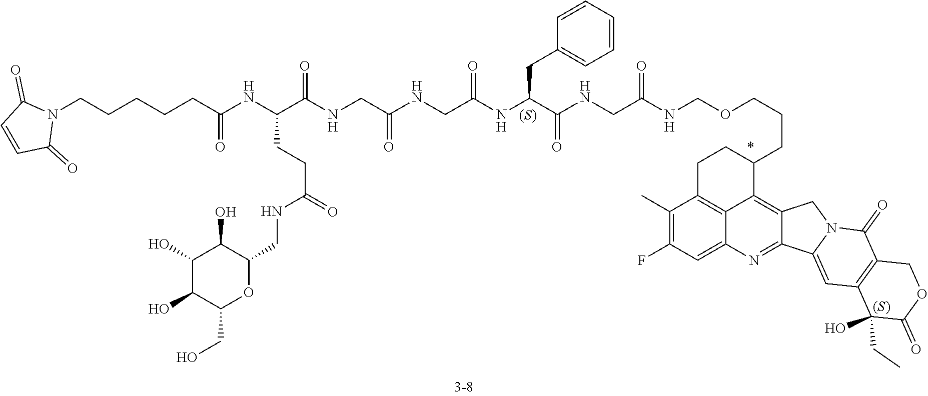

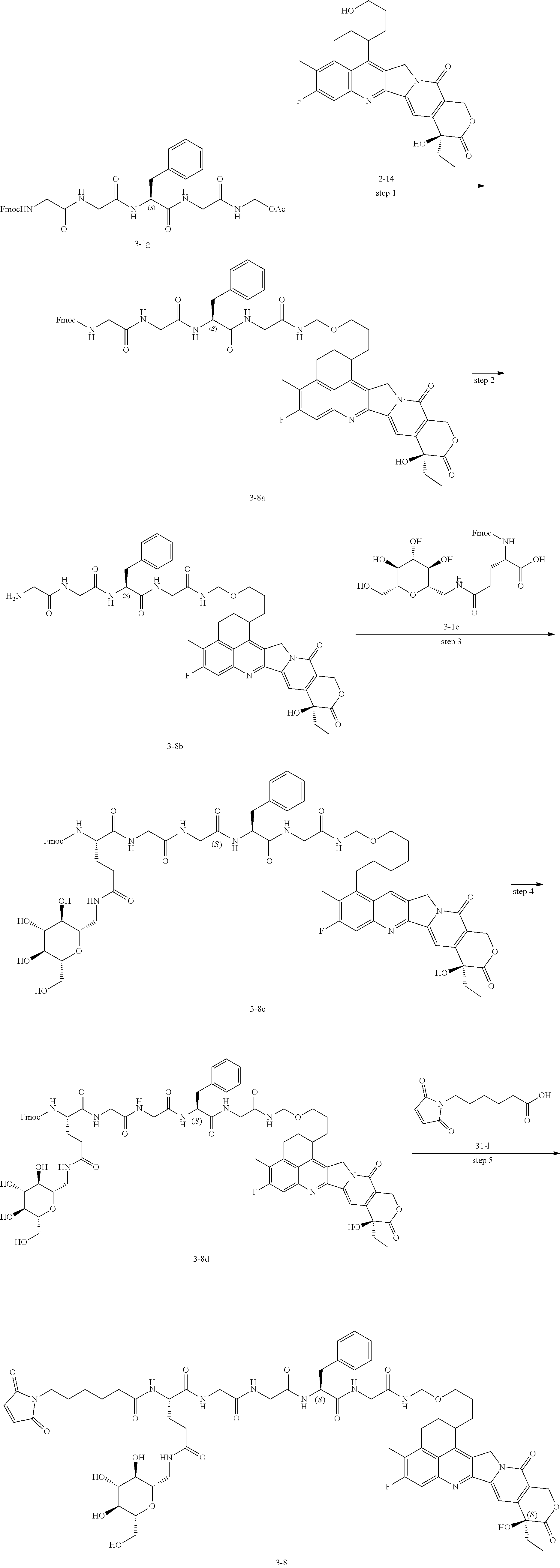

### Step 1

### (9H-fluoren-9-yl)methyl ((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)carbamate (3-8a)

Compound 3-8a (103 mg, 97% purity) was obtained according to the procedure described in step 1 of example 3-5.

MS (ESI) m/z: 1048.6 [M+H]⁺

### Step 2

### (2S)-2-(2-(2-aminoacetamido)acetamido)-N-(2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)-3-phenylpropanamide (3-8b)

Compound 3-8b (105 mg, crude) was obtained according to the procedure described in step 2 of example 3-5.

MS (ESI) m/z: 826.5 [M+H]⁺

### Step 3

### (9H-fluoren-9-yl)methyl ((6S,15S)-15-benzyl-25-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-8c)

Compound 3-8c (50 mg, 98% purity) was obtained according to the procedure described in step 3 of example 3-5.

MS (ESI) m/z: 1352.8 [M+H]⁺

### Step 4

### (2S)-2-amino-N′-((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)-N⁵-(((2S,3R,4R,5S,6R)-3,4,5-trihydroxy-6-(hydroxymethyl)tetrahydro-2H-pyran-2-yl)methyl)pentanediamide (3-8d)

Compound 3-8d (52 mg, crude) was obtained according to the procedure described in step 4 of example 3-5.

MS (ESI) m/z: 1130.7 [M+H]⁺

### Step 5

### (2S)—N′-((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (3-8)

Compound 3-8 (33 mg, 98% purity) was obtained according to the procedure described in step 5 of example 3-5.

MS (ESI) m/z: 1323.8 [M+H]⁺

### Example 3-9

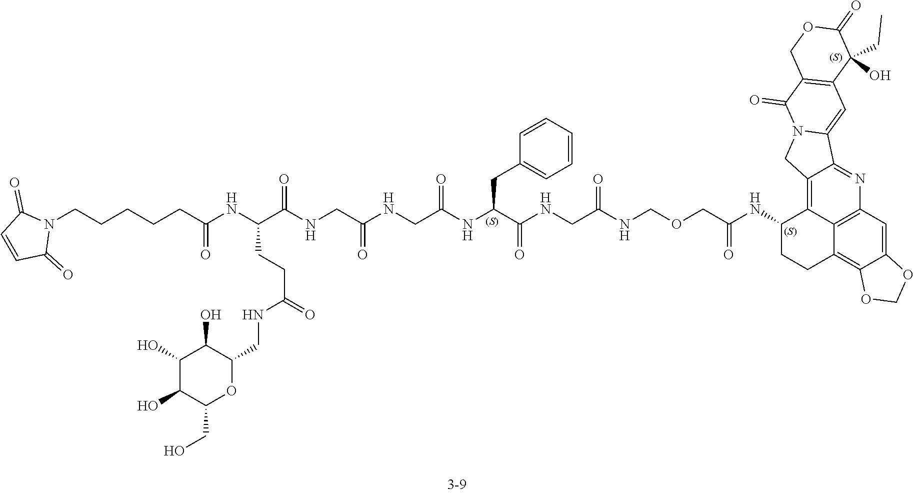

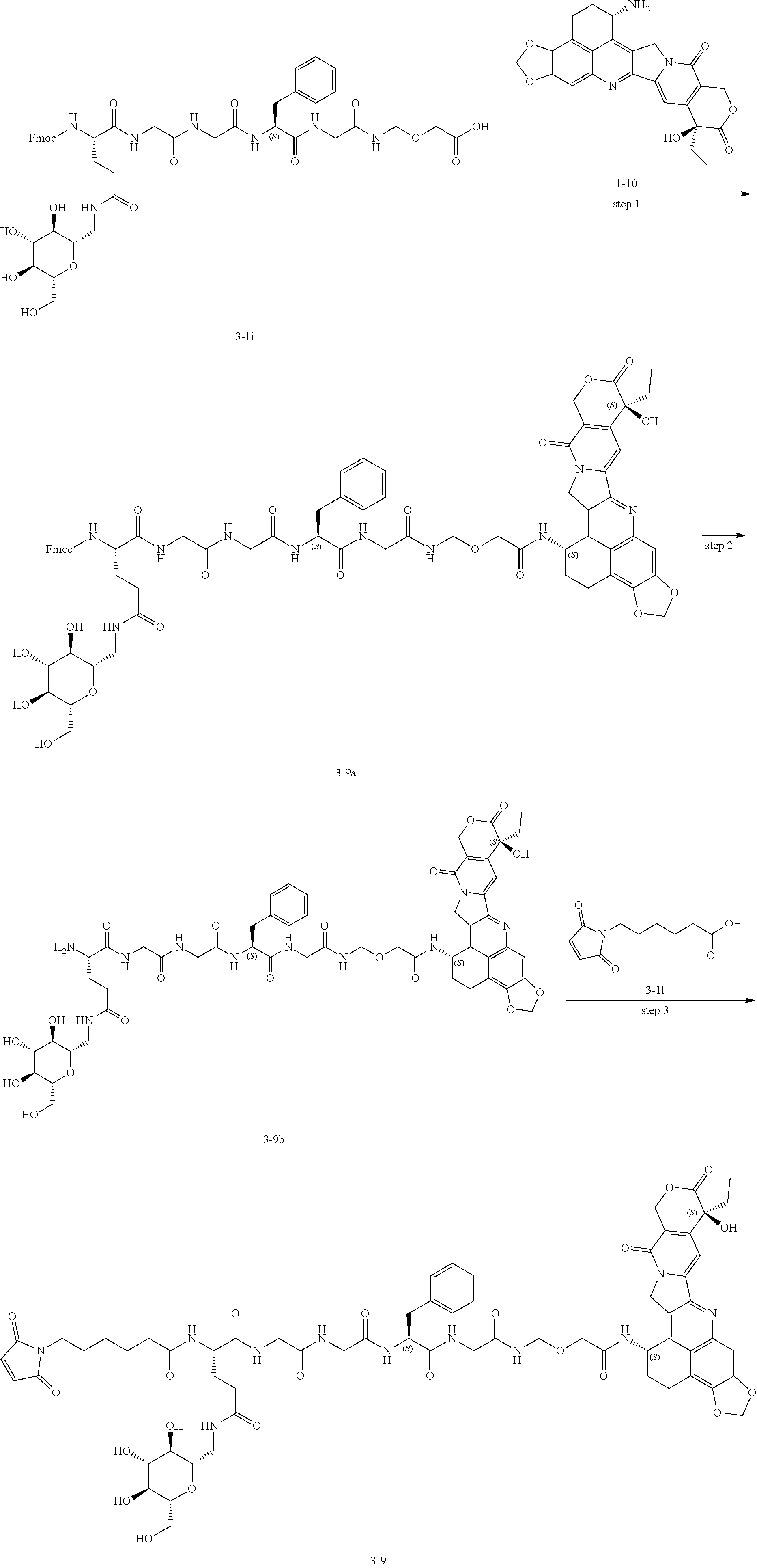

### Step 1

### (9H-fluoren-9-yl)methyl((6S,15S)-15-benzyl-24-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-9a)

To a solution of compound 3-1i (20 mg, 0.02 mmol) in DMF (2 mL) were added 1-10 (11.3 mg, 0.03 mmol), HATU (9.12 mg, 0.03 mmol) and DIEA (5.16 mg, 0.04 mmol). The mixture was stirred at 20° C. for 30 min. LCMS showed the reaction was completed. The mixture was filtered, the filtrate was purified using prep-HPLC (Method: column: XBridge Prep C18 OBD 5 um 19*150 mm; Mobile phase: A—water (0.1% trifluoroacetic acid): B-acetonitrile; Flow rate: 20 mL/min to provide compound 3-9a (12.2 mg, 42.1% yield) as white solid.

MS (ESI) m/z: 1401.7 [M+Na]⁺

### Step 2

### (S)-2-amino-N1-((S)-10-benzyl-1-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-9b)

To a solution of compound 3-9a (12.2 mg, 0.009 mmol) in DMF (2 mL) was added Et₂NH (9.7 mg, 0.13 mmol). The mixture was stirred at 20° C. for 30 min. The mixture was concentrated under high vacuum and co-evaporated with toluene (3 mL*3) to give 3-9b (10.2 mg, crude) as brown solid.

MS (ESI) m/z: 1179.6 [M+Na]⁺

### Step 3

### (S)—N1-((S)-10-benzyl-1-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-9)

To a solution of compound 3-9 (10.2 mg, crude) in DMF (1 mL) were added compound 3-11 (3.72 mg, 0.018 mmol), HATU (6.5 mg, 0.021 mmol) and DIEA (6.4 mg, 0.036 mmol). The mixture was stirred at 20° C. for 30 min. The mixture was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to compound 3-9 (4.4 mg, 36.9% yield).

MS (ESI) m/z: 1372.7 [M+Na]⁺

### Example 3-10

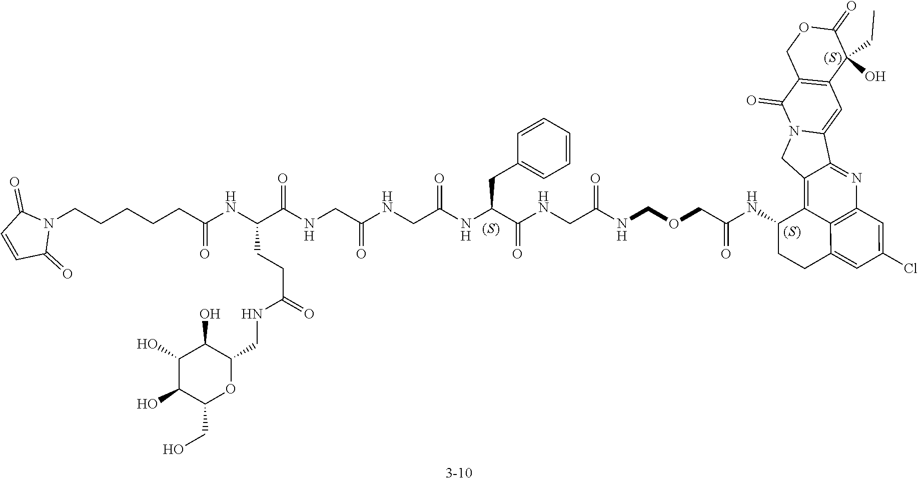

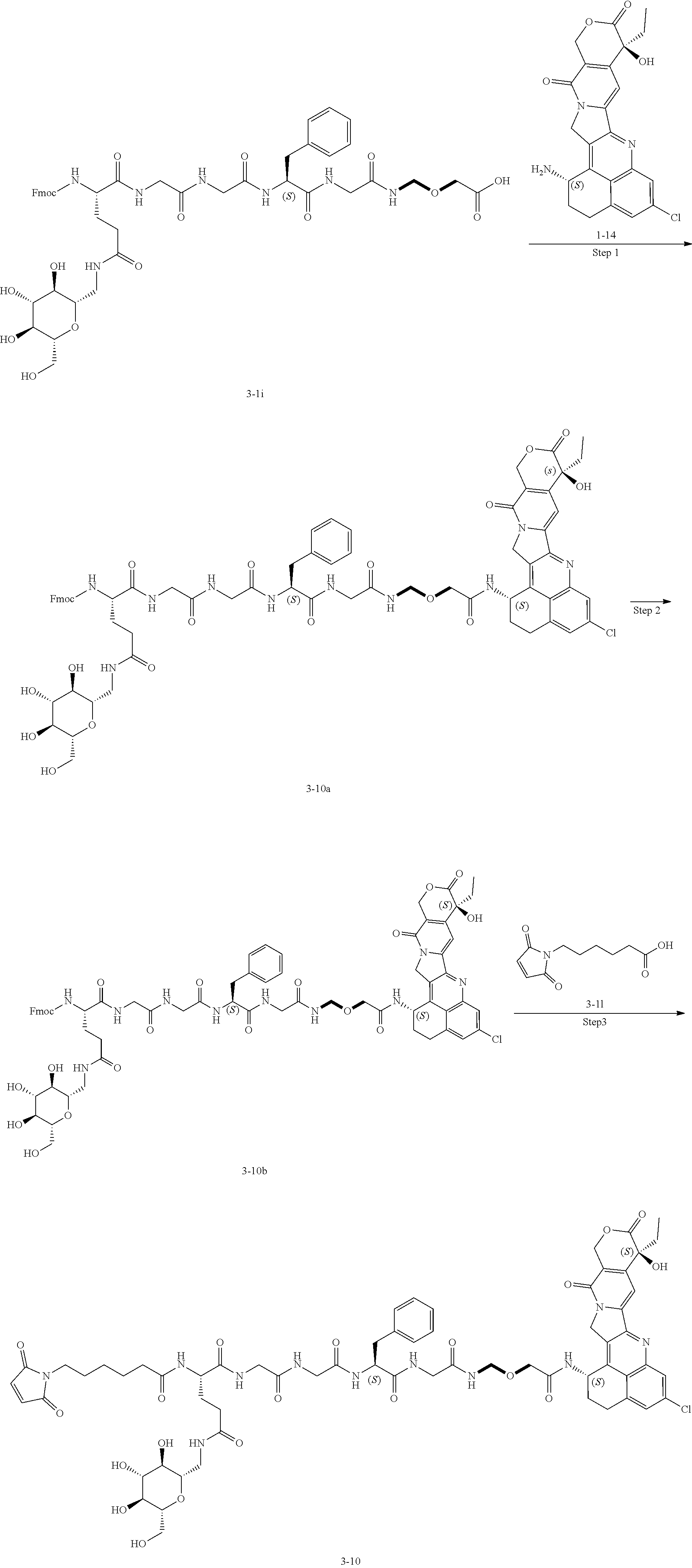

### Step 1

### ((9H-fluoren-9-yl)methyl ((6S,15S)-15-benzyl-24-(((1S,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-10a)

To a solution of 3-1i (44.89 mg, 0.10 mmol) in DMF (3 mL) and 1-14 (65.00 mg, 0.07 mmol) were added HBTU (38.92 mg, 0.10 mmol) and DIEA (26.55 mg, 0.21 mmol) was added into the mixture and reacted at the same temperature for another 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 3-10a (40 mg, 42.69% yield) was obtained as a white solid.

MS (ESI) m/z: 1391.7 [M+Na]⁺

### Step 2

### (S)-2-amino-N1-((S)-10-benzyl-1-(((1S,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-10b)

To a solution of compound 3-10a (40 mg, 0.03 mmol) in DMF (3 mL) was added Et₂NH (21.38 mg, 0.29 mmol). The mixture was stirred at r.t. for 1 h. The mixture was concentrated and co-evaporated with toluene (3*3 mL). The crude 3-10b (33.51 mg, 100% yield) was used for next step without further purification.

MS (ESI) m/z: 1147.6 [M+H]⁺

### Step 3

### (S)—N1-((S)-10-benzyl-1-(((1S,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-10)

To a solution of 3-10b (33.51 mg, 0.029 mmol) in DMF (3 mL) and 3-11 (12.34 mg, 0.06 mmol) were added HBTU (22.17 mg, 0.06 mmol) and DIEA (11.33 mg, 0.09 mmol) was added into the mixture and reacted at the same temperature for another 1 h. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 3-10 (19.4 mg, 49.55% yield) was obtained as a white solid.

MS (ESI) m/z: 1362.6 [M+Na]⁺

### Example 3-11

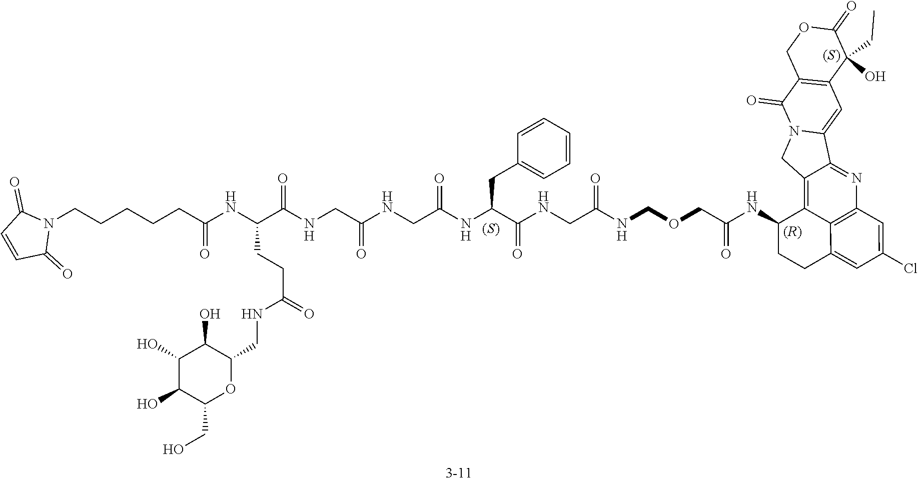

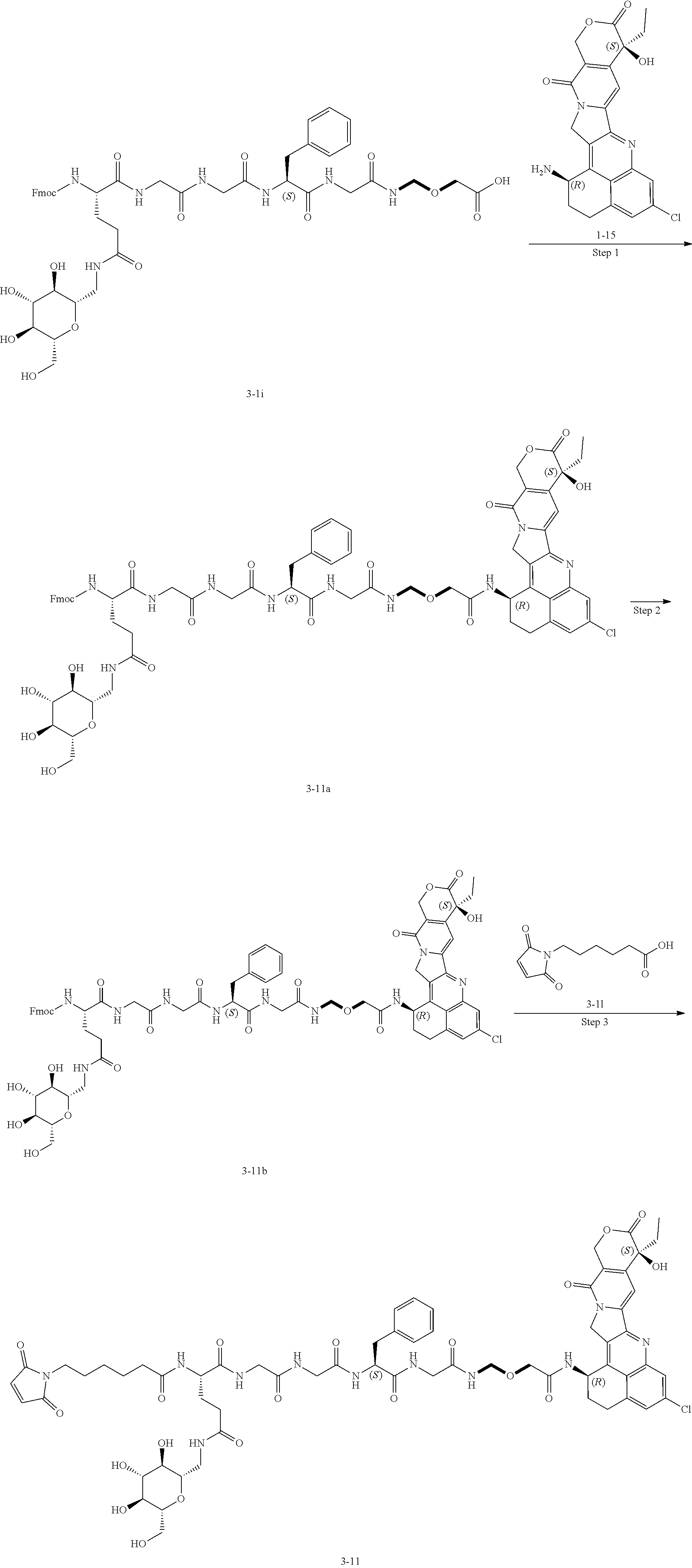

### Step 1

### (9H-fluoren-9-yl)methyl ((6S,15S)-15-benzyl-24-(((1R,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-11a)

Compound 3-11a (43.00 mg, 51.43% yield) was synthesized according to synthetic procedure of step 1 of example 3-10a.

MS (ESI) m/z: 1391.6 [M+Na]⁺

### Step 2

### (S)-2-amino-N1-((S)-10-benzyl-1-(((1R,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-11b)

Compound 3-11b (36.00 mg, crude) was synthesized according to synthetic procedure of step 2 of example 3-10b.

MS (ESI) m/z: 1147.6 [M+H]⁺

### Step 3

### (S)—N1-((S)-10-benzyl-1-(((1R,9S)-5-chloro-9-ethyl-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-11)

Compound 3-11 (17.50 mg, 41.58% yield) was synthesized according to synthetic procedure of step 3 of example 3-10.

MS (ESI) m/z: 1362.5 [M+Na]⁺

### Example 3-12

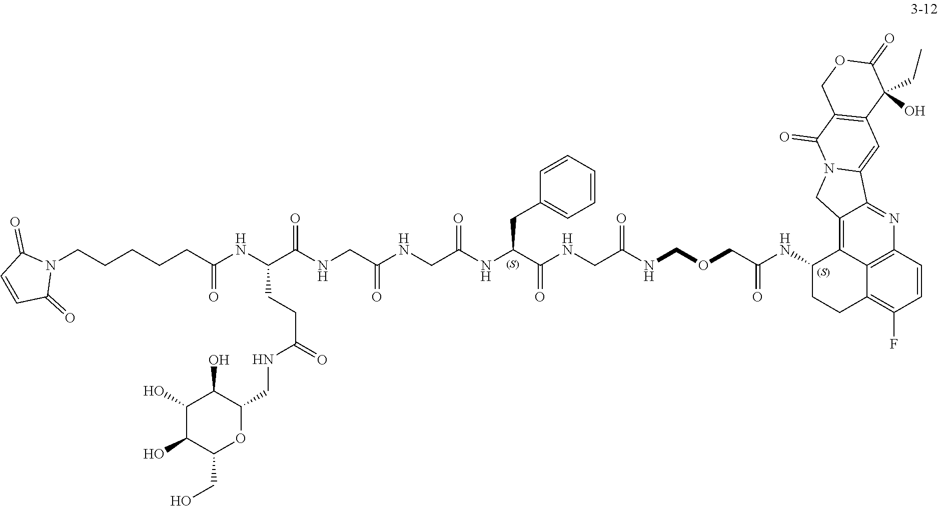

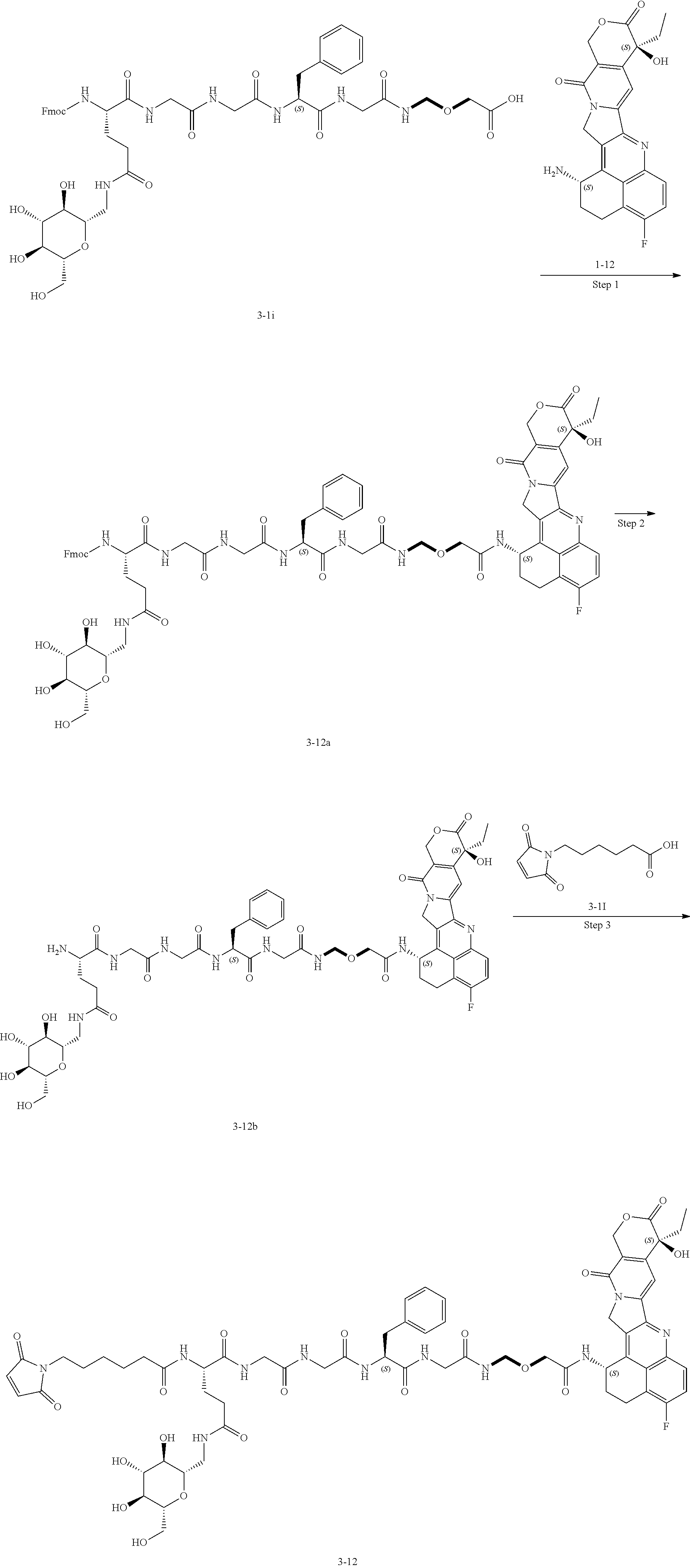

### Step 1

### (9H-fluoren-9-yl)methyl ((6S,15S)-15-benzyl-24-(((1S,9S)-9-ethyl-4-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-12a)

Compound 3-12a (20.00 mg, 56.15% yield) was synthesized according to synthetic procedure of step 1 of example 3-10a.

MS (ESI) m/z: 1375.7 [M+Na]⁺

### Step 2

### (S)-2-amino-N1-((S)-10-benzyl-1-(((1S,9S)-9-ethyl-4-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-12b)

Compound 3-12b (16.76 mg, crude) was synthesized according to synthetic procedure of step 2 of example 3-10b.

MS (ESI) m/z: 1131.6 [M+H]⁺

### Step 3

### (S)—N1-((S)-10-benzyl-1-(((1S,9S)-9-ethyl-4-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-12)

Compound 3-12 (4.50 mg, 23.29% yield) was synthesized according to synthetic procedure of step 3 of example 3-10.

MS (ESI) m/z: 1346.7 [M+Na]⁺

### Example 3-13

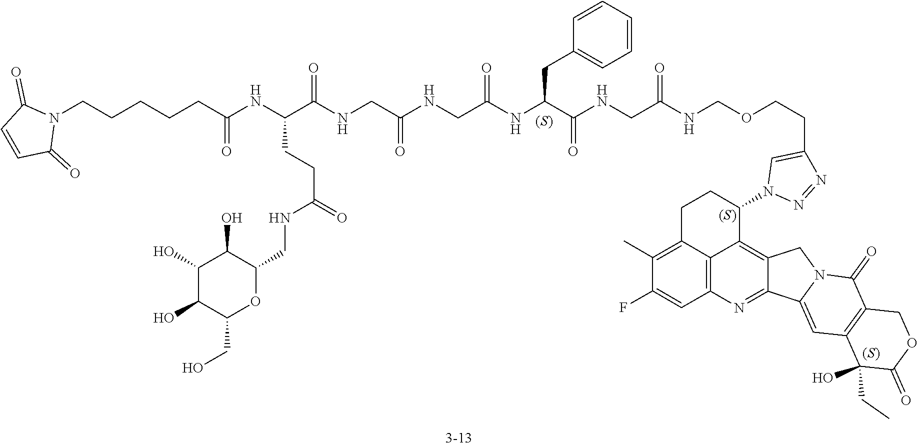

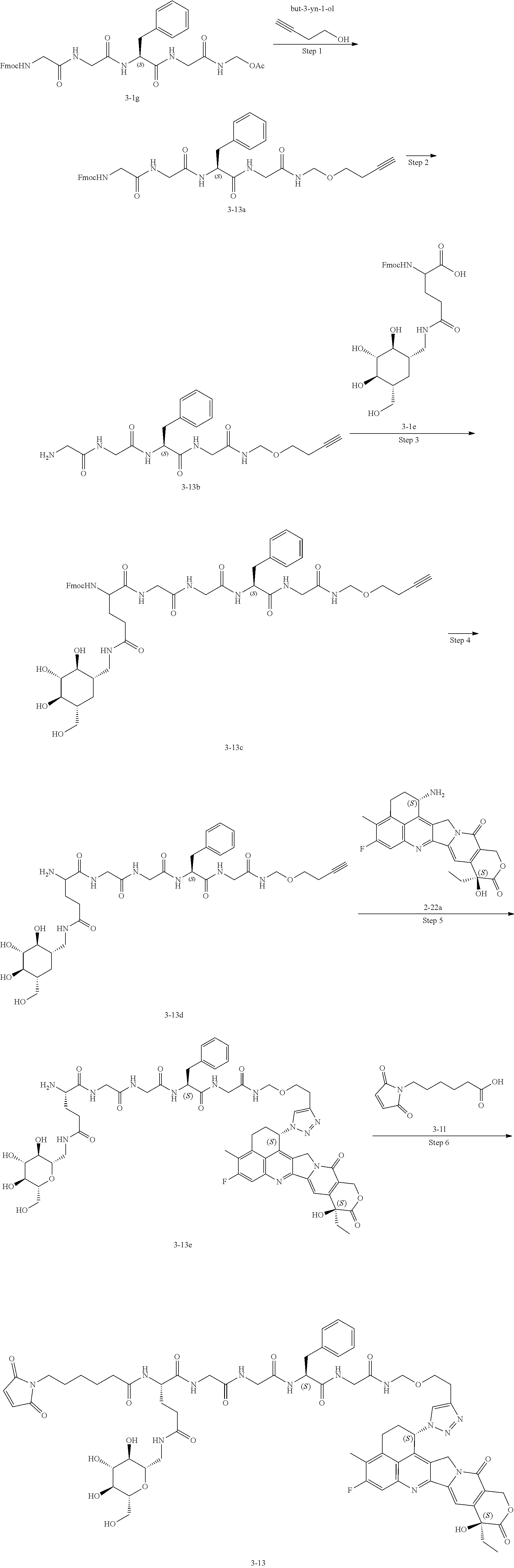

### Step 1

### (9H-fluoren-9-yl)methyl (S)-(7-benzyl-2,5,8,11-tetraoxo-14-oxa-3,6,9,12-tetraazaoctadec-17-yn-1-yl)carbamate (3-13a)

To a solution of 3-1g (126.4 mg, 0.2 mmol) in THF (5 mL) was added 3-Butyn-1-ol (42 mg, 0.6 mmol), Sc(OTf)₃ (196 mg, 0.4 mmol) and 4 Å Molecular sieves (200 mg). The mixture was stirred at r.t. under N₂ for 1.5 h. Upon completion of the reaction, the reaction was quenched with sat. NaHCO₃ (5 mL), the organic phase was extracted with EA (5 mL*3) washed with H₂O (5 mL) and brine (5 mL). The organic phase was collected and dried over Na₂SO₄, filtered and the filtrate was concentrated. The crude product was purified by silica column gel chromatography (eluent: CH₂Cl₂/MeOH=100/0 to 90/10) to afford 3-13a as a white solid (90 mg, yield 70.3%).

MS (ESI) m/z: 663.4 [M+Na]⁺

### Step 2

### (S)-2-(2-(2-aminoacetamido)acetamido)-N-(2-(((but-3-yn-1-yloxy)methyl)amino)-2-oxoethyl)-3-phenylpropanamide (3-13b)

To a solution of 3-13a (80 mg, 0.125 mmol) in DMF (1 mL) at 0° C. was added Et₂NH (129 μL, 92 mg, 1.25 mmol). The mixture was slowly warmed to r.t. and stirred at r.t. under N₂ for 0.5 h. Upon completion of the reaction, the mixture was concentrated under high vacuum to give 3-13b (15.3 mg, crude) as brown solid.

### Step 3

### (9H-fluoren-9-yl)methyl ((6S,15S)-15-benzyl-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazahexacos-25-yn-6-yl)carbamate (3-13c)

To a solution of compound 3-13b (15.3 mg, 0.037 mmol) in DMF (1 mL) were added compound 3-1e (20 mg, 0.037 mmol), HATU (21 mg, 0.055 mmol) and DIPEA (26 μL, 20 mg, 0.074 mmol). The mixture was stirred at r.t. for 30 min. The mixture was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to compound 3-13c (20 mg, 57.1% yield).

MS (ESI) m/z: 966.5 [M+Na]⁺

### Step 4

### (S)-2-amino-N1-((S)-7-benzyl-2,5,8,11-tetraoxo-14-oxa-3,6,9,12-tetraazaoctadec-17-yn-1-yl)-N5-(((2S,3R,4R,5S,6R)-3,4,5-trihydroxy-6-(hydroxymethyl)tetrahydro-2H-pyran-2-yl)methyl)pentanediamide (3-13d)

To a solution of 3-13c (20 mg, 0.02 mmol) in DMF (1 mL) at 0° C. was added Et₂NH (22 μL, 15.5 mg, 0.2 mmol). The mixture was slowly warmed to r.t. and stirred at r.t. under N₂ for 0.5 h. Upon completion of the reaction, the mixture was concentrated under high vacuum to give 3-13d (14.4 mg, crude) as brown solid. The crude product was used directly in the next step without further purification.

### Step 5

### (S)-2-amino-N1-((S)-10-benzyl-1-(1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1H-1,2,3-triazol-4-yl)-6,9,12,15-tetraoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-13e)

To a solution of 3-13d (14.4 mg, 0.02 mmol) in DMF (1 mL) was added 2-22a (10 mg, 0.022 mmol), CuI (1 mg, cat. amount) and DIPEA (17.5 μL, 12.9 mg, 0.1 mmol). The mixture was purged with N₂ balloon for 15 min, then stirred at r.t. under N₂ atmosphere for 3 h. On completion of the reaction, the mixture was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to compound 3-13e (19 mg, 79.1% yield) as a white solid.

MS (ESI) m/z: 1184.1 [M+H]⁺

### Step 6

### (S)—N1-((S)-10-benzyl-1-(1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-1H-1,2,3-triazol-4-yl)-6,9,12,15-tetraoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-13)

To a solution of compound 3-13e (19 mg, 0.016 mmol) in DMF (1.5 mL) were added compound 3-11 (4 mg, 0.019 mmol), HATU (12 mg, 0.032 mmol) and DIPEA (8.4 μL, 6.2 mg, 0.048 mmol). The mixture was stirred at r.t. for 30 min. The mixture was purified by prep-HPLC (FA) (Method: column: XBridge Prep C18 OBD 5 um 19*150 mm; Mobile phase: A-water (0.1% FA): B-acetonitrile; Flow rate: 20 mL/min, the fraction was lyophilized to compound 3-13 (4.5 mg, 20.5% yield).

MS (ESI) m/z: 1398.7 [M+Na]⁺. Retention time (3.96 min).

### Example 3-14

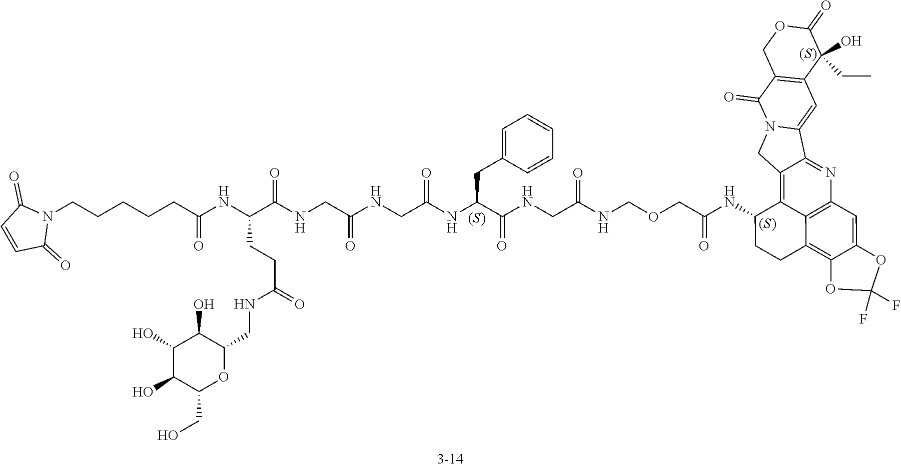

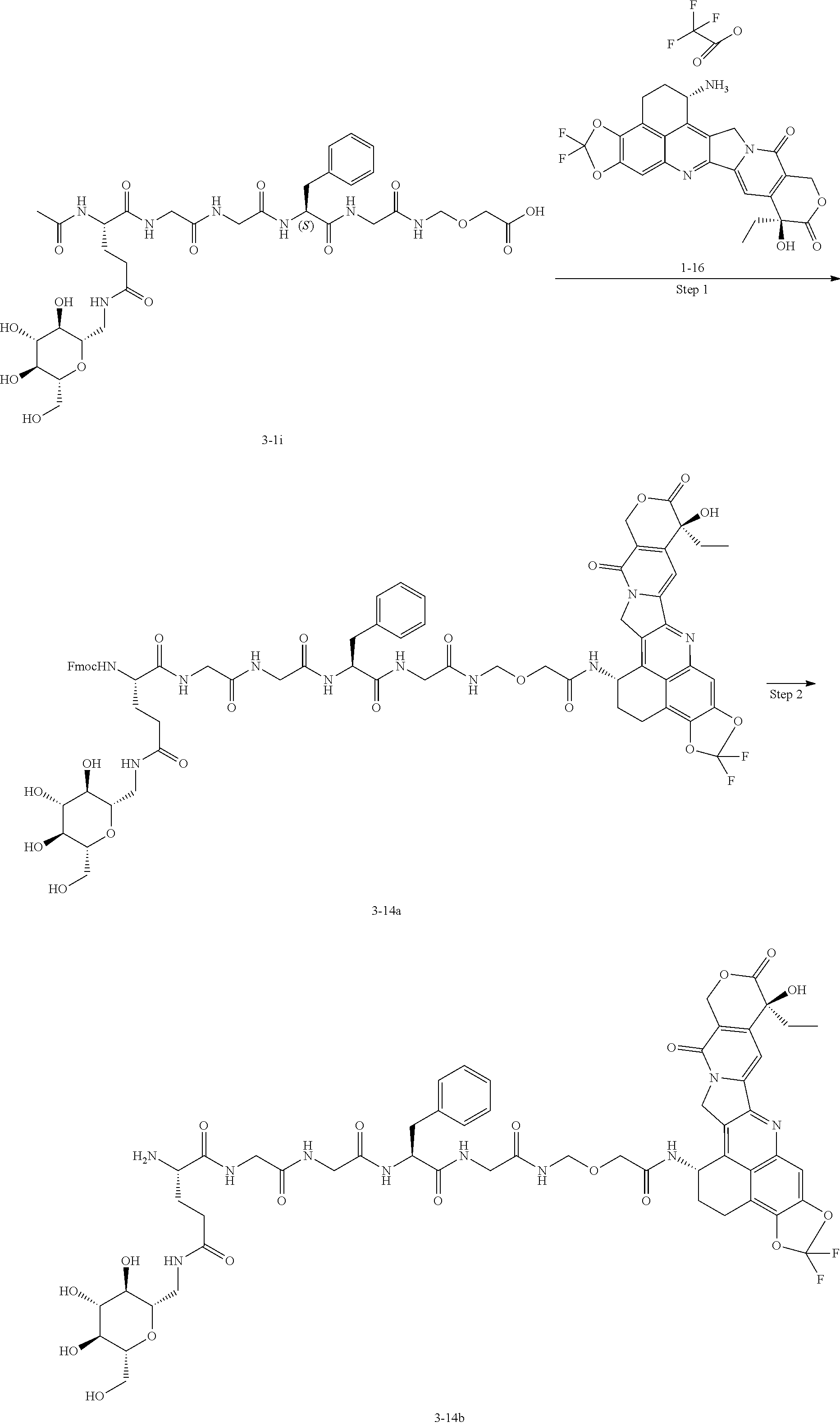

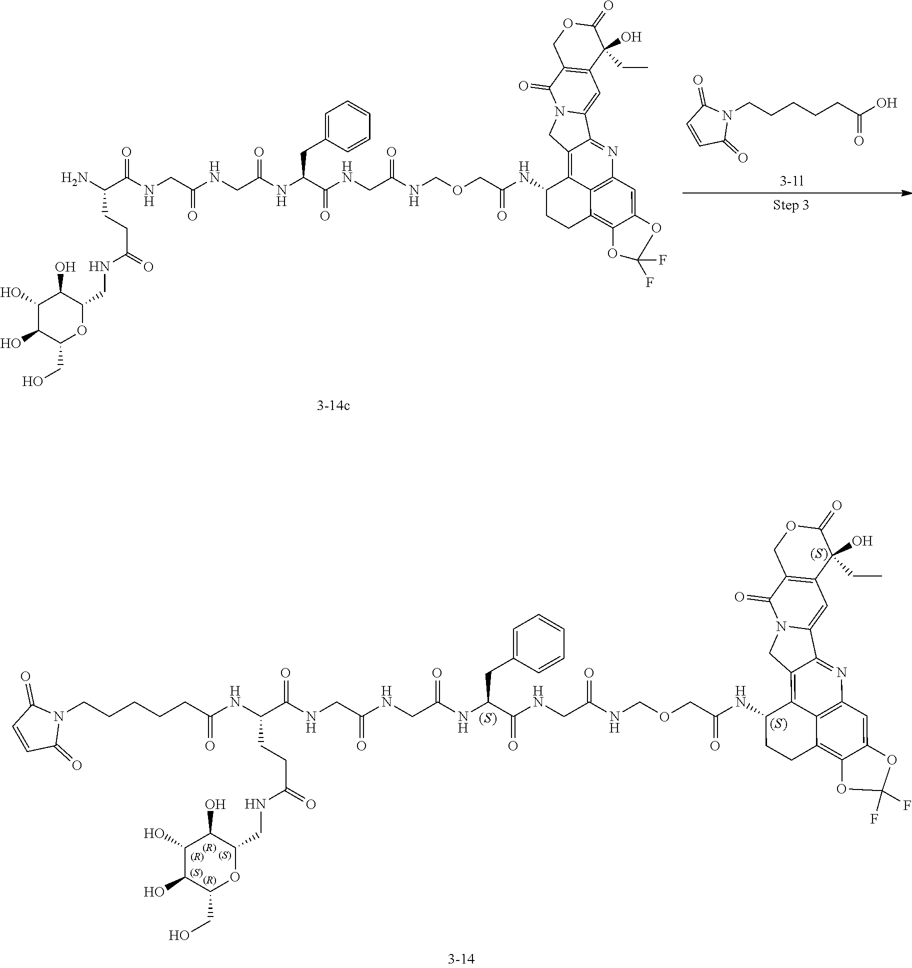

### Step 1

### (9H-fluoren-9-yl)methyl ((6S,15S)-15-benzyl-24-(((1S,10S)-10-ethyl-5,5-difluoro-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-14a)

To a mixture of 3-1i (20.776 mg, 0.035 mmol) and 1-16 (30 mg, 0.032 mmol) were added HATU (12.05 mg, 0.032 mmol) and DIPEA (16 μL, 12.26 mg, 0.095 mmol). The mixture was stirred at RT for 10 min. The mixture was filtered and the filtrate was purified by prep-HPLC (FA 0.1%), the fraction was concentrated under vacuum and co-evaporation with Tol one times to remove water to give 3-14a (40 mg, 81.3% yield) as a red solid.

MS (ESI) m/z: 1437.7 [M+Na]⁺;

### Step 2

### (S)-2-amino-N1-((S)-10-benzyl-1-(((1S,10S)-10-ethyl-5,5-difluoro-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-14b)

To a solution of 3-14a (40.0 mg, 0.028 mmol) in DMF (2 mL) was added Et₂NH (36.416 uL, 0.354 mmol). The mixture was stirred at RT for 30 min. The mixture was concentrated under vacuum and co-evaporated with Tol (10 mL) two times to remove DMF and Et₂N. To give 3-14b (40.0 mg, crude) as a yellow solid.

MS (ESI) m/z: 1193.7 [M+H]⁺;

### Step 3

### (S)—N1-((S)-10-benzyl-1-(((1S,10S)-10-ethyl-5,5-difluoro-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-14)

To a solution of 3-14b (7.087 mg, 0.034 mmol) and 3-11 (40.0 mg, crude) in DMF (1.5 mL) were added HATU (12.758 mg, 0.034 mmol) and DIPEA (6 uL, 4.770 mg, 0.037 mmol). The mixture was stirred at r.t. for 10 min. The mixture was purified by prep- HPLC (FA 0.1%), the fraction was lyophilized to give 3-14 (13.3 mg, 28.3% yield) as a white solid.

MS (ESI) m/z: 1408.9 [M+Na]⁺;

### Example 3-15

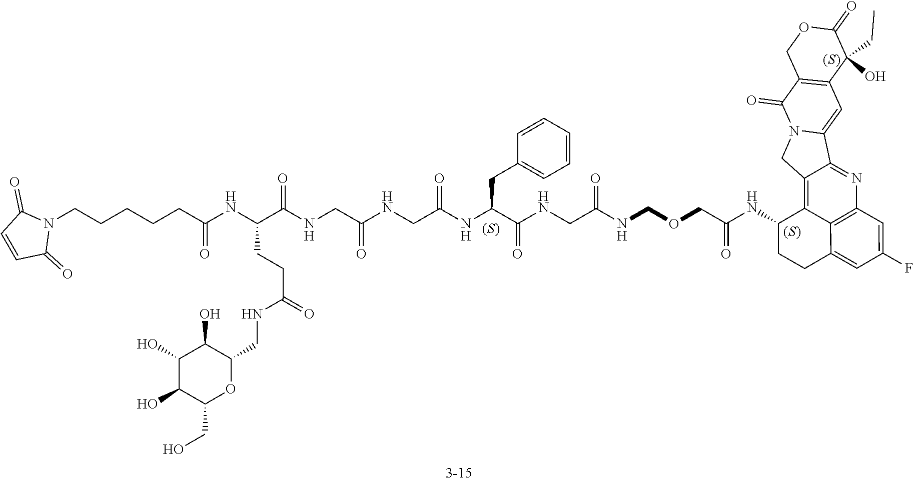

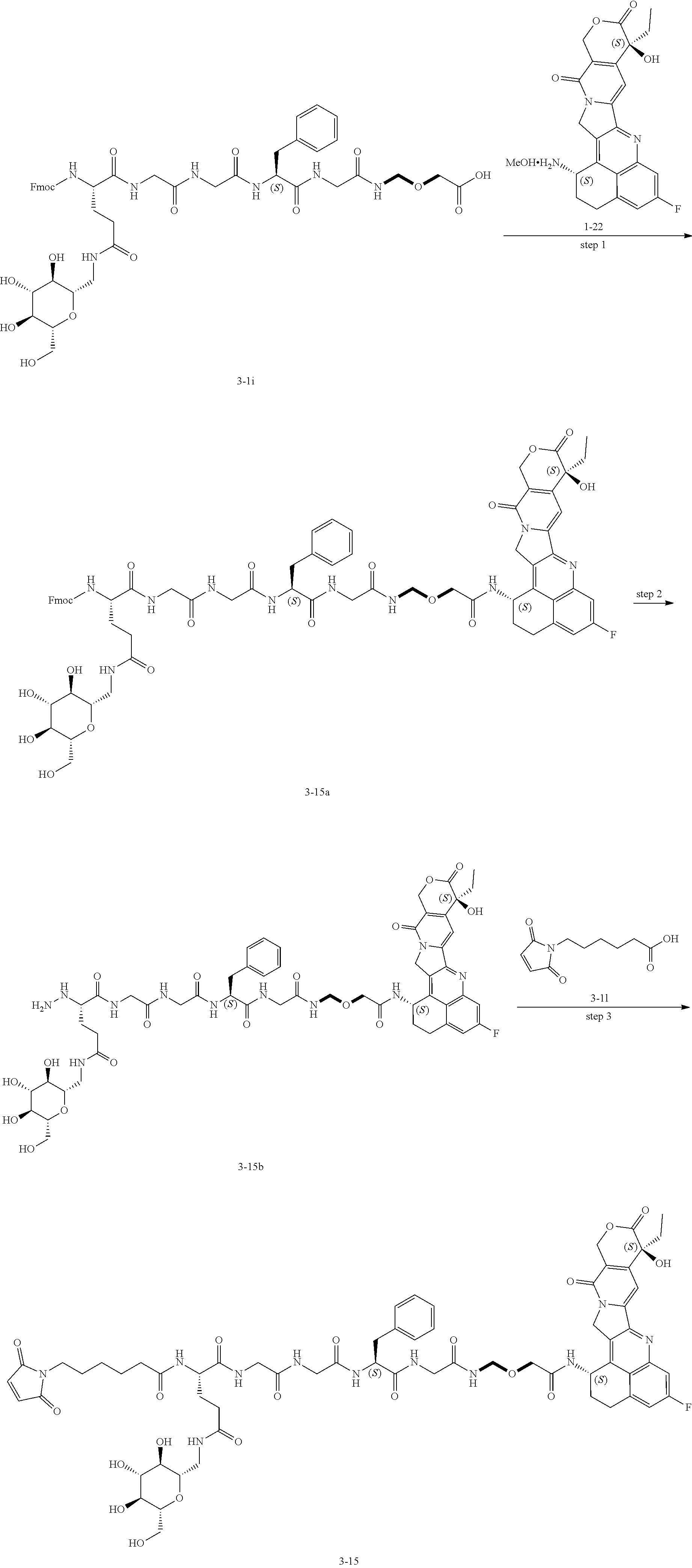

### Step 1˜Step 3

(S)—N1-((S)-10-benzyl-1-(((1S,9S)-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-15)

3-15 (5.1 mg, 38% yield) was synthesized according to synthetic procedure of example 3-1.

MS (ESI) m/z: 1324.9 [M+H]⁺

### Example 3-16

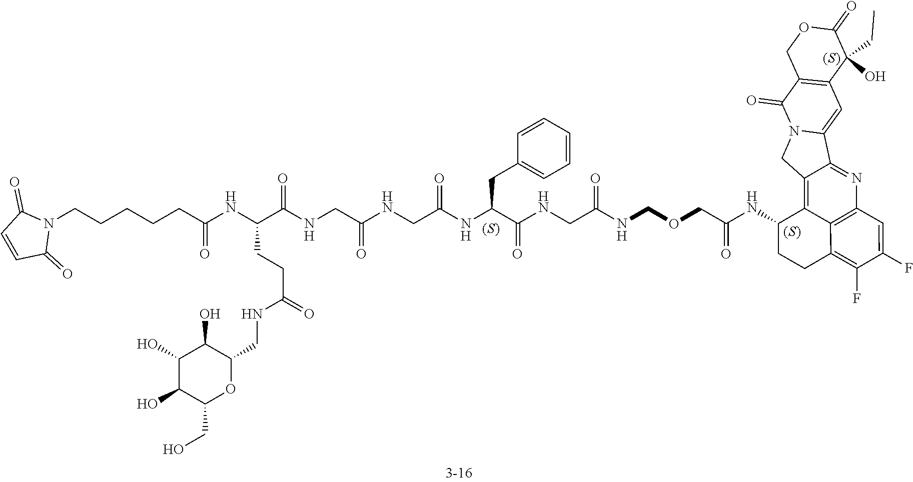

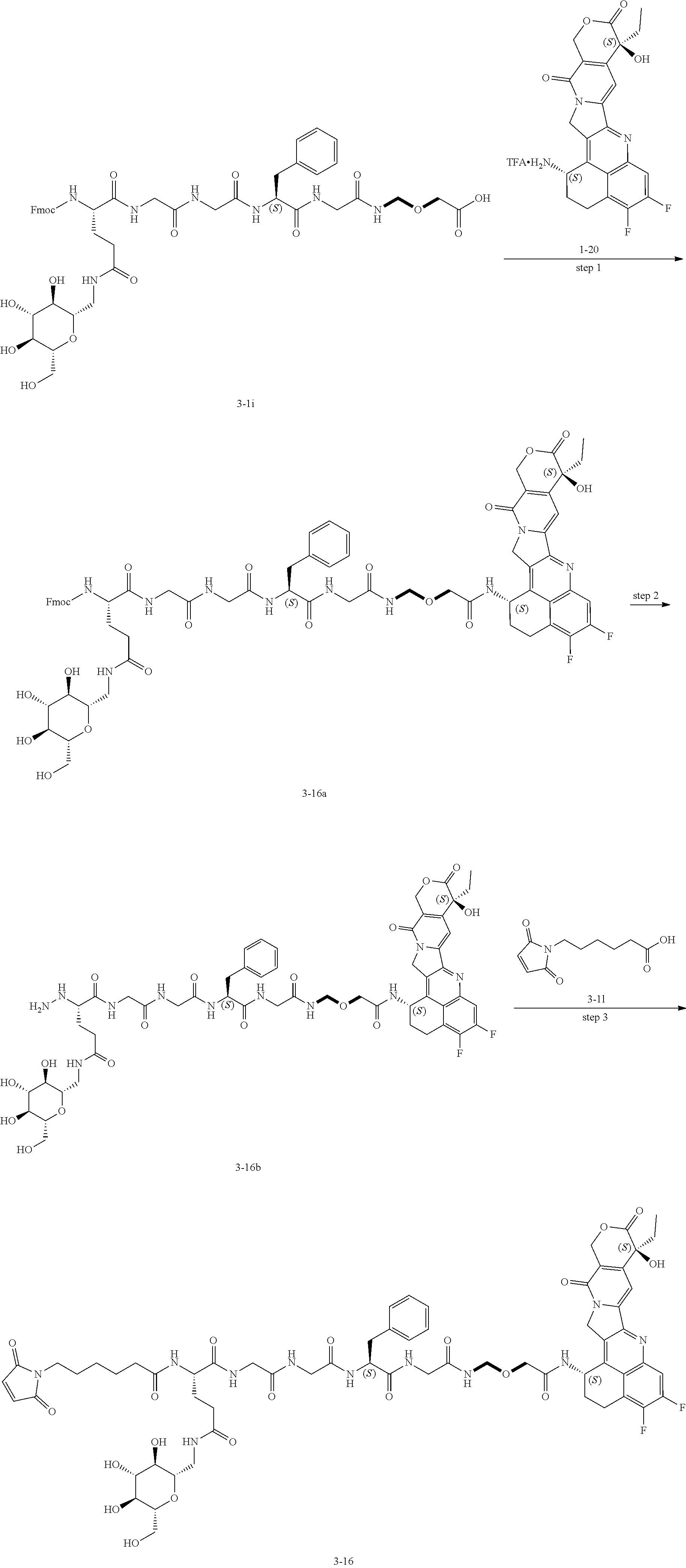

### Step 1˜Step 3

(S)—N1-((S)-10-benzyl-1-(((1S,9S)-9-ethyl-4,5-difluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2 S,3R,4R,5S,6R)-3,4,5-trihydroxy-6-(hydroxymethyl)tetrahydro-2H-pyran-2-yl)methyl)pentanediamide (3-16).

3-16 (13.1 mg, 53% yield) was synthesized according to synthetic procedure of example 3-1.

MS (ESI) m/z: 1364.7 [M+Na]⁺

### Example 3-17

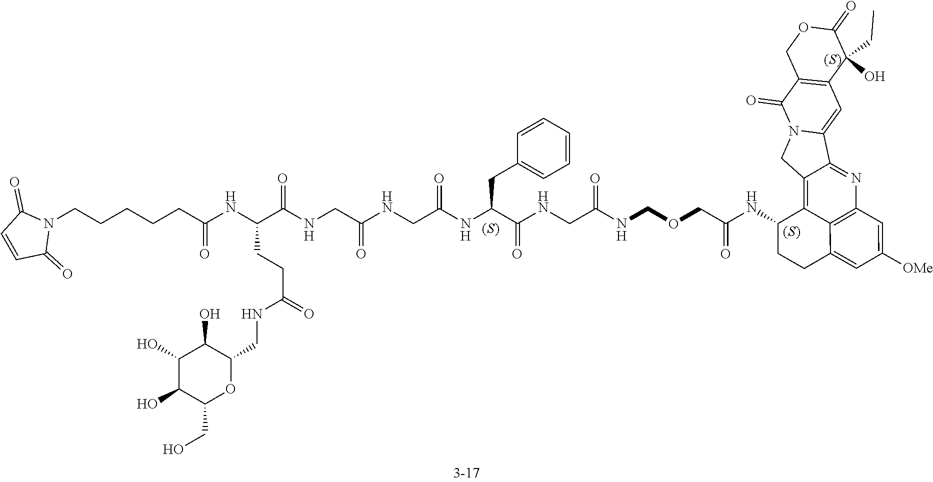

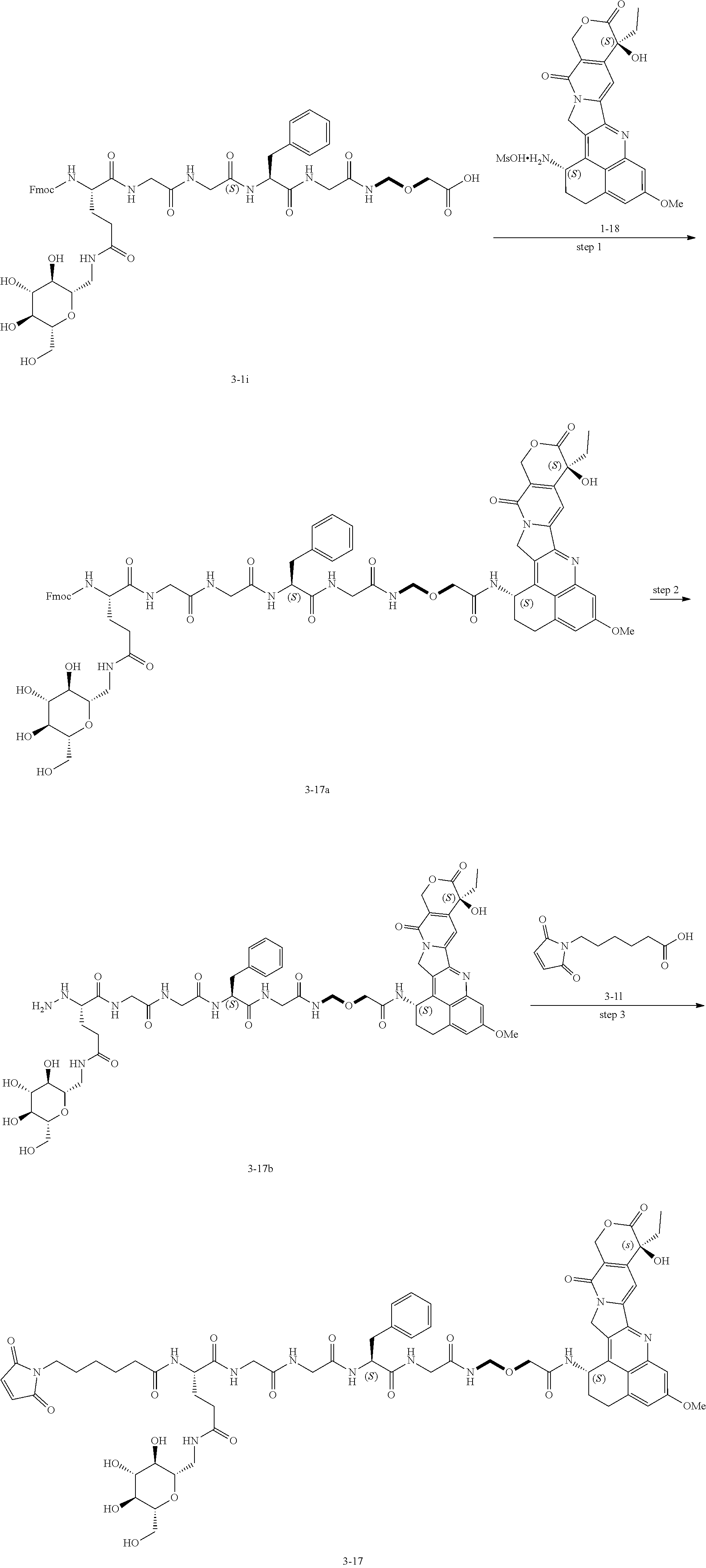

### Step 1˜Step 3

(S)—N1-((S)-10-benzyl-1-(((1S,9S)-9-ethyl-9-hydroxy-5-methoxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5 S,6R)-3,4,5-trihydroxy-6-(hydroxymethyl)tetrahydro-2H-pyran-2-yl)methyl)pentanediamide (3-17)

3-17 (5.7 mg, 46% yield) was synthesized according to synthetic procedure of example 3-1.

MS (ESI) m/z: 1336.7 [M+H]⁺

### Example 3-18

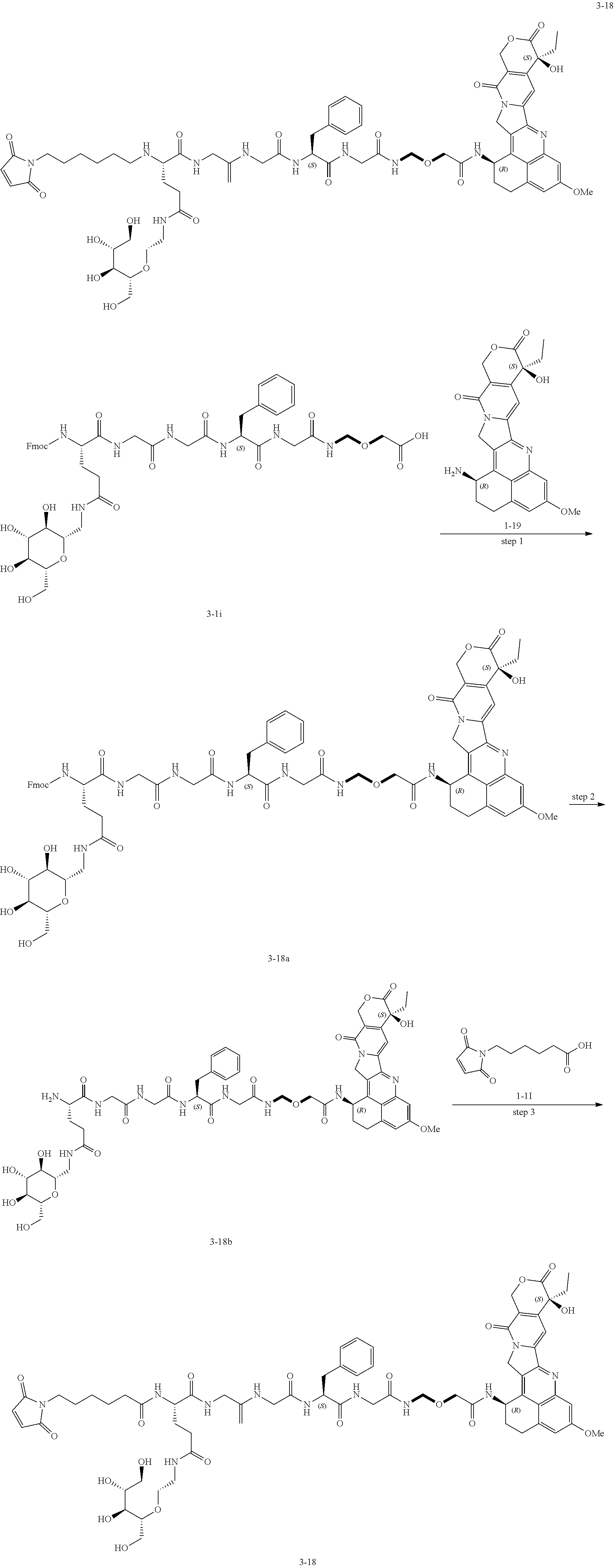

### Step 1˜Step 3

(S)—N1-((S)-10-benzyl-1-(((1R,9S)-9-ethyl-9-hydroxy-5-methoxy-10,13-dioxo-2,3,9,10,13,15-hexahydro-1H,12H-benzo[de]pyrano[3′,4′:6,7]indolizino[1,2-b]quinolin-1-yl)amino)-15-methylene-1,6,9,12-tetraoxo-3-oxa-5,8,11,14-tetraazahexadecan-16-yl)-2-(6-(2,5-dioxo-2,5-dihydro-1H-pyrrol-1-yl)hexanamido)-N5-(2-(((2R,3S,4R)-1,3,4,5-tetrahydroxypentan-2-yl)oxy)ethyl)pentanediamide (3-18)

3-18 (7.4 mg, 42% yield) was synthesized according to synthetic procedure of example 3-1.

MS (ESI) m/z: 1336.7 [M+H]⁺

### Example 3-20

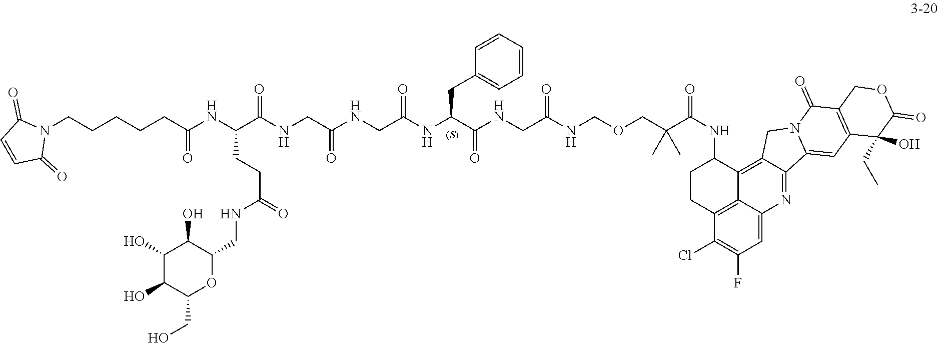

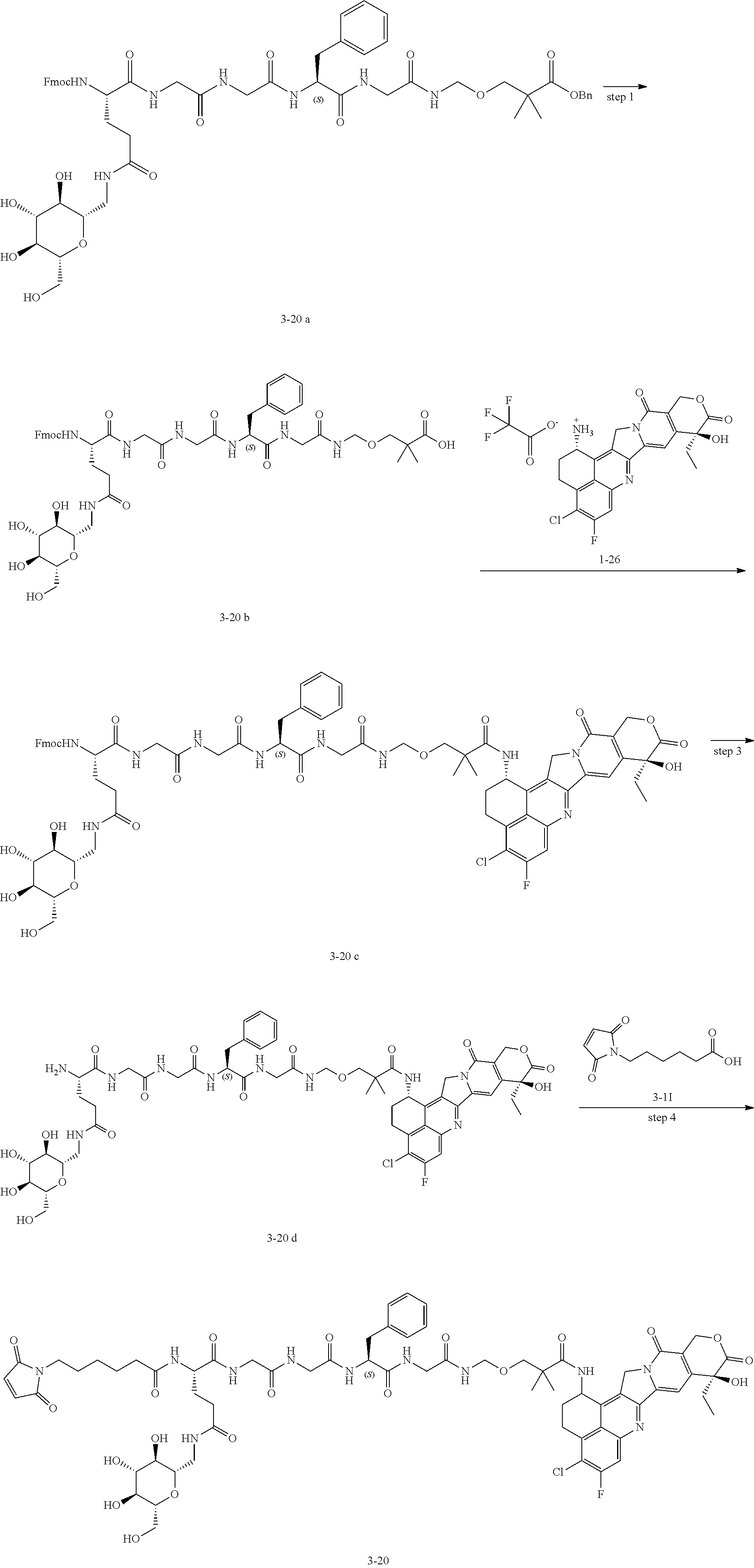

### (S)—N1-((S)-7-benzyl-17-(((1S,9S)-4-chloro-9-ethyl-5-fluoro-9-hydroxy-10,13-dioxo-2,3,9,10,13,15-hexahydro-1H, 12H-benzo[de]pyrano[3′,4′: 6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl) hexanamido)-N5-(((2S,3R,4R,5S,6R)-3,4,5-trihydroxy-6-(hydroxymethyl)tetrahydro-2H-pyran-2-yl)methyl) pentanediamide (3-20)

3-20 (5.1 mg, 97.94% purity, 24.4% yield) was synthesized according to a similar procedure as that of example 3-1.

MS (ESI) m/z: 1422.7 [M+Na]⁺

### Example 3-21

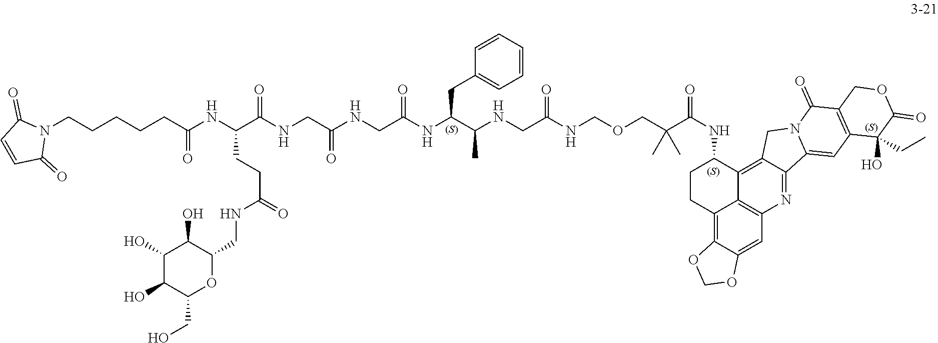

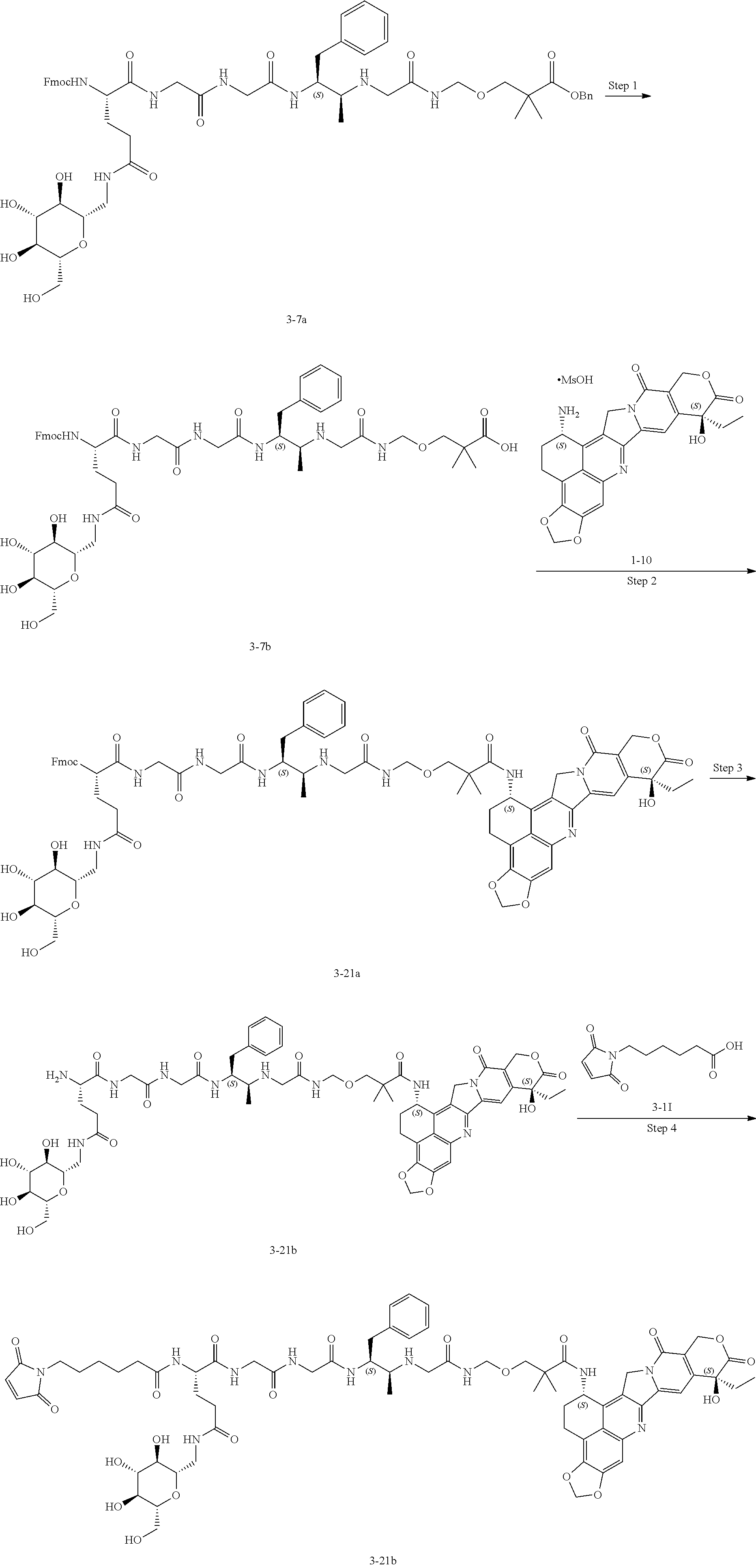

### Step 1

### (5S,14S)-14-benzyl-1-(9H-fluoren-9-yl)-23,23-dimethyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (3-7b)

Compound 3-7b (38.5 mg, crude) was synthesized according to synthetic procedure of step 6 of example 3-1.

MS (ESI) m/z: 1014.6 [M+Na]⁺

### Step 2

### (9H-fluoren-9-yl)methyl ((6S,15S)-15-benzyl-25-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-21a)

Compound 3-21a (22 mg, 31.4% yield) was synthesized according to synthetic procedure of step 7 of example 3-1.

MS (ESI) m/z: 1443.7 [M+Na]⁺

### Step 3

### (S)-2-amino-N1-((S)-7-benzyl-17-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-21b)

Compound 3-21b (18.5 mg, crude) was synthesized according to synthetic procedure of step 8 of example 3-1.

MS (ESI) m/z: 1221.7 [M+Na]⁺

### Step 4

### (S)—N1-((S)-7-benzyl-17-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-21)

Compound 3-21 (7.0 mg, 32.6% yield) was synthesized according to synthetic procedure of step 9 of example 3-1.

MS (ESI) m/z: 1414.7 [M+Na]⁺

### Example 3-22

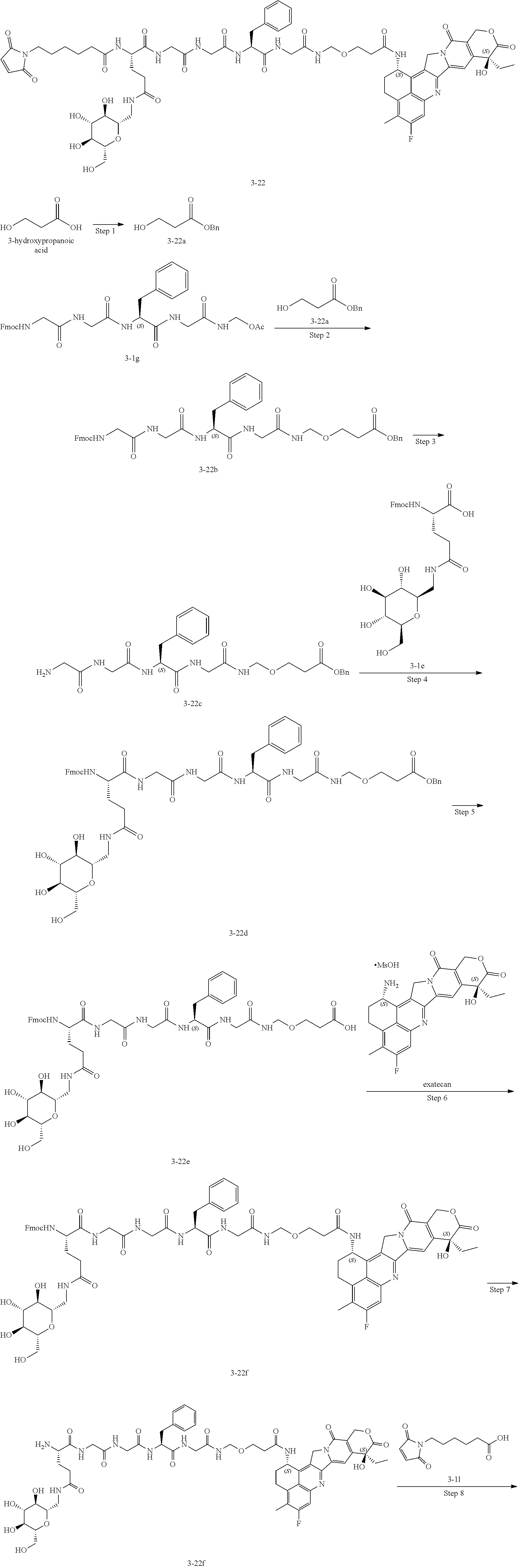

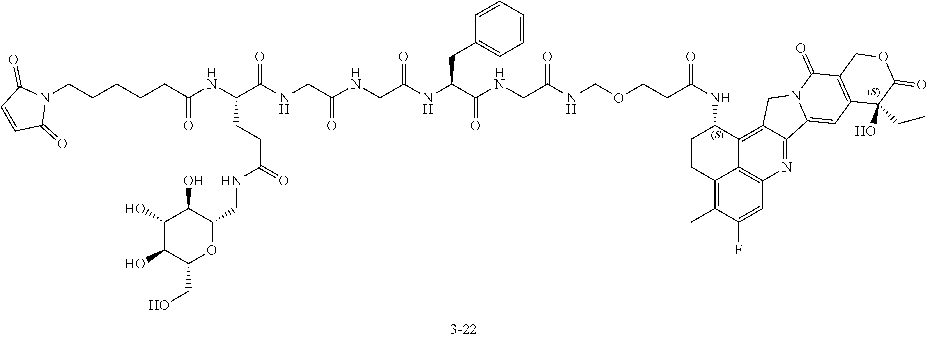

### Step 1

### benzyl 3-hydroxypropanoate (3-22a)

3-hydroxypropanoic acid (1 g, 3.3 mmol, 30 in H₂ solution) and KOH (187 mg, 3.3 mmol) was stirred at rt for 30 min, the mixture was concentrated in vacuo to give a white solid. The solid was suspended in DMF (10 mL) was added BnBr (570 mg, 3.3 mmol) via a syringe at rt. After stirring at 80° C. for 5 h, the reaction mixture concentrated in vacuo. The residue was purified by a silica gel column chromatography (eluent: PE/EA=100/0 to 30/70) to give the title compound 3-22a (420 mg, 70% yield) colorless oil.

¹H NMR (400 MHz, CDCl₃) δ 7.39-7.33 (m, 5H), 5.16 (s, 2H), 3.88 (t, J=5.6H, 2H), 2.66 (t, J=5.6H, 2H), 2.38 (br s, 1H).

### Step 2

### benzyl(S)-11-benzyl-1-(9H-fluoren-9-yl)-3,6,9,12,15-pentaoxo-2,18-dioxa-4,7,10,13,16-pentaazahenicosan-21-oate (3-22b)

Compound 3-22b (1.2 g, 83.9% yield) was synthesized according to synthetic procedure of step 5 of example 3-1.

MS (ESI) m/z: 773.5 [M+Na]⁺

### Step 3

### Benzyl(S)-1-amino-7-benzyl-2,5,8,11-tetraoxo-14-oxa-3,6,9,12-tetraazaheptadecan-17-oate (3-22c)

Compound 3-22c (150 mg, crude) was synthesized according to synthetic procedure of step 8 of example 3-1.

MS (ESI) m/z: 528.3 [M+H]⁺

### Step 4

### benzyl(5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oate (3-22d)

Compound 3-22d (170 mg, 80.9% yield) was synthesized according to synthetic procedure of step 5 of example 3-1.

MS (ESI) m/z: 1076.6 [M+Na]⁺

### Step 5

### (5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (3-22e)

Compound 3-22e (150 mg, crude) was synthesized according to synthetic procedure of step 6 of example 3-1.

MS (ESI) m/z: 986.5 [M+Na]⁺

### Step 6

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-22f)

Compound 3-22f (190 mg, 88.4% yield) was synthesized according to synthetic procedure of step 7 of example 3-1.

MS (ESI) m/z: 1404.5 [M+Na]⁺

### Step 7

### (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-22g)

Compound 3-22g (190 mg, crude) was synthesized according to synthetic procedure of step 8 of example 3-1.

### Step 8

### (S)—N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-22)

Compound 3-22 (50.3 mg, 54.1% yield) was synthesized according to synthetic procedure of step 9 of example 3-1.

¹H NMR (400 MHz, DMSO-d₆) δ 8.51 (d, J=9.2 Hz, 2H), 8.32 (s, 1H), 8.13 (d, J=8.0 Hz, 2H), 8.03 (d, J=7.2 Hz, 2H), 7.80 (d, J=11.2 Hz, 2H), 7.32 (s, 1H), 7.28-7.19 (m, 4H), 7.17 (d, J=6.6 Hz, 1H), 7.00 (d, J=8.0 Hz, 2H), 6.54 (s, 1H), 5.56 (d, J=8.4 Hz, 1H), 5.43 (s, 2H), 5.22 (d, J=4.8 Hz, 2H), 5.01 (d, J=5.2 Hz, 1H), 4.94-4.84 (m, 2H), 4.65-4.44 (m, 3H), 4.43-4.33 (m, 1H), 4.19 (d, J=6.0 Hz, 1H), 3.84-3.51 (m, 10H), 3.22-2.93 (m, 8H), 2.88 (s, 2H), 2.83-2.70 (m, 1H), 2.37 (d, J=29.2 Hz, 6H), 2.10 (dd, J=14.6, 7.3 Hz, 7H), 1.97-1.78 (m, 3H), 1.72 (d, J=8.0 Hz, 1H), 1.59-1.37 (m, 4H), 1.22-1.10 (m, 2H), 0.87 (t, J=7.2 Hz, 3H).

MS (ESI) m/z: 1374.9 [M+Na]⁺; UPLC-MS Retention time: 3.84 min.

### Example 3-23

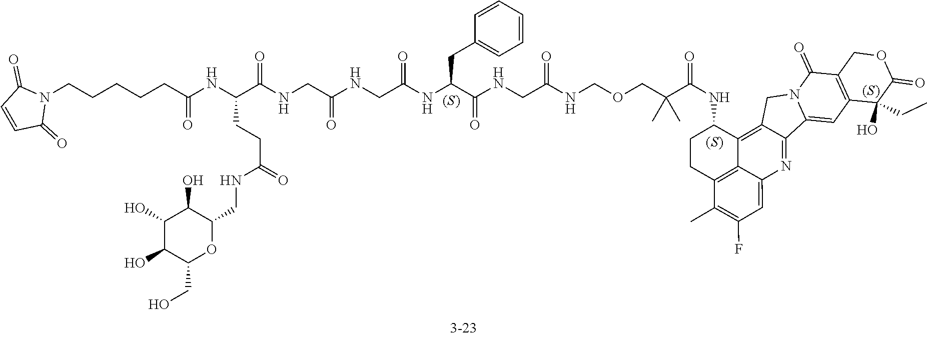

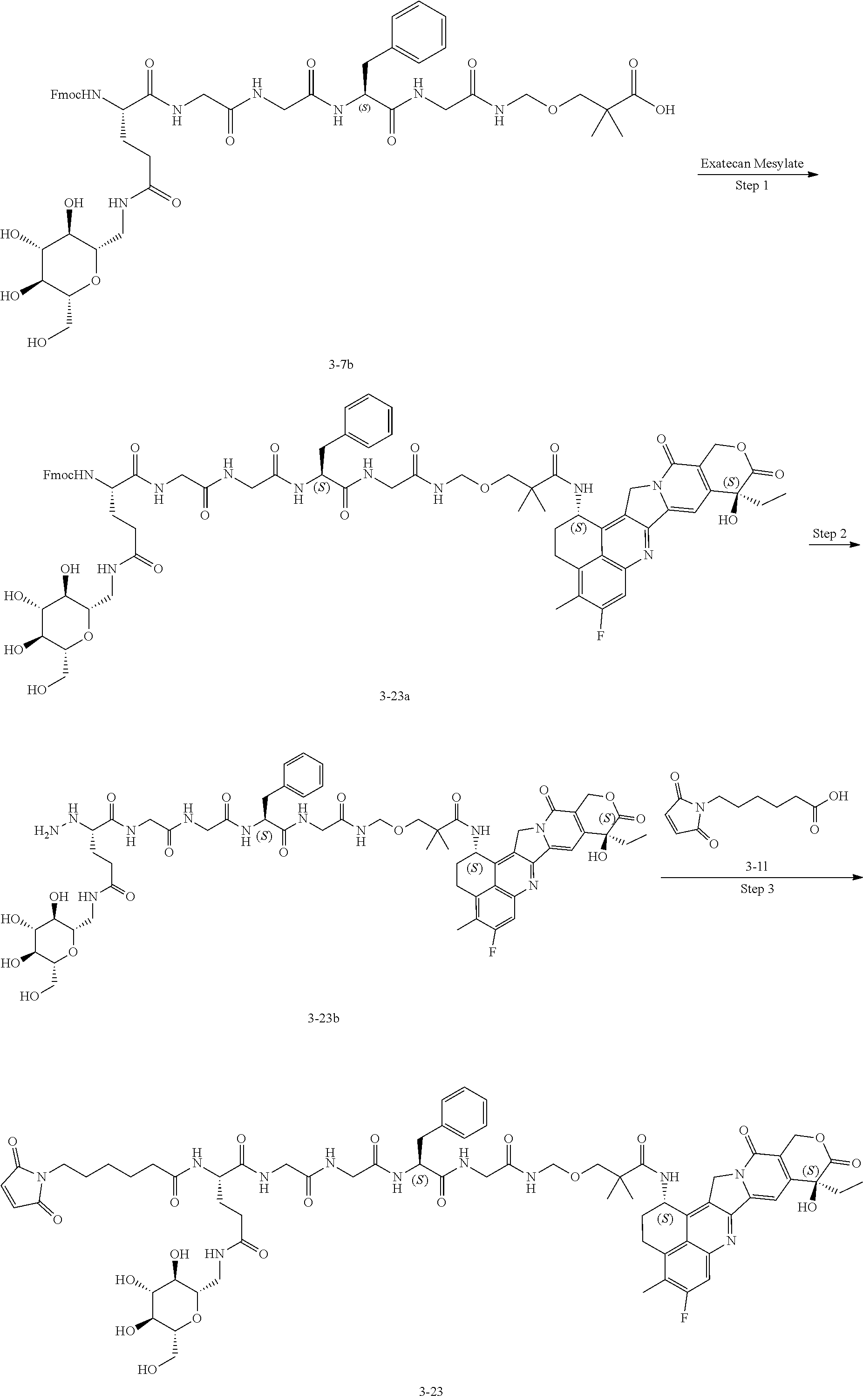

(S)—N¹—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (3-23) (32 mg, 26.3% yield) was synthesized according to synthetic procedure of example 3-20 as a white powder.

MS (ESI) m/z: 1381.1 [M+H]

### Example 3-24

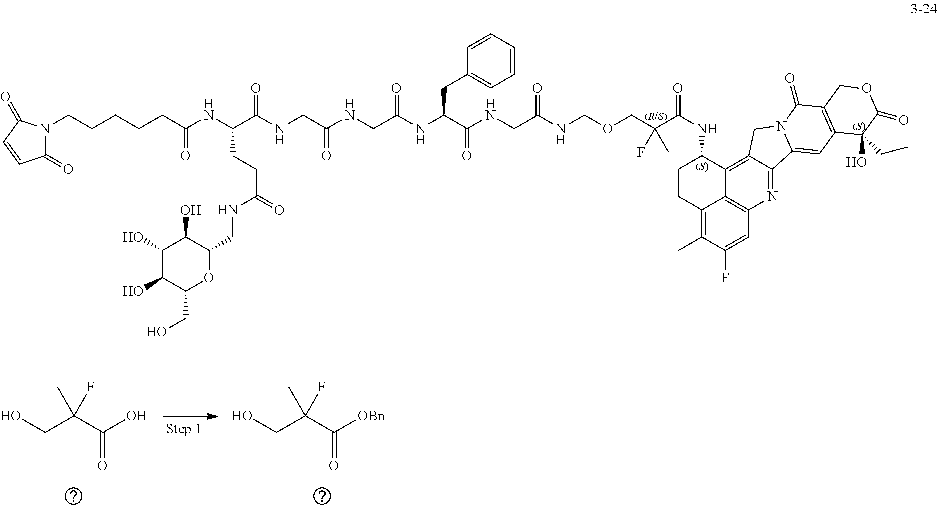

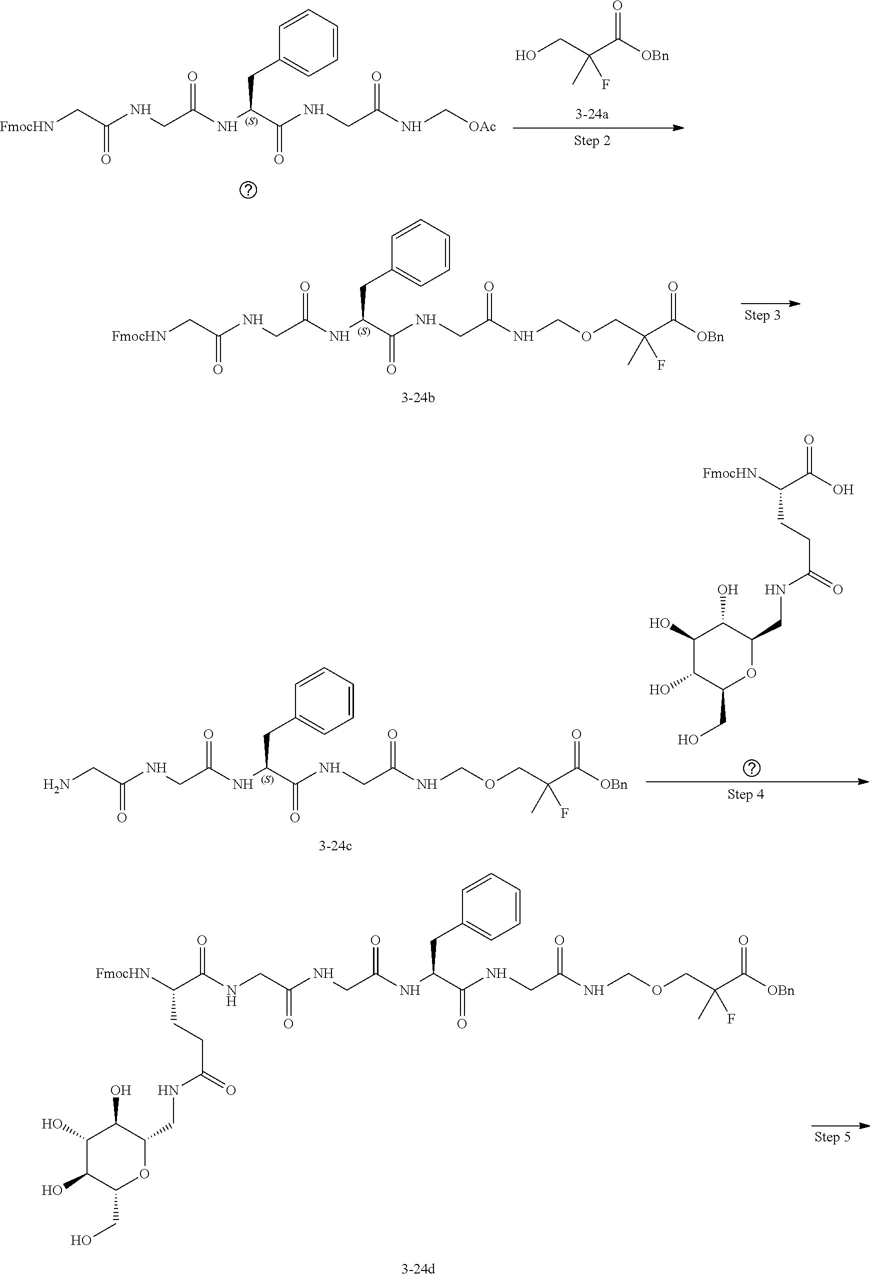

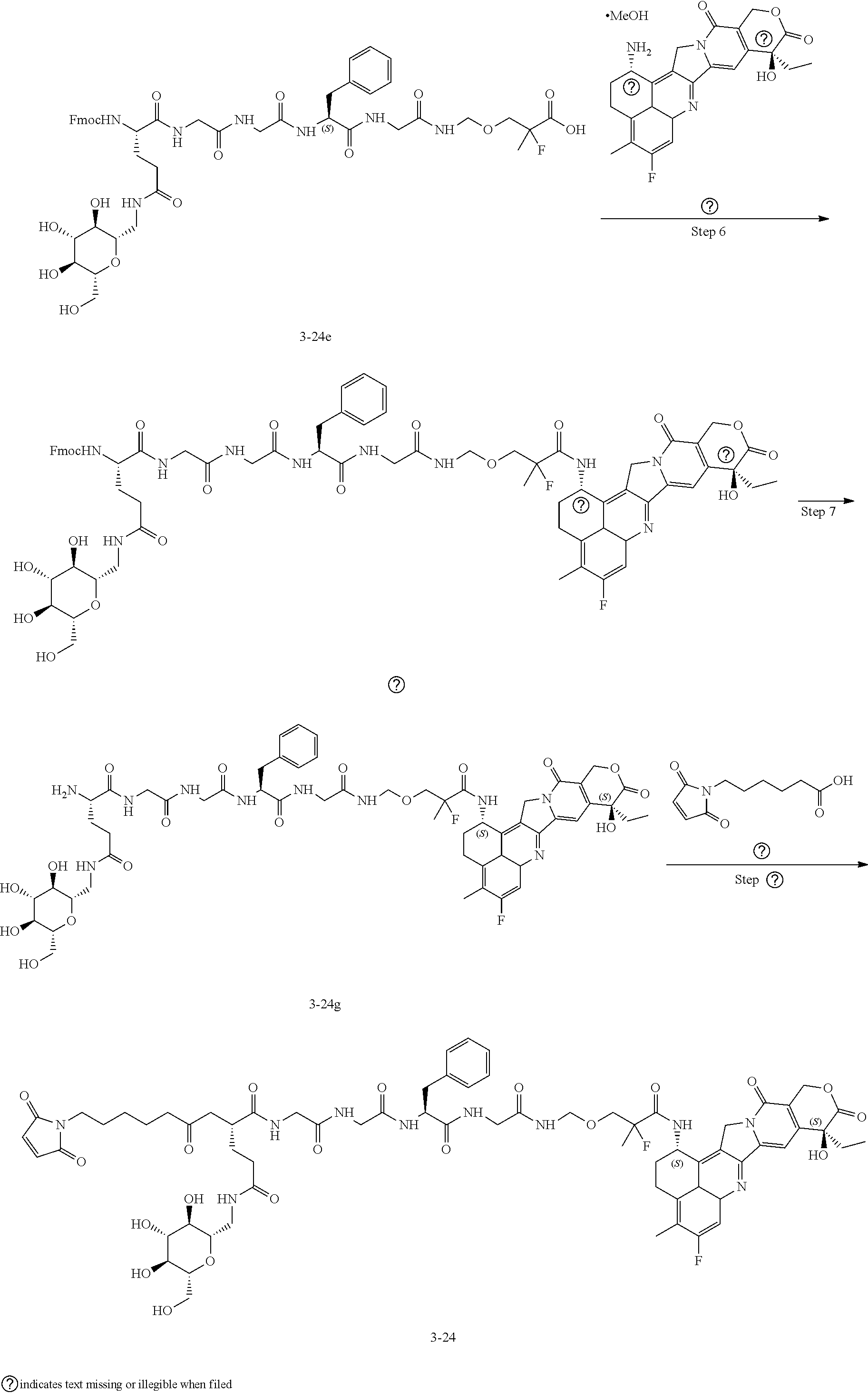

### Step 1

### benzyl 2-fluoro-3-hydroxy-2-methylpropanoate (3-24a)

To a solution of compound 2-41c (450 mg, 3.69 mmol) in DMF (10 mL) were added K₂CO₃ (1.02 g, 7.37 mmol) and BnBr (945 mg, 5.53 mmol). The mixture was stirred at 35° C. for 16 h. The mixture was diluted with EtOAc (200 mL), washed by brine (50 mL*4). The organic layer was dried over anhydrous Na₂SO₄, filtered and concentrated. The residue was purified by silica column gel chromatography (eluent: PE/EtOAc=100/0 to 30/70) to afford 3-24a (270 mg, 34.5% yield) as colorless oil.

¹H NMR (400 MHz, CDCl₃) δ 7.42-7.33 (m, 5H), 5.26 (s, 2H), 4.06-3.61 (m, 2H), 2.08 (br s, 1H), 1.55 (d, J 21.2 Hz, 3H).

### Step 2

### benzyl (11S)-1-benzyl-1-(9H-fluoren-9-yl)-20-fluoro-20-methyl-3,6,9,12,15-pentaoxo-2,18-dioxa-4,7,10,13,16-pentaazahenicosan-21-oate (3-24b)

To the mixture of 3-1g (250 mg, 0.40 mmol), 3-24a (253 mg, 1.19 mmol) and dried 4 Å molecular sieves (500 mg) in dry THF (8 mL) was added scandium trifluoromethanesulfonate (293 mg, 0.60 mmol), stirred at r.t. under N₂ atmosphere overnight. The solution was filtered through Celite, diluted with EtOAc (100 mL), washed by saturated NaHCO₃ (40 mL*3). The organic layer was dried over anhydrous Na₂SO₄, filtered and concentrated. The residue was purified by silica column gel chromatography (eluent: PE/EtOAc=100/0 to 50/50) to afford 3-24b (210 mg, 67.7% yield).

MS (ESI) m/z: 652.3 [M+Na]7

### Step 3

### benzyl (7S)-1-amino-7-benzyl-16-fluoro-16-methyl-2,5,8,11-tetraoxo-14-oxa-3,6,9,12-tetraazaheptadecan-17-oate (3-24c)

To a solution of compound 3-24b (210 mg, 0.27 mmol) in DMF (4 mL) was added Et₂NH (392 mg, 5.37 mmol). The mixture was stirred at r.t. for 30 met. The mixture was concentrated under high vacuum and co-evaporated with toluene (3 mL*3) to give 3-24c (210 mg, crude) as brown solid.

MS (ESI) m/z: 582.3 [M+Na]⁺

### Step 4

benzyl (5S,14S)-14-benzyl-1-(9H-fluoren-9-yl)-23-fluoro-23-methyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oate (3-24d)

To a solution of compound 3-24c (210 mg, crude) in DMF (4 mL) were added 3-1e (161 mg, 0.29 mol), HATU (153 mg, 0.40 mmol) and DIEA (69 mg, 0.63 mmol). The mixture was stirred at r.t. for 30 min. The mixture was filtered and the filtrate was purified using prep-HPLC (Method: column: XBridge Prep C18 OBD 5 um 19*150 mm; Mobile phase: A-water (0.1% formic acid): B-acetonitrile; Flow rate: 20 mL/min to provide 3-24d (154 mg, 52.9% yield) as white solid.

MS (ESI) m/z: 1108.7 [M+Na]⁺

### Step 5

### (5S,14S)-14-benzyl-1-(9H-fluoren-9-yl)-23-fluoro-23-methyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (3-24e)

To a solution of compound 3-24d (154 mg, 0.14 mmol) in MeOH (6 mL) was added Pd/C (10%, 30 mg). The mixture was stirred at H₂ atmosphere (15 psi) for 4 h. The mixture was filtered through a pad of celite, concentrated to give compound 3-24e (141 mg, 100% yield) as white solid.

MS (ESI) m/z: 1018.6 [M+Na]⁺

### Step 6

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24-fluoro-24-methyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-24f)

To a solution of compound 3-24e (141 mg, 0.15) in DMF (4 mL) were added exatecan mesylate (86 mg, 0.16 mol), HATU (88 mg, 0.23 mmol) and DIEA (100 mg, 0.77 mmol). The mixture was stirred at r.t. for 30 min. The mixture was filtered and the filtrate was purified using prep-HPLC (Method: column: XBridge Prep C18 OBD 5 um 19*150 mm; Mobile phase: A—water (0.1% trifluoroacetic acid): B-acetonitrile; Flow rate: 20 mL/min to provide compound 3-24f (62 mg, 28.3% yield) was obtained as white solid.

MS (ESI) m/z: 1436.7 [M+Na]⁺

### Step 7

### (2S)-2-amino-N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-24g)

To a solution of compound 3-24f (62 mg, 0.44 mmol) in DMF (4 mL) was added Et₂NH (64 mg, 0.88 mmol). The mixture was stirred at r.t. for 30 min. The mixture was concentrated under high vacuum and co-evaporated with toluene (3 mL*3) to give 3-24g (62 mg, crude) as brown solid.

MS (ESI) m/z: 1191.7 [M+Na]⁺

### Step 8

### (2S)—N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-24)

To a solution of compound 3-24g (52 mg, 0.44 mmol) in DMF (3 mL) were added 3-11 (19 mg, 0.088 mol), HATU (33 mg, 0.088 mmol) and DIEA (11 mg, 0.088 mmol). The mixture was stirred at r.t. for 30 min. The mixture was filtered and the filtrate was purified using prep-HPLC (Method: column: XBridge Prep C18 OBD 5 um 19*150 mm; Mobile phase: A—water (0.1% trifluoroacetic acid): B-acetonitrile; Flow rate: 20 mL/min to provide compound 3-24 (18.4 mg, 36.2% yield) as white solid. UPLC-MS Retention time: 3.99 min.

¹H NMR (400 MHz, DMSO-d₆) δ 8.91 (d, J=9.2 Hz, 1H), 8.59 (t, J=6.8 Hz, 1H), 8.33 (t, J=5.8 Hz, 1H), 8.12 (d, J=7.2 Hz, 2H), 8.03 (t, J=7.6 Hz, 2H), 7.77 (d, J=10.8 Hz, 2H), 7.30 (s, 1H), 7.27-7.21 (m, 4H), 7.20-7.11 (m, 1H), 7.00 (d, J=8.4 Hz, 2H), 6.52 (s, 1H), 5.58 (d, J=7.8 Hz, 1H), 5.41 (s, 2H), 5.15 (dd, J=63.4, 18.8 Hz, 2H), 4.99 (d, J=5.4 Hz, 1H), 4.89 (dd, J=6.4, 5.2 Hz, 2H), 4.62 (d, J=6.8 Hz, 2H), 4.51 (dd, J=12.8, 9.2 Hz, 1H), 4.38 (dd, J=7.6, 4.6 Hz, 1H), 4.20 (d, J=6.0 Hz, 1H), 3.37 (dt, J=13.8, 6.8 Hz, 4H), 3.25 (d, J=18.0 Hz, 2H), 3.09 (ddd, J=20.8, 13.6, 9.2 Hz, 5H), 3.00-2.64 (m, 5H), 2.35 (d, J=18.8 Hz, 4H), 2.26-1.94 (m, 7H), 1.87 (dt, J=15.6, 7.2 Hz, 3H), 1.73 (d, J=8.0 Hz, 1H), 1.61 (d, J=21.8 Hz, 3H), 1.52-1.35 (m, 4H), 1.31-1.09 (m, 3H), 0.87 (t, J=7.2 Hz, 3H);

MS (ESI) m/z: 1406.7 [M+Na]⁺;

### Example 3-25

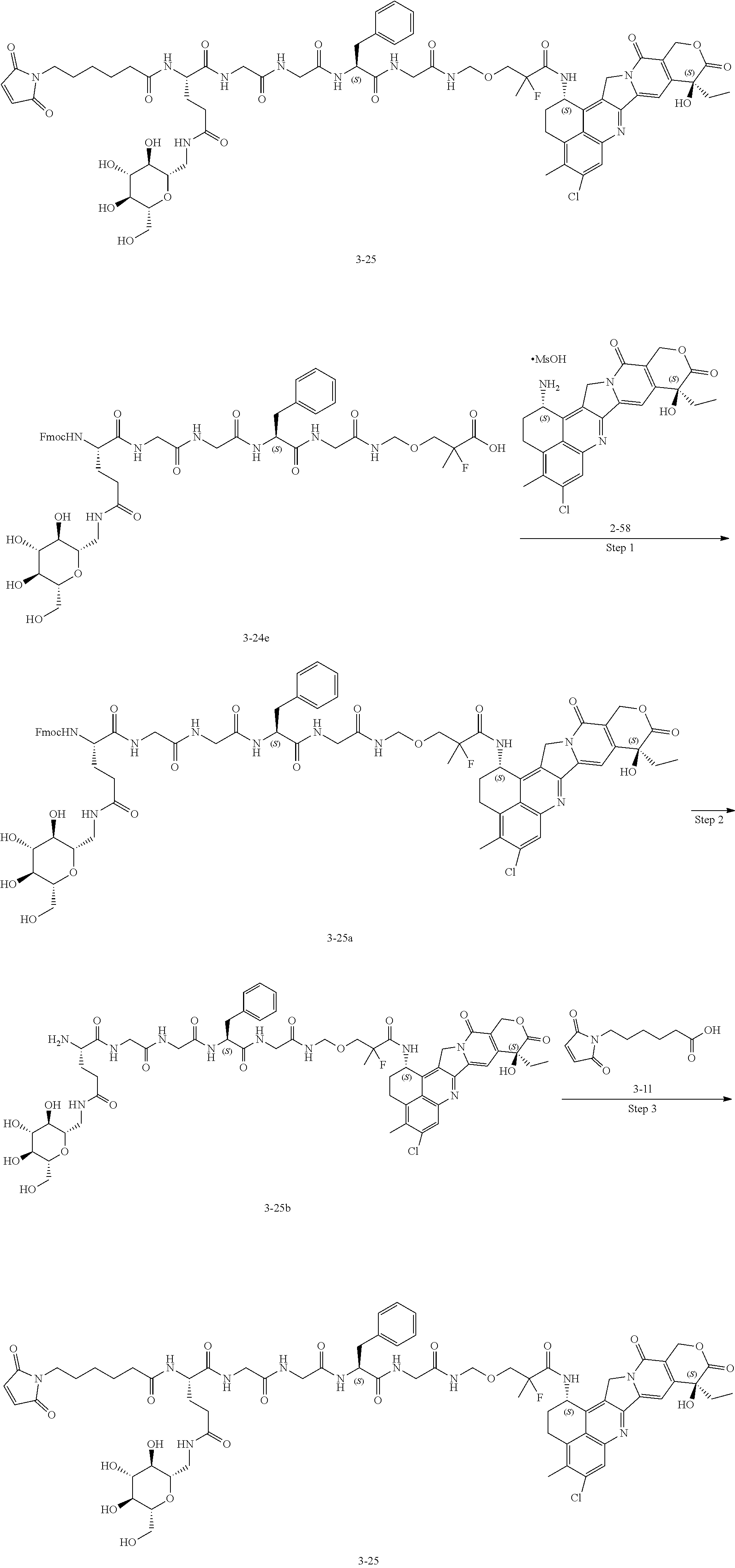

### Step 1

### (9H-fluoren-9-yl)methyl ((6S,15S)-15-benzyl-25-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24-fluoro-24-methyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (3-25a)

Compound 3-25a (35.0 mg, 81.5% yield) was synthesized according to synthetic procedure of step 6 of example 3-1.

MS (ESI) m/z: 1453.9 [M+Na]⁺

### Step 2

### (2S)-2-amino-N1-((7S)-7-benzyl-17-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-25b)

Compound 3-25b (29.6 mg, crude) was synthesized according to synthetic procedure of step 7 of example 3-1.

MS (ESI) m/z: 1231.9 [M+Na]⁺

### Step 3

### (2S)—N1-((7S)-7-benzyl-17-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-25)

Compound 3-25 (12.6 mg, 36.7% yield) was synthesized according to synthetic procedure of step 8 of example 3-1.

MS (ESI) m/z: 1442.8 [M+Na]⁺

### Example 3-26

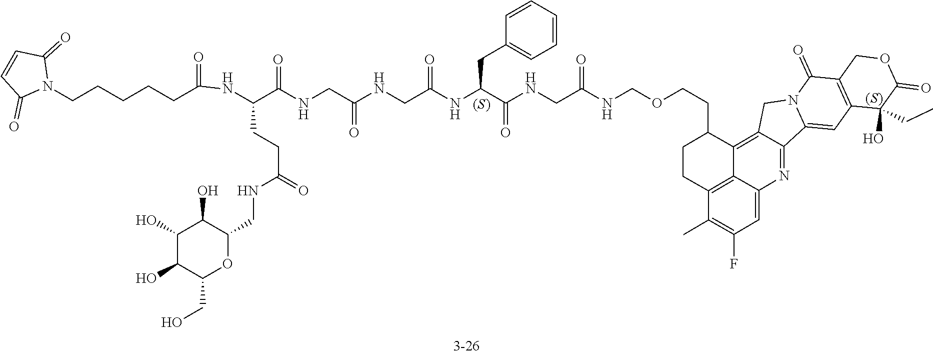

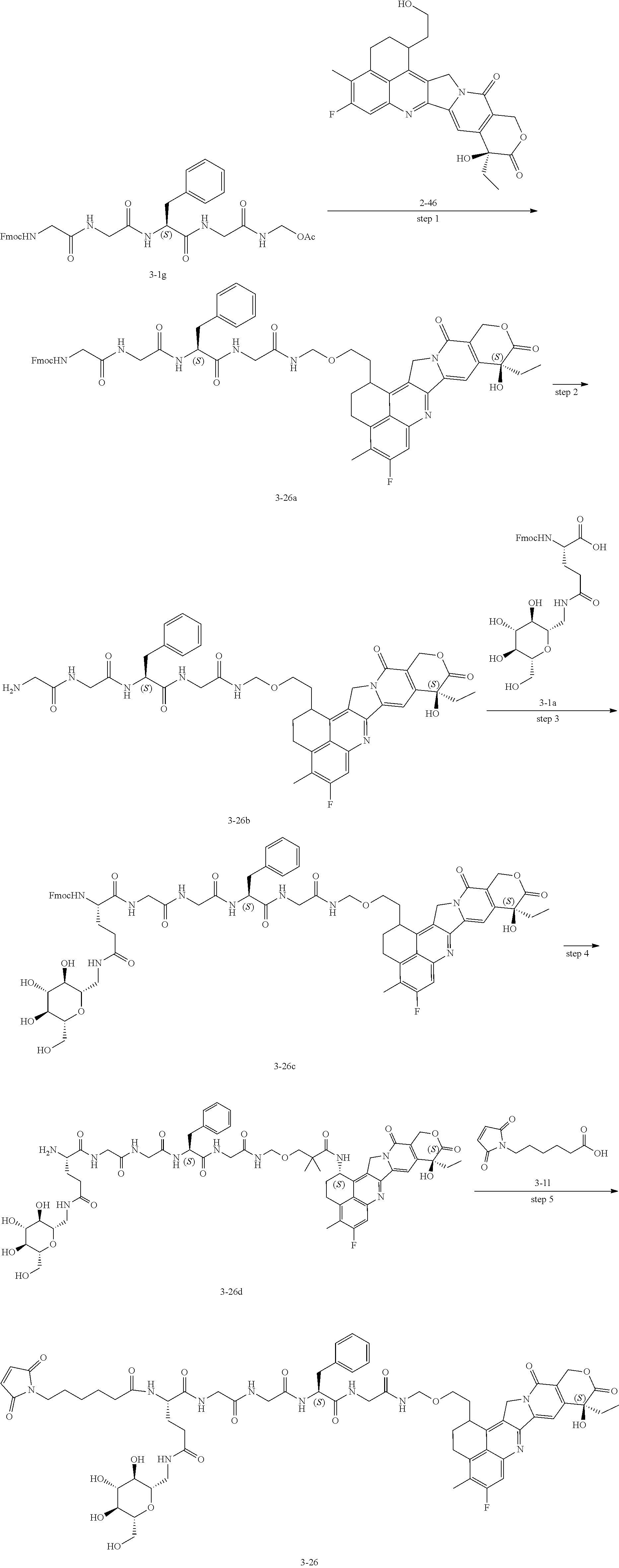

### Step 1

### (9H-fluoren-9-yl)methyl((10S)-10-benzyl-1-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-6,9,12,15-tetraoxo-3-oxa-5,8,11,14-tetraazahexadecan-16-yl)carbamate (3-26a)

To a solution of 3-1g (100 mg, 0.16 mmol) in THF (5 mL) was added 2-46 (73.77 mg. 0.16 mmol) and 4 Å MS (400 mg). The resulting mixture was stirred at 20° C. for 30 min. Then Sc(OTf)₃ (118.08 mg, 0.24 mmol) was added. The reaction mixture was stirred at 20° C. for 1 h. LCMS showed the reaction was completed. After filtration through celite, the filtrate was poured into saturated NaHCO₃ aq. (30 mL) and was extracted with EtOAc (30 mL*3). The organic layer was dried over Na₂SO₄ and filtered. The filtrate was concentrated under vacuum and the residue was purified by silica column gel chromatography (eluent: CH₂Cl₂/MeOH=100/0 to 90/10) to afford 3-26a (110 mg, 66% yield) as a white solid.

MS (ESI) m/z: 1034.4 [M+H]⁺

### Step 2

### (2S)-2-(2-(2-aminoacetamido)acetamido)-N-(2-(((2-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)ethoxy)methyl)amino)-2-oxoethyl)-3-phenylpropanamide (3-26b)

A solution of 3-26a (110 g, 0.11 mmol) in DMF (3 mL) was added Et₂NH (142.5 mg, 0.19 mmol). The mixture was stirred at r.t. for 1 h. The mixture was concentrated and co-evaporated with toluene (3*3 mL). The crude 3-26b (86.3 mg, 100% yield) was used for next step without further purification.

MS (ESI) m/z: 812.3 [M+H]⁺

### Step 3

### (9H-fluoren-9-yl)methyl((6S,15S)-15-benzyl-24-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (3-26c)

To a solution of compound 3-26b (86.3 mg, 0.11 mmol) in DMF (3 mL) were added 3-1e (57.8 mg, 0.11 mmol), HATU (50.16 mg, 0.13 mmol) and DIEA (28.38 mg, 0.22 mmol). The mixture was stirred at 20° C. for 30 min. LCMS showed the reaction was completed. The mixture was filtered, the filtrate was purified using prep-HPLC (Method: column: XBridge Prep C18 OBD 5 um 19*150 mm; Mobile phase: A—water (0.1% trifluoroacetic acid): B-acetonitrile; Flow rate: 20 mL/min to provide compound 3-26c (15 mg, 10.2% yield) as white solid.

MS (ESI) m/z: 1338.5 [M+H]⁺

### Step 4

### (2S)-2-amino-N1-((10S)-10-benzyl-1-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-6,9,12,15-tetraoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (3-26d)

A solution of 3-26c (15 mg, 0.01 mmol) in DMF (1 mL) was added Et₂NH (14.25 mg, 0.02 mmol). The mixture was stirred at r.t. for 1 h. The mixture was concentrated and co-evaporated with toluene (3*3 mL). The crude 3-26d (11.16 mg, 100% yield) was used for next step without further purification.

MS (ESI) m/z: 1116.4 [M+H]⁺

### Step 5

### (2S)—N1-((10S)-10-benzyl-1-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-6,9,12,15-tetraoxo-3-oxa-5,8,11,14-tetraazahexadecan-16-yl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-26)

To a solution of 3-26d (11.16 mg, 0.01 mmol) in DMF (1 mL) and 3-11 (2.11 mg, 0.01 mmol) were added HATU (3.8 mg, 0.01 mmol) and DIEA (2.58 mg, 0.02 mmol) was added into the mixture and reacted at the same temperature for 15 min. The mixture was purified by prep-HPLC (Method: column: XBridge Prep C18 OBD 5 um 19*150 mm; Mobile phase: A—water (0.1% formic acid): B-acetonitrile; Flow rate: 20 mL/min). 3-26 (3.2 mg, 24.5% yield) was obtained as a white solid.

MS (ESI) m/z: 1309.5 [M+H]⁺

### Example 3-27

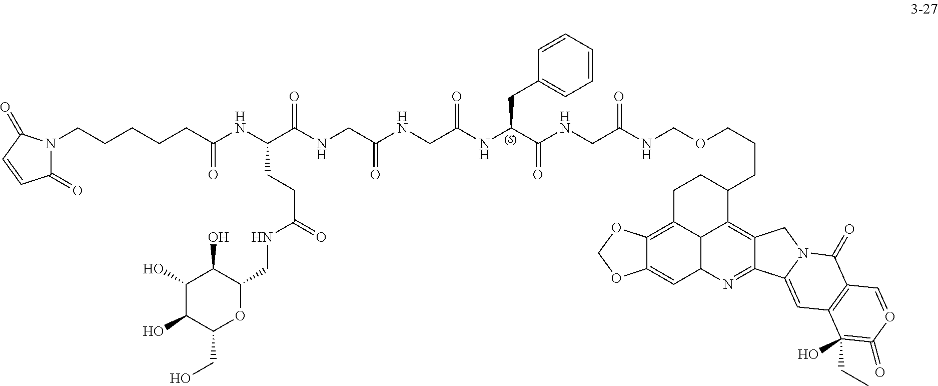

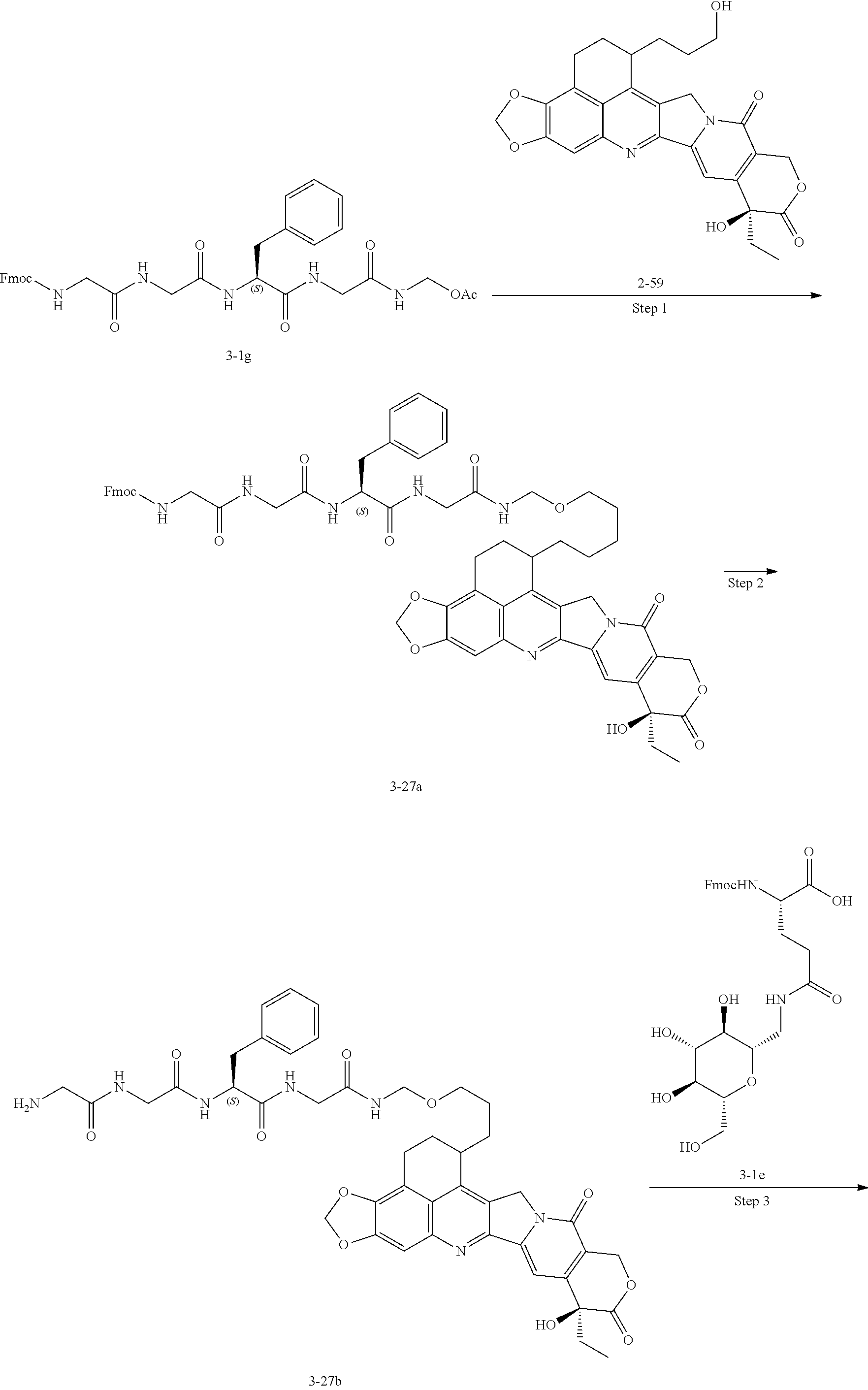

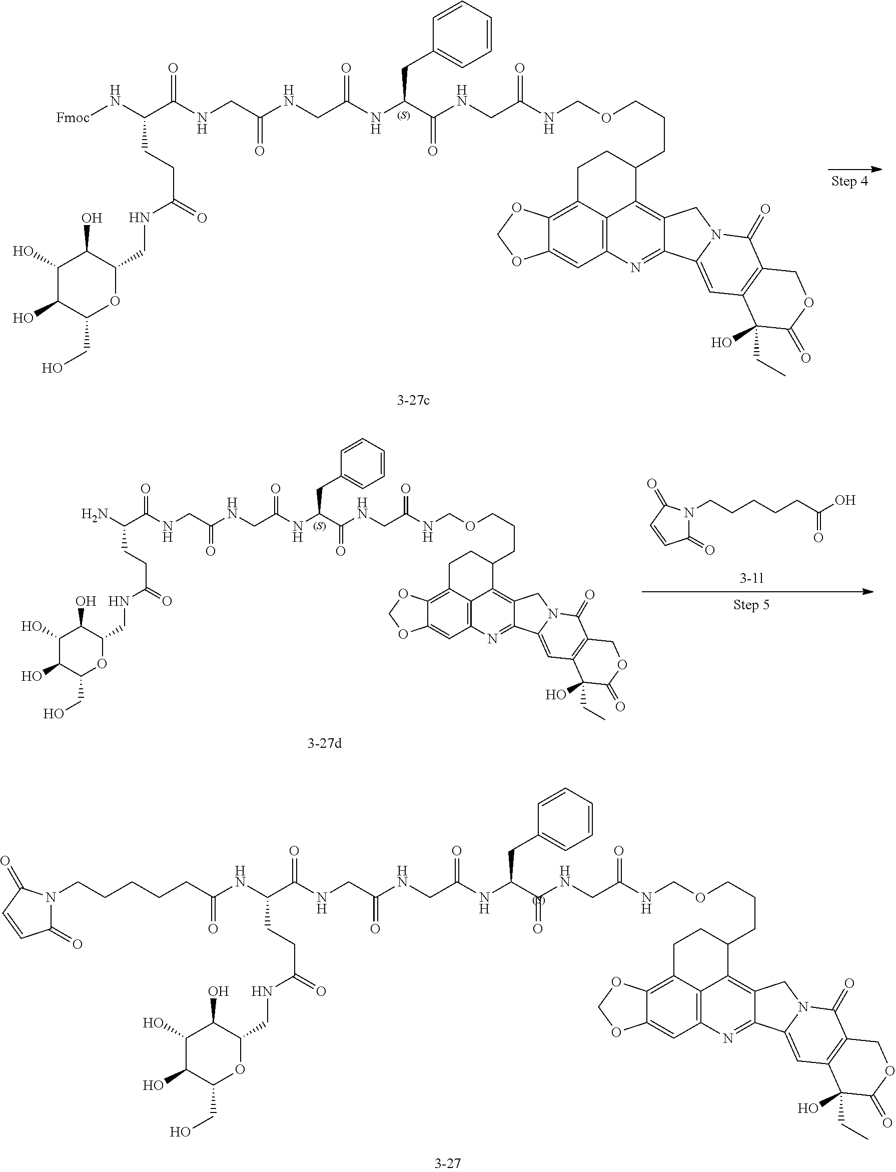

### Step 1

### (9H-fluoren-9-yl)methyl ((S)-7-benzyl-17-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-2,5,8,11-tetraoxo-14-oxa-3,6,9,12-tetraazaheptadecyl)carbamate (3-27a)

compound 3-27a (25 mg, 58.1% yield) was synthesized according to synthetic procedure of step 1 of example 3-26a.

MS (ESI) m/z: 1060.9 [M+H]

### Step 2

### (S)-2-(2-(2-aminoacetamido)acetamido)-N-(2-(((3-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)propoxy)methyl)amino)-2-oxoethyl)-3-phenylpropanamide (3-27b)

give 3-27b (26 mg, crude) was synthesized according to synthetic procedure of step 2 of example 3-26b as brown solid.

MS (ESI) m/z: 838.7 [M+H]⁺

### Step 3

### (9H-fluoren-9-yl)methyl ((6S,15S)-15-benzyl-25-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-3,7,10,13,16,19-hexaoxo-1-((2S,3R,4R,5S,6R)-3,4,5-trihydroxy-6-(hydroxymethyl)tetrahydro-2H-pyran-yl)-22-oxa-2,8,11,14,17,20-hexaazapentacosan-6-yl)carbamate (3-27c)

compound 3-27c (20 mg, 47.6% yield) was synthesized according to synthetic procedure of step 3 of example 3-26c as a yellow solid.

MS (ESI) m/z: 1365.1 [M+H]⁺

### Step 4

### (S)-2-amino-N1-((S)-7-benzyl-17-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-2,5,8,11-tetraoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (3-27d)

compound 3-27d (22 mg, crude) was synthesized according to synthetic procedure of step 4 of example 3-26d as a yellow solid.

MS (ESI) m/z: 1142.9 [M+H]⁺

### Step 5

### (S)—N1-((S)-7-benzyl-17-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-2,5,8,11-tetraoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-27)

compound 3-27 was synthesized according to synthetic procedure of step 5 of example 3-26. The crude product was purified by prep-HPLC (Method: column: XBridge Prep C18 OBD 5 um 19*150 mm; Mobile phase: A—water (0.1% formic acid): B-acetonitrile; Flow rate: 20 mL/min) as a white solid (4 mg, 10.5% yield).

MS (ESI) m/z: 1337.0 [M+H]⁺

### Example 3-28

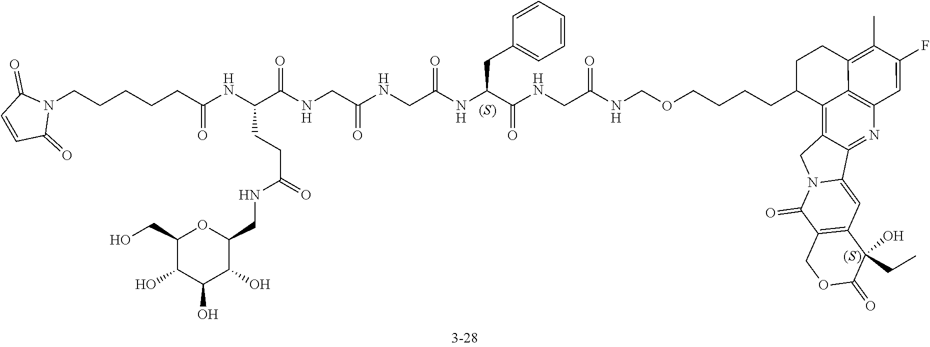

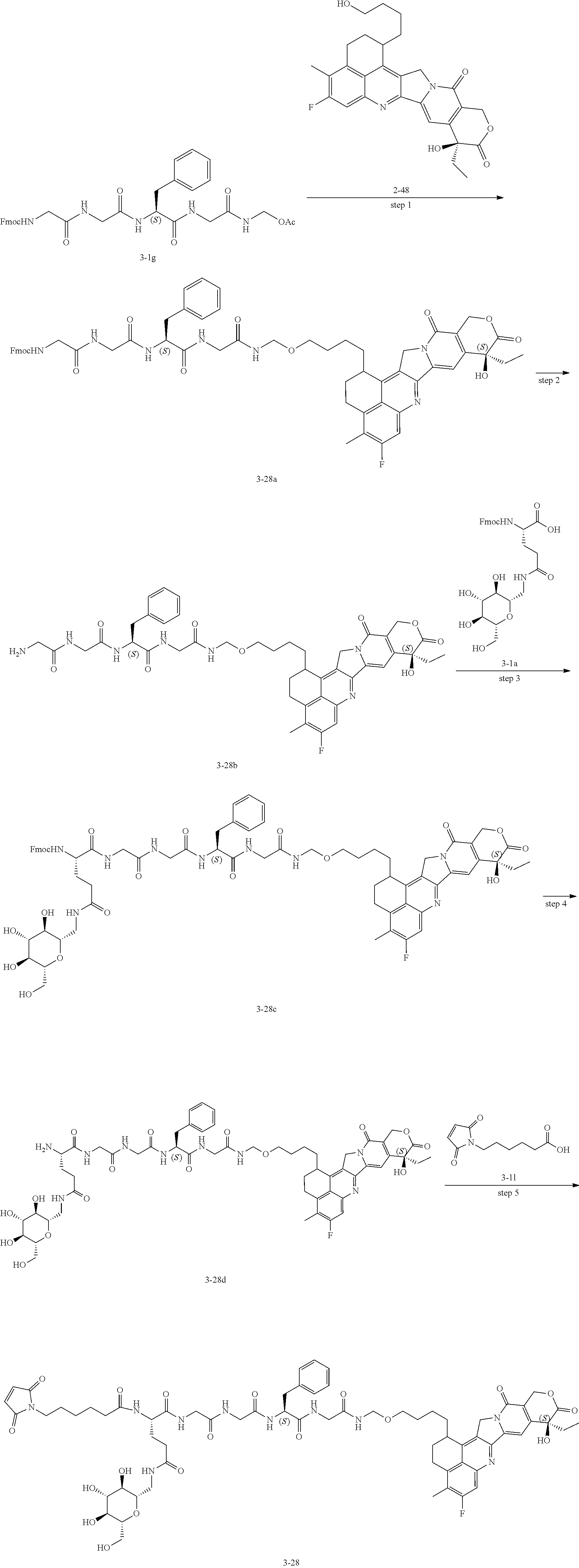

### Step 1

### (9H-fluoren-9-yl)methyl ((7S)-7-benzyl-18-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaoctadecyl)carbamate (3-28a)

Compound 3-28a (85.0 mg, 62.5% yield) was synthesized according to synthetic procedure of step 1 of example 3-26a.

MS (ESI) m/z: 1062.8 [M+H]⁺

### Step 2

### (2S)-2-(2-(2-aminoacetamido)acetamido)-N-(2-(((4-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)butoxy)methyl)amino)-2-oxoethyl)-3-phenylpropanamide (3-28b)

Compound 3-28b (61.7 mg, crude) was synthesized according to synthetic procedure of step 2 of example 3-26b.

MS (ESI) m/z: 840.7 [M+H]⁺

### Step 3

### (9H-fluoren-9-yl)methyl ((6S,15S)-15-benzyl-26-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazahexacosan-6-yl)carbamate (3-28c)

Compound 3-28c (38.0 mg, 42.06% yield) was synthesized according to synthetic procedure of step 3 of example 3-26c.

MS (ESI) m/z: 1367.1 [M+H]⁺

### Step 4

### (2S)-2-amino-N1-((7S)-7-benzyl-18-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaoctadecyl)-N5-(((2S,3R,4R,5S,6R)-3,4,5-trihydroxy-6-(hydroxymethyl)tetrahydro-2H-pyran-2-yl)methyl)pentanediamide (3-28d)

Compound 3-28d (35.0 mg, crude) was synthesized according to synthetic procedure of step 4 of example 3-26d.

MS (ESI) m/z: 1144.9 [M+H]⁺

### Step 5

### (2S)—N1-((7S)-7-benzyl-18-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaoctadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (3-28)

Compound 3-28 (16.8 mg, 45.6% yield) was synthesized according to synthetic procedure of step 5 of example 3-26.

MS (ESI) m/z: 1338.1 [M+H]⁺

TABLE 1
Exatecan analogues

| Examples | Payload structure | Cellular |
|---|---|---|
| 1-A | 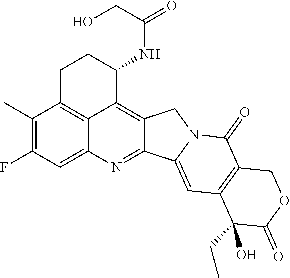 [SMILES: CC[C@@]1(O)C(=O)OCc2c1cc1n(c2=O)Cc2c-1nc1cc(F)c(C)c3c1c2[C@@H](NC(=O)CO)CC3] | |
| 1-B | 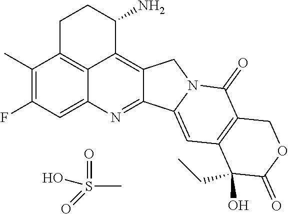 | |
| 1-C | 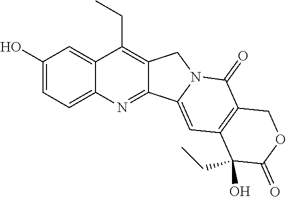 [SMILES: CCc1c2c(nc3ccc(O)cc13)-c1cc3c(c(=O)n1C2)COC(=O)[C@]3(O)CC] | |
| 1-1 | 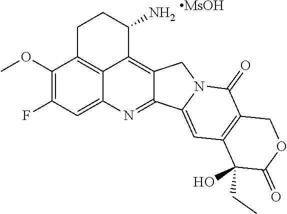 [SMILES: "·MsOH" ; CC[C@@]1(O)C(=O)OCc2c1cc1n(c2=O)Cc2c-1nc1cc(F)c(OC)c3c1c2[C@@H](N)CC3] | |
| 1-2 | 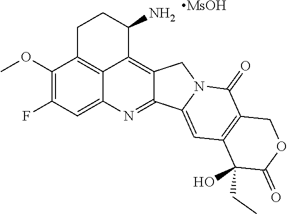 [SMILES: "·MsOH" ; CC[C@@]1(O)C(=O)OCc2c1cc1n(c2=O)Cc2c-1nc1cc(F)c(OC)c3c1c2[C@H](N)CC3] | |
| 1-3 | 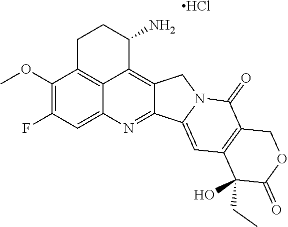 [SMILES: Cl ; CC[C@@]1(O)C(=O)OCc2c1cc1n(c2=O)Cc2c-1nc1cc(F)c(OC)c3c1c2[C@@H](N)CC3] | |
| 1-4 | 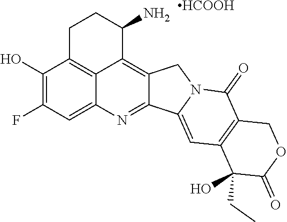 [SMILES: CC[C@@]1(O)C(=O)OCc2c1cc1n(c2=O)Cc2c-1nc1cc(F)c(O)c3c1c2[C@H](N)CC3 ; "·HCOOH"] | |
| 1-5 | 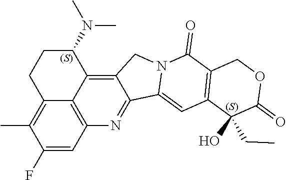 | |
| 1-6 | 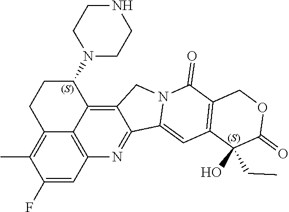 | |
| 1-7 | 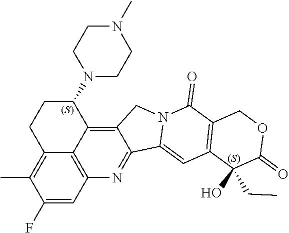 | |
| 1-8 | 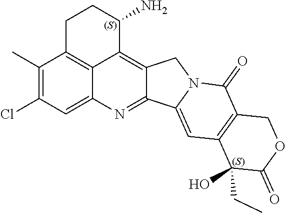 | |
| 1-9 | 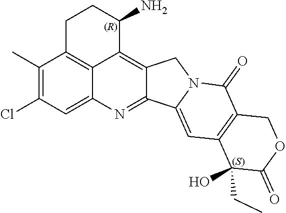 | |
| 1-10 | 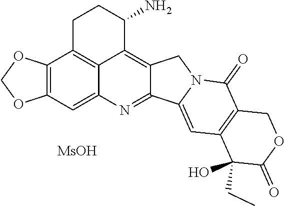 [SMILES: "MsOH" ; CC[C@@]1(O)C(=O)OCc2c1cc1n(c2=O)Cc2c-1nc1cc3c(c4c1c2[C@@H](N)CC4)OCO3] | |
| 1-11 | 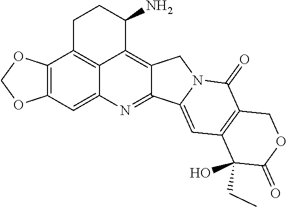 [SMILES: CC[C@@]1(O)C(=O)OCc2c1cc1n(c2=O)Cc2c-1nc1cc3c(c4c1c2[C@H](N)CC4)OCO3] | |
| 1-12 | 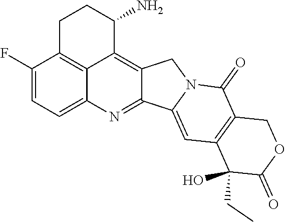 [SMILES: CC[C@@]1(O)C(=O)OCc2c1cc1n(c2=O)Cc2c-1nc1ccc(F)c3c1c2[C@@H](N)CC3] | |
| 1-13 | 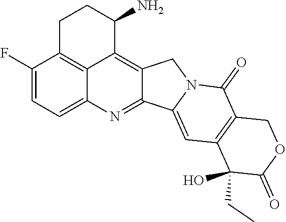 [SMILES: CC[C@@]1(O)C(=O)OCc2c1cc1n(c2=O)Cc2c-1nc1ccc(F)c3c1c2[C@H](N)CC3] | |
| 1-14 | 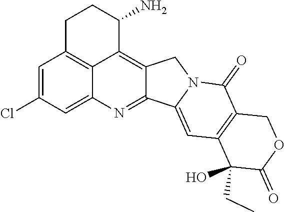 [SMILES: CC[C@@]1(O)C(=O)OCc2c1cc1n(c2=O)Cc2c-1nc1cc(Cl)cc3c1c2[C@@H](N)CC3] | |
| 1-15 | 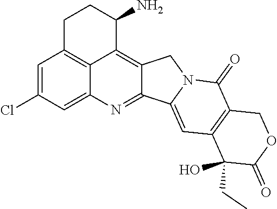 [SMILES: CC[C@@]1(O)C(=O)OCc2c1cc1n(c2=O)Cc2c-1nc1cc(Cl)cc3c1c2[C@H](N)CC3] | |
| 1-16 | 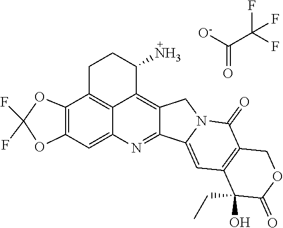 [SMILES: CC[C@@]1(O)C(=O)OCc2c1cc1n(c2=O)Cc2c-1nc1cc3c(c4c1c2[C@@H](N)CC4)OC(F)(F)O3 ; O=C([O-])C(F)(F)F] | |
| 1-17 | 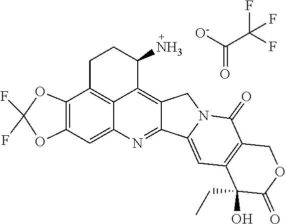 [SMILES: CC[C@@]1(O)C(=O)OCc2c1cc1n(c2=O)Cc2c-1nc1cc3c(c4c1c2[C@H](N)CC4)OC(F)(F)O3 ; O=C([O-])C(F)(F)F] | |
| 1-18 | 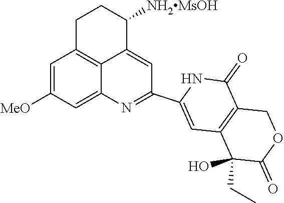 | |
| 1-19 | 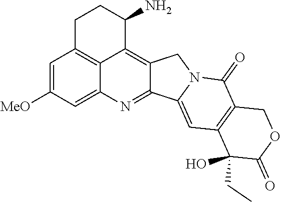 [SMILES: CC[C@@]1(O)C(=O)OCc2c1cc1n(c2=O)Cc2c-1nc1cc(OC)cc3c1c2[C@H](N)CC3] | |
| 1-20 | 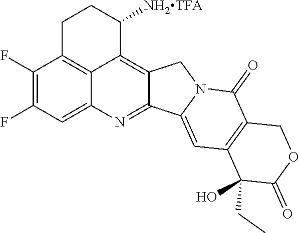 | |
| 1-21 | 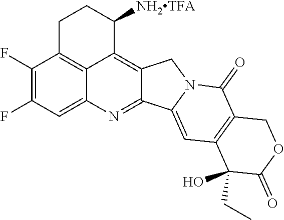 | |
| 1-22 | 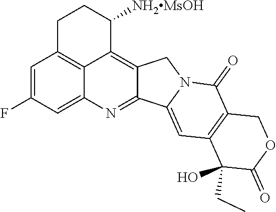 | |
| 1-23 | 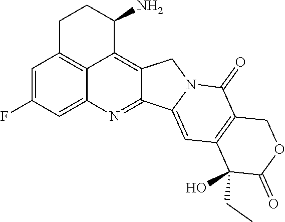 [SMILES: CC[C@@]1(O)C(=O)OCc2c1cc1n(c2=O)Cc2c-1nc1cc(F)cc3c1c2[C@H](N)CC3] | |
| 1-24 | 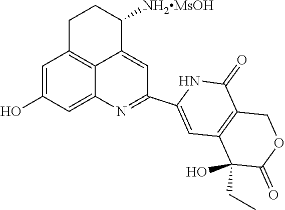 | |
| 1-25 | 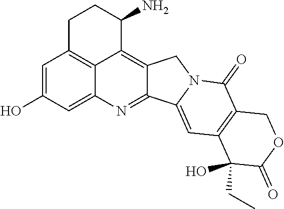 [SMILES: CC[C@@]1(O)C(=O)OCc2c1cc1n(c2=O)Cc2c-1nc1cc(O)cc3c1c2[C@H](N)CC3] | |
| 1-26 | 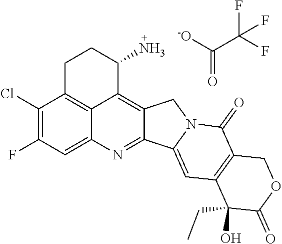 | |
| 1-27 | 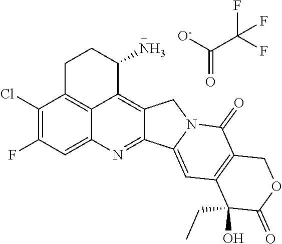 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F] | |

### Cellular Killing Assay

TABLE 2
Exatecan and Camptothecin analogues

| Compound | |
|---|---|
| number | Payload structure |
| 2-1 | 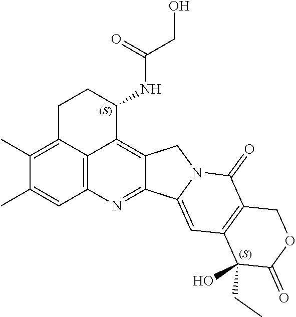 |
| 2-2 | 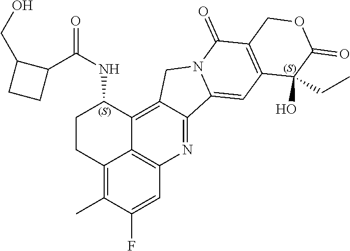 |
| 2-3 | 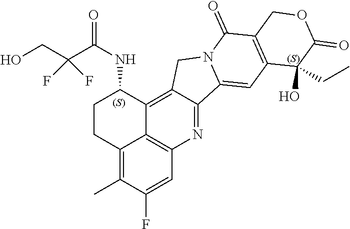 |
| 2-4 | 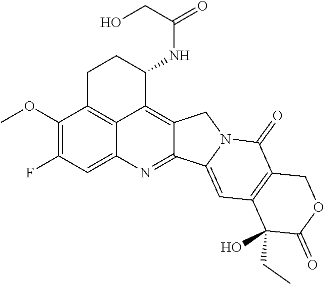 [SMILES: CC[C@@]1(O)C(=O)OCc2c1cc1n(c2=O)Cc2c-1nc1cc(F)c(OC)c3c1c2[C@@H](NC(=O)CO)CC3] |
| 2-5 | 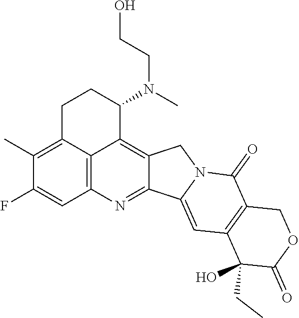 [SMILES: CC[C@@]1(O)C(=O)OCc2c1cc1n(c2=O)Cc2c-1nc1cc(F)c(C)c3c1c2[C@@H](N(C)CCO)CC3] |
| 2-6 | 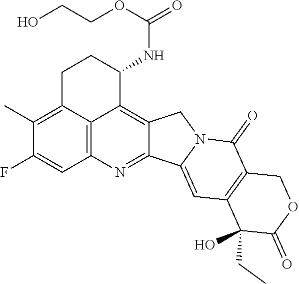 [SMILES: CC[C@@]1(O)C(=O)OCc2c1cc1n(c2=O)Cc2c-1nc1cc(F)c(C)c3c1c2[C@@H](NC(=O)OCCO)CC3] |
| 2-7 | 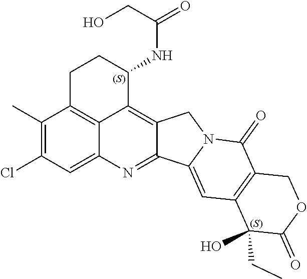 |
| 2-8 | 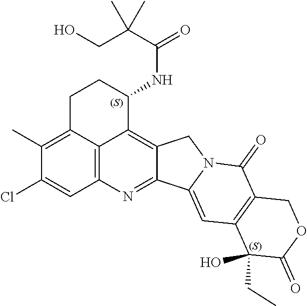 |
| 2-9 | 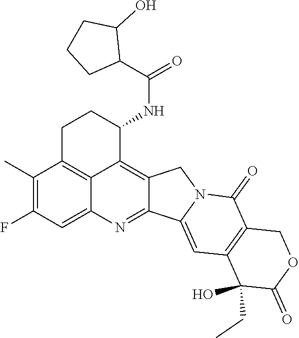 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| | isomer 1 |
| 2-10 | 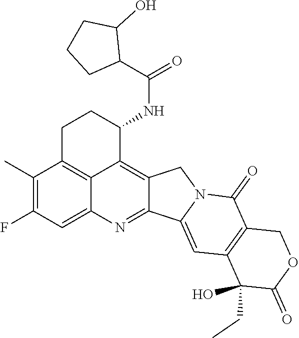 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| | isomer 2 |
| 2-11 | 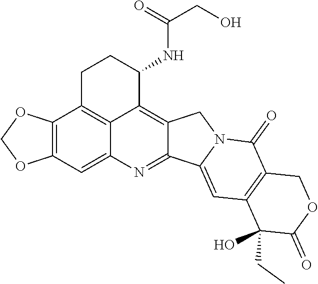 [SMILES: CC[C@@]1(O)C(=O)OCc2c1cc1n(c2=O)Cc2c-1nc1cc3c(c4c1c2[C@@H](NC(=O)CO)CC4)OCO3] |
| 2-12 | 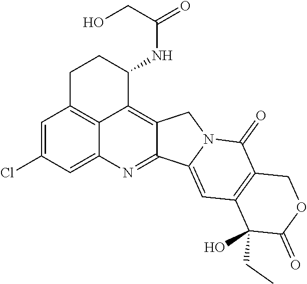 [SMILES: CC[C@@]1(O)C(=O)OCc2c1cc1n(c2=O)Cc2c-1nc1cc(Cl)cc3c1c2[C@@H](NC(=O)CO)CC3] |
| 2-13 | 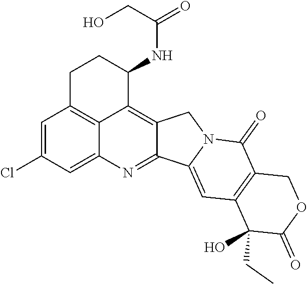 [SMILES: CC[C@@]1(O)C(=O)OCc2c1cc1n(c2=O)Cc2c-1nc1cc(Cl)cc3c1c2[C@H](NC(=O)CO)CC3] |
| 2-14 | 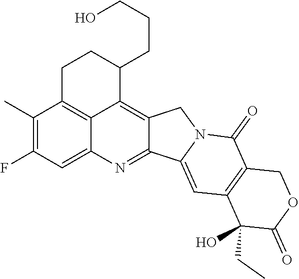 [SMILES: CC[C@@]1(O)C(=O)OCc2c1cc1n(c2=O)Cc2c-1nc1cc(F)c(C)c3c1c2C(CCCO)CC3] |
| 2-15 | 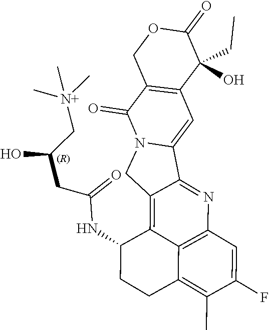 |
| 2-16 | 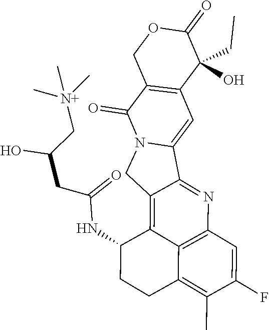 |
| 2-17 | 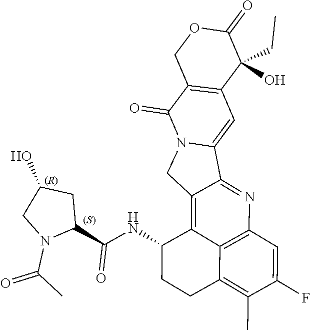 |
| 2-18 | 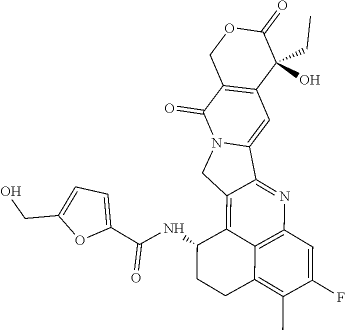 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-19 | 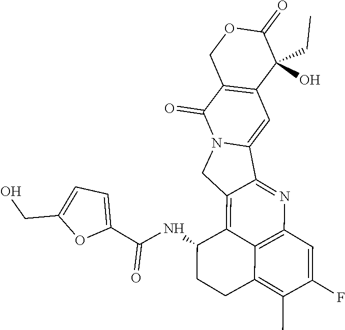 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-20 | 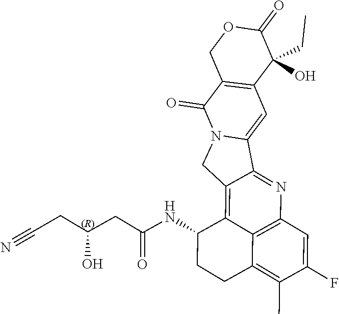 |
| 2-21 | 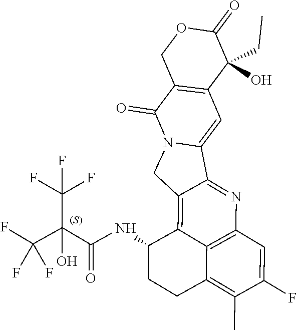 |
| 2-22 | 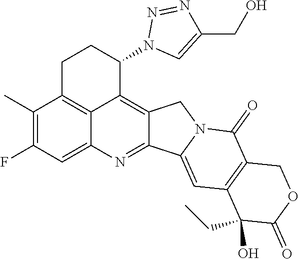 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-23 | 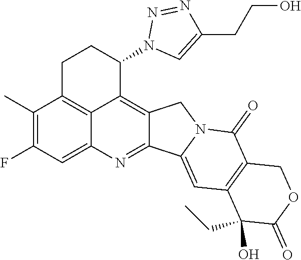 [SMILES: CC[C@@]1(O)C(=O)OCc2c1cc1n(c2=O)Cc2c-1nc1cc(F)c(C)c3c1c2[C@@H](n1cc(CCO)nn1)CC3] |
| 2-24 | 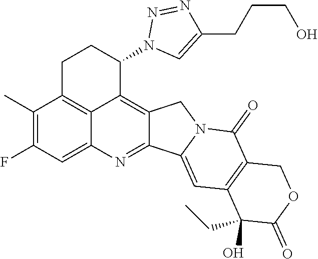 [SMILES: CC[C@@]1(O)C(=O)OCc2c1cc1n(c2=O)Cc2c-1nc1cc(F)c(C)c3c1c2[C@@H](n1cc(CCCO)nn1)CC3] |
| 2-25 | 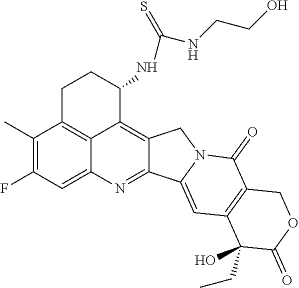 [SMILES: CC[C@@]1(O)C(=O)OCc2c1cc1n(c2=O)Cc2c-1nc1cc(F)c(C)c3c1c2[C@@H](NC(=S)NCCO)CC3] |
| 2-26 | 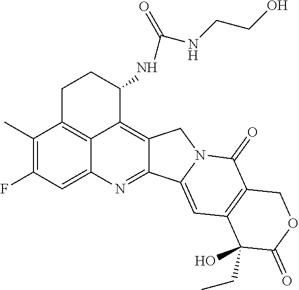 [SMILES: CC[C@@]1(O)C(=O)OCc2c1cc1n(c2=O)Cc2c-1nc1cc(F)c(C)c3c1c2[C@@H](NC(=O)NCCO)CC3] |
| 2-27 | 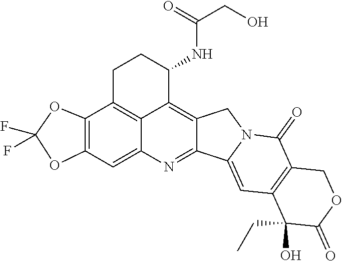 [SMILES: CC[C@@]1(O)C(=O)OCc2c1cc1n(c2=O)Cc2c-1nc1cc3c(c4c1c2[C@@H](NC(=O)CO)CC4)OC(F)(F)O3] |
| 2-28 | 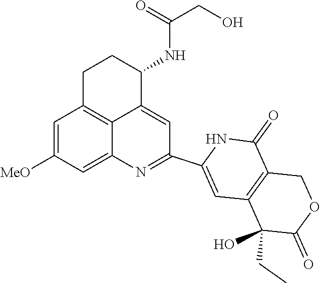 [SMILES: CC[C@@]1(O)C(=O)OCc2c1cc(-c1cc3c4c(cc(OC)cc4n1)CC[C@@H]3NC(=O)CO)[nH]c2=O] |
| 2-29 | 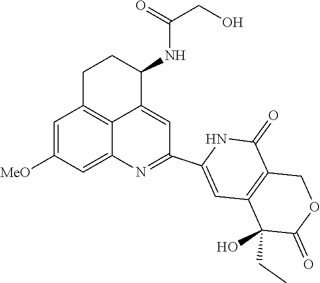 [SMILES: CC[C@@]1(O)C(=O)OCc2c1cc(-c1cc3c4c(cc(OC)cc4n1)CC[C@H]3NC(=O)CO)[nH]c2=O] |
| 2-30 | 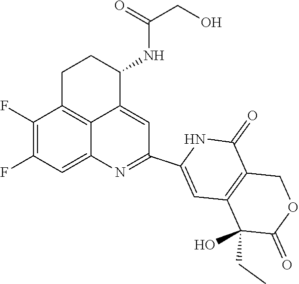 [SMILES: CC[C@@]1(O)C(=O)OCc2c1cc(-c1cc3c4c(c(F)c(F)cc4n1)CC[C@@H]3NC(=O)CO)[nH]c2=O] |
| 2-31 | 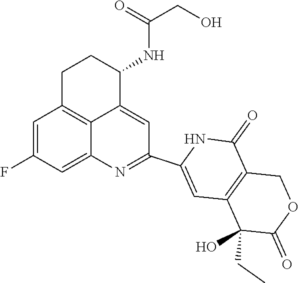 [SMILES: CC[C@@]1(O)C(=O)OCc2c1cc(-c1cc3c4c(cc(F)cc4n1)CC[C@@H]3NC(=O)CO)[nH]c2=O] |
| 2-32 | 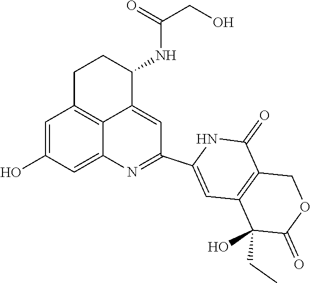 [SMILES: CC[C@@]1(O)C(=O)OCc2c1cc(-c1cc3c4c(cc(O)cc4n1)CC[C@@H]3NC(=O)CO)[nH]c2=O] |
| 2-34 | 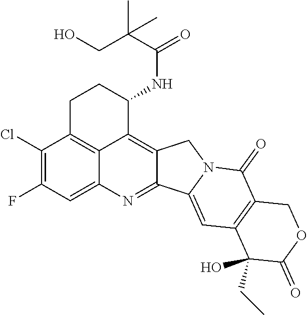 [SMILES: CC[C@@]1(O)C(=O)OCc2c1cc1n(c2=O)Cc2c-1nc1cc(F)c(Cl)c3c1c2[C@@H](NC(=O)C(C)(C)CO)CC3] |
| 2-35 | 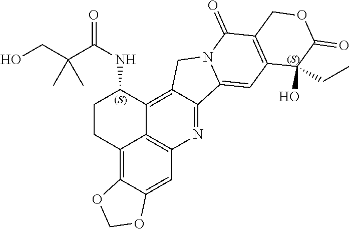 |
| 2-36 | 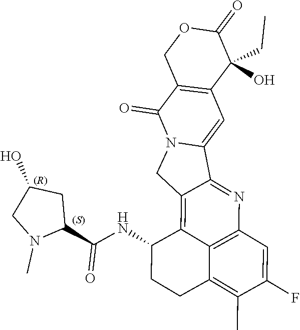 |
| 2-37 | 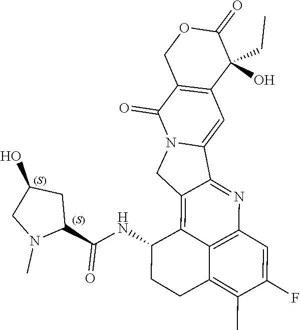 |
| 2-38 | 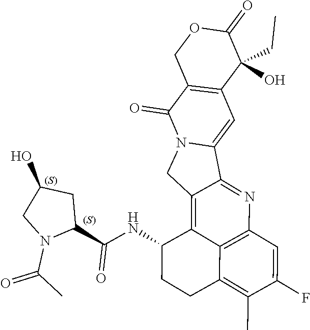 |
| 2-39 | 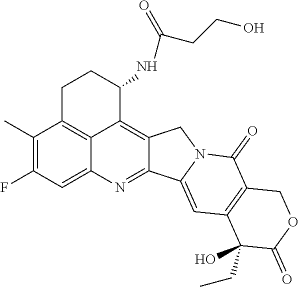 [SMILES: CC[C@@]1(O)C(=O)OCc2c1cc1n(c2=O)Cc2c-1nc1cc(F)c(C)c3c1c2[C@@H](NC(=O)CCO)CC3] |
| 2-40 | 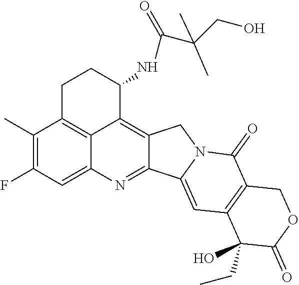 [SMILES: CC[C@@]1(O)C(=O)OCc2c1cc1n(c2=O)Cc2c-1nc1cc(F)c(C)c3c1c2[C@@H](NC(=O)C(C)(C)CO)CC3] |
| 2-41 | 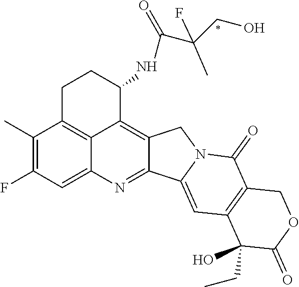 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] |
| 2-42 | 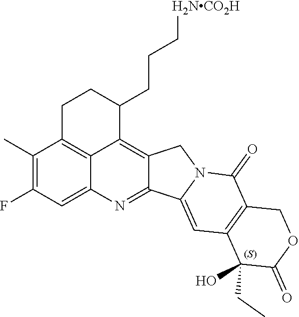 |
| 2-43 | 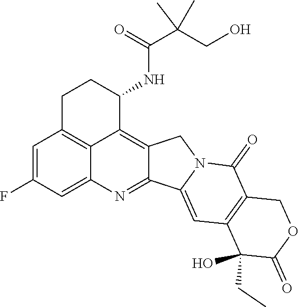 [SMILES: CC[C@@]1(O)C(=O)OCc2c1cc1n(c2=O)Cc2c-1nc1cc(F)cc3c1c2[C@@H](NC(=O)C(C)(C)CO)CC3] |
| 2-44 (peak 1) 2-45 (peak2) | 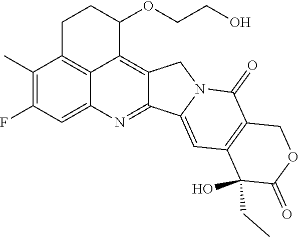 [SMILES: CC[C@@]1(O)C(=O)OCc2c1cc1n(c2=O)Cc2c-1nc1cc(F)c(C)c3c1c2C(OCCO)CC3] |
| 2-46 | 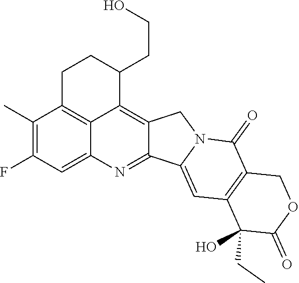 [SMILES: CC[C@@]1(O)C(=O)OCc2c1cc1n(c2=O)Cc2c-1nc1cc(F)c(C)c3c1c2C(CCO)CC3] |
| 2-47 | 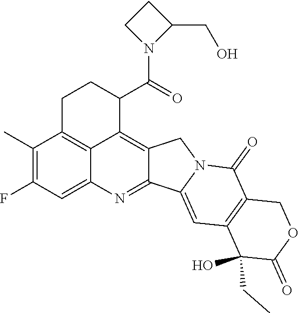 [SMILES: CC[C@@]1(O)C(=O)OCc2c1cc1n(c2=O)Cc2c-1nc1cc(F)c(C)c3c1c2C(C(=O)N1CCC1CO)CC3] |
| 2-48 | 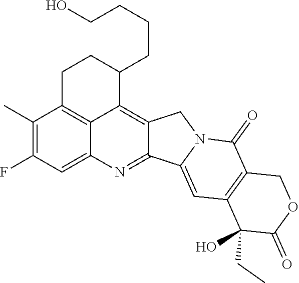 [SMILES: CC[C@@]1(O)C(=O)OCc2c1cc1n(c2=O)Cc2c-1nc1cc(F)c(C)c3c1c2C(CCCCO)CC3] |
| 2-49 (peak 1) 2-50 (peak 2) | 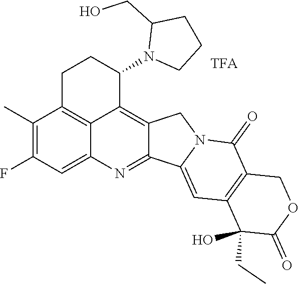 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-51 (peak 1) 2-52 (peak 2) | 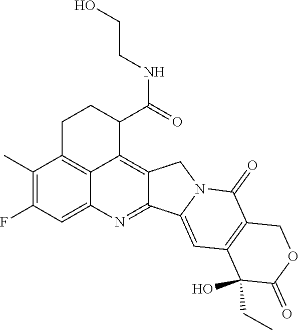 [SMILES: CC[C@@]1(O)C(=O)OCc2c1cc1n(c2=O)Cc2c-1nc1cc(F)c(C)c3c1c2C(C(=O)NCCO)CC3] |
| 2-53 | 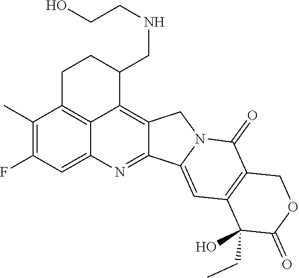 [SMILES: CC[C@@]1(O)C(=O)OCc2c1cc1n(c2=O)Cc2c-1nc1cc(F)c(C)c3c1c2C(CNCCO)CC3] |
| 2-54 | 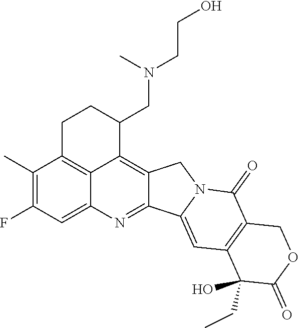 [SMILES: CC[C@@]1(O)C(=O)OCc2c1cc1n(c2=O)Cc2c-1nc1cc(F)c(C)c3c1c2C(CN(C)CCO)CC3] |
| 2-55 | 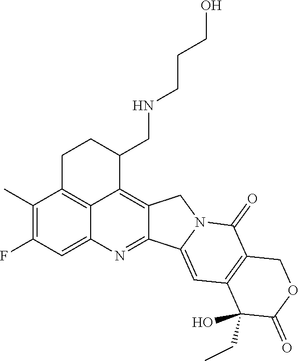 [SMILES: CC[C@@]1(O)C(=O)OCc2c1cc1n(c2=O)Cc2c-1nc1cc(F)c(C)c3c1c2C(CNCCCO)CC3] |
| 2-56 (peak 1) 2-57 (peak 2) | 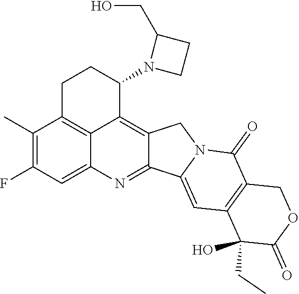 [SMILES: CC[C@@]1(O)C(=O)OCc2c1cc1n(c2=O)Cc2c-1nc1cc(F)c(C)c3c1c2[C@@H](N1CCC1CO)CC3] |
| 2-58 | 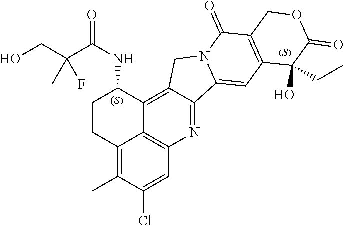 |
| 2-59 (peak 1) 2-60 (peak 2) | 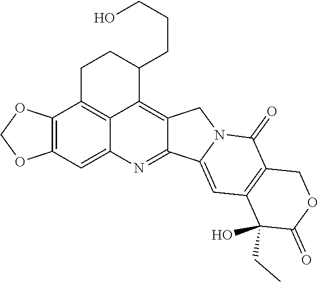 [SMILES: CC[C@@]1(O)C(=O)OCc2c1cc1n(c2=O)Cc2c-1nc1cc3c(c4c1c2C(CCCO)CC4)OCO3] |
| 2-61 | 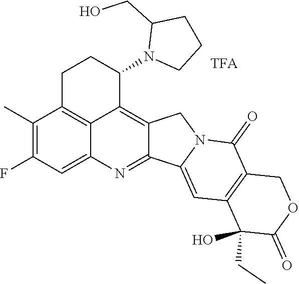 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-62 | 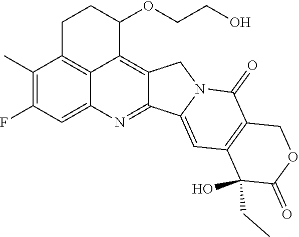 [SMILES: CC[C@@]1(O)C(=O)OCc2c1cc1n(c2=O)Cc2c-1nc1cc(F)c(C)c3c1c2C(OCCO)CC3] |
| 2-63 | 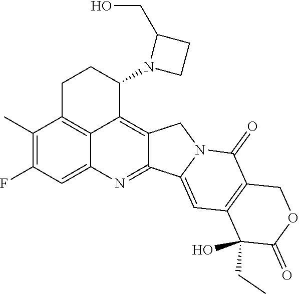 [SMILES: CC[C@@]1(O)C(=O)OCc2c1cc1n(c2=O)Cc2c-1nc1cc(F)c(C)c3c1c2[C@@H](N1CCC1CO)CC3] |
| 2-64 | 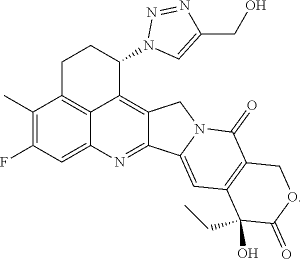 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-65 | 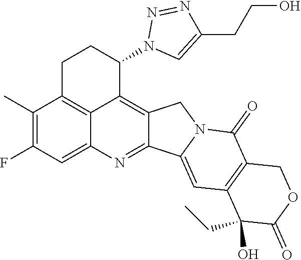 [SMILES: CC[C@@]1(O)C(=O)OCc2c1cc1n(c2=O)Cc2c-1nc1cc(F)c(C)c3c1c2[C@@H](n1cc(CCO)nn1)CC3] |

TABLE 3
Linker + Toxin

| Linker-Payload | |
|---|---|
| number | Linker-Payload Structure |
| 3-1 | 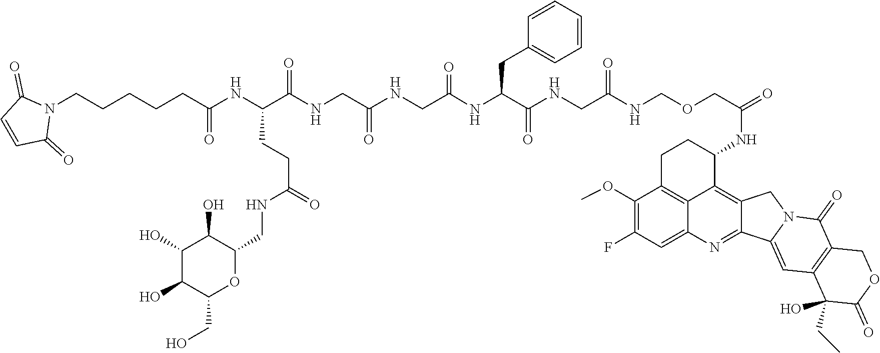 [SMILES: CC[C@@]1(O)C(=O)OCc2c1cc1n(c2=O)Cc2c-1nc1cc(F)c(OC)c3c1c2[C@@H](NC(=O)COCNC(=O)CNC(=O)[C@H](Cc1ccccc1)NC(=O)CNC(=O)CNC(=O)[C@H](CCC(=O)NC[C@@H]1O[C@H](CO)[C@@H](O)[C@H](O)[C@H]1O)NC(=O)CCCCCN1C(=O)C=CC1=O)CC3] |
| 3-2 | 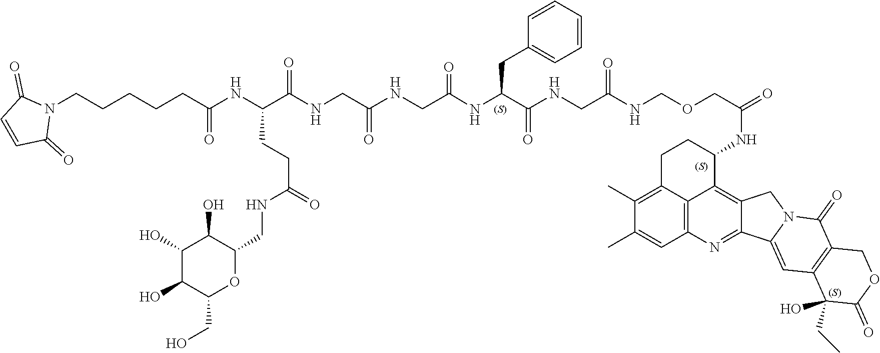 |
| 3-3 | 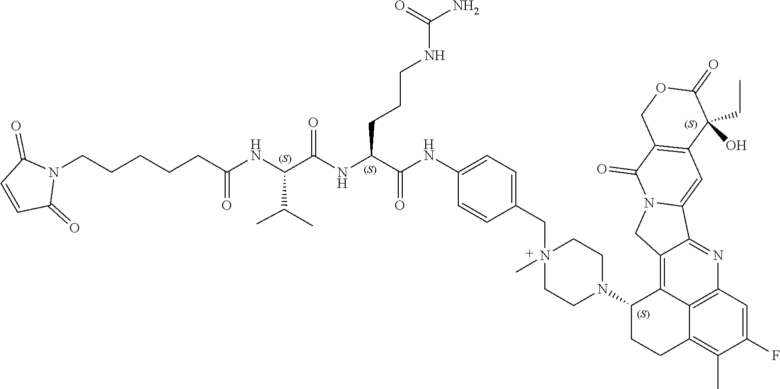 |
| 3-4 | 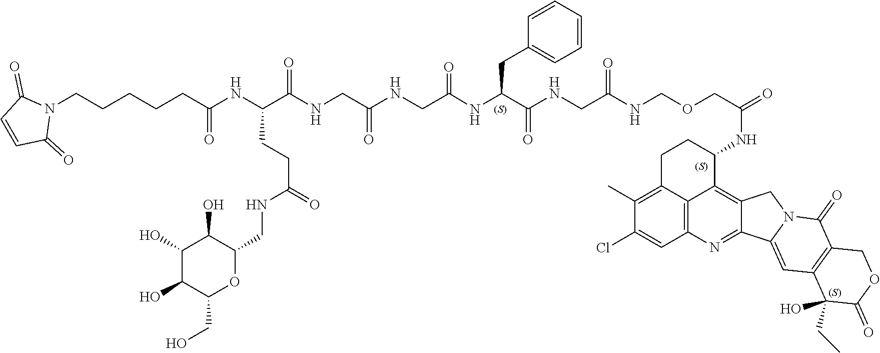 |
| 3-5 | 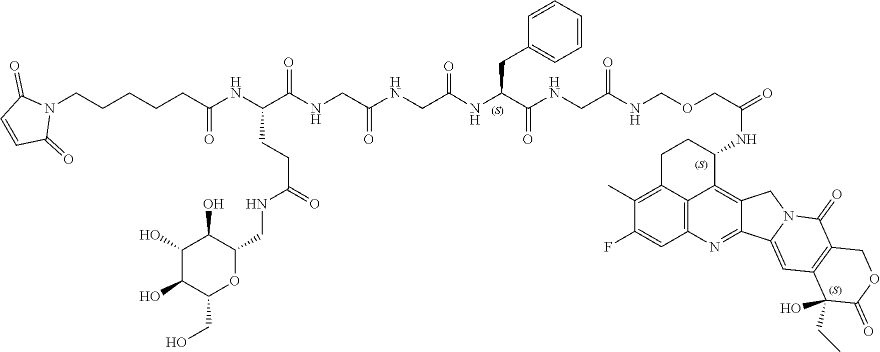 |
| 3-6 | 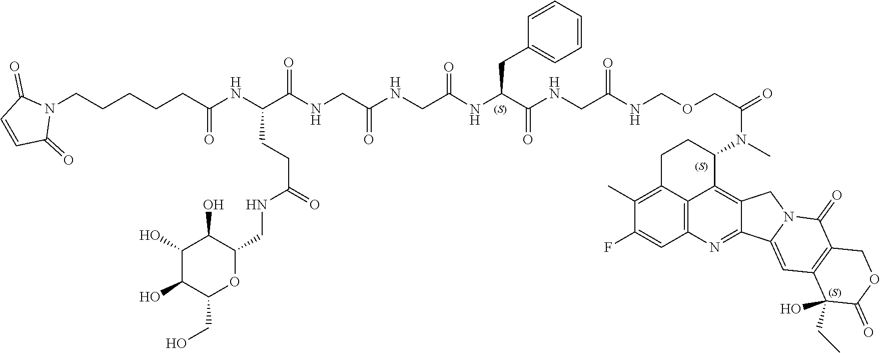 |
| 3-7 | 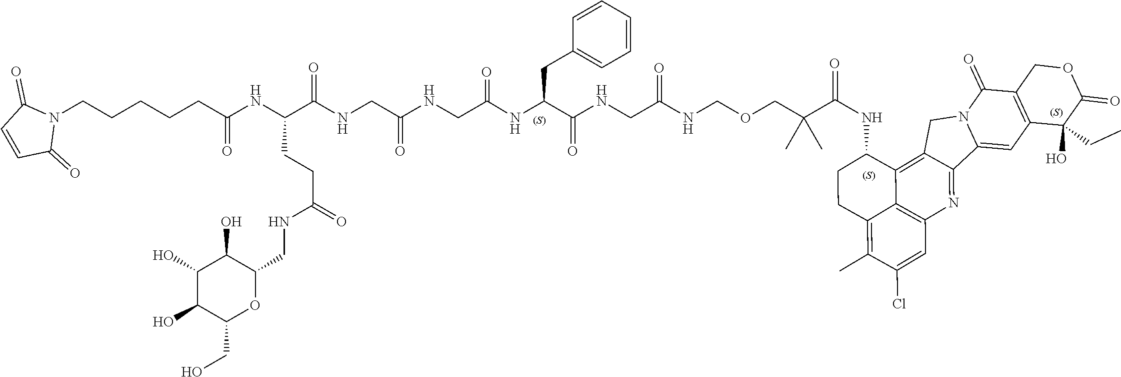 |
| 3-8 | 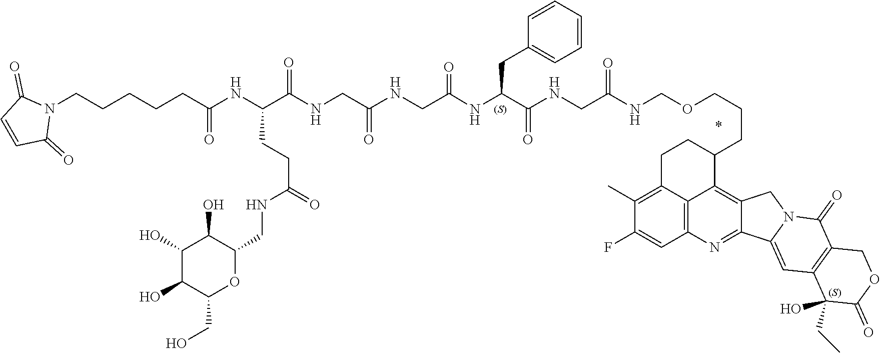 |
| 3-9 | 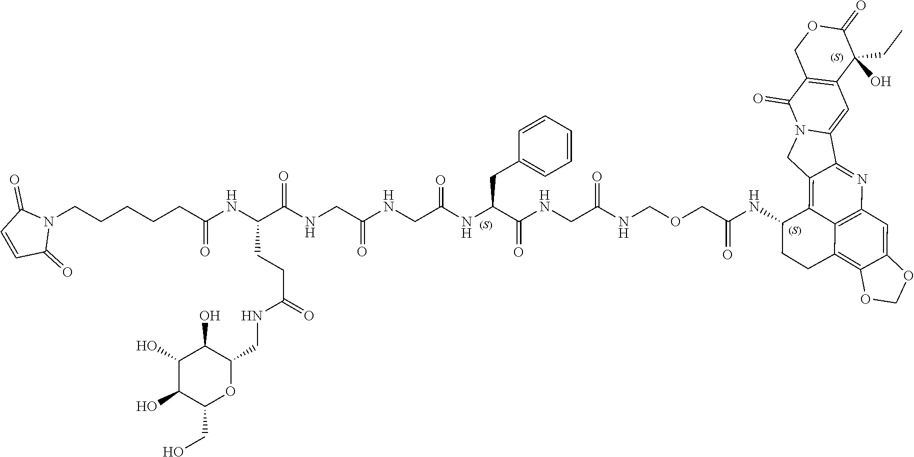 |
| 3-10 | 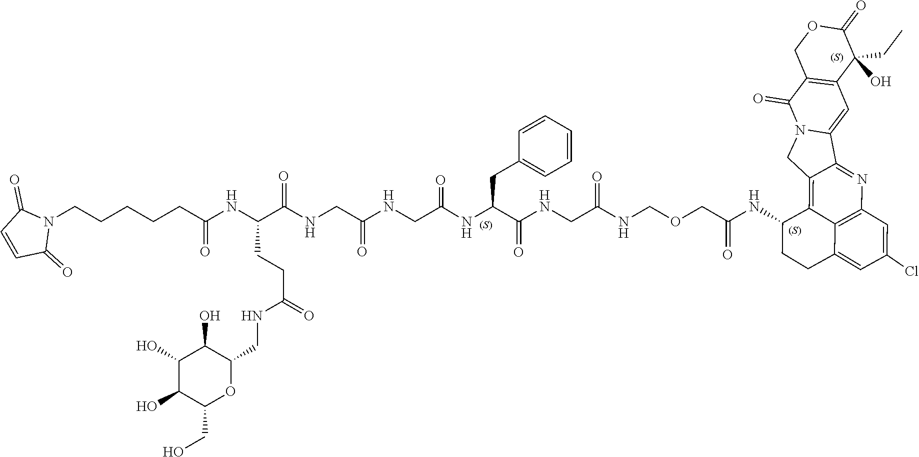 |
| 3-11 | 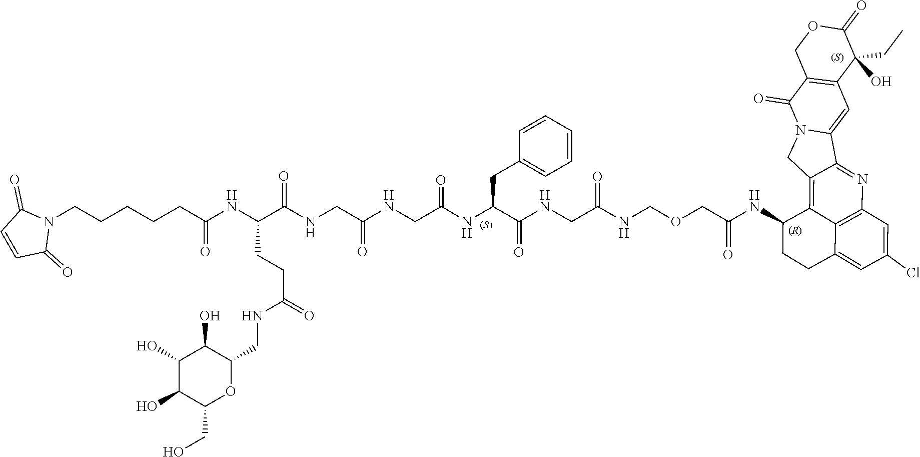 |
| 3-12 | 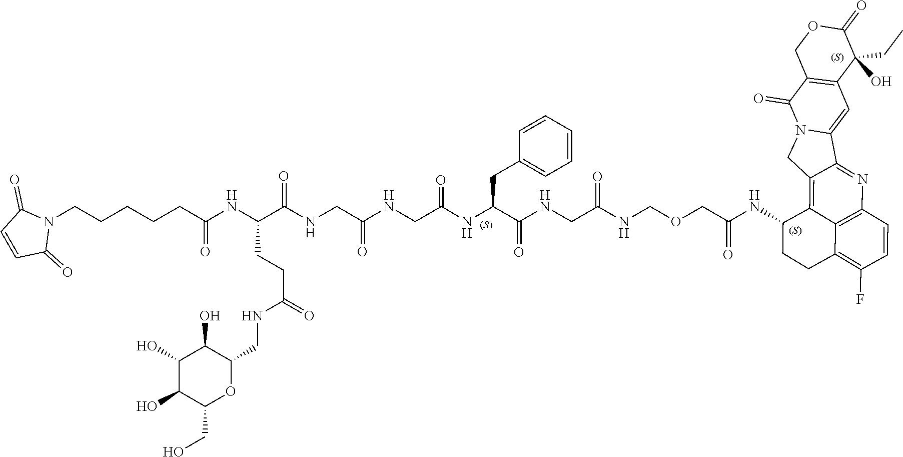 |
| 3-13 | 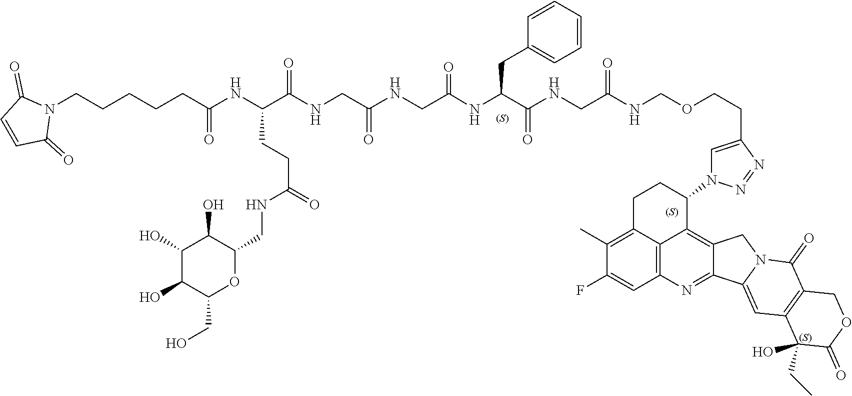 |
| 3-14 | 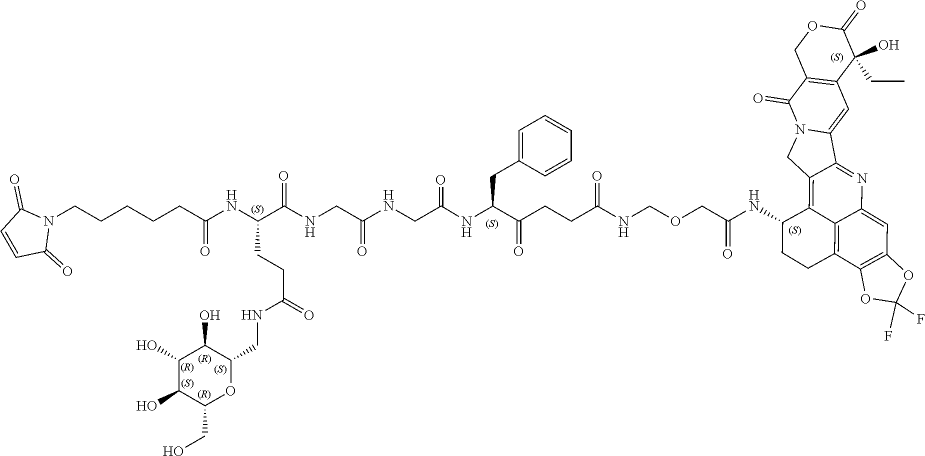 |
| 3-15 | 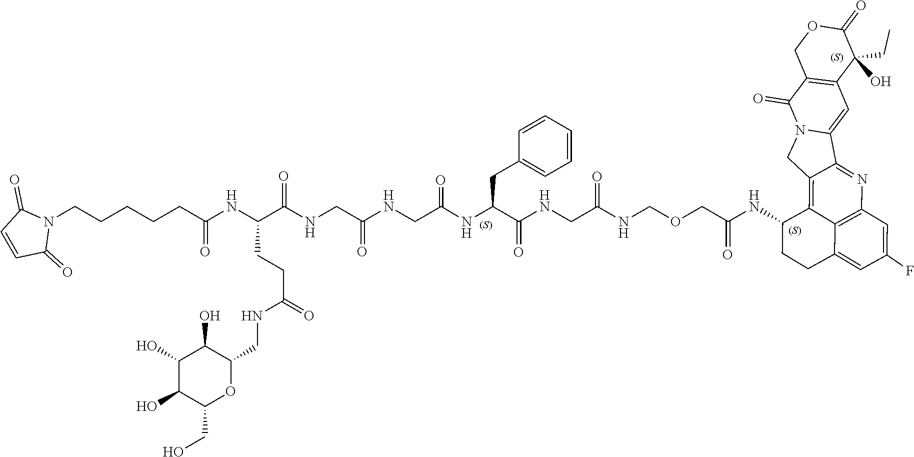 |
| 3-16 | 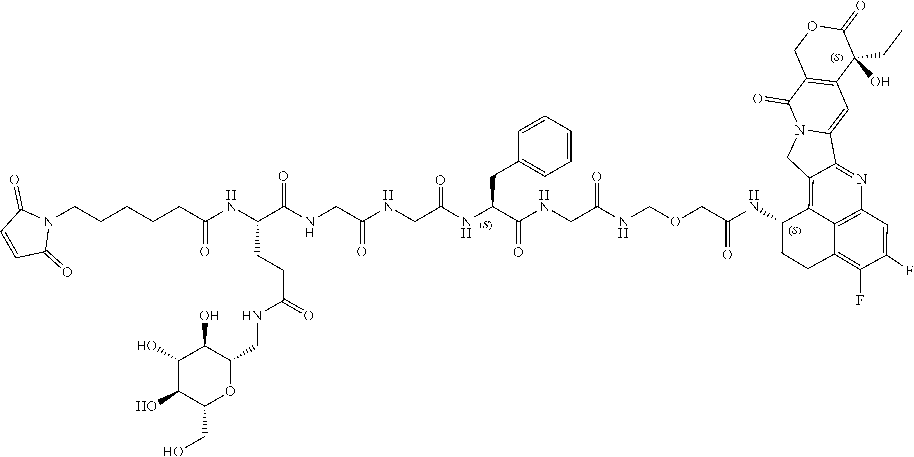 |
| 3-17 | 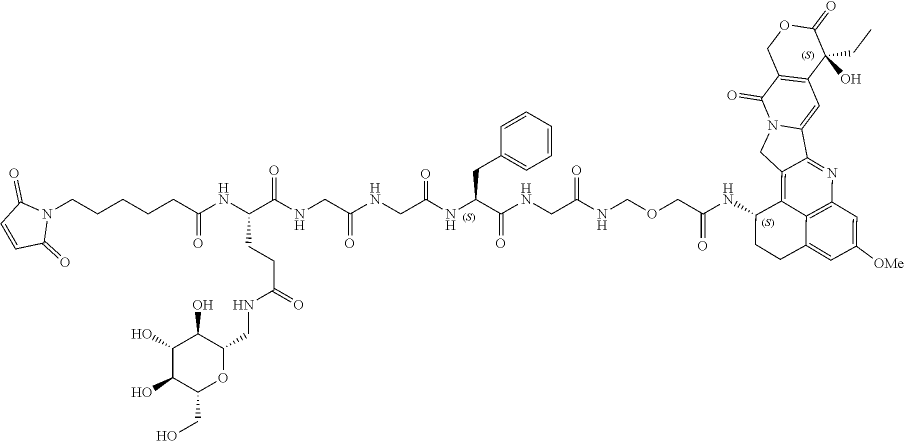 |
| 3-18 | 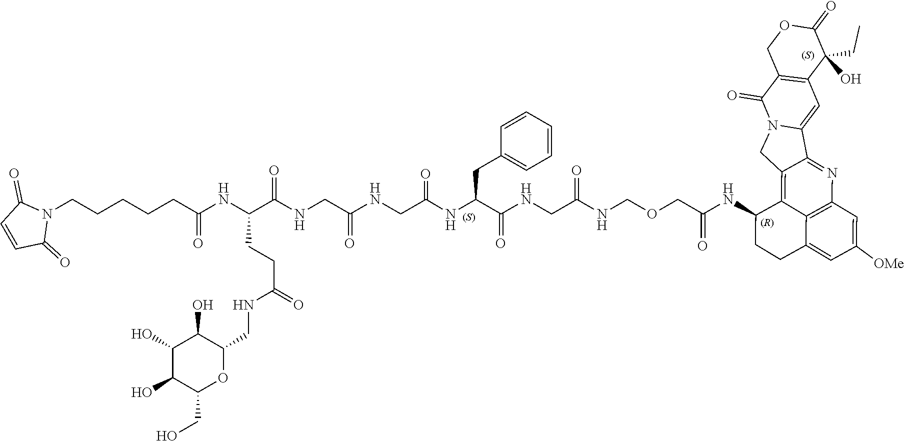 |
| 3-20 | 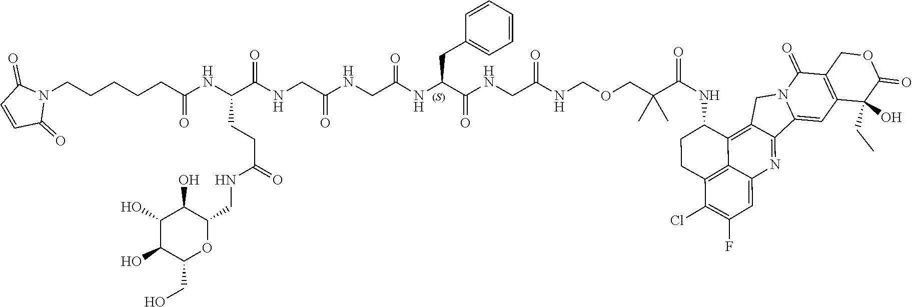 |
| 3-21 | 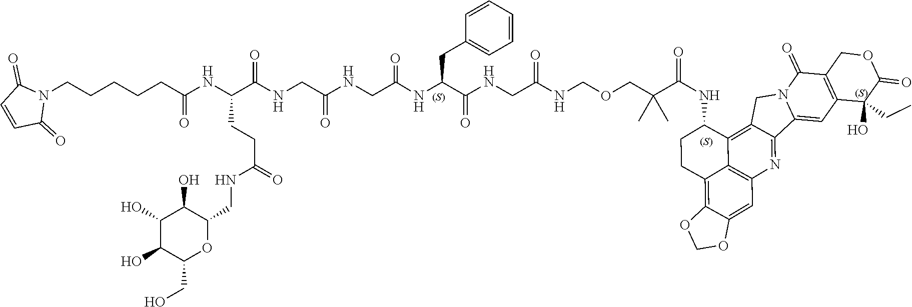 |
| 3-22 | 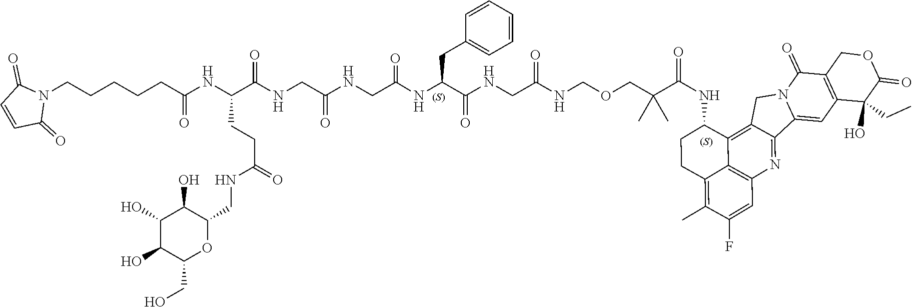 |
| 3-23 (41127) | 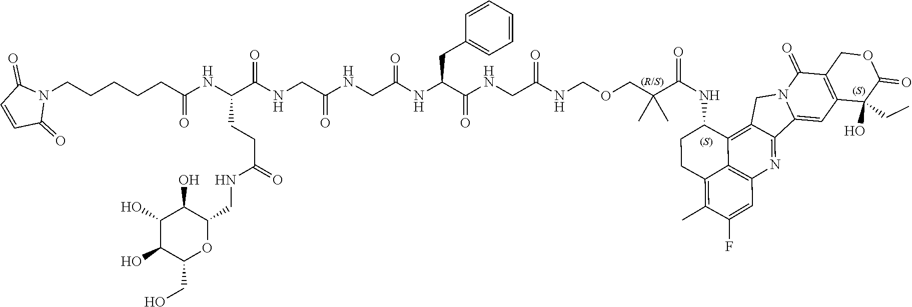 |
| 3-24 | 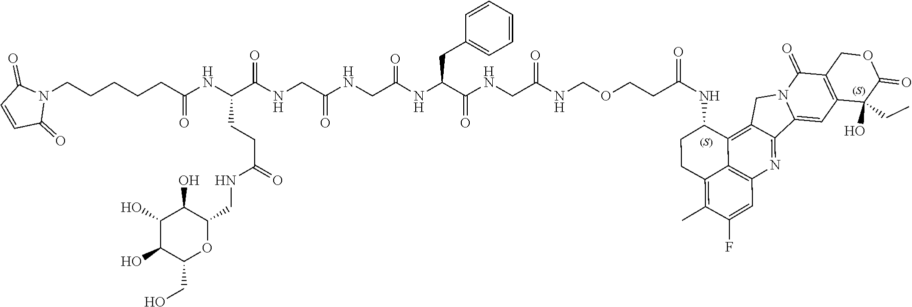 |
| 3-25 | 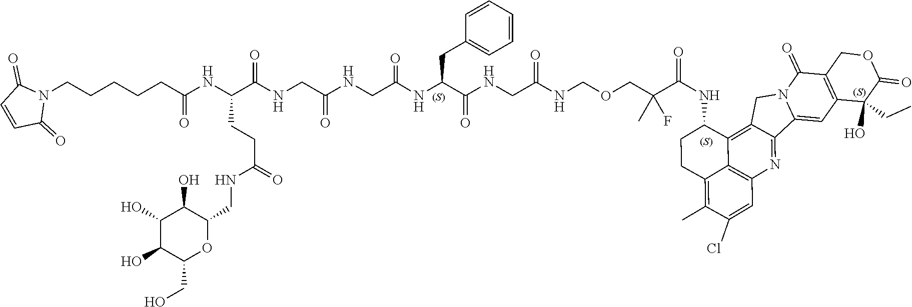 |
| 3-26 | 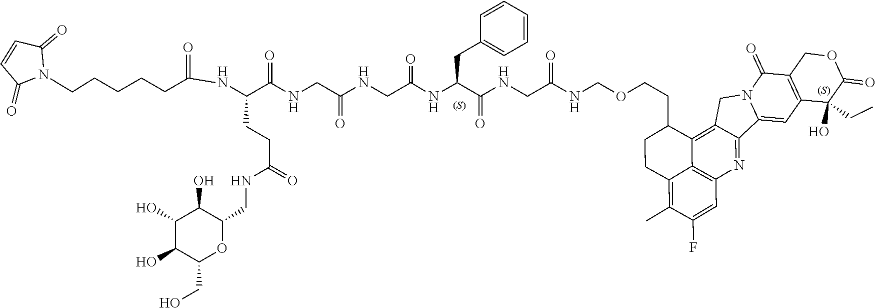 |
| 3-27 | 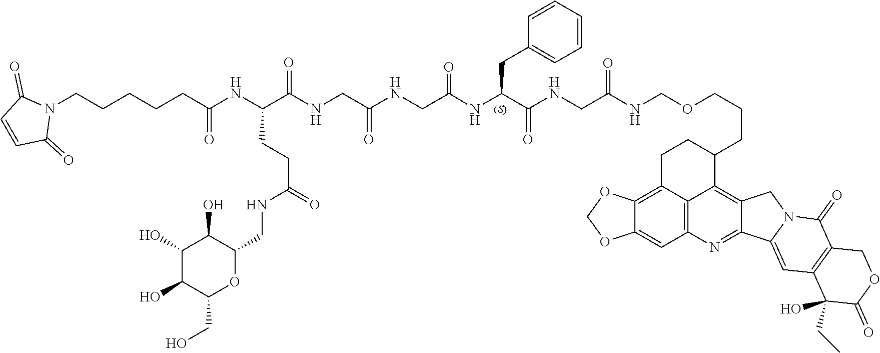 |
| 3-28 | 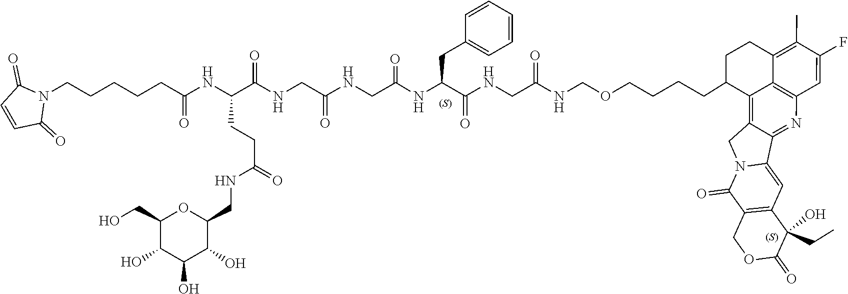 |

### ADC Preparation and Characterization

### Antibody Drug Conjugate Preparation

### DAR8 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction can be quenched with N-acetyl Cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80).

### ADC Characterization

ADC examples in this invention were prepared by following above procedures with DAR 8 profile. All ADCs in this invention were characterized via following analytical methods.

Drug to antibody ratio (DAR) of the ADCs in this invention were determined by LCMS method or HIC method.

SEC purity of ADCs made in this invention are all >95% purity.

### Drug to Antibody Ratio (DAR) Determination

### LCMS Method

LC-MS analysis was carried out under the following measurement conditions:

LC-MS system: Vanquish Flex UHPLC and Orbitrap Exploris 240 Mass Spectrometer

Column: MAbPac™ RP, 2.1*50 mm, 4 μm, 1,500 Å, Thermo Scientific™

Column temperature: 80° C.

Mobile phase A: 0.1% formic acid (FA) aqueous solution

Mobile phase B: Acetonitrile solution containing 0.1% formic acid (FA)

Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)

Injected sample amount: 1 μg

MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

### Hic Method

HPLC analysis was carried out under the following measurement conditions:

HPLC system: Waters ACQUITY ARC HPLC System

Detector: measurement wavelength: 280 nm

Column: Tosoh Bioscience 4.6 μm ID×3.5 cm, 2.5 μm butyl-nonporous resin column

Column temperature: 25° C.

Mobile phase A: 1.5 M ammonium sulfate, 50 mM Phosphate buffer, pH 7.0

Mobile phase B: 50 mM Phosphate buffer, 25% (V/V) Isopropanol, pH 7.0

Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)

Injected sample amount: 20 g

### ADC Purity

### SEC Method

HPLC analysis was carried out under the following measurement conditions:

HPLC system: Waters H-Class UPLC System

Detector: measurement wavelength: 280 nm

Column: ACQUITY UPLC BEH200 SEC 1.7 um 4.6×150 mm, Waters

Column temperature: room temperature

Mobile phase A: 200 mM Phosphate buffer, 250 mM potassium chloride, 15% isopropyl alcohol, PH 7.0

Gradient program: under 10 min isocratic elutions with the flow rate of 0.3 mL/min

Injected sample amount: 20 g

### ADC Hydrophobicity Evaluation

ADC with more hydrophobic property would appear with later retention time from HIC (hydrophobicity interaction column) chromatography. In this invention, the DAR8 (antibody with 8 drug-loading) peak of the example ADCs for this comparison.

### Hic Method

### Method 1

HPLC analysis was carried out under the following measurement conditions:

HPLC system: Waters ACQUITY ARC HPLC System

Detector: measurement wavelength: 280 nm

Column: Tosoh Bioscience 4.6 μm ID×3.5 cm, 2.5 μm butyl-nonporous resin column

Column temperature: 25° C.

Mobile phase A: 1.5 M ammonium sulfate, 50 mM Phosphate buffer, pH 7.0

Mobile phase B: 50 mM Phosphate buffer, 25% (V/V) Isopropanol, pH 7.0

Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)

Injected sample amount: 20 g

### Method 2

HPLC analysis was carried out under the following measurement conditions:

HPLC system: Waters ACQUITY ARC HPLC System

Detector: measurement wavelength: 280 nm

Column: MABPac HIC-10, 5 m, 4.6×10 mm (Thermo)

Column temperature: 25° C.

Mobile phase A: 1.5 M ammonium sulfate, 50 mM sodium phosphate, pH 7.0

Mobile phase B: 50 mM sodium phosphate, pH 7.0

Gradient program: 20% B-20% B (0 min-1 min), 0% B-0% B (1 min-35 min), 20% B-20% B (35 min-40 min)

Flow rate: 0.5 mL/min

Sample preparation: The sample was diluted with initial mobile phase to 0.5 mg/mL.

TABLE 4
ADC Examples

| | | | DAR8 peak HIC |
|---|---|---|---|
| Examples | Antibody | Structures | retention time (min) |
| ADC-P1 BGB-C- 269 | mAb10 | 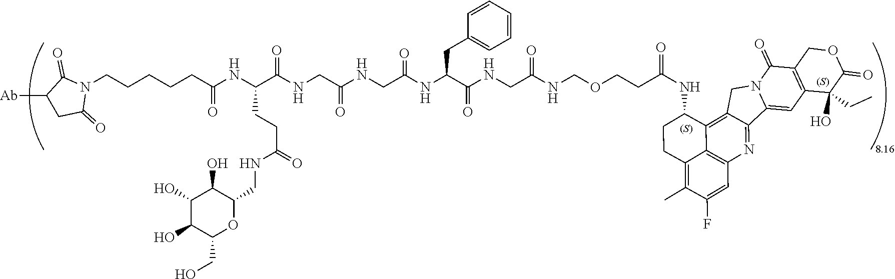 | Method 1: 9.983 |
| ADC-P2 BGB-C- 276 | mAb10 | 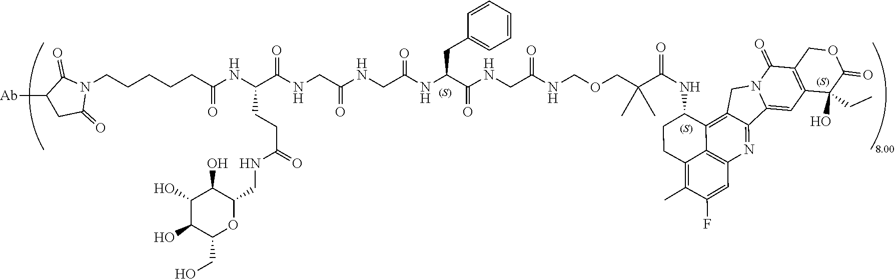 | Method 1: 10.116 |
| ADC-P3 BGB-C- 290 | mAb10 | 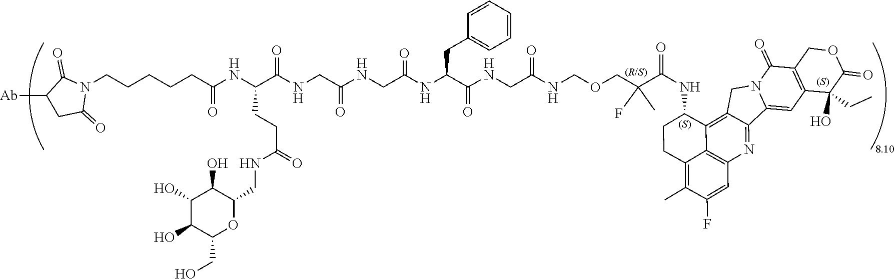 | Method 1: 9.956 |
| ADC-1 BGB-C- 81 | Patrituma b | 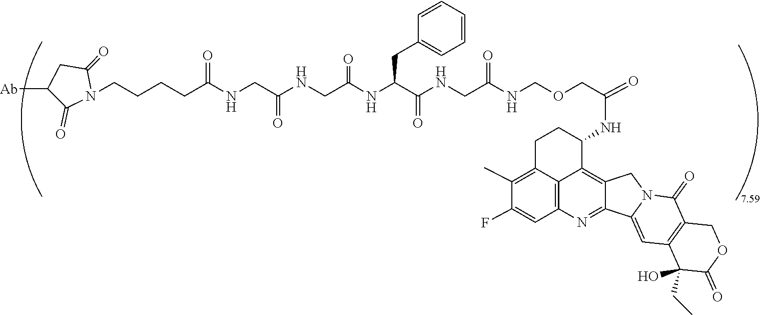 | Method 1: 10.714 |
| ADC-2 BGB-C- 115 | Patrituma b | 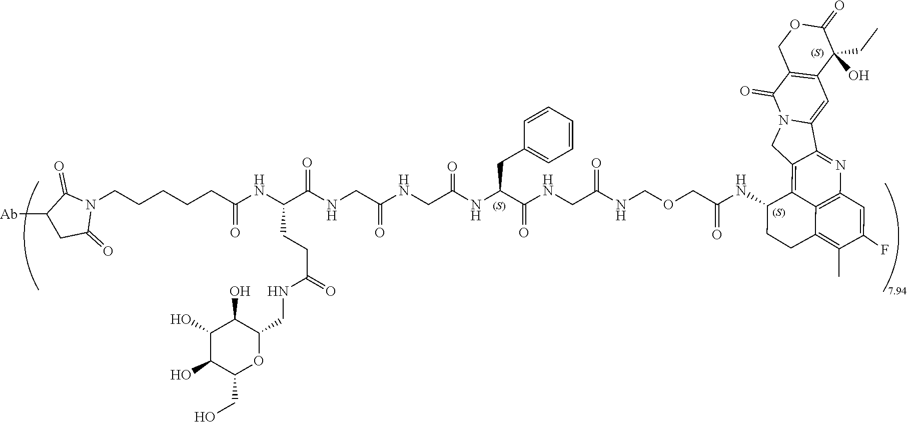 | Method 1: 10.233 |
| ADC-3 BGB-C- 245 | mAb10 | 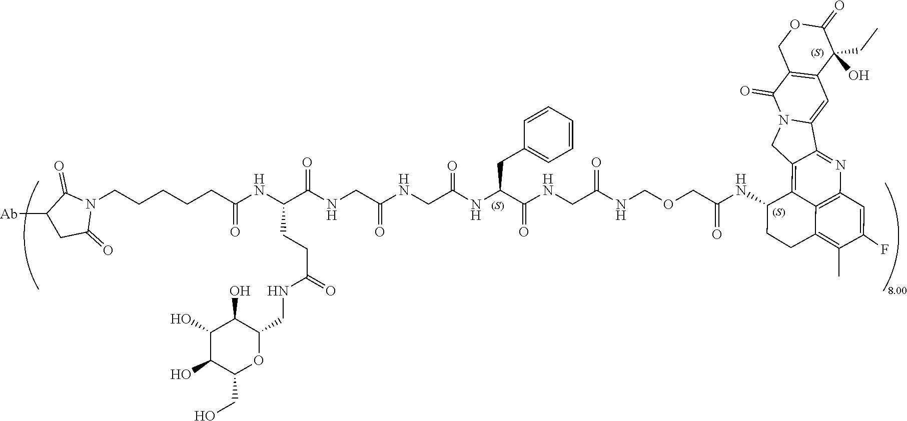 | Method 1: 9.852 |
| ADC-4 BGB-C- 318 | mAb10 | 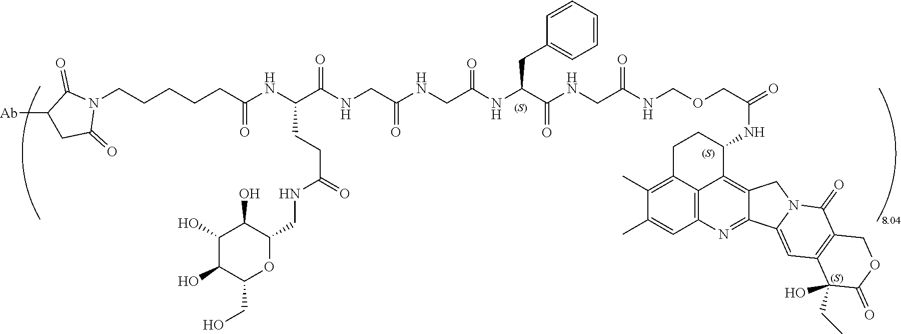 | Method 1: 9.473 |
| ADC-5 BGB-C- 277 | mAb10 | 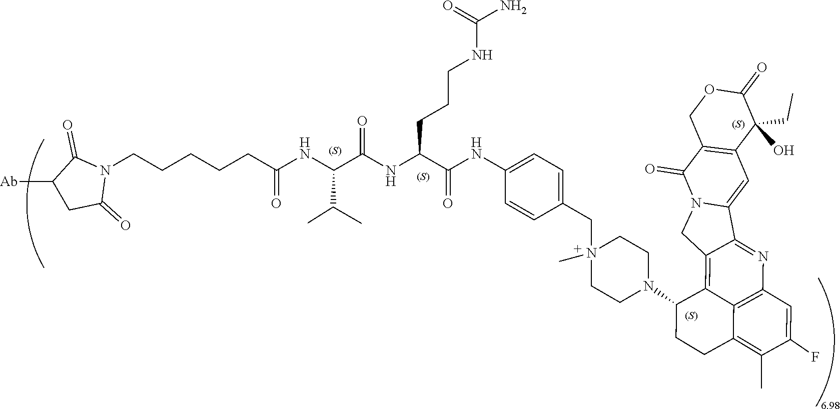 | Method 1: 9.298 |
| ADC-6 BGB-C- 306 | mAb10 | 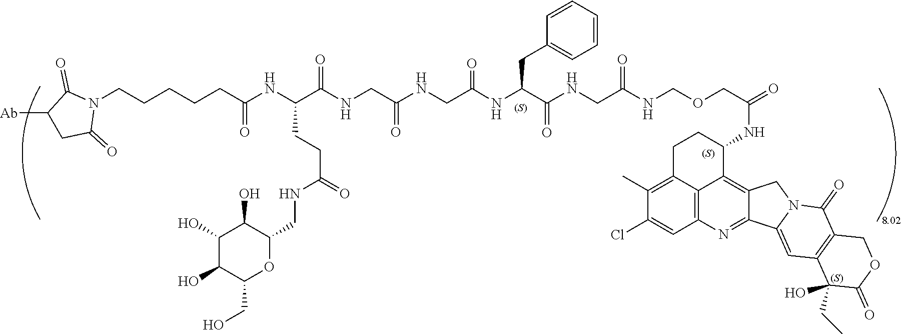 | Method 1: 9.580 |
| ADC-7 BGB-C- 329 | mAb10 | 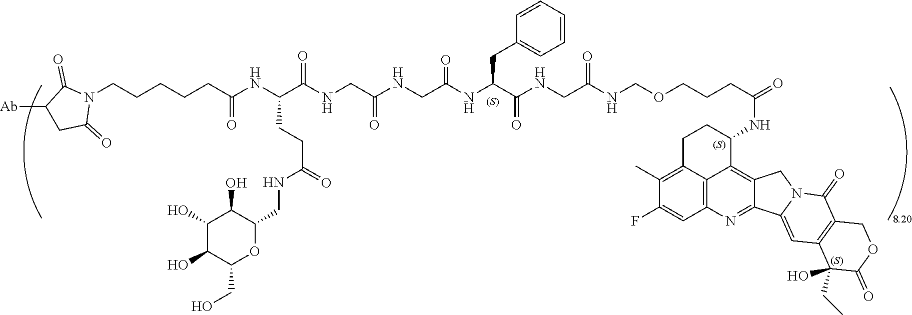 | Method 1: 9.694 |
| ADC-8 BGB-C- 323 | mAb10 | 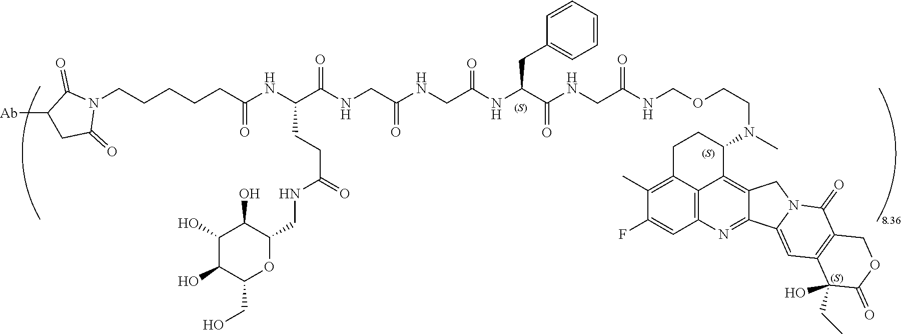 | Method 1: 10.376 |
| ADC-9 BGB-C- 331 | mAb10 | 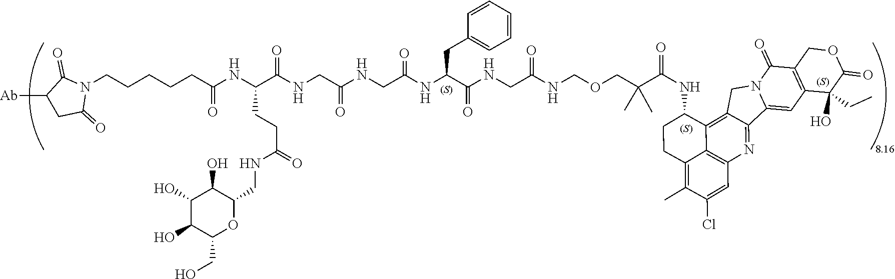 | Method 1: 10.271 |
| ADC-10 BGB-C- 325 | mAb10 | 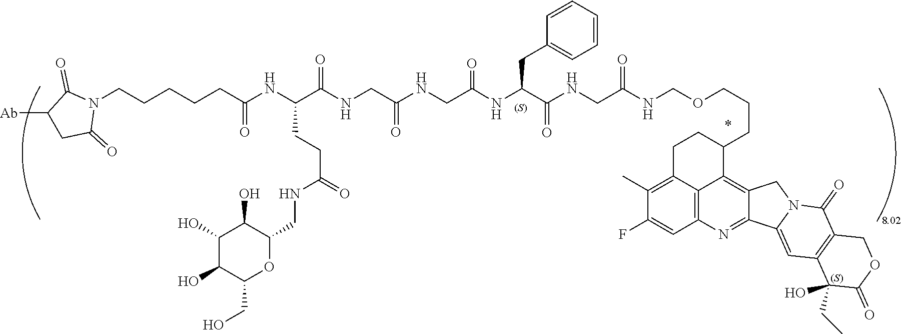 | Method 1: 10.066 |
| ADC-11 BGB-C- 307 | mAb10 | 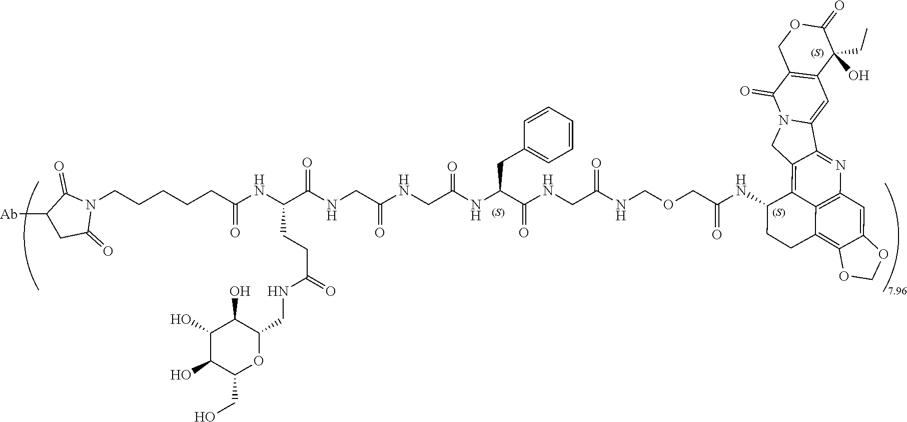 | Method 1: 9.107 |
| ADC-12 BGB-C- 287 | mAb10 | 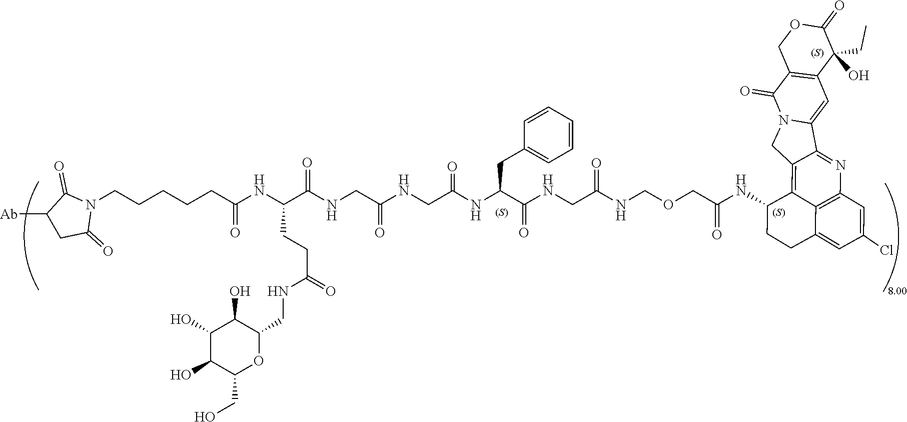 | Method 1 9.820 |
| ADC-13 BGB-C- 288 | mAb10 | 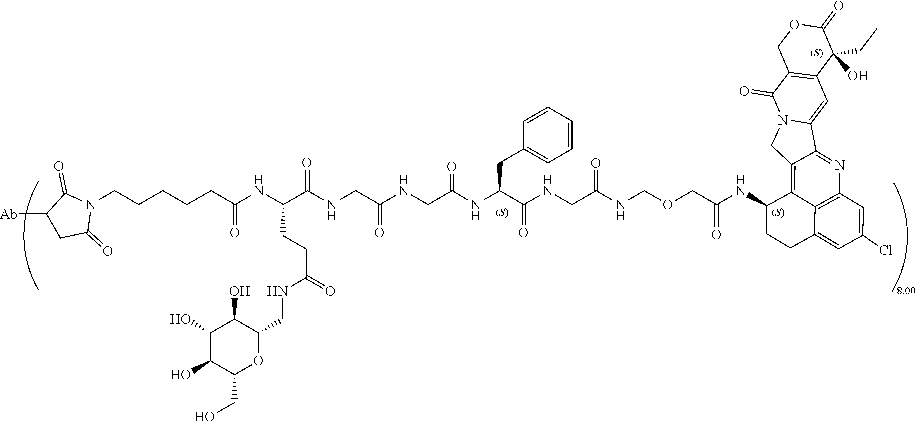 | Method 1: 9.648 |
| ADC-14 BGB-C- 317 | mAb10 | 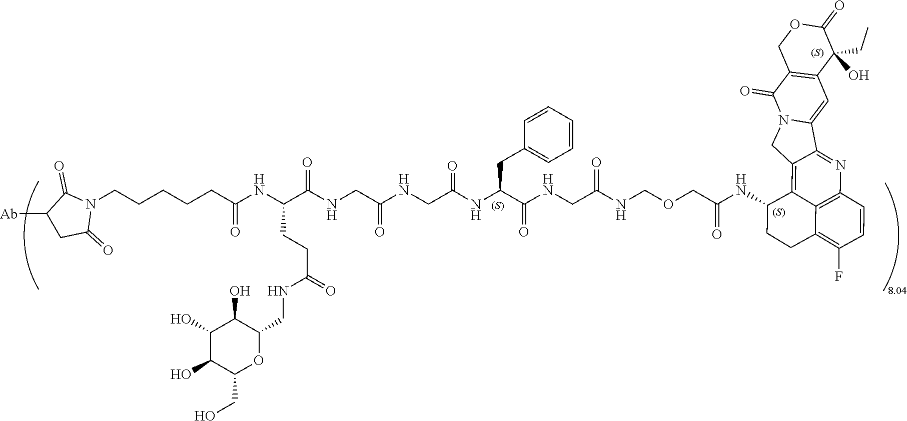 | Method 1: 9.600 |
| ADC-15 BGB-C- 292 | mAb10 | 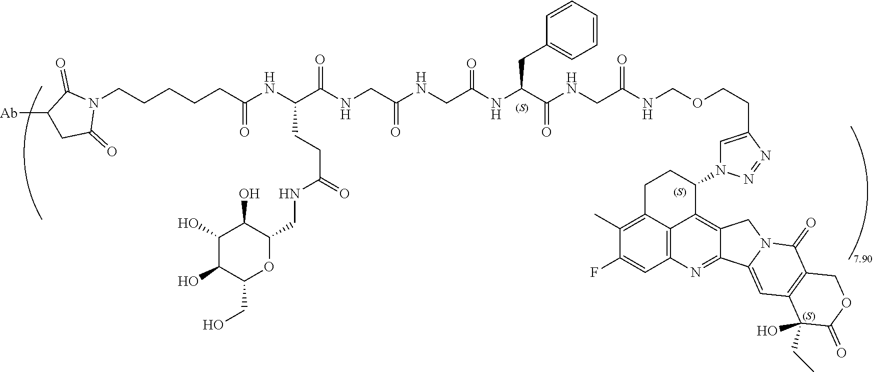 | Method 1: 9.876 |
| ADC-16 BGB-C- 348 | mAb10 | 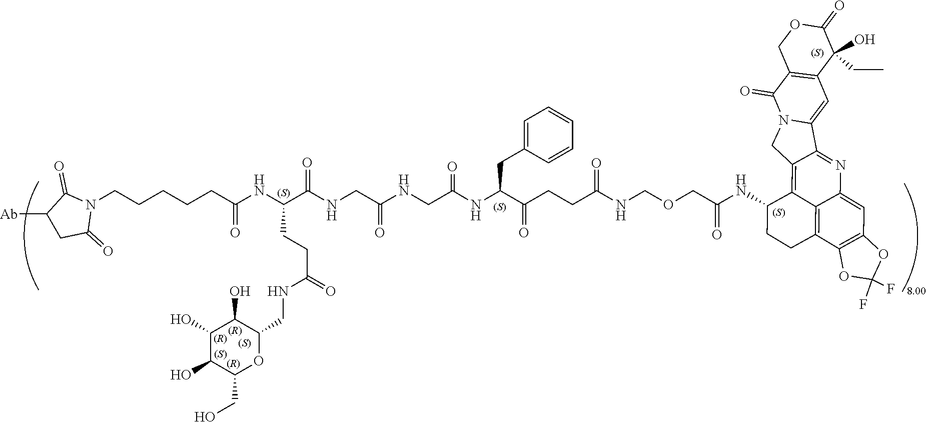 | Method 1: 10.565 |
| ADC-17 BGB-C- 319 | mAb10 | 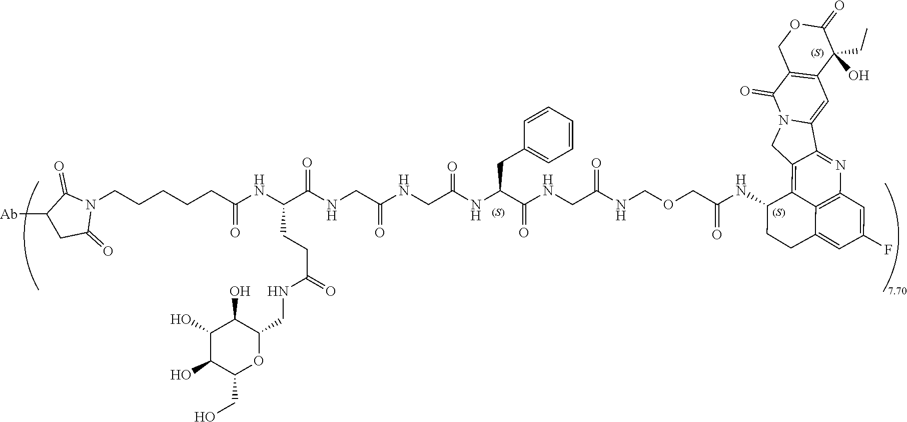 | Method 1: 9.574 |
| ADC-18 BGB-C- 343 | mAb10 | 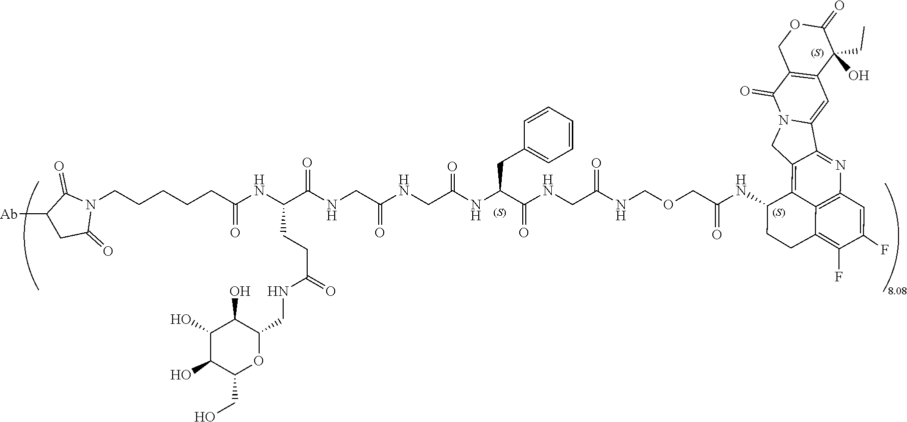 | Method 1: 9.922 |
| ADC-19 BGB-C- 302 | mAb10 | 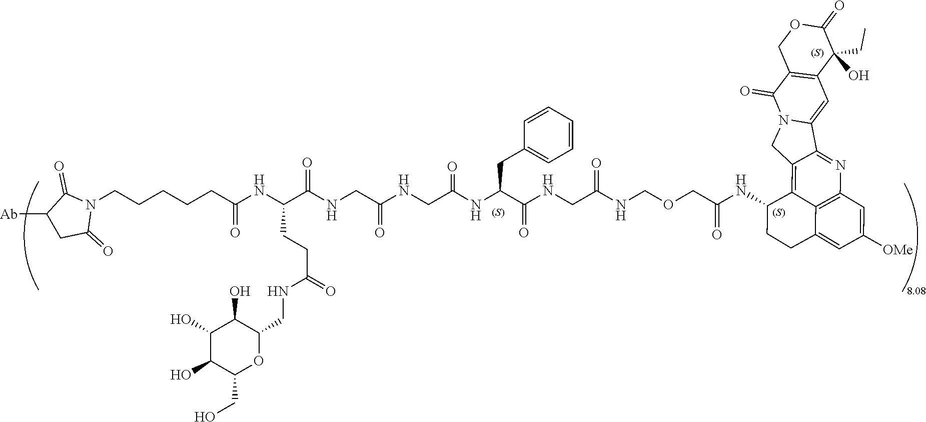 | Method 1: 9.374 |
| ADC-20 BGB-C- 320 | mAb10 | 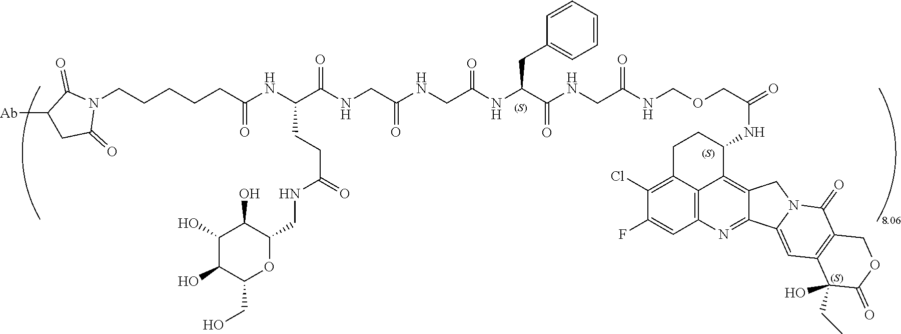 | Method 1: 9.979 |
| ADC-21 BGB-C- 330 | mAb10 | 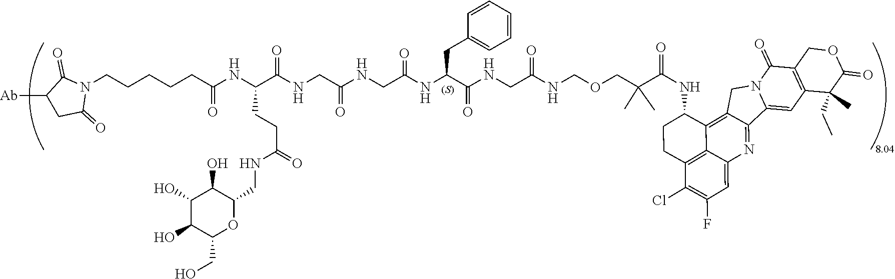 | Method 1: 10.572 |
| ADC-22 BGB-C- 346 | mAb10 | 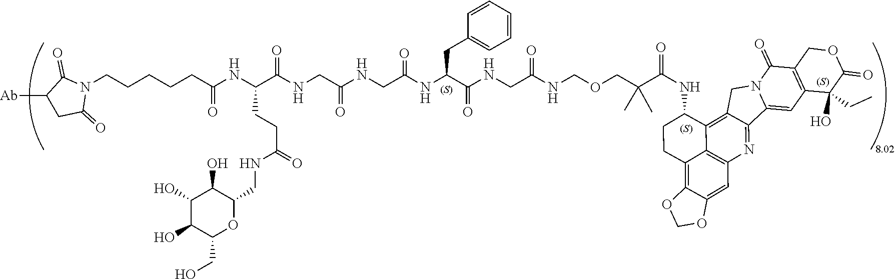 | Method 1: 9.643 |
| ADC-23 BGB-C- 358 | mAb10 | 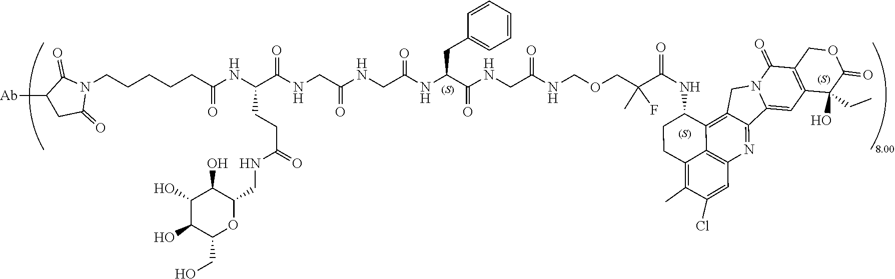 | Method 1: 9.554 |
| ADC-24 BGB-C- 373 | mAb10 | 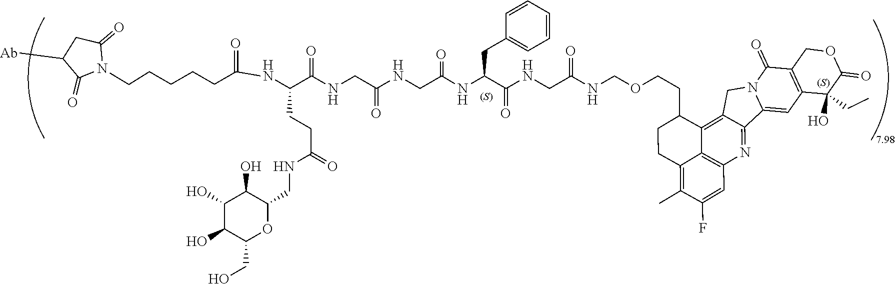 | Method 1: 9.932 |
| ADC-25 BGB-C- 421 | mAb10 | 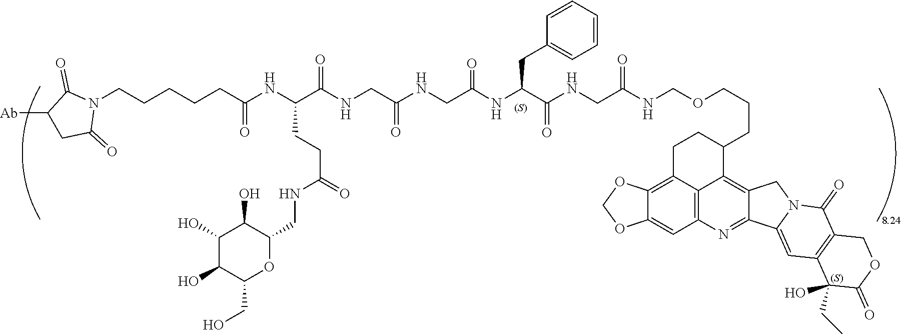 | Method 1: 9.820 |
| ADC-26 BGB-C- 395 | mAb10 | 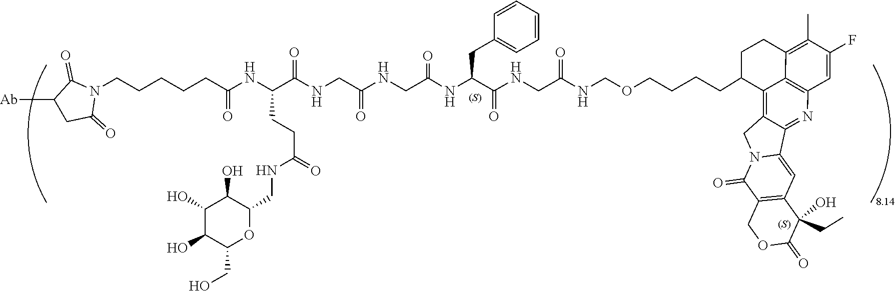 | Method 1: 9.967 |

mAb10

Patritumab has relatively short half-lives in mouse and human. And it tends to aggregate during the conjugation process. It was speculated that short half-lives and observed conjugation instability of patritumab (data not shown) might be related to its suboptimal biophysical properties (relatively lower aggregation temperature and self-association tendency as shown in AC-SiNS assay.

The VH and VK frameworks of patritumab were swap to germline frameworks with more popularity and further with several back-mutations. mAb 10 (VH framework swap from IGHV4-34 to IGHV3-23) was selected among those framework-swap patritumab variants due to its overall improvement on biophysical properties and maintained binding affinity to Her3 (see the table below).

TABLE 5
Summary of biochemical and biophysical
characterization of mAb10

| | | | | | Binding to | |
|---|---|---|---|---|---|---|
| | | | | | human HER3 | |
| | | | | | Cell | |
| | Biophysical properties | | | | binding | KD |
| | | | AC- | | to A375 | (M) to |
| | Tm | Tagg | SINS | HIC-RT | Ec50 | hHER3- |
| Ab ID | (° C.) | (° C.) | DI (nm) | (min) | (nM) | his (SPR) |
| Patritumab | 68.90 | 62.50 | 13.20 | 16.74 | 0.59 nM | 1.51E−09 |
| mAb10 | 70.10 | 68.70 | 10.90 | 15.87 | 0.83 nM | 2.52E−09 |

### Tm/Tagg Measurements by Nano-DSF

Nano-DSF is a method for measuring ultra-high-resolution protein stability using intrinsic fluorescence. All nano-DSF studies were performed using the UNCLE instrument. Samples (˜9 μL at 1 mg/mL) were loaded into Uni, and subjected to a temperature ramping of 0.3° C./minute from 15° C. to 95° C. The melting point (Tm) (° C.) values indicate the structural stability of the samples and were obtained by monitoring the intrinsic tryptophan and tyrosine fluorescence at the emission wavelengths of 330 nm and 350 nm. To generate an unfolding curve, the ratio of the fluorescence intensities (F350 nm/F330 nm) was plotted vs. temperature or time. The thermal stability of a sample was described by the thermal unfolding transition midpoint Tm (° C.), at which half of the protein population is unfolded. The Tm corresponds to the inflection point of the unfolding curve and was determined via the derivative of the curve. The aggregation point Tagg (° C.) is representative of the colloidal stability of the samples and was obtained by monitoring the onset of aggregation by the intensity of scattered light at 266 nm.

### Affinity-Capture Self-Interaction Nanoparticle Spectroscopy

The AC-SINS assay measures protein self-interaction by capturing the mAb on the surface of a gold colloid that displays surface resonance oscillations in frequency with visible light. As the immobilized antibodies self-interact, the colloids aggregate, changing the oscillation frequency to absorb at a longer wavelength. Gold nanoparticles were incubated with an 80/20 (v/v) capture antibody/non-capture antibody mixture. The coated gold nanoparticles were then spun down and resuspended into PBS. The samples were diluted to 0.05 mg/ml into the conjugation buffers and 45 ul of each dilution was loaded onto a 384-well plate. Five ul of previously prepared gold nanoparticles were then added to each well of the plate including mAbs and buffer controls. The plate was then covered with an aluminum lid, incubated at room temperature for 2 hours, and quickly spun down at 3000 rpm prior to reading the absorbance spectra of each well from 450 to 650 nm using a plate reader. Each sample spectra was recorded and analyzed for red-shifting of the maximum of absorption peak compared to buffers and mAb controls, the red-shifting and its strength is indicative of the self-interaction propensity of the tested mAb sample.

### Hydrophobic Chromatography

To determine the hydrophobicity of a given mAb using HIC, 100 μg of sample at 1 mg/ml was filtered through a 0.22 um PVDF membrane prior to loading 20 ul on a MacPac HIC-10 (4.6*100 mm, Cat.088480) column equilibrated in 100 mM sodium phosphate, 1.5 M ammonium sulfate pH 7.0 (mobile phase A). The samples were eluted using an inverted gradient from mobile phase A to 100 mM sodium phosphate pH 7.0 (mobile phase B). The elution was followed by recording the A280 nm as a function of time, the data was then exported and analyzed using the Empower software. The retention time of each sample was compared to a reference and is characteristic of the mAb hydrophobicity with longer elution times correlating with higher degree of hydrophobicity.

### Binding FACS

The binding affinity of patritumab and Mab10 to native Her3 were further compared by FACS using tumor cell line A375. A375 cells (10⁵ cells/well) were incubated with a series of concentrations of patritumab or mab10, followed by binding with Alexa Fluro-647 labeled mouse anti-human IgG antibody (Cat.: 410714, Biolegend, US). Cell fluorescence was quantified using a flow cytometer (Guava easyCyte 8HT, Merck-Millipore, USA). EC50 values for dose-dependent binding to human native Her3 were determined by fitting the dose-response data to the four-parameter logistic model with GraphPad Prism.

### Binding Kinetics by SPR (BIAcore)

Binding kinetics of the mAbs to the target was determined by SPR on a BIAcore 8K (GE Healthcare). The running buffer, 10 mM HEPES, 150 mM NaCl, 0.05% v/v Surfactant P20, 3 mM EDTA, pH 7.4 (HBS-EP+, GE Healthcare) was used for immobilization and reagent dilutions. All binding kinetics were measured at 25° C. For each injection cycle, mAbs were first captured in different flow cells with an anti-human Fc antibody (Human Antibody Capture Kit, GE Healthcare) immobilized to the sensor chip (Series S CM5, GE Healthcare). Reference flow cell with no captured mAb was also used. Serial dilutions of Her3-his(cat10201-H08H, SinoBiological), and buffer blanks were injected in multiple cycles over the captured mAbs and reference surfaces for association followed by dissociation. The surfaces were regenerated with an injection of 3 M MgCl2 after each cycle. Double-referenced titration data was globally fit to a 1:1 Langmuir binding model to determine the association rate constant, ka (M−1 s−1), and the dissociation rate constant, kd (s−1), using the BIAcore Insight Evaluation Software version 2.0 (GE Healthcare). The equilibrium dissociation constant was calculated as KD(M)=kd/ka.

Compared with Patritumab, mAb10 unexpectedly showed better biophysical properties, e.g., higher aggregation temperature and reduced self-association, indicating superior isothermal stability, meanwhile maintaining comparable binding affinity.

Production of mAb10 for ADC Conjugation

mAb10 was produced by transient transfection of heavy chain and kappa chain containing plasmids in CHO cells. The conditioned medium was harvested and mAb10 was purified using a protein A column, followed by an ion-exchange column and a buffer-exchange column.

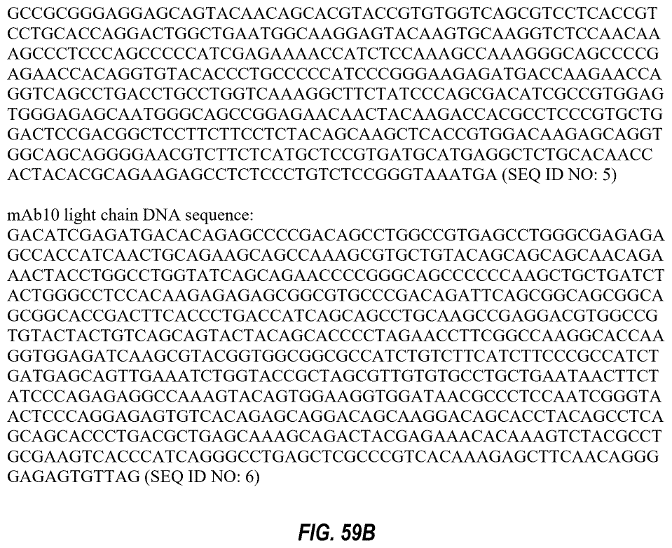

The sequences related to mAb10 are provided in FIGS. 59A and 59B.

### Cell Lines Information

### A375 (ATCC, CRL-1619)

A-375 is a cell line exhibiting epithelial morphology that was isolated from the skin of a 54-year-old, female patient with malignant melanoma, and A375 was purchased from ATCC. The base medium for A375 is DMEM, high glucose, GlutaMAX™ Supplement (Gibco, 10566024). To make the complete growth medium, ass the following components to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### Calu-6 (ATCC, HTB-56)

Calu-6 is a cell line exhibiting epithelial morphology that was isolated from the carcinoma and anaplastic, and Calu-6 was purchased from ATCC. The base medium for Calu-6 is Eagle's Minimum Essential Medium (ATCC, 30-2003). To make the complete growth medium, ass the following components to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### MDA-MB-453 (SIBS)

MDA-MB-453 was derived from an effusion of a 48 year old female patient with metastatic carcinoma of the breast, involving the nodes, brain and both pleural and pericardial cavities, and MDA-MB-453 was purchased from SIBS. The base medium for MDA-MB-453 is RPMI 1640 Medium, HEPES (Gibco, 22400105). To make the complete growth medium, ass the following components to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

TABLE 6
Cell lines: HER3 expression level

| Cell lines | Molecular No. (QSC kit) |
|---|---|
| MDA-MB-453 | 90366 |
| A375 | 55054 |
| Calu-6 | 0 |

### Payload Cellular Killing Potency

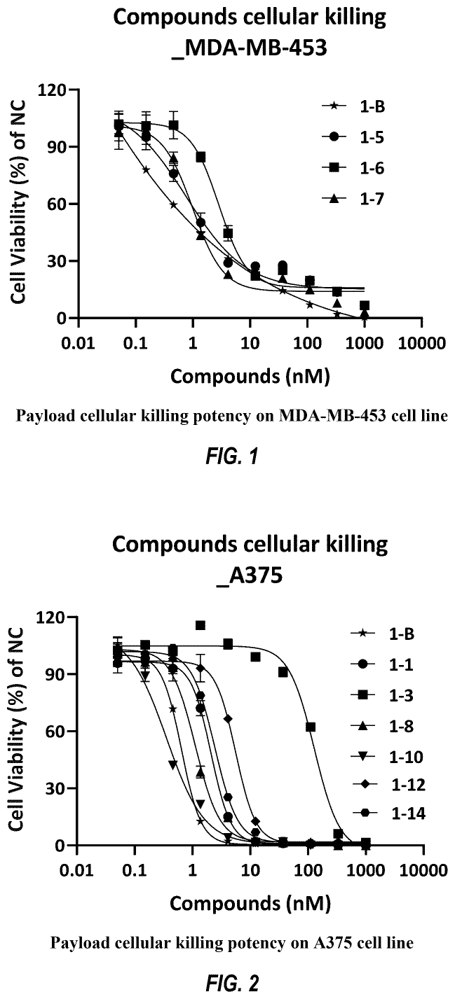

### Group 1 (Table 1-Table 2 and FIG. 1-5)

### Compounds Cellular Killing in MDA-MB-453 Cancer Line

### Method: Compounds Cellular Killing

Seeding cells MDA-MB-453 (2E3/well) into 96-well plate (Greiner: 655090), 100 ul/well. Incubation at 37° C., 5% CO₂, overnight.

Adding the fresh growth-medium containing the varying concentrations of compounds, 50 ul/well.

Incubation at 37° C., 5% CO₂, 6 days.

The cell viability detected by Cell Titer-Glo (Promega, G7573). Add 70 L/well of CellTiter-Glo® Reagent in each well.

Allow the plates to incubate at room temperature for 10 minutes to stabilize luminescent signal.

Analyze with Microplate Reader.

### Result

As shown in FIG. 1, 1-5 & 1-7 show comparable killing activity to 1-B.

As shown in FIG. 1, 1-6 is less potent than 1-B. The data is provided in the following table, Table 7:

TABLE 7

MDA-MB-453

| Compounds | Emax (%) | EC50 (nM) |
|---|---|---|
| 1-B | 99.4 | 0.9 |
| 1-5 | 93.3 | 1.3 |
| 1-6 | 93.4 | 3.3 |
| 1-7 | 96.8 | 1.2 |

### Compounds Cellular Killing in A375 and Calu-6 Cancer Lines

### Method: Compounds Cellular Killing

Seeding cells A375 (1E3/well) or Calu-6 (2E3/well) into 96-well plate (Greiner: 655090), 100 ul/well. Incubation at 37° C., 5% CO₂, overnight.

Adding the fresh growth-medium containing the varying concentrations of compounds, 50 ul/well.

Incubation at 37° C., 5% CO₂, 6 days.

The cell viability detected by Cell Titer-Glo (Promega, G7573). Add 70 L/well of CellTiter-Glo® Reagent in each well.

Allow the plates to incubate at room temperature for 10 minutes to stabilize luminescent signal.

Analyze with Microplate Reader.

FIG. 2 illustrates the payload cellular killing potency on A375 cell lines.

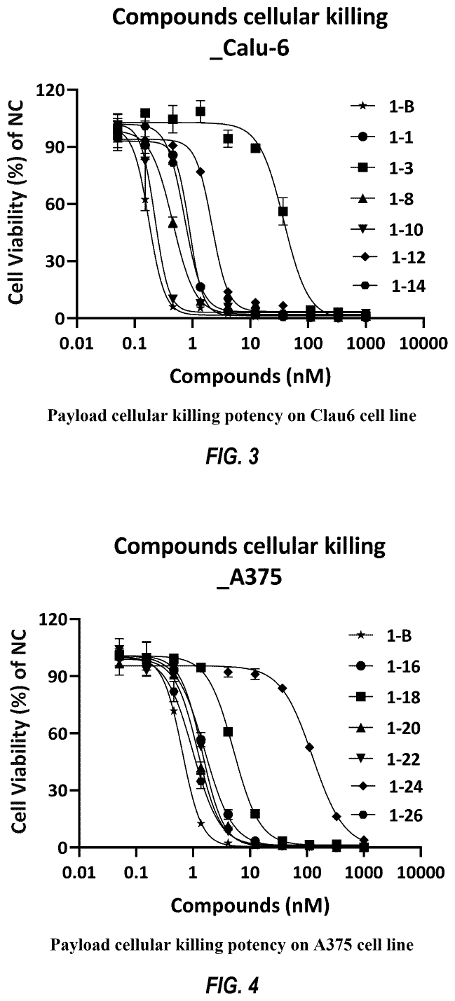

FIG. 3 illustrates the payload cellular killing potency on Clau6 cell lines.

The data is provided in the following tables, Table 8:

TABLE 8

| | A375 | | Calu-6 | |
|---|---|---|---|---|
| Compounds | Emax (%) | EC50 (nM) | Emax (%) | EC50 (nM) |
| 1-B | 100.0 | 0.7 | 98.4 | 0.2 |
| 1-1 | 100.0 | 2.0 | 99.6 | 0.9 |
| 1-3 | 98.4 | 129.7 | 98.8 | 38.5 |
| 1-8 | 98.8 | 1.2 | 98.8 | 0.5 |
| 1-10 | 97.7 | 0.4 | 97.7 | 0.3 |
| 1-12 | 99.8 | 5.5 | 99.5 | 2.1 |
| 1-14 | 100.0 | 2.5 | 98.8 | 0.8 |

FIG. 4 illustrates the payload cellular killing potency on A375 cell lines.

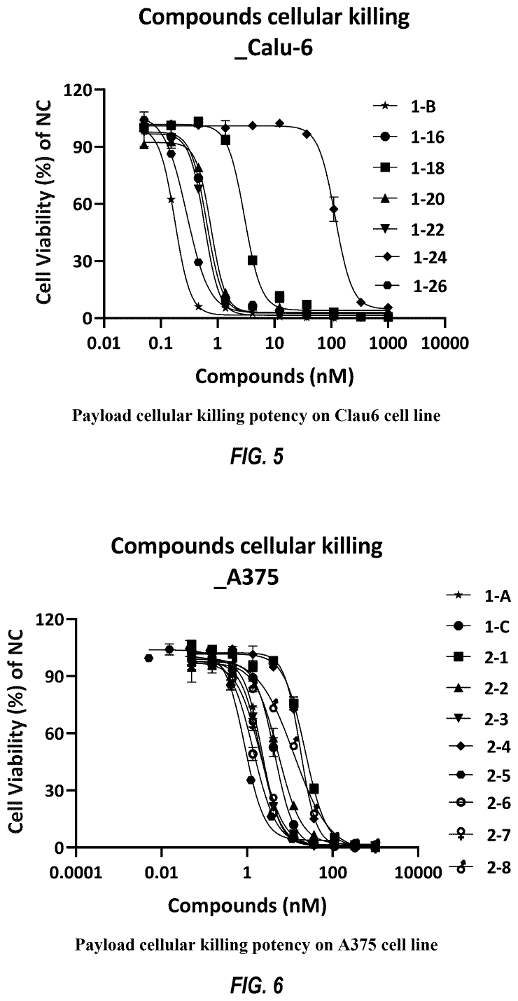

FIG. 5 illustrates the payload cellular killing potency on Clau6 cell lines.

The data is provided in the following table:

TABLE 9

| | A375 | | Calu-6 | |
|---|---|---|---|---|
| Compounds | Emax (%) | EC50 (nM) | Emax (%) | EC50 (nM) |
| 1-B | 100.0 | 0.7 | 98.4 | 0.2 |
| 1-16 | 98.5 | 1.6 | 98.5 | 0.6 |
| 1-18 | 99.9 | 5.3 | 99.5 | 3.1 |
| 1-20 | 98.9 | 1.2 | 98.9 | 0.7 |
| 1-22 | 100.0 | 1.4 | 99.2 | 0.6 |
| 1-24 | 96.0 | 111.8 | 94.4 | 117.9 |
| 1-26 | 98.1 | 1.0 | 98.1 | 0.3 |

### Group 2 (Table 10-Table 16, FIG. 6-FIG. 19)

### Compounds Cellular Killing in A375 and Calu-6 Cancer Lines

### Method: Compounds Cellular Killing

Seeding cells A375 ((E3/well) or Calu-6 (2E3/well) into 96-well plate (Greiner: 655090), 100 ul/well. Incubation at 37° C., 500 CO₂, overnight.

Adding the fresh growth-medium containing the varying concentrations of compounds, 50 ul/well.

Incubation at 37° C., 5% CO₂, 6 days.

The cell viability detected by Cell Titer-Glo (Promega, G7573). Add 70 L/well of CellTiter-Glo® Reagent in each well.

Allow the plates to incubate at room temperature for 10 minutes to stabilize luminescent signal.

Analyze with Microplate Reader.

FIG. 6 illustrates the payload cellular killing potency on A375 cell lines.

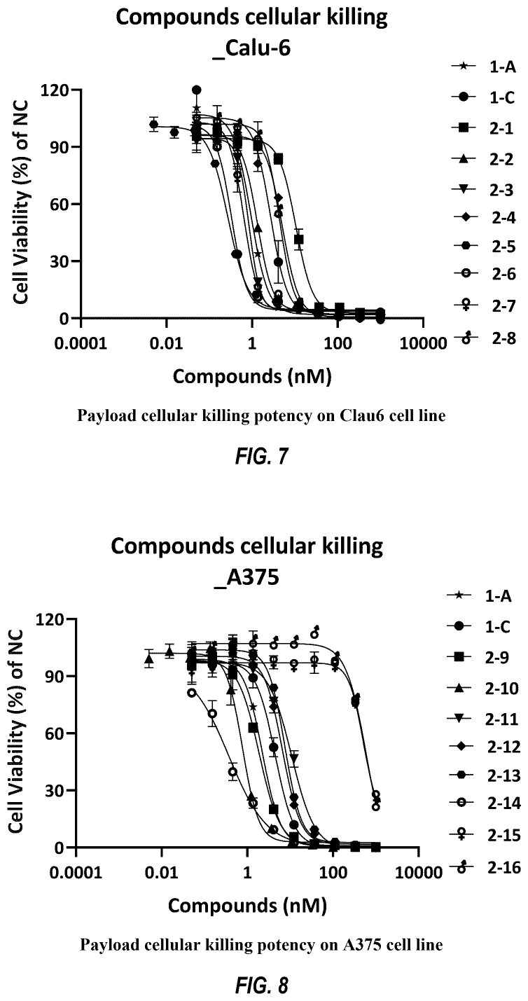

FIG. 7 illustrates the payload cellular killing potency on Clau6 cell lines.

The data is provided in the following table, Table 10:

TABLE 10

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-1 | 98.0 | 23.0 | 98.0 | 10.0 |
| 2-2 | 99.9 | 5.2 | 98.7 | 1.3 |
| 2-3 | 100.0 | 2.1 | 98.5 | 0.8 |
| 2-4 | 99.3 | 18.0 | 99.7 | 4.9 |
| 2-5 | 98.8 | 0.9 | 98.9 | 0.3 |
| 2-6 | 99.9 | 1.4 | 96.9 | 0.3 |
| 2-7 | 99.1 | 2.0 | 99.1 | 0.7 |
| 2-8 | 98.6 | 1.2 | 99.3 | 0.4 |

FIG. 8 illustrates the payload cellular killing potency on A375 cell lines.

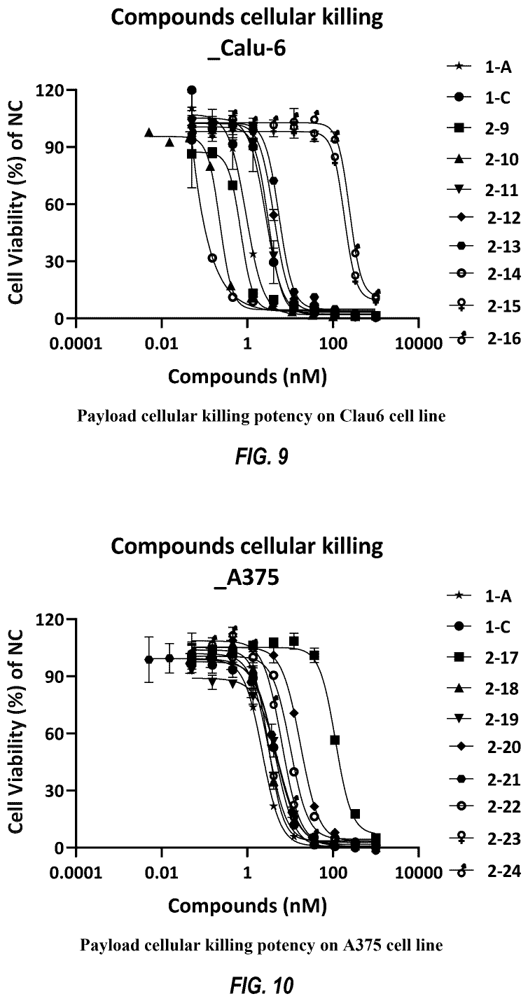

FIG. 9 illustrates the payload cellular killing potency on Clau6 cell lines.

The data is provided in the following table, Table 11:

TABLE 11

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-9 | 100.0 | 1.9 | 98.6 | 0.7 |
| 2-10 | 99.9 | 0.8 | 98.9 | 0.2 |
| 2-11 | 99.7 | 10.2 | 99.6 | 3.2 |
| 2-12 | 99.9 | 6.8 | 98.9 | 4.4 |
| 2-13 | 99.6 | 7.9 | 98.4 | 5.8 |
| 2-14 | 100.0 | 0.3 | 99.0 | 0.1 |
| 2-15 | 73.5 | 441.5 | 90.1 | 179.3 |
| 2-16 | 77.1 | 487.1 | 87.9 | 246.9 |

FIG. 10 illustrates the payload cellular killing potency on A375 cell lines.

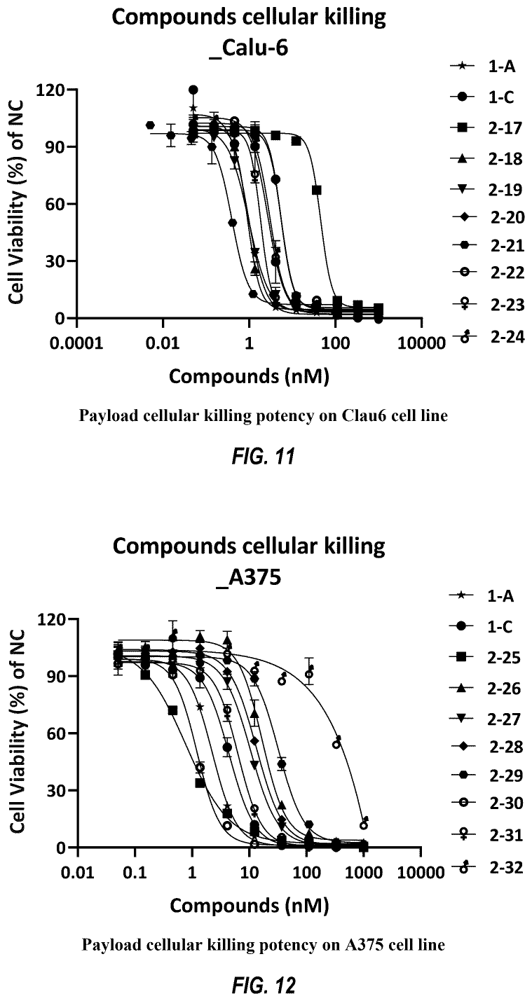

FIG. 11 illustrates the payload cellular killing potency on Clau6 cell lines.

The data is provided in the following table, Table 12:

TABLE 12

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-17 | 94.9 | 122.7 | 94.6 | 44.9 |
| 2-18 | 99.8 | 3.3 | 98.0 | 0.9 |
| 2-19 | 99.7 | 4.6 | 99.3 | 1.0 |
| 2-20 | 98.0 | 18.5 | 98.1 | 5.6 |
| 2-21 | 98.3 | 4.5 | 96.7 | 0.4 |
| 2-22 | 97.7 | 10.4 | 98.0 | 5.6 |
| 2-23 | 99.9 | 3.3 | 98.9 | 1.9 |
| 2-24 | 99.9 | 7.1 | 99.2 | 3.2 |

FIG. 12 illustrates the payload cellular killing potency on A375 cell lines.

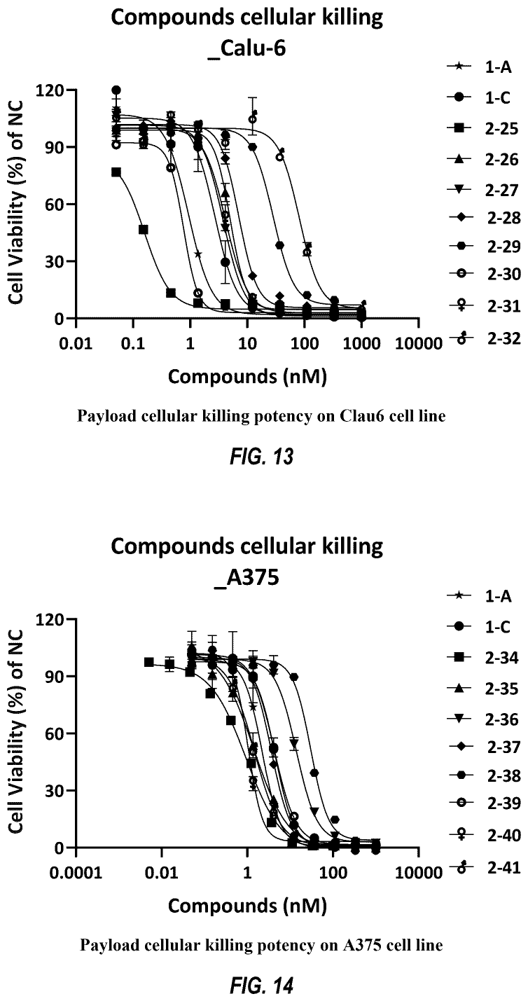

FIG. 13 illustrates the payload cellular killing potency on Clau6 cell lines.

The data is provided in the following table, Table 13:

TABLE 13

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-25 | 99.9 | 0.9 | 98.2 | 0.1 |
| 2-26 | 97.7 | 18.6 | 98.4 | 5.1 |
| 2-27 | 99.5 | 10.7 | 99.5 | 3.9 |
| 2-28 | 98.8 | 13.8 | 98.7 | 7.4 |
| 2-29 | 98.3 | 32.7 | 96.7 | 29.4 |
| 2-30 | 98.9 | 1.2 | 98.9 | 0.7 |
| 2-31 | 98.9 | 6.2 | 98.9 | 4.2 |
| 2-32 | 93.7 | 368.4 | 93.7 | 81.5 |

FIG. 14 illustrates the payload cellular killing potency on A375 cell lines.

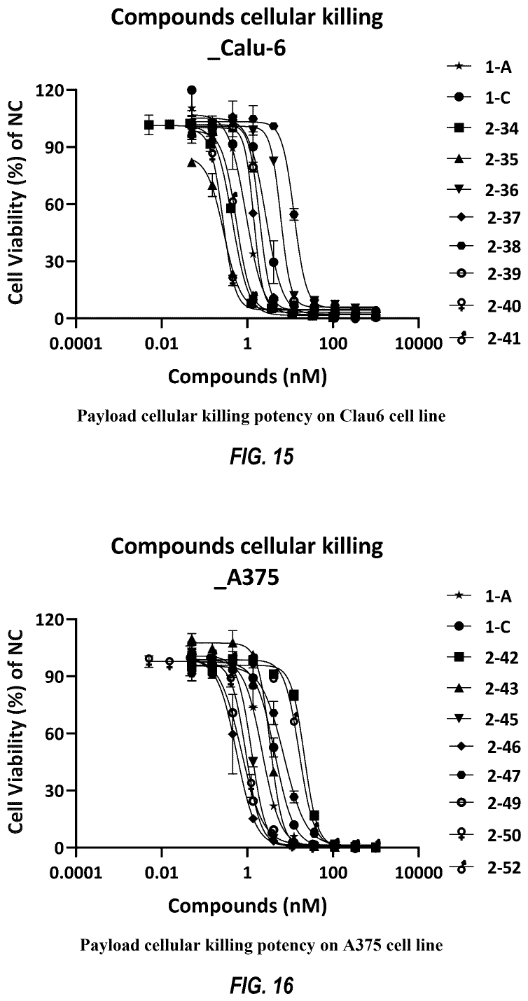

FIG. 15 illustrates the payload cellular killing potency on Clau6 cell lines.

The data is provided in the following table, Table 14:

TABLE 14

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-34 | 98.9 | 0.8 | 99.4 | 0.5 |
| 2-35 | 100.0 | 1.6 | 98.6 | 0.2 |
| 2-36 | 97.8 | 13.9 | 97.7 | 6.2 |
| 2-37 | 99.9 | 3.7 | 98.8 | 1.5 |
| 2-38 | 98.7 | 30.9 | 96.4 | 12.9 |
| 2-39 | 99.9 | 4.4 | 98.3 | 2.0 |
| 2-40 | 99.9 | 1.1 | 97.3 | 0.3 |
| 2-41 | 99.9 | 1.5 | 98.2 | 0.6 |

FIG. 16 illustrates the payload cellular killing potency on A375 cell lines.

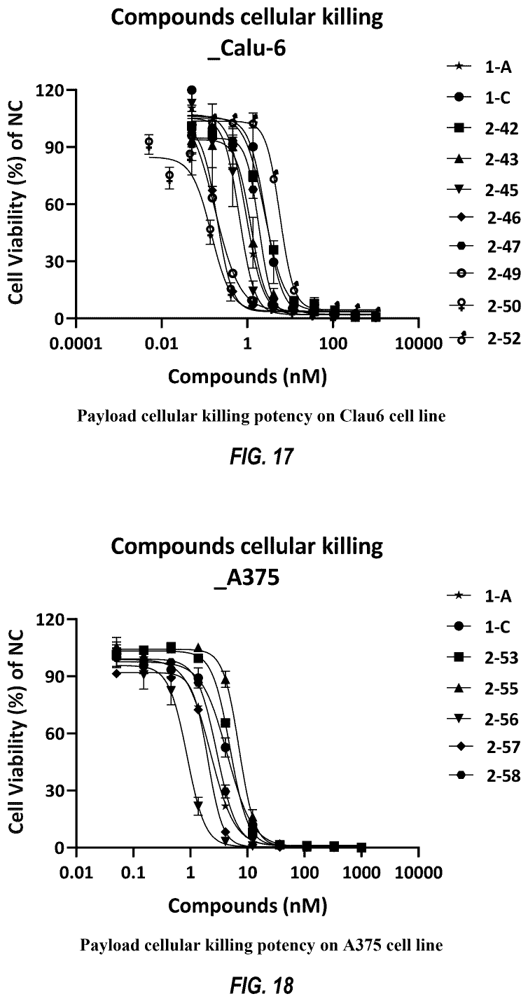

FIG. 17 illustrates the payload cellular killing potency on Clau6 cell lines.

The data is provided in the following table, Table 15:

TABLE 15

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-42 | 99.6 | 20.7 | 99.2 | 2.8 |
| 2-43 | 100.0 | 3.6 | 99.2 | 1.1 |
| 2-45 | 100.0 | 1.3 | 99.2 | 0.7 |
| 2-46 | 100.0 | 0.7 | 98.1 | 0.2 |
| 2-47 | 99.7 | 6.7 | 99.5 | 1.8 |
| 2-49 | 99.9 | 0.7 | 99.1 | 0.2 |
| 2-50 | 99.5 | 0.3 | 99.2 | 0.0 |
| 2-52 | 98.9 | 16.1 | 97.3 | 6.0 |

FIG. 18 illustrates the payload cellular killing potency on A375 cell lines.

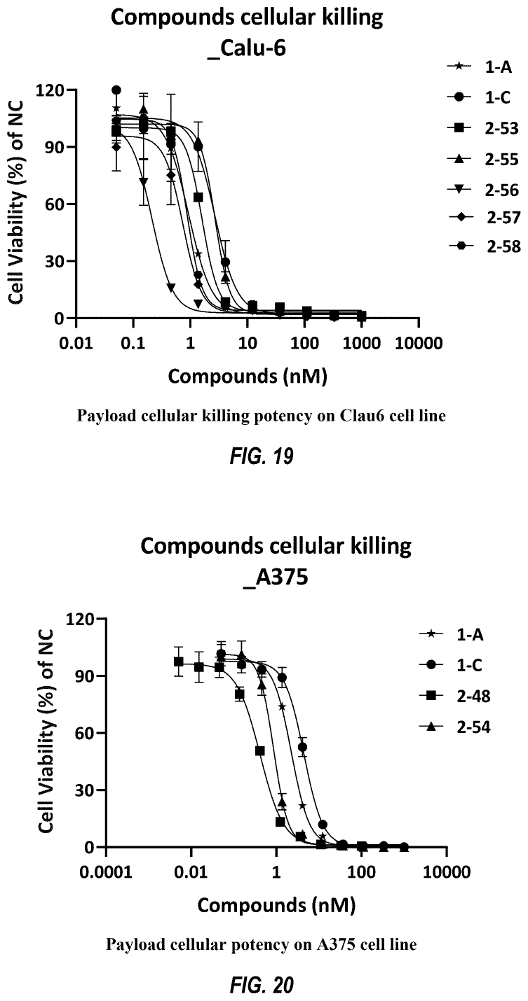

FIG. 19 illustrates the payload cellular killing potency on Clau6 cell lines.

The data is provided in the following table, Table 16:

TABLE 16

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-53 | 99.9 | 5.1 | 98.7 | 1.6 |
| 2-55 | 99.8 | 7.3 | 99.3 | 2.8 |
| 2-56 | 100.0 | 0.9 | 99.0 | 0.2 |
| 2-57 | 100.0 | 1.9 | 99.3 | 0.7 |
| 2-58 | 99.9 | 2.9 | 98.8 | 0.9 |

FIG. 20 illustrates the payload cellular killing potency on A375 cell lines.

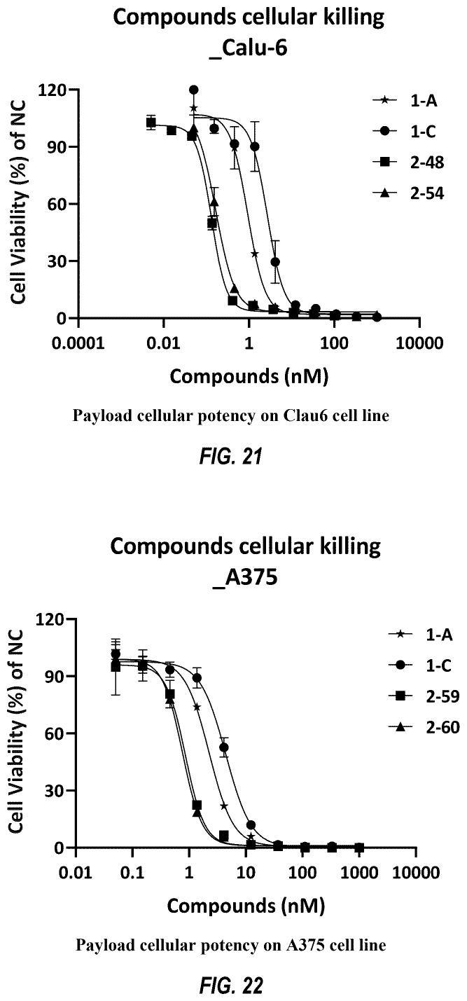

FIG. 21 illustrates the payload cellular killing potency on Clau6 cell lines.

The data is provided in the following table, Table 17:

TABLE 17

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-48 | 99.5 | 0.4 | 98.7 | 0.1 |
| 2-54 | 100.0 | 0.9 | 98.2 | 0.2 |

FIG. 22 illustrates the payload cellular killing potency on A375 cell lines.

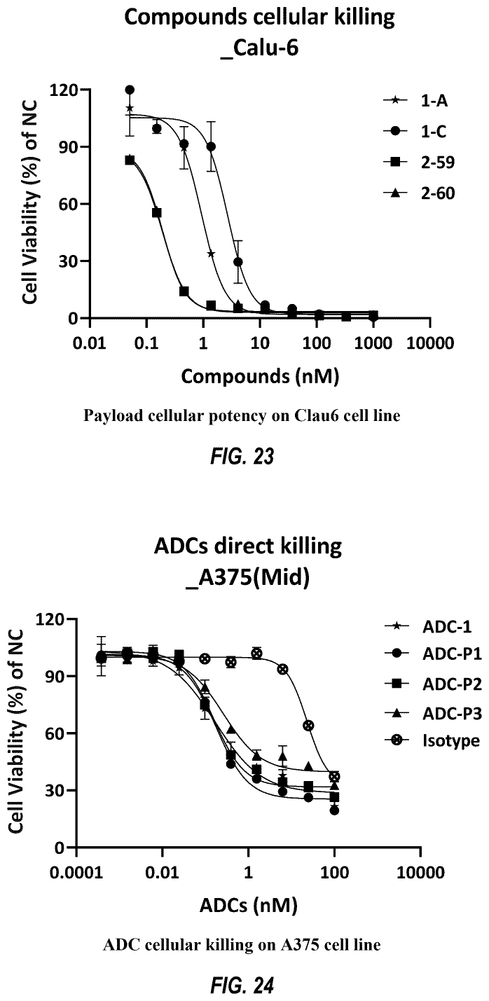

FIG. 23 illustrates the payload cellular killing potency on Clau6 cell lines.

The data is provided in the following table, Table 18:

TABLE 18

| | A375 | | Calu-6 | |
|---|---|---|---|---|
| Compounds | Emax (%) | EC50 (nM) | Emax (%) | EC50 (nM) |
| 1-A | 98.9 | 2.2 | 98.9 | 1.0 |
| 1-C | 99.5 | 4.3 | 99.5 | 2.6 |
| 2-59 | 100.0 | 0.8 | 98.4 | 0.2 |
| 2-60 | 100.0 | 0.8 | 98.2 | 0.2 |

Compounds 2-59 and 2-60 appeared higher potency than compound 1-A and 1-C.

ADCs direct killing in A375 and Calu-6 cancer lines

Method: ADCs direct killing

Seeding cells A375 (1E3/well) into 3D-96-well plate (Corning: 4520), 80 ul/well. And Calu-6 (2E3/well) into 2D-96-well plate (Greiner: 655090), 100 ul/well. Incubation at 37° C., 5% CO₂, overnight.

Adding the fresh growth-medium containing the varying concentrations of ADCs, 40 ul/well into A375 and 50 ul/well into Calu-6.

Incubation at 37° C., 5% CO₂, 6 days.

The A375 cell viability detected by 3D reagent (Promega, G9683), 100 ul/well of 3D reagent. Calu-6 cell viability detected by Cell Titer-Glo (Promega, G7573), 70 L/well of Cell Titer-Glo® Reagent.

Allow the 3D-plates to incubate at room temperature for 30 minutes to stabilize luminescent signal, and the 2D-plates to incubate at room temperature for 10 minutes to stabilize luminescent signal.

Analyze with Microplate Reader.

### Result

All ADCs show cellular killing activity in A375.

ADC-5 and ADC-8 show stronger non-specific killing than ADC-1 in Calu-6 (HER3 negative cell).

FIG. 24 illustrates the ADC direct cellular killing potency on A375 cell lines.

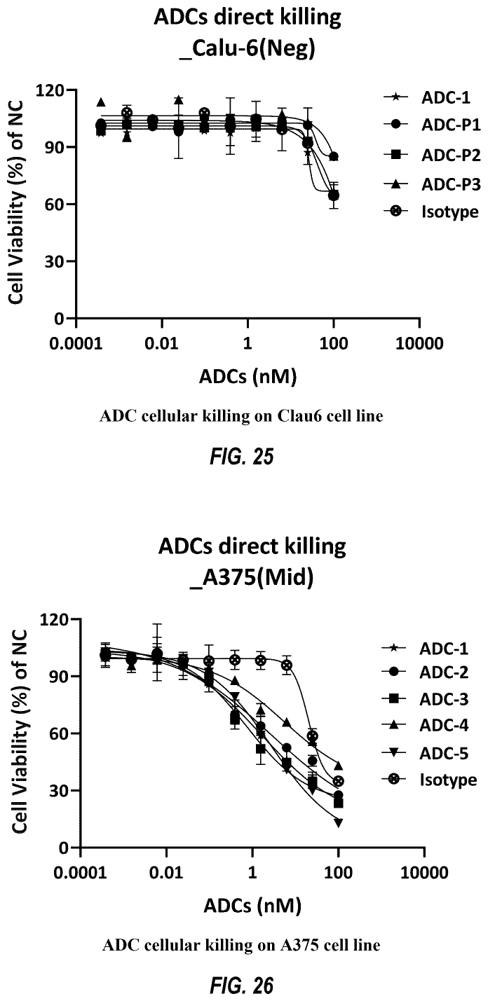

FIG. 25 illustrates the ADC direct cellular killing potency on Clau6 cell lines.

The data is provided in the following table, Table 19:

TABLE 19

A375 (Mid), 3D

| ADCs | Emax (%) | EC50 (nM) |
|---|---|---|
| ADC-1 | 78.5 | 0.2 |
| ADC-P1 | 80.6 | 0.2 |
| ADC-P2 | 73.5 | 0.2 |
| ADC-P3 | 67.3 | 0.3 |

FIG. 26 illustrates the ADC direct cellular killing potency on A375 cell lines.

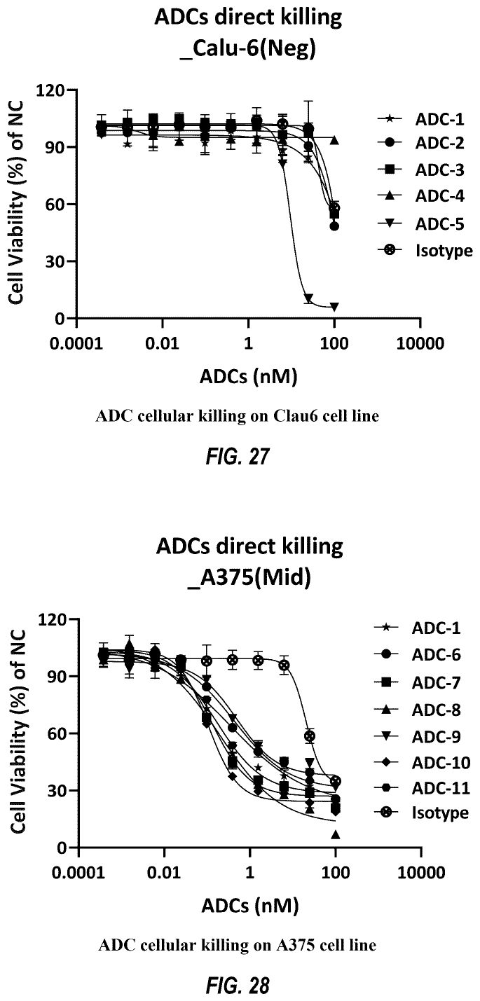

FIG. 27 illustrates the ADC direct cellular killing potency on Clau6 cell lines.

The data is provided in the following table, Table 20:

TABLE 20

A375 (Mid), 3D

| ADCs | Emax (%) | EC50 (nM) |
|---|---|---|
| ADC-1 | 76.8 | 1.3 |
| ADC-2 | 77.8 | 1.1 |
| ADC-3 | 76.6 | 0.9 |
| ADC-4 | 56.9 | 2.9 |
| ADC-5 | 87.3 | 2.2 |

FIG. 28 illustrates the ADC direct cellular killing potency on A375 cell lines.

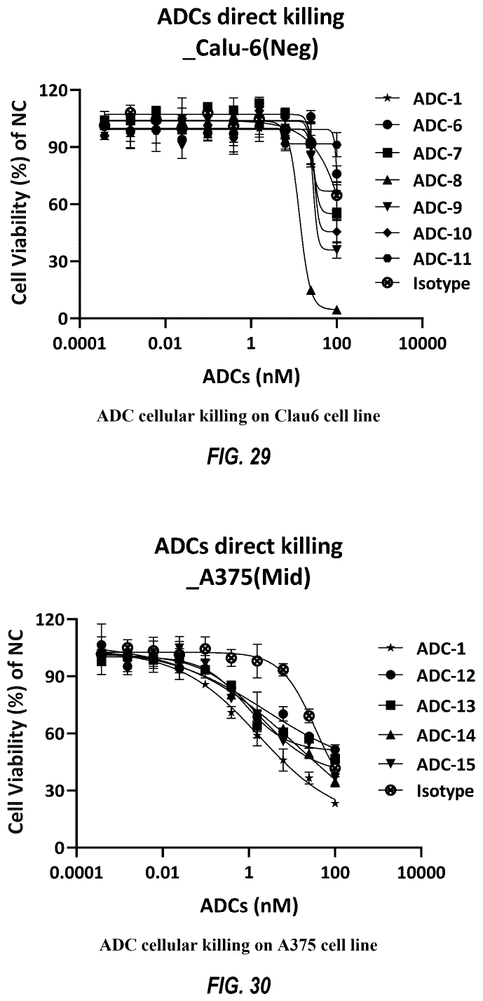

FIG. 29 illustrates the ADC direct cellular killing potency on Clau6 cell lines.

The data is provided in the following table:

TABLE 21

A375 (Mid), 3D

| ADCs | Emax (%) | EC50 (nM) |
|---|---|---|
| ADC-1 | 78.5 | 0.2 |
| ADC-6 | 74.4 | 0.6 |
| ADC-7 | 79.1 | 0.2 |
| ADC-8 | 92.9 | 0.3 |
| ADC-9 | 68.3 | 0.6 |
| ADC-10 | 81.2 | 0.1 |
| ADC-11 | 77.9 | 0.5 |

FIG. 30 illustrates the ADC direct cellular killing potency on A375 cell lines.

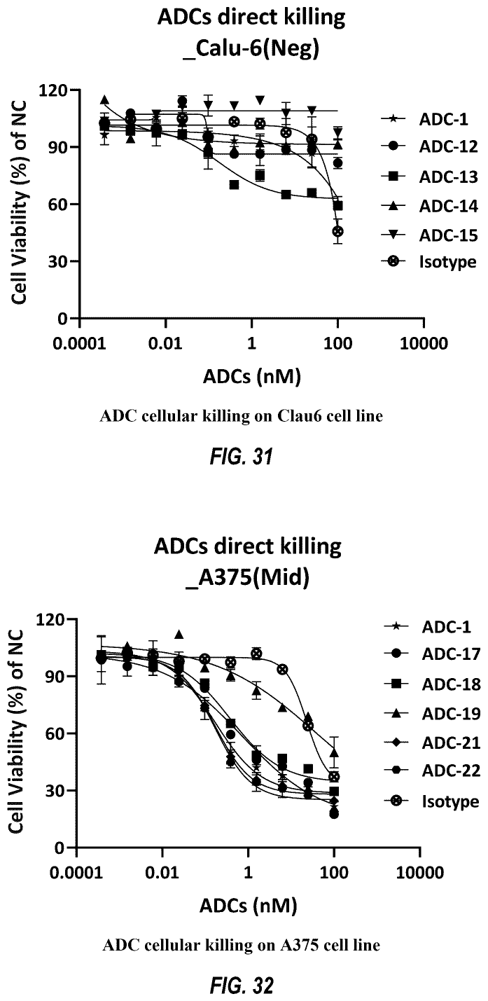

FIG. 31 illustrates the ADC direct cellular killing potency on Clau6 cell lines.

The data is provided in the following table:

TABLE 22

A375 (Mid), 3D

| ADCs | Emax (%) | EC50 (nM) |
|---|---|---|
| ADC-1 | 76.8 | 1.3 |
| ADC-12 | 48.4 | 1.4 |
| ADC-13 | 53.5 | 1.0 |
| ADC-14 | 65.3 | 2.7 |
| ADC-15 | 63.4 | 1.9 |

FIG. 32 illustrates the ADC direct cellular killing potency on A375 cell lines.

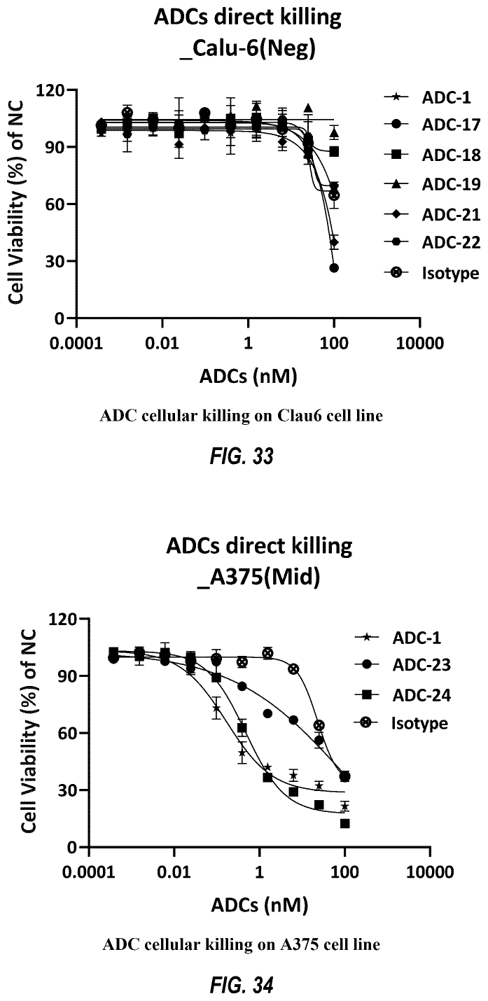

FIG. 33 illustrates the ADC direct cellular killing potency on Clau6 cell lines.

The data is provided in the following table:

TABLE 23

A375 (Mid), 3D

| ADCs | Emax (%) | EC50 (nM) |
|---|---|---|
| ADC-1 | 78.5 | 0.2 |
| ADC-17 | 82.4 | 0.7 |
| ADC-18 | 70.5 | 0.5 |
| ADC-19 | 49.9 | 7.0 |
| ADC-21 | 75.4 | 0.2 |
| ADC-22 | 81.1 | 0.2 |

FIG. 34 illustrates the ADC direct cellular killing potency on A375 cell lines.

FIG. 35 illustrates the ADC direct cellular killing potency on Clau6 cell lines.

The data is provided in the following table:

TABLE 24

A375 (Mid), 3D

| ADCs | Emax (%) | EC50 (nM) |
|---|---|---|
| ADC-1 | 78.5 | 0.2 |
| ADC-23 | 63.5 | 4.7 |
| ADC-24 | 87.6 | 0.6 |

ADCs bystander killing_MDA-MB-453 or A375 co-culture with Calu-6-nanoLuc

Method: Calu-6-nanoLuc cell line construction

PT67-nanoLuc cell culture, then collect the cell-culture medium (contain the virus _nano-Luc gene) and do the filtration.

Seeding the Calu-6 in the 6-well plate, 1E5 cells/well. Incubation at 37° C., 5% CO₂, overnight.

Adding the PT67-nanoLuc cell medium, and 8 ug/ml polybrene.

The infection was repeated for 3 times, every one day.

Then culture the Calu-6-nanoLuc cells, adding 1 mg/ml Geneticin for 5 days.

Collect the Calu-6-nanoLuc cells, adding Nano-Glo reagent (Promega: N1120) to test the nano-Luc transfection efficiency.

Method: ADCs bystander killing

Co-culture MDA-MB-453 & Calu-6-nanoLuc (10:1), A375 & Calu-6-nanoLuc (10:1), or Calu-6-nanoLuc only into 3D-96-well plate (Corning: 4520), 80 ul/well, Incubation at 37° C., 5% CO2, overnight.

Adding the fresh growth-medium containing the varying concentrations of ADCs, 40 ul/well into cells.

Incubation at 37° C., 5% CO₂, 6 days.

Centrifuge the 3D-plates at 1500 rpm, 25° C., 5 min, then discard the supernatant.

The Calu-6-nanoLuc cell viability detected by Nano-Glo reagent (Promega: N1120), 150 ul/well.

Allow the 3D-plates to incubate at room temperature for 10 minutes to stabilize luminescent signal.

Analyze with Microplate Reader.

### Result

ADC-P2, P3, 5, 7, 8, 9, 10, 21 have stronger bystander killing activity in MDA-MB-453 & Calu-6-nanoLuc system than ADC-1 (no bystander killing in this system).

ADC-P1, P2, P3, 1, 2, 3, 5, 6, 7, 8, 9, 10, 15, 21, 22 show bystander killing potency in A375 & Calu-6-nanoLuc system.

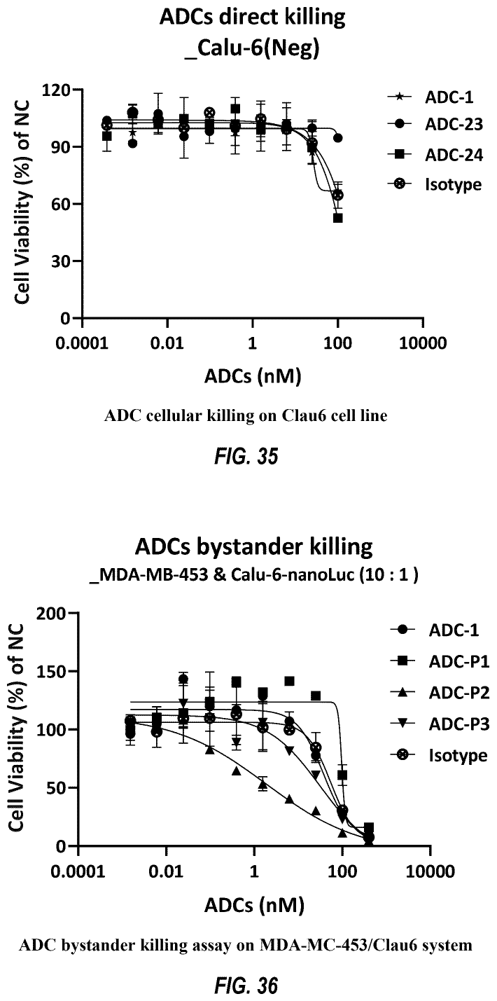

FIG. 36 illustrates the ADC bystander cellular killing activity in the MDA-MB-453 & Calu-6-nanoLuc system.

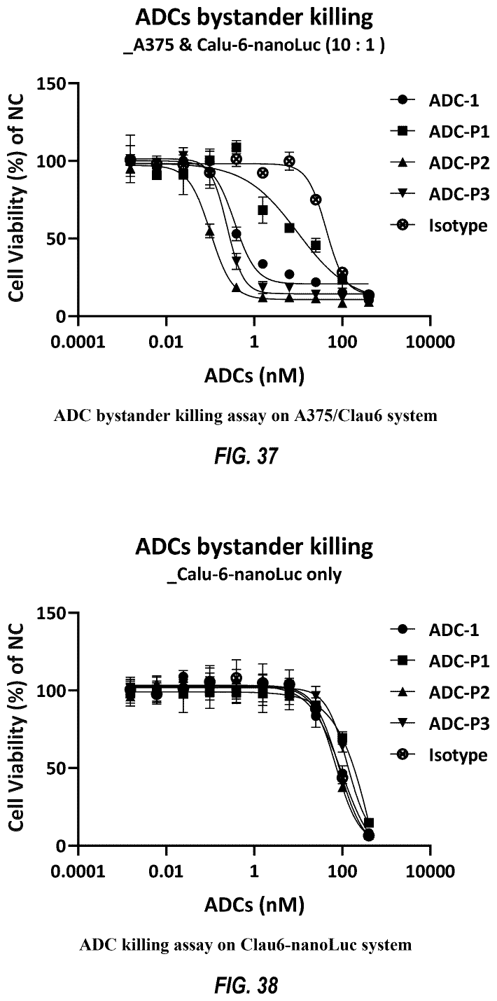

FIG. 37 illustrates the ADC bystander cellular killing potency in the A375 & Calu-6-nanoLuc system.

FIG. 38 illustrates the ADC bystander cellular killing activity in the Calu-6-nanoLuc only system.

The data is provided in the following table, Table 25:

TABLE 25

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |
| ADC-1 | NA | | 89.7 | 0.4 |
| ADC-P1 | NA | | 87.3 | 8.3 |
| ADC-P2 | 95.2 | 2.0 | 90.8 | 0.1 |
| ADC-P3 | 91.1 | 27.4 | 89.0 | 0.3 |

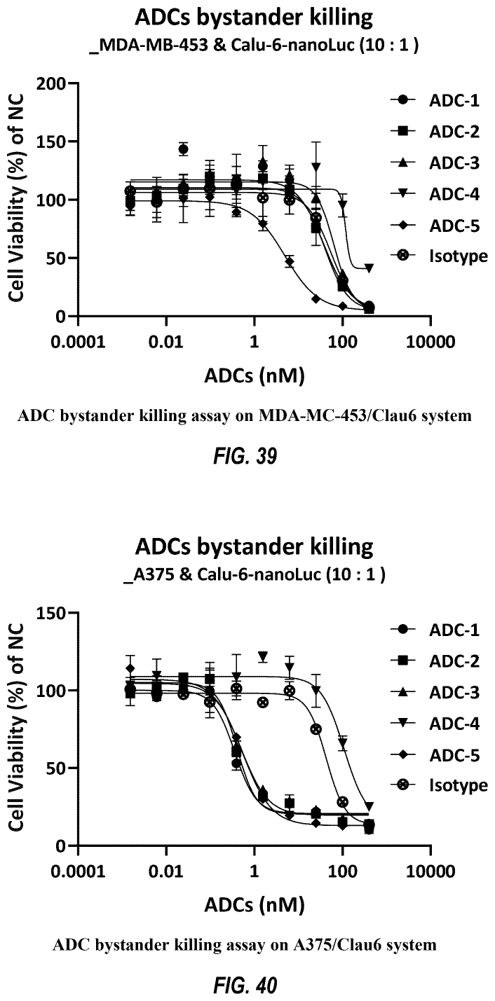

FIG. 39 illustrates the ADC bystander cellular killing potency in the MDA-MB-453 & Calu-6-nanoLuc system.

FIG. 40 illustrates the ADC bystander cellular killing activity in the A375 & Calu-6-nanoLuc system.

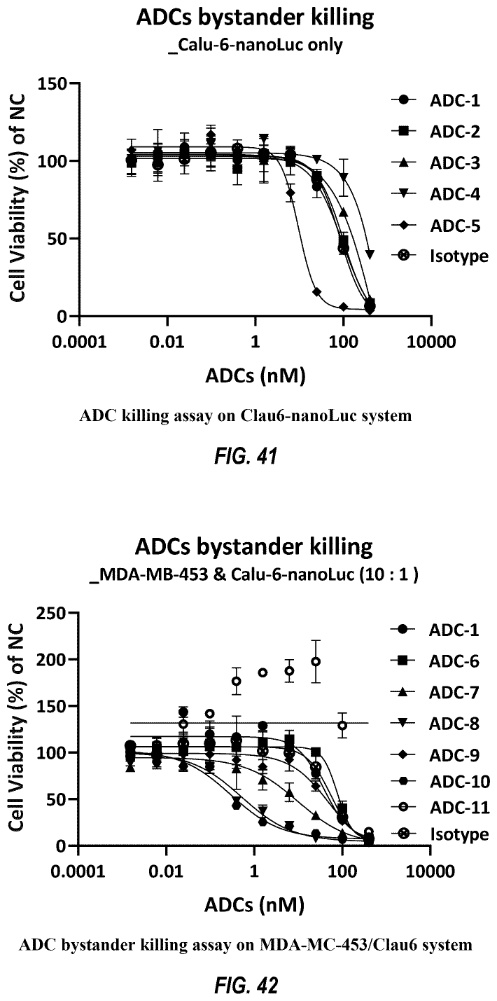

FIG. 41 illustrates the ADC bystander cellular killing potency in the Calu-6-nanoLuc only system.

The data is provided in the following table:

TABLE 26

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |
| ADC-1 | No bystander killing | | 89.7 | 0.4 |
| ADC-2 | No bystander killing | | 91.4 | 0.5 |
| ADC-3 | No bystander killing | | 89.0 | 0.6 |
| ADC-4 | No bystander killing | | No bystander killing | |
| ADC-5 | 93.9 | 4.7 | 89.7 | 0.6 |

ADC-5 has shown bystander killing on MDA-MB-453/Clau6-nanoLuc system. ADC-2, ADC-3, and ADC5 has shown comparable bystander killing with ADC-1.

FIG. 42 illustrates the ADC bystander cellular killing activity in the MDA-MB-453 & Calu-6-nanoLuc system.

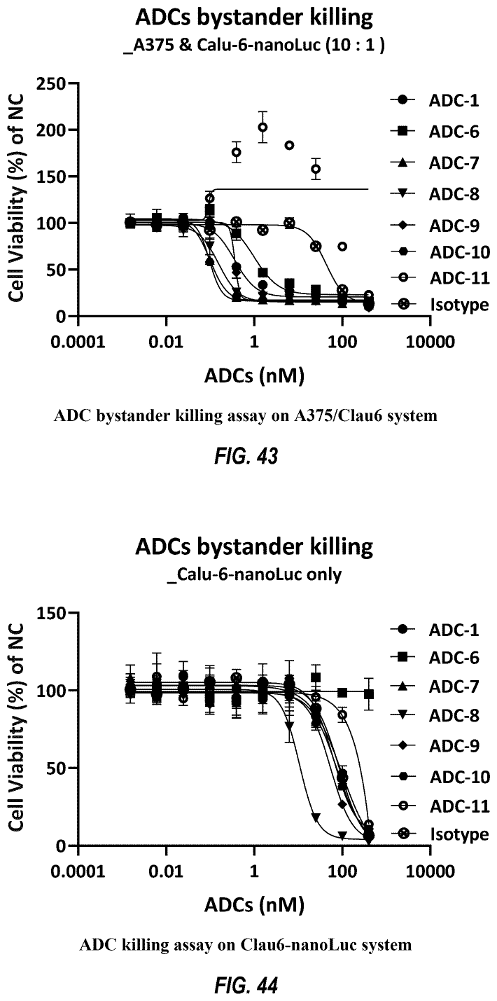

FIG. 43 illustrates the ADC bystander cellular killing potency in the A375 & Calu-6-nanoLuc system.

FIG. 44 illustrates the ADC bystander cellular killing activity in the Calu-6-nanoLuc only system.

The data is provided in the following table:

TABLE 27

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |
| ADC-1 | No bystander killing | | 89.7 | 0.4 |
| ADC-6 | No bystander killing | | 84.3 | 1.2 |
| ADC-7 | 93.4 | 6.7 | 87.3 | 0.1 |
| ADC-8 | 96.6 | 0.4 | 90.7 | 0.2 |
| ADC-9 | 94.0 | 38.4 | 90.8 | 0.4 |
| ADC-10 | 96.0 | 0.3 | 88.5 | 0.1 |

| | | |
|---|---|---|
| ADC-11 | No bystander killing | No bystander killing |

ADC-7, ADC-8, ADC-9, ADC-10 have demonstrated moderate to strong bystander killing on MDA-MB-453/Calu6-nanoLuc system and comparable bystander killing with ADC-1 on A375/Calu6-nanoLuc system.

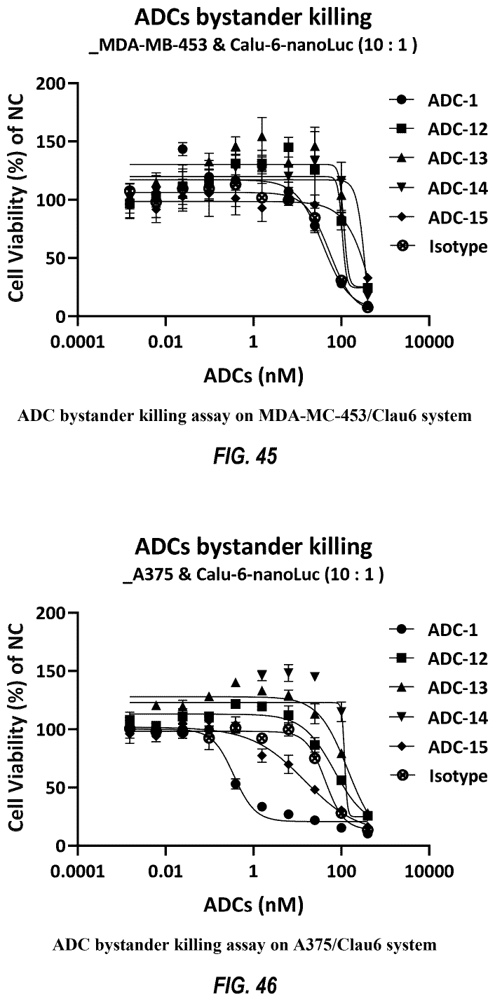

FIG. 45 illustrates the ADC bystander cellular killing potency in the MDA-MB-453 & Calu-6-nanoLuc system.

FIG. 46 illustrates the ADC bystander cellular killing potency in the A375 & Calu-6-nanoLuc system.

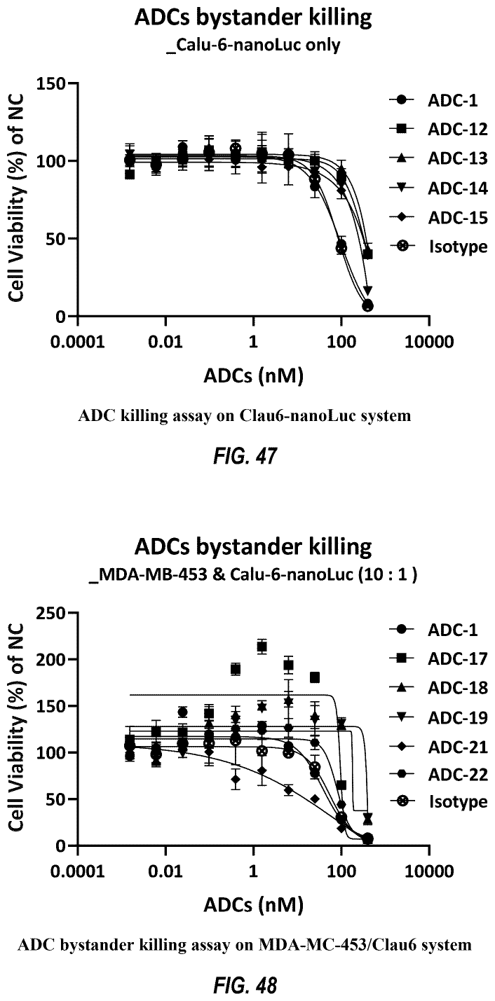

FIG. 47 illustrates the ADC bystander cellular killing activity in the Calu-6-nanoLuc only system.

The data is provided in the following table:

TABLE 28

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |

| ADC-1 | No bystander killing | 89.7 | 0.4 |
|---|---|---|---|
| ADC-12 | No bystander killing | No bystander killing | |
| ADC-13 | No bystander killing | No bystander killing | |
| ADC-14 | No bystander killing | No bystander killing | |
| ADC-15 | No bystander killing | 83.8 | 12.8 |

FIG. 48 illustrates the ADC bystander cellular killing potency in the MDA-MB-453 & Calu-6-nanoLuc system.

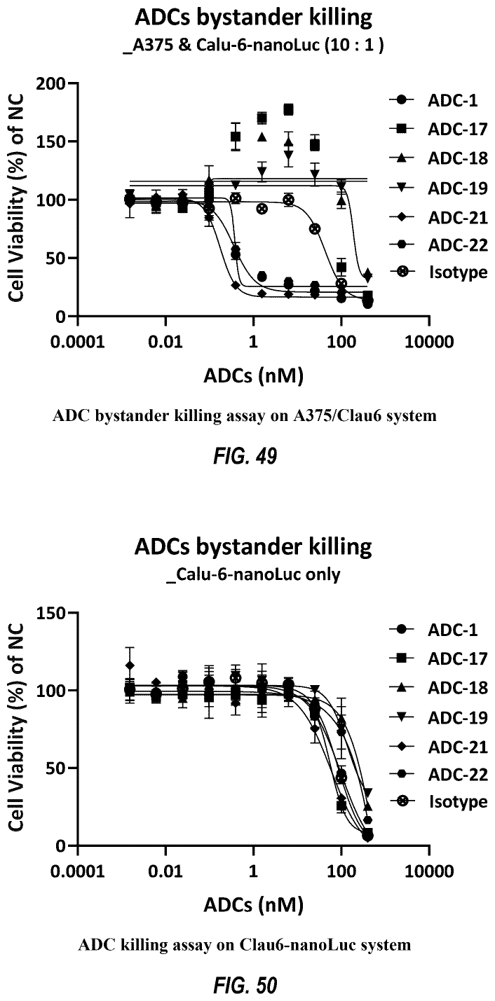

FIG. 49 illustrates the ADC bystander cellular killing potency in the A375 & Calu-6-nanoLuc system.

FIG. 50 illustrates the ADC bystander cellular killing activity in the Calu-6-nanoLuc only system.

The data is provided in the following table:

TABLE 29

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |
| ADC-1 | No bystander killing | | 89.7 | 0.4 |
| ADC-17 | No bystander killing | | No bystander killing | |
| ADC-18 | No bystander killing | | No bystander killing | |
| ADC-19 | No bystander killing | | No bystander killing | |
| ADC-21 | 94.6 | 12.9 | 89.5 | 0.2 |

| | | | |
|---|---|---|---|
| ADC-22 | No bystander killing | 84.7 | 0.4 |

ADC-21 has shown stronger bystander killing than ADC-1 on MDA-MB-453/Calu6-nanoLuc system. ADC-21 and ADC-22 have demonstrated similar bystander killing with ADC-1 on A375/Calu6-nanoLuc system.

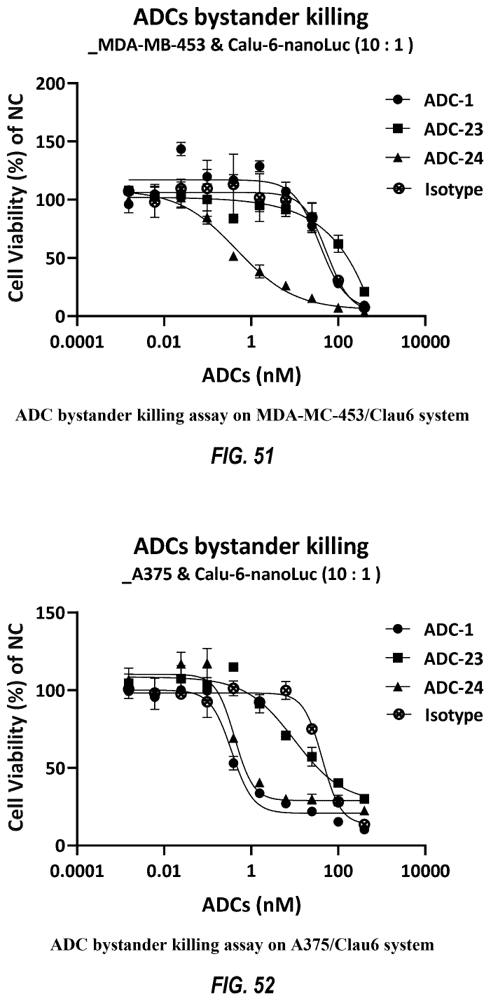

FIG. 51 illustrates the ADC bystander cellular killing potency in the MDA-MB-453 & Calu-6-nanoLuc system.

FIG. 52 illustrates the ADC bystander cellular killing potency in the A375 & Calu-6-nanoLuc system.

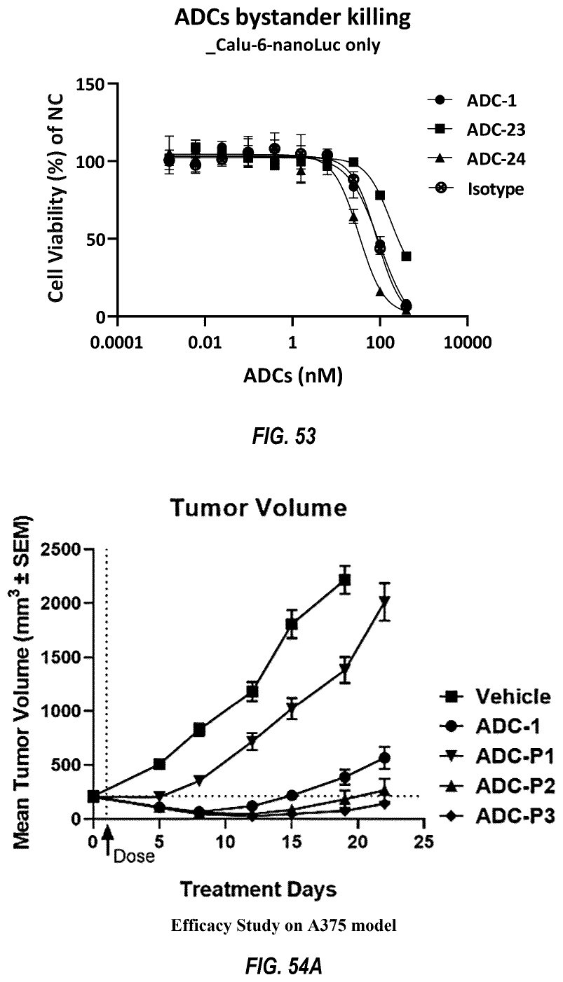

FIG. 53 illustrates the ADC bystander cellular killing activity in the Calu-6-nanoLuc only system.

The data is provided in the following table:

TABLE 30

| | MDA-MB-453:Calu-6- | | A375:Calu-6- | |
|---|---|---|---|---|
| | nanoLuc = 10:1, 3D | | nanoLuc = 10:1, 3D | |
| ADCs | Emax (%) | EC50 (nM) | Emax (%) | EC50 (nM) |
| ADC-1 | No bystander killing | | 89.7 | 0.4 |
| ADC-23 | No bystander killing | | No bystander killing | |
| ADC-24 | 96.9 | 0.6 | 77.5 | 0.5 |

ADC-24 has shown stronger bystander killing than ADC-1 on MDA-MB-453/Calu6-nanoLuc system and comparable bystander killing on A375/calu6-nanoLuc system.

### Efficacy Study 1

### Methods

Female BALB/c nude mice were subcutaneously implanted with 3×10⁶ A-375 cells per 200 μL PBS in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 210 mm³ in size, mice were randomly allocated into 5 groups with 7 animals in each group, and were intravenously treated with vehicle, ADC-1, ADC-P1, ADC-P2 or ADC-P3 at 10 mg/kg on day 1. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated {t}_{0} )}{( placebo t ) - ( placebo {t}_{0} )} ) )$$

treated t=treated tumor volume at time t

treated t₀=treated tumor volume at time 0

placebo t=placebo tumor volume at time t

placebo t₀=placebo tumor volume at time 0

### Results

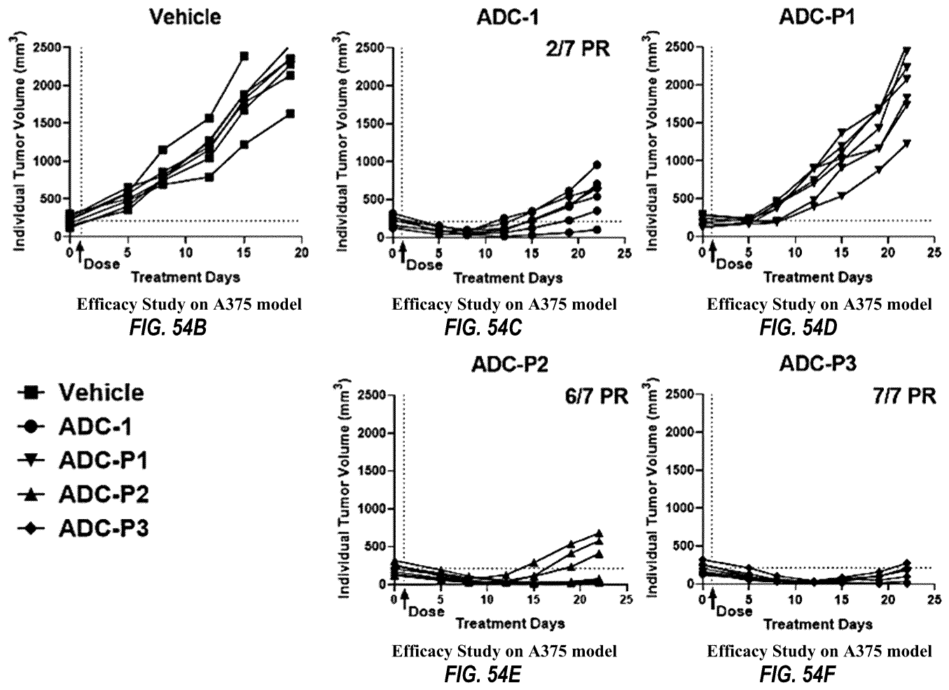

The in vivo efficacy of different ADCs were compared in A-375 melanoma xenografts grown subcutaneously in BALB/c nude mice. Results are shown in FIGS. 54A-F. Treatment with ADC-1, ADC-P1, ADC-P2, or ADC-P3 at 10 mg/kg resulted in 91%, 41%, 101%, or 107% tumor growth inhibition (TGI) on day 19, respectively (FIG. 54A). For individual animal tumor growth inhibition, ADC-1 induced 2/7 PR, while ADC-P2 and ADC-P3 induced 6/7 PR and 7/7 PR (FIGS. 54B-F). Meanwhile all four ADCs were well tolerated without any sign of toxicity or significant body weight decrease. Taken together, ADC-P2 and ADC-P3 demonstrated comparable tumor growth inhibition with ADC-1, while better PR and CR ratio than ADC-1.

### Efficacy Study 2

### Methods

Female BALB/c nude mice were subcutaneously implanted with 3×10⁶ A-375 cells per 200 L PBS in the right flank. When tumors reached a mean volume of approximately 260 mm³ in size, mice were randomly allocated into 5 groups with 7 animals in each group, and were intravenously treated with vehicle, ADC-1, or ADC-3 at 3 mg/kg or 10 mg/kg on day 1 and day 7, respectively.

### Result

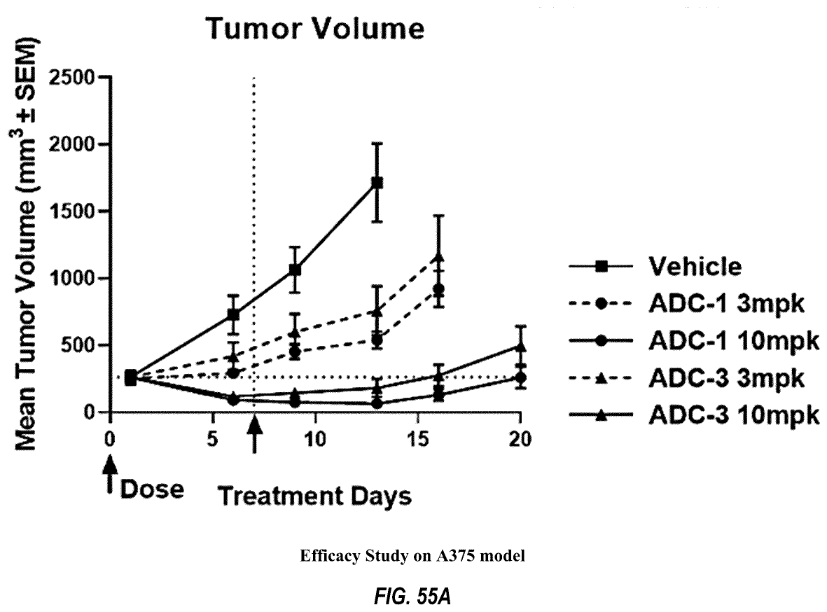

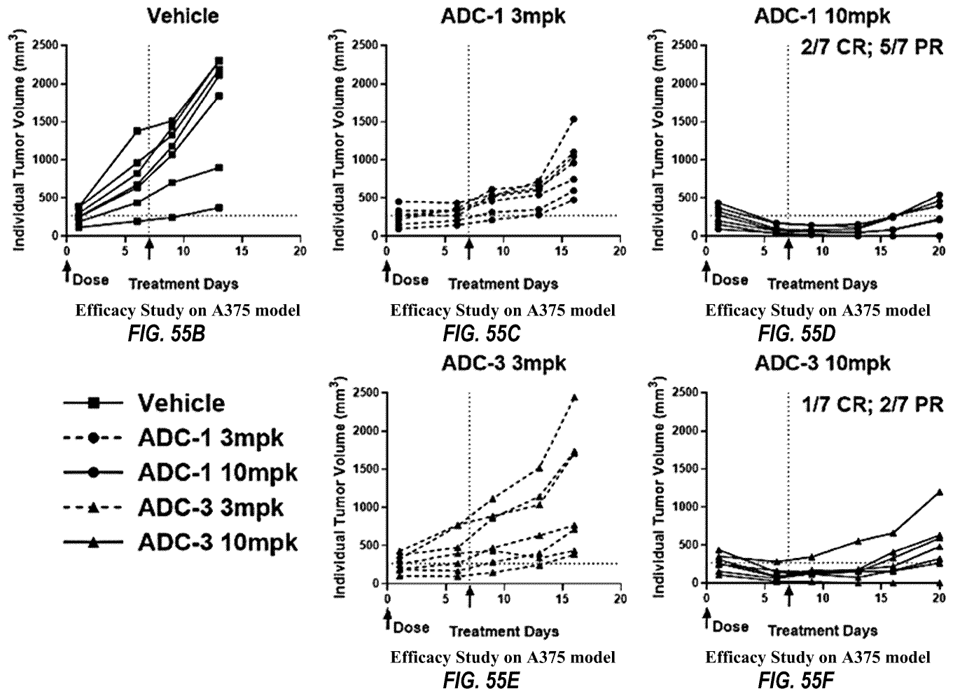

The in vivo efficacy of different ADCs were compared in A-375 melanoma xenografts grown subcutaneously in BALB/c nude mice. Treatment with ADC-1 and ADC-3 resulted in dose dependent anti-tumor effect. Treatment with ADC-1 or ADC-3 at 3 mg/kg resulted in 80%, or 66% TGI on day 13, at 10 mg/kg resulted in 114%, or 106% TGI on day 13, respectively (FIGS. 55A-F). ADC-3 demonstrated comparable efficacy with ADC-1 in A375 xenograft model both at 3 mg/kg and 10 mg/kg. Both ADCs were well tolerated without any sign of toxicity or significant body weight decreased.

### Efficacy Study 3

### Methods

Female BALB/c nude mice were subcutaneously implanted with 3×10⁶ A-375 cells per 200 L PBS in the right flank. When tumors reached a mean volume of approximately 275 mm³ in size, mice were randomly allocated into 8 groups with 8 animals in each group, and were intravenously treated with vehicle, ADC-1, ADC-6, ADC-7, ADC-9, ADC-10, ADC-11 or ADC-21 at 10 mg/kg on day 1, respectively.

### Results

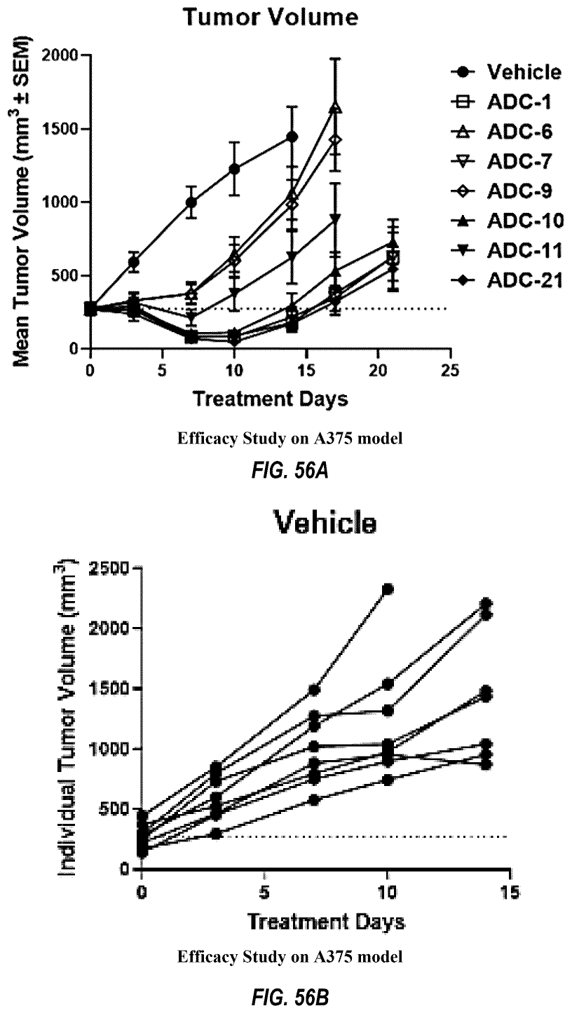

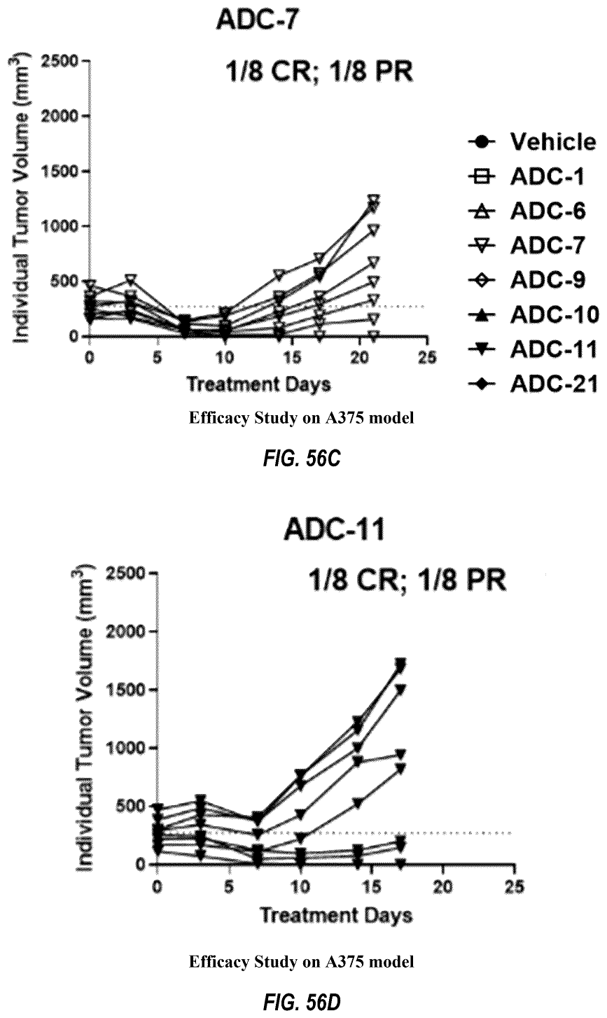

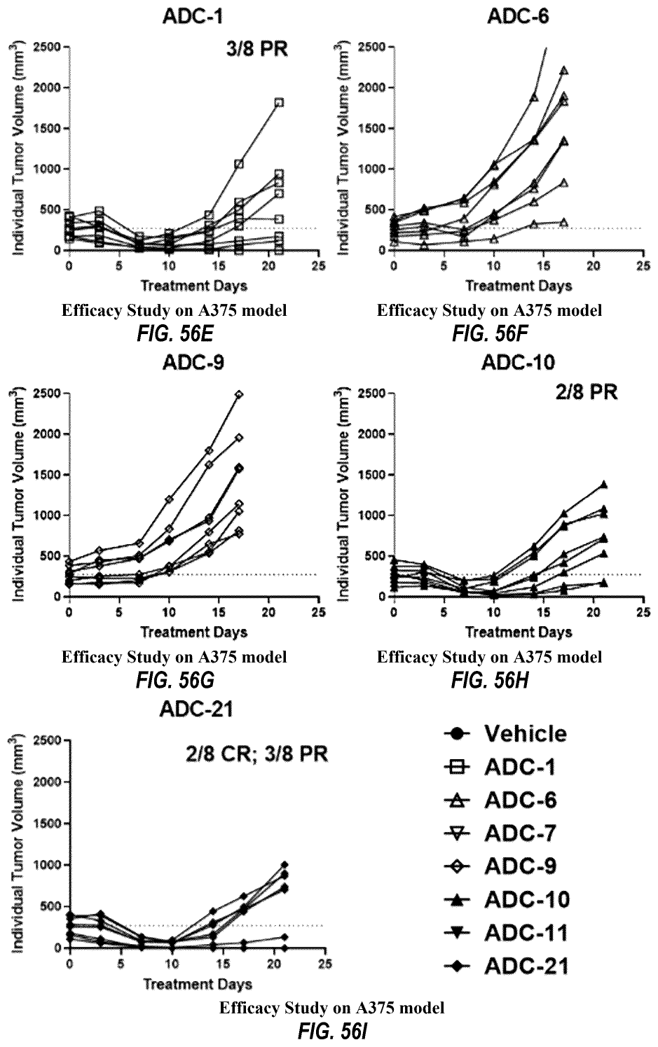

The in vivo efficacy of different ADCs were compared in A-375 melanoma xenografts grown subcutaneously in BALB/c nude mice. Results are shown in FIGS. 56A-I. Treatment with ADC-1, ADC-6, ADC-7, ADC-9, ADC-10, ADC-11 or ADC-21 at 10 mg/kg resulted in 108%, 33%, 105%, 39%, 98%, 71% or 108% TGI on day 14, respectively (FIG. 56A). For individual animal tumor growth inhibition, ADC-1 also induced 3/8 PR, ADC-7 induced 1/8 CR and 1/8 PR, ADC-11 induced 1/8 CR and 1/8 PR, while ADC-21 induced 2/8 CR and 3/8 PR (FIGS. 56B-I). Taken together, ADC-7, ADC-10, ADC-11 or ADC-21 demonstrated comparable anti-tumor efficacy with ADC-1 in A-375 xenograft model. Besides, all ADCs were well tolerated without any sign of toxicity or significant body weight decreased.

### Efficacy Study 4

### Methods

Female BALB/c nude mice were subcutaneously implanted with 3×10⁶ A-375 cells per 200 L PBS in the right flank. When tumors reached a mean volume of approximately 213 mm³ in size, mice were randomly allocated into 3 groups with 8 animals in each group, and were intravenously treated with vehicle, ADC-1, or ADC-22 at 10 mg/kg on day 1, respectively.

### Result

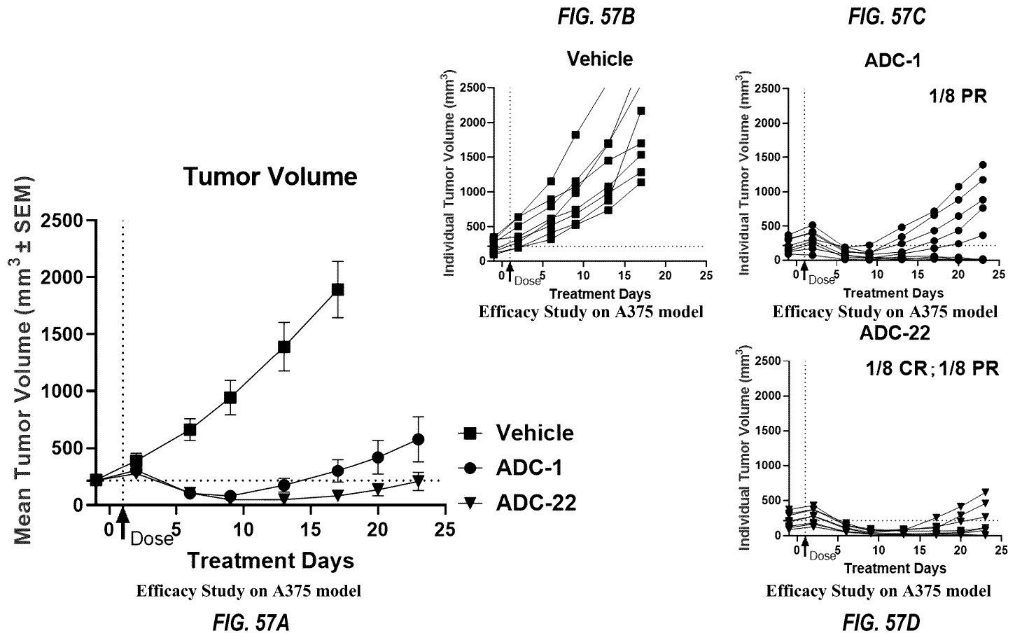

The in vivo efficacy of different ADCs was compared in A-375 melanoma xenografts grown subcutaneously in BALB/c nude mice. Treatment with ADC-1 and ADC-22 resulted in 95%, or 108% TGI on day 17, respectively (FIG. 57A). For individual animal tumor growth inhibition, ADC-1 induced 1/8 PR, ADC-22 induced 1/8 PR and 1/8 CR (FIGS. 57B-D). Taken together, ADC-22 demonstrated comparable efficacy with ADC-1 in A375 xenograft model at 10 mg/kg. Both ADCs were well tolerated without any sign of toxicity or significant body weight decreased.

### Efficacy Study 5 (Plan to do)

### Methods

Female BALB/c nude mice were subcutaneously implanted with 1×10⁷ MX-1 cells per 200 L PBS/matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 210 mm³ in size, mice were randomly allocated into 5 groups with 7 animals in each group.

Blood samples were collected into 0, 2, 4, 8, 24, 72, and 168 h after 10 mg/kg intravenously administration of ADCs from A-375 tumor bearing mice, followed by centrifugation (4° C., 3500×g, 2 min) to separate serum.

### Pharmacokinetic Study (PK)

### Methods

Blood samples were collected at 2, 4, 8, 24, 72, and 168 h after 10 mg/kg intravenously administration of ADC-1, ADC-3, ADC-6, ADC-7, ADC-9, ADC-10, ADC-11, ADC-21, ADC-P1, ADC-P2 or ADC-P3 from A375 tumor bearing mice, followed by centrifugation (4° C., 3500×g, 2 min) to separate serum. The concentrations of ADCs and total Abs were measured by in-house developed Meso Scale Discovery (MSD) ligand binding methods. Briefly, his tagged Her3 extracellular domain fusion protein was used as a capture reagent, biotin labelled anti-DXd Ab, or goat anti-human kappa Ab were used as the detection reagents for ADCs or total Ab measurement, respectively.

### Results

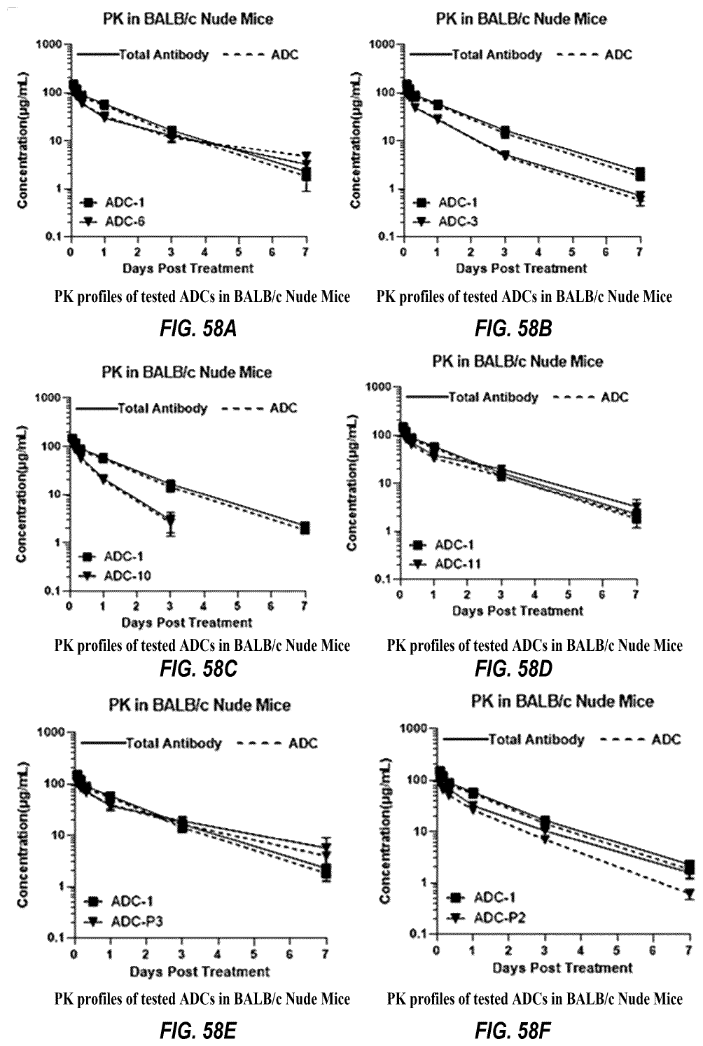

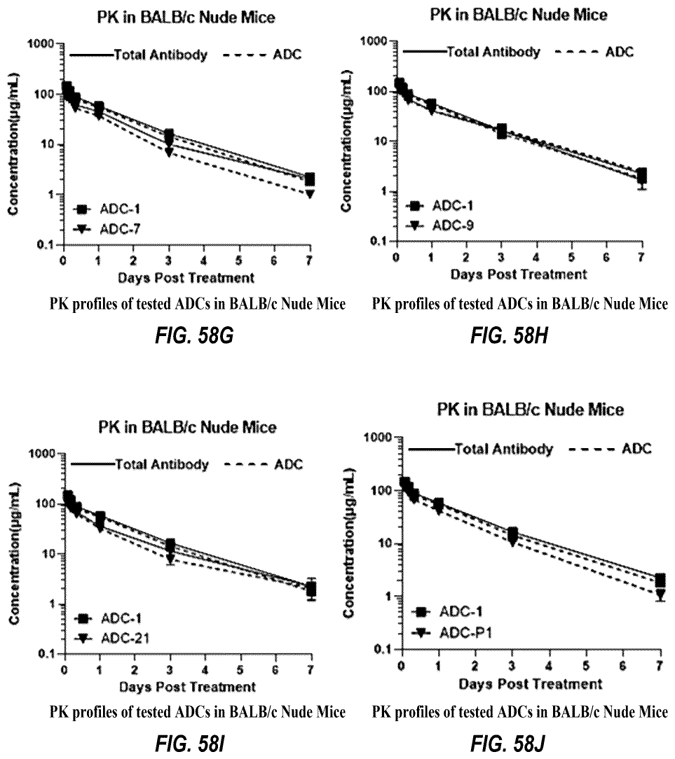

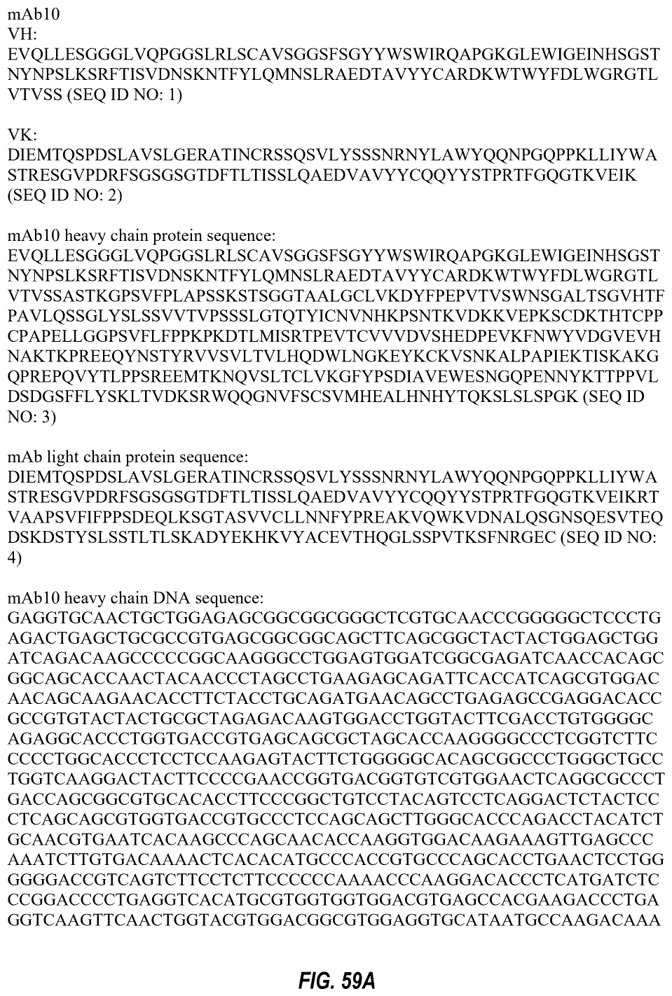

Overlapped ADC and total Ab concentration curves suggesting very good linker stability of those three ADCs in mouse serum. ADC-3, ADC-6, ADC-7, ADC-9, ADC-10, ADC-11, ADC-21, ADC-P1, ADC-P2 or ADC-P3 exhibited comparable ADC or total Ab exposure and clearance with ADC-1 as illustrated in FIGS. 58A-J, Table 31, Table 32, and Table 33.

TABLE 31

| ADC | ADC-1 | | ADC-3 | | ADC-6 | | ADC-7 | |
|---|---|---|---|---|---|---|---|---|
| Detection | ADC | TAB | ADC | TAB | ADC | TAB | ADC | TAB |
| t_{1/2} (h) | 29.2 | 30.3 | 25.1 | 26.6 | 53.8 | 45.2 | 27.8 | 30.2 |
| C0 (μg/mL) | 179 | 185 | 89.7 | 93.3 | 117 | 108 | 95.4 | 104 |
| Cmax (μg/mL) | 145 | 148 | 95.9 | 111 | 98 | 106 | 87.7 | 93.9 |
| AUCₗₐₛₜ (h*ug/mL) | 4484 | 4885 | 2287.0 | 2289.8 | 3235 | 3254 | 2770 | 3437 |
| AUC_{INF} (h*ug/mL) | 4559 | 4983 | 2307.9 | 2317.2 | 3597 | 3461 | 2811 | 3527 |
| AUC_%Extrap(%) | 1.7 | 2.0 | 0.9 | 1.2 | 10.1 | 6.0 | 1.4 | 2.5 |
| Vz (mL/kg) | 92.3 | 87.7 | 157 | 165 | 216 | 188 | 142 | 124 |
| Cl (mL/h/kg) | 2.19 | 2.01 | 4.33 | 4.32 | 2.78 | 2.89 | 3.56 | 2.84 |
| MRTINF (h) | 33.9 | 35.7 | 26.0 | 27.5 | 60.3 | 49.1 | 30.5 | 36.5 |
| Vss (mL/kg) | 74.4 | 71.7 | 113 | 119 | 168 | 142 | 109 | 103 |

TABLE 32

| ADC | ADC-9 | | ADC-10 | | ADC-11 | | ADC-21 | |
|---|---|---|---|---|---|---|---|---|
| Detection | ADC | TAB | ADC | TAB | ADC | TAB | ADC | TAB |
| t_{1/2} (h) | 34.4 | 30.2 | 14.8 | 15.1 | 35.5 | 39.7 | 31.8 | 36.8 |
| C0 (μg/mL) | 137 | 110 | 143 | 144 | 124 | 162 | 120 | 140 |
| Cmax (μg/mL) | 117 | 102 | 110 | 110 | 102 | 125 | 98.9 | 111 |
| AUCₗₐₛₜ (h *ug/mL) | 4062 | 3874 | 1872 | 1976 | 3378 | 4210 | 2880 | 3389 |
| AUC_{INF} (h*ug/mL) | 4184 | 3946 | 1928 | 2039 | 3483 | 4393 | 2977 | 3509 |
| AUC_%Extrap(%) | 2.9 | 1.8 | 2.9 | 3.1 | 3.0 | 4.2 | 3.3 | 3.4 |
| Vz (mL/kg) | 119 | 111 | 111 | 107 | 147 | 130 | 154 | 151 |
| Cl (mL/h/kg) | 2.39 | 2.53 | 5.19 | 4.90 | 2.87 | 2.28 | 3.36 | 2.85 |
| MRTINF (h) | 42.6 | 38.7 | 16.0 | 16.6 | 41.2 | 47.0 | 36.6 | 39.4 |
| Vss (mL/kg) | 102 | 98.0 | 82.8 | 81.3 | 118 | 107 | 123 | 112 |

TABLE 33

| | ADC | | | | |
|---|---|---|---|---|---|
| | ADC-P1 | ADC-P2 | | ADC-P3 | |
| Detection | ADC | ADC | TAB | ADC | TAB |
| t_{1/2} (h) | 26.9 | 26.6 | 33.4 | 43.6 | 52.9 |
| C0 (μg/mL) | 143 | 116 | 120 | 113 | 116 |
| Cmax (μg/mL) | 116 | 86.4 | 94.8 | 98 | 103 |
| AUCₗₐₛₜ (h*ug/mL) | 3476 | 2362 | 3036 | 3877 | 4156 |
| AUC_{INF} (h*ug/mL) | 3518 | 2385 | 3111 | 4124 | 4582 |
| AUC_% Extrap(%) | 1.18 | 0.98 | 2.41 | 5.98 | 9.30 |
| Vz (mL/kg) | 110 | 161 | 155 | 153 | 166 |
| Cl (mL/h/kg) | 2.84 | 4.19 | 3.21 | 2.43 | 2.18 |
| MRTINF (h) | 31.5 | 29.7 | 36.4 | 51.1 | 62.1 |
| Vss (mL/kg) | 89.6 | 125 | 117 | 124 | 135 |

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

1. (canceled)

2. A compound of formula (II): 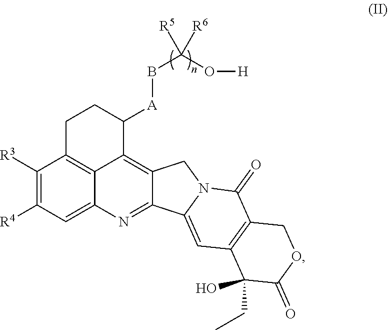
or a pharmaceutically acceptable salt, tautomer, isotopologue, stereoisomer, or prodrug thereof, wherein
A is CR¹R² or N—R¹;
B is a bond, —C(═O)—, —C(═O)O—, or —OC(═O)—;
each of R¹ and R² is, independently, H or C₁₋₄ alkyl;
each of R³ and R⁴ is, independently, hydrogen, halogen, substituted or unsubstituted alkyl, or substituted or unsubstituted alkoxyl; or R³ and R⁴, together with the atoms to which they are attached, form unsubstituted or substituted cycloalkyl, unsubstituted or substituted heterocyclyl, unsubstituted or substituted aryl, or unsubstituted or substituted heteroaryl;
each of R⁵ and R⁶ is, independently, hydrogen, halogen, substituted or unsubstituted alkyl, or substituted or unsubstituted alkoxyl;
n is 1, 2, 3, 4, or 5; and
when R³ is methyl and R⁴ is F, A-B is not —NH—C(═O)—.

3. The compound of claim 2, wherein A is CH₂ and B is a bond.

4. The compound of claim 3, wherein R⁵ and R⁶ are hydrogen and n is 1, 2, or 3.

5. The compound of claim 4, wherein R³ is methyl and R⁴ is F.

6. The compound of claim 5, wherein the compound is

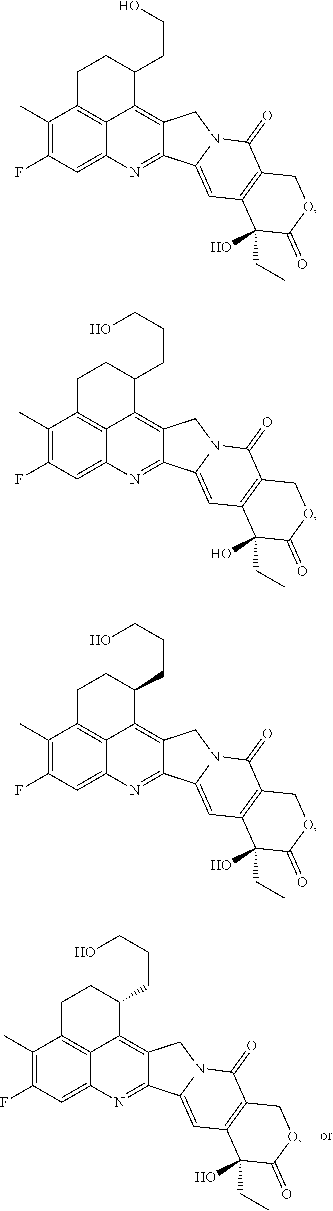 [SMILES: CC[C@@]1(O)C(=O)OCc2c1cc1n(c2=O)Cc2c-1nc1cc(F)c(C)c3c1c2C(CCO)CC3 ; CC[C@@]1(O)C(=O)OCc2c1cc1n(c2=O)Cc2c-1nc1cc(F)c(C)c3c1c2C(CCCO)CC3 ; CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3 ; "or"]

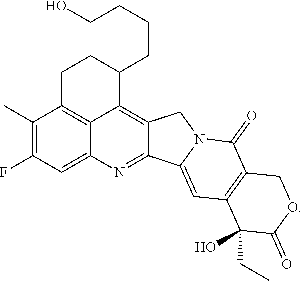 [SMILES: CC[C@@]1(O)C(=O)OCc2c1cc1n(c2=O)Cc2c-1nc1cc(F)c(C)c3c1c2C(CCCCO)CC3]

7. The compound of claim 4, wherein R³ and R⁴, together with the atoms to which they are attached, form an unsubstituted or substituted dioxole ring.

8. The compound of claim 7, wherein the compound is

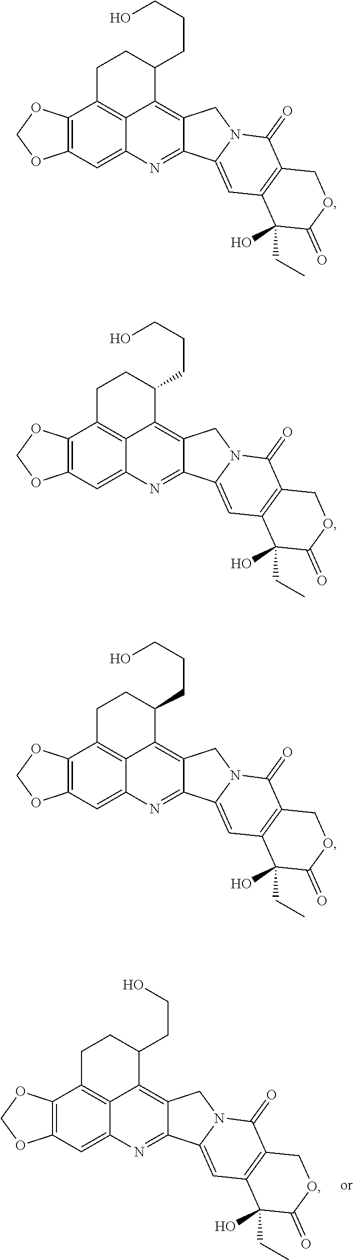 [SMILES: CC[C@@]1(O)C(=O)OCc2c1cc1n(c2=O)Cc2c-1nc1cc3c(c4c1c2C(CCCO)CC4)OCO3 ; CC[C@@]1(O)C(=O)OCc2c1cc1n(c2=O)Cc2c-1nc1cc3c(c4c1c2[C@@H](CCCO)CC4)OCO3 ; CC[C@@]1(O)C(=O)OCc2c1cc1n(c2=O)Cc2c-1nc1cc3c(c4c1c2[C@H](CCCO)CC4)OCO3 ; CC[C@@]1(O)C(=O)OCc2c1cc1n(c2=O)Cc2c-1nc1cc3c(c4c1c2C(CCO)CC4)OCO3 ; "or"]

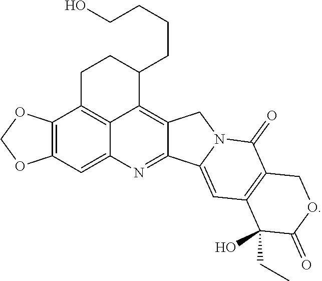 [SMILES: CC[C@@]1(O)C(=O)OCc2c1cc1n(c2=O)Cc2c-1nc1cc3c(c4c1c2C(CCCCO)CC4)OCO3]

9. The compound of claim 2, wherein R³ is methyl, R⁴ is F, A is N(CH₃), and B is a bond.

10. (canceled)

11. The compound of claim 9, wherein the compound is

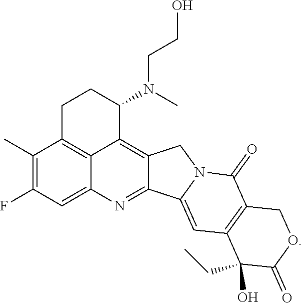 [SMILES: CC[C@@]1(O)C(=O)OCc2c1cc1n(c2=O)Cc2c-1nc1cc(F)c(C)c3c1c2[C@@H](N(C)CCO)CC3]

12. The compound of claim 2, wherein R³ is methyl, R⁴ is F, A is NH, and B is —C(═O)O—.

13. (canceled)

14. The compound of claim 12, wherein the compound is

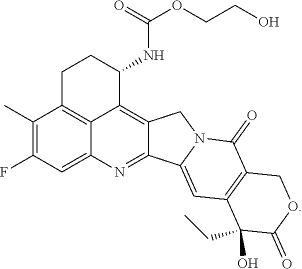 [SMILES: CC[C@@]1(O)C(=O)OCc2c1cc1n(c2=O)Cc2c-1nc1cc(F)c(C)c3c1c2[C@@H](NC(=O)OCCO)CC3]

15. (canceled)

16. The compound of claim 2, wherein the compound is

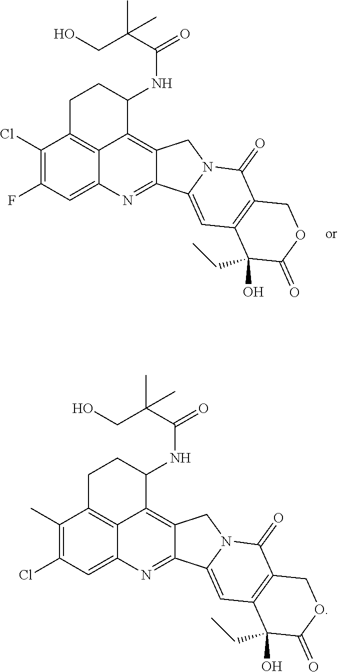 [SMILES: CC[C@@]1(O)C(=O)OCc2c1cc1n(c2=O)Cc2c-1nc1cc(F)c(Cl)c3c1c2C(NC(=O)C(C)(C)CO)CC3 ; "or" ; CC[C@@]1(O)C(=O)OCc2c1cc1n(c2=O)Cc2c-1nc1cc(Cl)c(C)c3c1c2C(NC(=O)C(C)(C)CO)CC3]

17. (canceled)

18. (canceled)

19. The compound of claim 2, wherein R³ and R⁴, together with the atoms to which they are attached, form unsubstituted or substituted heterocyclyl.

20. The compound of claim 19, wherein R³ and R⁴, together with the atoms to which they are attached, form an unsubstituted or substituted dioxole ring, and B is —C(═O)—.

21. The compound of claim 20, wherein the compound is

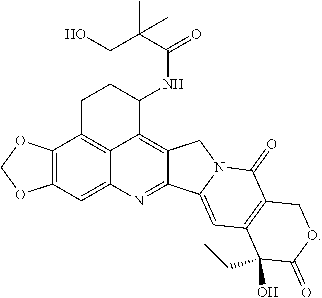 [SMILES: CC[C@@]1(O)C(=O)OCc2c1cc1n(c2=O)Cc2c-1nc1cc3c(c4c1c2C(NC(=O)C(C)(C)CO)CC4)OCO3]

22. The compound of claim 2, wherein the compound is a compound of formula (III): 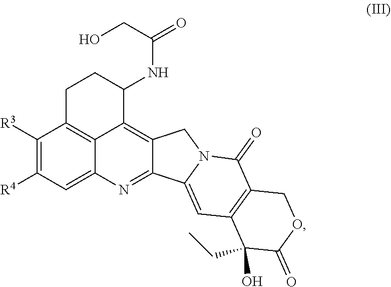
or a pharmaceutically acceptable salt, tautomer, isotopologue, stereoisomer, or prodrug thereof,
wherein R³ and R⁴ together are not methyl and F, respectively.

23. The compound of claim 22, wherein the compound is

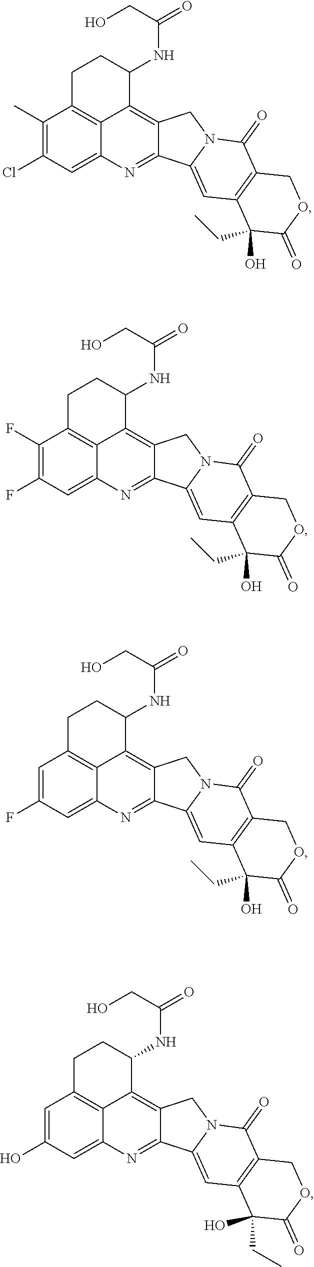 [SMILES: CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(F)c(F)c3c1c2C(NC(=O)CO)CC3 ; CC[C@@]1(O)C(=O)OCc2c1cc1n(c2=O)Cc2c-1nc1cc(F)cc3c1c2C(NC(=O)CO)CC3 ; CC[C@@]1(O)C(=O)OCc2c1cc1n(c2=O)Cc2c-1nc1cc(O)cc3c1c2[C@@H](NC(=O)CO)CC3]

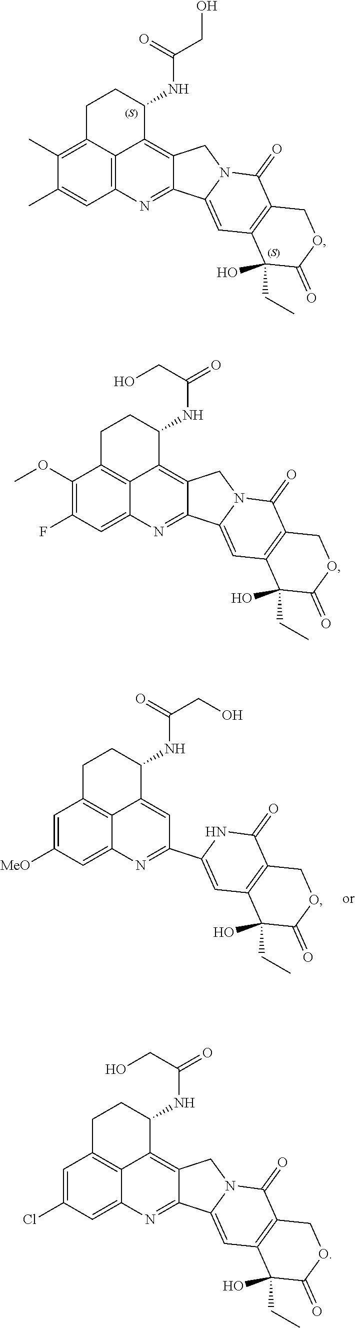

24-30. (canceled)

31. The compound of claim 22, wherein R³ and R⁴, together with the atoms to which they are attached, form unsubstituted or substituted heterocyclyl.

32. The compound of claim 31, wherein R³ and R⁴, together with the atoms to which they are attached, form an unsubstituted or substituted dioxole ring.

33. The compound of claim 32, wherein the compound is

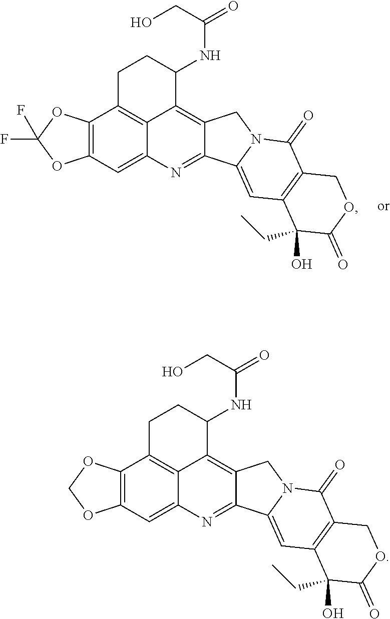 [SMILES: CC[C@@]1(O)C(=O)OCc2c1cc1n(c2=O)Cc2c-1nc1cc3c(c4c1c2C(NC(=O)CO)CC4)OC(F)(F)O3 ; "or" ; CC[C@@]1(O)C(=O)OCc2c1cc1n(c2=O)Cc2c-1nc1cc3c(c4c1c2C(NC(=O)CO)CC4)OCO3]

34. (canceled)

35. The compound of claim 2, wherein the compound is

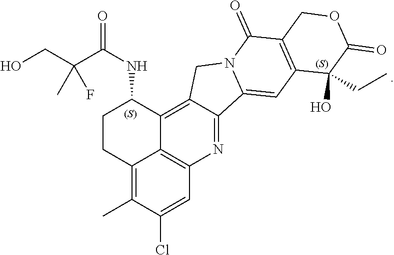

36. (canceled)

37. (canceled)

38. A ligand-drug conjugate or a pharmaceutically acceptable salt or solvate thereof, wherein the ligand-drug conjugate comprises a residue of the compound of claim 2.

39. (canceled)

40. The ligand-drug conjugate of claim 38, wherein the ligand is an antibody selected from patritumab, cofetuzumab, trastuzumab, ifinatamab, and mAb10.

41. A compound of formula (VII): 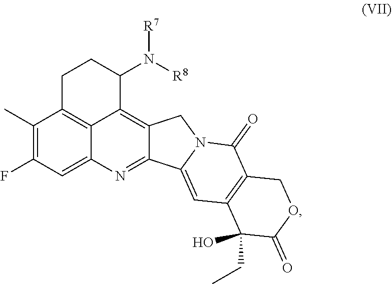
or a pharmaceutically acceptable salt, tautomer, isotopologue, stereoisomer, or prodrug thereof, wherein
each of R⁷ and R⁸ is, independently, hydrogen or substituted or unsubstituted alkyl; or R⁷ and R⁸, together with the nitrogen atom to which they are attached, form unsubstituted or substituted heterocyclyl or unsubstituted or substituted heteroaryl.

42. The compound of claim 41, wherein the compound is

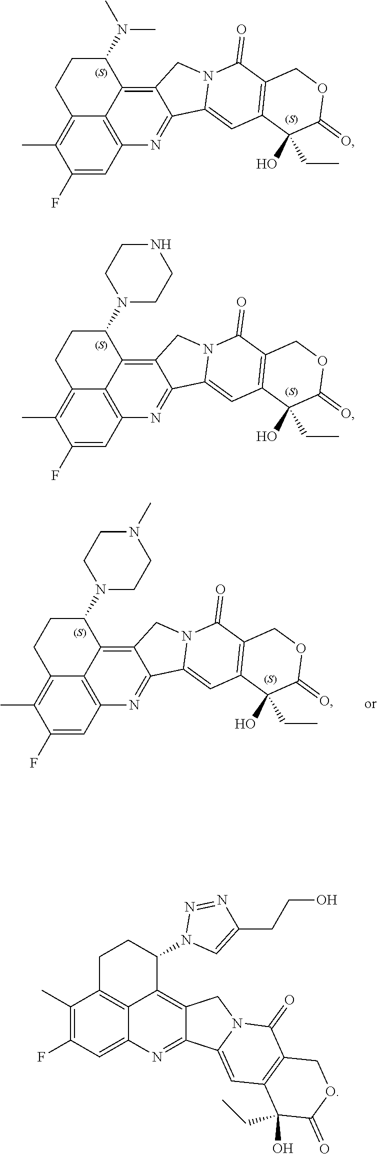

43. A ligand-drug conjugate or a pharmaceutically acceptable salt or solvate thereof, wherein the ligand-drug conjugate comprises a residue of the compound of claim 41.

44-48. (canceled)

49. An antibody or antigen binding fragment thereof that specifically binds human HER3, comprising:
(i) a heavy chain variable region that comprises the amino acid sequence of SEQ ID NO: 1; and
(ii) a light chain variable region that comprises the amino acid sequence of SEQ ID NO: 2.

50-54. (canceled)

55. The compound of claim 2, wherein A is N—R¹; B is —C(═O)O—; R¹, R⁵, and R⁶ are each hydrogen; R³ and R⁴, together with the atoms to which they are attached, form an unsubstituted or substituted dioxole ring; and n is 2.

56. The compound of claim 20, wherein A is N—R¹; R¹ is H; and —C(R⁵)(R⁶)ₘ—OH is —C(CH₃)(F)—CH₂—OH.


SEQUENCE LISTING (6 sequences)

SEQ ID NO: 1 - protein, 117 aa, synthetic construct
EVQLLESGGGLVQPGGSLRLSCAVSGGSFSGYYWSWIRQAPGKGLEWIGEINHSGSTNYNPSLKSRFTISVDNSKNTFYLQMNSLRAEDTAVYYCARDKWTWYFDLWGRGTLVTVSS

SEQ ID NO: 2 - protein, 113 aa, synthetic construct [sequence omitted]

SEQ ID NO: 3 - protein, 447 aa, synthetic construct
EVQLLESGGGLVQPGGSLRLSCAVSGGSFSGYYWSWIRQAPGKGLEWIGEINHSGSTNYNPSLKSRFTISVDNSKNTFYLQMNSLRAEDTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1344 bp, synthetic construct
  source 1..1344: mol_type = other DNA
gaggtgcaactgctggagagcggcggcgggctcgtgcaacccgggggctccctgagactgagctgcgccgtgagcggcggcagcttcagcggctactactggagctggatcagacaagcccccggcaagggcctggagtggatcggcgagatcaaccacagcggcagcaccaactacaaccctagcctgaagagcagattcaccatcagcgtggacaacagcaagaacaccttctacctgcagatgaacagcctgagagccgaggacaccgccgtgtactactgcgctagagacaagtggacctggtacttcgacctgtggggcagaggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - DNA, 663 bp, synthetic construct
  source 1..663: mol_type = other DNA
gacatcgagatgacacagagccccgacagcctggccgtgagcctgggcgagagagccaccatcaactgcagaagcagccaaagcgtgctgtacagcagcagcaacagaaactacctggcctggtatcagcagaaccccgggcagccccccaagctgctgatctactgggcctccacaagagagagcggcgtgcccgacagattcagcggcagcggcagcggcaccgacttcaccctgaccatcagcagcctgcaagccgaggacgtggccgtgtactactgtcagcagtactacagcacccctagaaccttcggccaaggcaccaaggtggagatcaagcgtacggtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag